(19) United States
(12) Patent Application Publication
(10) Pub. No.: US 2026/0053859 A1
(43) Pub. Date: Feb. 26, 2026

(54) GENETICALLY ENGINEERED CELLS

(71) Applicant: Vertex Pharmaceuticals Incorporated, Boston, MA (US)

(72) Inventors: Michael Cooke, Boston, MA (US); Michael Conway, Boston, MA (US); Stephan Kissler, Boston, MA (US); Bryce Carey, Boston, MA (US); Suyash Raj, Boston, MA (US)

(73) Assignee: Vertex Pharmaceuticals Incorporated, Boston, MA (US)

(21) Appl. No.: 19/196,113

(22) Filed: May 1, 2025

Related U.S. Application Data
(63) Continuation of application No. PCT/US2023/078272, filed on Oct. 31, 2023.
Application No. PCT/US2023/078272 is a continuation-in-part of application No. PCT/US2022/079017, filed on Nov. 1, 2022.
(60) Provisional application No. 63/491,032, filed on Mar. 17, 2023.
(60) Provisional application No. 63/493,880, filed on Apr. 3, 2023.
(60) Provisional application No. 63/507,793, filed on Jun. 13, 2023.

(51) Int. Cl. A61K 35/28; C12N 9/22; C12N 15/11; C12N 15/90

(52) U.S. Cl. CPC A61K 35/28; C12N 9/226; C12N 15/11; C12N 15/907; C12N 2310/20

(57) ABSTRACT

Disclosed herein are compositions and methods related to genetically engineered mammalian cells comprising adjusted expression of select genes. The genetically engineered mammalian cells described herein advantageously possess improved survivability.

### CROSS REFERENCE TO RELATED APPLICATIONS

This application is a continuation of International Application No. PCT/US2023/078272, filed Oct. 31, 2023, which claims the benefit of International Application No. PCT/US2022/079017, filed Nov. 1, 2022; U.S. Provisional Patent Application No. 63/491,032, filed Mar. 17, 2023; U.S. Provisional Application No. 63/493,880, filed Apr. 3, 2023; and U.S. Provisional Application No. 63/507,793, filed Jun. 13, 2023, the contents of each of which are incorporated herein by reference in their entireties.

### INCORPORATION OF ELECTRONIC SEQUENCE LISTING

The Sequence Listing is submitted as an XML file in the form of the file named “10644-112022-15_ST26.xml” (˜340,513 bytes), which was created on Apr. 18, 2025 which is incorporated by reference herein.

### BACKGROUND

Transplantation of tissues such as pancreas or pancreatic islets has been used for treating diseases such as diabetes, such as type I diabetes. However, transplanted cells and tissues often encounter a stressful and hostile environment once transplanted into a host subject. Thus, there is a need for engineering cells and tissues (e.g., stem cells or cells differentiated from stem cells) that have improved survivability and/or reduced immunogenicity in host subjects.

### SUMMARY

The present disclosure is directed to genetically engineered mammalian cells comprising adjusted expression of select genes. The genetically engineered mammalian cells described herein advantageously possess improved survivability and/or reduced immunogenicity.

In a first aspect, the present disclosure is directed to a genetically engineered mammalian cell engineered to have decreased or no expression of the renalase gene, and wherein the engineered mammalian cell has also been genetically engineered to have decreased or no expression of the ABO gene; decreased or no expression of the CXCL10 gene, decreased or no expression of the beta-2 microglobulin (B2M), decreased or no expression for the tissue factor 3(F3) gene, and/or increased expression of CD47 or expression of a mutant CD47 when compared to the expression levels of the corresponding genes in the same cell type where the cell has not been genetically engineered.

In some embodiments, the mammalian cell has been genetically engineered to have decreased or no expression of the renalase gene, and the cell has been further genetically engineered to have decreased or no expression of the ABO gene as compared to the expression level of the same cell type that has not been genetically engineered.

In some embodiments, the mammalian cell has been genetically engineered to have decreased or no expression of the renalase gene, and the cell has been further genetically engineered to have decreased or no expression of the ABO gene and/or the cell has been genetically engineered to have decreased or no expression of the CXCL10 gene as compared to the expression level of the same cell type that has not been genetically engineered.

In some embodiments, the mammalian cell has been genetically engineered to have decreased or no expression of the renalase gene, and the cell has been further genetically engineered to have decreased or no expression of the ABO gene; the cell has been genetically engineered to have decreased or no expression of the CXCL10 gene; and/or the cell has been genetically engineered to have decreased or no expression of the B2M gene as compared to the expression level of the same cell type that has not been genetically engineered.

In some embodiments, the mammalian cell has been genetically engineered to have decreased or no expression of the renalase gene, and the cell has been further genetically engineered to have decreased or no expression of the ABO gene; the cell has been genetically engineered to have decreased or no expression of the CXCL10 gene; the cell has been genetically engineered to have decreased or no expression of the B2M gene; and/or the cell has been genetically engineered to have decreased or no expression of the F3 gene as compared to the expression level of the same cell type that has not been genetically engineered.

In some embodiments, the mammalian cell has been genetically engineered to have decreased or no expression of the renalase gene, and the cell has been further genetically engineered to have decreased or no expression of the ABO gene; the cell has been genetically engineered to have decreased or no expression of the CXCL 10 gene; the cell has been genetically engineered to have decreased or no expression of the B2M gene; the cell has been genetically engineered to have decreased or no expression of the F3 gene; and/or the cell has been genetically engineered to have increased expression of CD47 as compared to the expression level of the same cell type that has not been genetically engineered. In some embodiments the engineered cell comprises an insertion of an exogenous CD47 gene.

In some embodiments, the mammalian cell has been genetically engineered to have decreased or no expression of the renalase gene, and the cell has been further genetically engineered to have decreased or no expression of the ABO gene; the cell has been genetically engineered to have decreased or no expression of the CXCL10 gene; the cell has been genetically engineered to have decreased or no expression of the B2M gene; the cell has been genetically engineered to have decreased or no expression of the F3 gene; and/or the cell has been genetically engineered to express a mutant CD47. In some embodiments, the CD47 protein comprises at least 3 amino acids added to the N-terminus of the mature CD47 protein, wherein the added 3 amino acids has the formula X₃—X₂—X₁, wherein X₃ is W; X₂ is selected from Q, A and G; and X₁ is selected from R, P, L, T, F, I, and M. In some embodiments, the mutant CD47 protein comprises an amino acid sequence that is at least 85%, 90%, 91%, 92%, 93%, 94%, 95%, 96%, 97%, 98%, 99%, or 100% identical to the amino acid sequence of SEQ ID NO: 145 or 146, but wherein the Q at position 1 is replaced with at least 3 amino acids. In some embodiments, the Q at position 1 is replaced with any one of WQR, WAP, WQL, WQP, WQPP, WQT, WQF, WQI, WGP, and WQM. In some embodiments, the cell comprises a gene encoding the mutant CD47 protein, wherein the gene encodes a CD47 protein in which at least three amino acids are added between the CD47 leader sequence (e.g., the amino acid sequence of SEQ ID NO: 244) and the start of the mature CD47 amino acid sequence (e.g., an amino acid sequence that is at least 85%, 90%, 91%, 92%, 93%, 94%, 95%, 96%, 97%, 98%, 99%, or 100% identical to SEQ ID NO: 145 or 146). In some embodiments, the cell comprises a gene encoding the mutant CD47 protein, wherein the gene encodes a CD47 protein in which the “Q” at the position corresponding to position 19 of an amino acid sequence that is at least 85%, 90%, 91%, 92%, 93%, 94%, 95%, 96%, 97%, 98%, 99%, or 100% identical to SEQ ID NO: 14 or 243 is replaced with at least three amino acids. In some embodiments, the at least three amino acids are selected from any of WQR, WAP, WQL, WQP, WQPP, WQT, WQF, WQI, WGP, or WQM. In some embodiments, the at least three amino acids are WQPP. In some embodiments, the Q at position 1 is replaced with WQPP. In some embodiments, the at least three amino acids comprise the formula X₃—X₂—X₁, wherein X₃ is W; X₂ is selected from Q, A and G; and X₁ is selected from R, P, L, T, F, I, and M.

In some embodiments, the CD47 protein comprises an amino acid sequence that is at least 80%, 85%, 90%, 91%, 92%, 93%, 94%, 95%, 96%, 97%, 98%, 99% or 100% identical to the sequence of SEQ ID NO: 145 or 146, and wherein the CD47 protein comprises at least 3 amino acids added to the N-terminus of the mature CD47 protein, wherein the added 3 amino acids has the formula X₃—X₂—X₁, wherein X₃ is W; X₂ is selected from Q, A and G; and X₁ is selected from R, P, L, T, F, I, and M. In some embodiments, the three amino acids are selected from WQR, WAP, WQL, WQP, WQT, WQF, WQI, WGP, and WQM.

Some aspects of the present disclosure provide a mammalian cell that has been genetically engineered to have decreased or no expression of the CXCL10 gene, and wherein the cell also has been genetically engineered to have decreased or no expression of the ABO gene and/or the tissue factor (F3) gene.

In some embodiments, the mammalian cell has been genetically engineered to have decreased or no expression of the CXCL10 gene, and wherein the cell has further been genetically engineered to have decreased or no expression of the ABO gene as compared to the expression level of the same cell type that has not been genetically engineered.

In some embodiments, the mammalian cell has been genetically engineered to have decreased or no expression of the CXCL10 gene; the cell has further been genetically engineered to have decreased or no expression of the ABO gene; and/or the cell has been genetically engineered to have decreased or no expression of the tissue factor (TF3) gene as compared to the expression level of the same cell type that has not been genetically engineered.

In some embodiments, the mammalian cell has been has been genetically engineered to have decreased or no expression of the CXCL10 gene; the cell has further been genetically engineered to have decreased or no expression of the ABO gene; the cell has been genetically engineered to have decreased or no expression of the tissue factor (TF3) gene; and/or the cell has been genetically engineered to have decreased or no expression of the beta-2-microglobulin (B2M) gene as compared to the expression level of the same cell type that has not been genetically engineered.

In some embodiments, the mammalian cell has been has been genetically engineered to have decreased or no expression of the CXCL10 gene; the cell has further been genetically engineered to have decreased or no expression of the ABO gene; the cell has been genetically engineered to have decreased or no expression of the tissue factor (TF3) gene; the cell has been genetically engineered to have decreased or no expression of the beta-2-microglobulin (B2M) gene; and/or the cell has been genetically engineered to have decreased or no expression of the renalase gene as compared to the expression level of the same cell type that has not been genetically engineered.

In some embodiments, the mammalian cell has been has been genetically engineered to have decreased or no expression of the CXCL10 gene; the cell has further been genetically engineered to have decreased or no expression of the ABO gene; the cell has been genetically engineered to have decreased or no expression of the tissue factor (TF3) gene; the cell has been genetically engineered to have decreased or no expression of the beta-2-microglobulin (B2M) gene; the cell has been genetically engineered to have decreased or no expression of the renalase gene; and/or the cell has been genetically engineered to have increased expression of CD47 as compared to the expression level of the same cell type that has not been genetically engineered. In some embodiments the engineered cell comprises an insertion of an exogenous CD47 gene.

In some embodiments, the mammalian cell has been has been genetically engineered to have decreased or no expression of the CXCL10 gene; the cell has further been genetically engineered to have decreased or no expression of the ABO gene; the cell has been genetically engineered to have decreased or no expression of the tissue factor (TF3) gene; the cell has been genetically engineered to have decreased or no expression of the beta-2-microglobulin (B2M) gene; the cell has been genetically engineered to have decreased or no expression of the renalase gene; and/or the cell has been genetically engineered to express a mutant CD47 protein. In some embodiments, the CD47 protein comprises at least 3 amino acids added to the N-terminus of the mature CD47 protein, wherein the added 3 amino acids has the formula X₃—X₂—X₁, wherein X₃ is W; X₂ is selected from Q, A and G; and X₁ is selected from R, P, L, T, F, I, and M. In some embodiments, the CD47 protein comprises an amino acid sequence that is at least 80%, 85%, 90%, 91%, 92%, 93%, 94%, 95%, 96%, 97%, 98%, 99% or 100% identical to the sequence of SEQ ID NO: 145 or 146, and wherein the CD47 protein comprises at least 3 amino acids added to the N-terminus of the mature CD47 protein, wherein the added 3 amino acids has the formula X₃—X₂—X₁, wherein X₃ is W; X₂ is selected from Q, A and G; and X₁ is selected from R, P, L, T, F, I, and M. In some embodiments, the three amino acids are selected from WQR, WAP, WQL, WQP, WQT, WQF, WQI, WGP, and WQM.

In some embodiments, the cell expresses a membrane-bound CD47 protein, wherein the membrane-bound CD47 protein comprises an amino acid sequence that is at least 85%, 90%, 91%, 92%, 93%, 94%, 95%, 96%, 97%, 98%, 99%, or 100% identical to the amino acid sequence of SEQ ID NO: 145 or 146, but wherein the Q at position 1 is replaced with at least 3 amino acids. In some embodiments, the Q at position 1 is replaced with any one of WQR, WAP, WQL, WQP, WQPP, WQT, WQF, WQI, WGP, and WQM. In some embodiments, the cell comprises a gene encoding the membrane-bound CD47 protein, wherein the gene encodes a CD47 protein in which at least three amino acids are added between the CD47 leader sequence (e.g., the amino acid sequence of SEQ ID NO: 244) and the start of the mature CD47 amino acid sequence (e.g., an amino acid sequence that is at least 85%, 90%, 91%, 92%, 93%, 94%, 95%, 96%, 97%, 98%, 99%, or 100% identical to SEQ ID NO: 145 or 146). In some embodiments, the cell comprises a gene encoding the membrane-bound CD47 protein, wherein the gene encodes a CD47 protein in which the “Q” at the position corresponding to position 19 of an amino acid sequence that is at least 85%, 90%, 91%, 92%, 93%, 94%, 95%, 96%, 97%, 98%, 99%, or 100% identical to SEQ ID NO: 14 or 243 is replaced with at least three amino acids. In some embodiments, the at least three amino acids are selected from any of WQR, WAP, WQL, WQP, WQPP, WQT, WQF, WQI, WGP, or WQM. In some embodiments, the at least three amino acids are WQPP. In some embodiments, the Q at position 1 is replaced with WQPP. In some embodiments, the at least three amino acids comprise the formula X₃—X₂—X₁, wherein X₃ is W; X₂ is selected from Q, A and G; and X₁ is selected from R, P, L, T, F, I, and M.

In some embodiments of the disclosure, the mammalian cell has been genetically engineered to have decreased or no expression of the B2M, CXCL10, renalase, ABO, and F3 genes, and to have increased expression of CD47 as compared to the expression level of the same mammalian cell type that has not been genetically engineered.

In some embodiments of the disclosure, the mammalian cell has been genetically engineered to have decreased or no expression of the B2M, CXCL10, renalase, ABO, and F3 genes, and to express a mutant CD47 as compared to the expression level of the same mammalian cell type that has not been genetically engineered. In some embodiments, the CD47 protein comprises at least 3 amino acids added to the N-terminus of the mature CD47 protein, wherein the added 3 amino acids has the formula X₃—X₂—X₁, wherein X₃ is W; X₂ is selected from Q, A and G; and X₁ is selected from R, P, L, T, F, I, and M. In some embodiments, the CD47 protein comprises an amino acid sequence that is at least 80%, 85%, 90%, 91%, 92%, 93%, 94%, 95%, 96%, 97%, 98%, 99% or 100% identical to the sequence of SEQ ID NO: 145 or 146, and wherein the CD47 protein comprises at least 3 amino acids added to the N-terminus of the mature CD47 protein, wherein the added 3 amino acids has the formula X₃—X₂—X₁, wherein X₃ is W; X₂ is selected from Q, A and G; and X₁ is selected from R, P, L, T, F, I, and M. In some embodiments, the three amino acids are selected from WQR, WAP, WQL, WQP, WQT, WQF, WQI, WGP, and WQM.

Certain aspects of the disclosure comprise a mammalian cell which is ABO blood group type O, wherein the cell has been genetically engineered to have reduced or no expression of the renalase gene. In some embodiments, the cell has been genetically engineered to have reduced or no expression of the CXCL 10 gene, and/or express a mutant CD47 protein.

In some embodiments, the mammalian cell is ABO blood group type O, wherein the cell has been genetically engineered to have reduced or no expression of the renalase gene; the cell has been genetically engineered to have reduced or no expression of the CXCL 10 gene; the cell has been genetically engineered to have reduced or no expression of the B2M gene; the cell has been genetically engineered to have reduced or no expression of the F3 gene; and the cell has been genetically engineered to have increased expression of the CD47 gene as compared to the expression level of the same cell type that has not been genetically engineered.

Certain aspects of the disclosure comprise a mammalian cell wherein the cell expresses a membrane-bound CD47 protein, wherein the CD47 protein comprises at least 3 amino acids added to the N-terminus of the mature CD47 protein, wherein the added 3 amino acids has the formula X₃—X₂—X₁, wherein X₃ is W; X₂ is selected from Q, A and G; and X₁ is selected from R, P, L, T, F, I, and M.

In some embodiments, the cell expresses a membrane-bound CD47 protein, wherein the the CD47 protein comprises an amino acid sequence that is at least 80%, 85%, 90%, 91%, 92%, 93%, 94%, 95%, 96%, 97%, 98%, 99% or 100% identical to the sequence of SEQ ID NO: 145 or 146, and wherein the CD47 protein comprises at least 3 amino acids added to the N-terminus of the mature CD47 protein, wherein the added 3 amino acids has the formula X₃—X₂—X₁, wherein X₃ is W; X₂ is selected from Q, A and G; and X₁ is selected from R, P, L, T, F, I, and M. In some embodiments, the three amino acids are selected from WQR, WAP, WQL, WQP, WQT, WQF, WQI, WGP, and WQM.

In some embodiments, the mammalian cell expresses a membrane-bound CD47 protein, wherein the cell has also been genetically engineered to have decreased or no expression of the B2M gene.

In some embodiments, the mammalian cell expresses a membrane-bound CD47 protein, wherein the cell has also been genetically engineered to have decreased or no expression of the B2M gene and/or genetically engineered to have decreased or no expression of the B2M gene F3 gene.

In some embodiments, the mammalian cell expresses a membrane-bound CD47 protein, wherein the cell has also been genetically engineered to have decreased or no expression of the B2M gene; the cell has been genetically engineered to have decreased or no expression of the B2M gene F3 gene; and/or the cell has been genetically engineered to have decreased or no expression of the CXCL 10 gene.

In some embodiments, the mammalian cell expresses a membrane-bound CD47 protein, wherein the cell has also been genetically engineered to have decreased or no expression of the B2M gene; the cell has been genetically engineered to have decreased or no expression of the B2M gene F3 gene; the cell has been genetically engineered to have decreased or no expression of the CXCL10 gene; and/or the cell has been genetically engineered to have decreased or no expression of the renalase gene.

In some embodiments, the mammalian cell expresses a membrane-bound CD47 protein, wherein the cell has also been genetically engineered to have decreased or no expression of the B2M gene; the cell has been genetically engineered to have decreased or no expression of the B2M gene F3 gene; the cell has been genetically engineered to have decreased or no expression of the CXCL10 gene; the cell has been genetically engineered to have decreased or no expression of the renalase gene; and/or the cell is ABO blood group type O.

In some embodiments, the mammalian cell expresses a membrane-bound CD47 protein, wherein the cell has also been genetically engineered to have decreased or no expression of the B2M gene; the cell has been genetically engineered to have decreased or no expression of the B2M gene F3 gene; the cell has been genetically engineered to have decreased or no expression of the CXCL10 gene; the cell has been genetically engineered to have decreased or no expression of the renalase gene; the cell is ABO blood group type O; and/or the cell has been genetically engineered to have decreased or no expression of the ABO gene.

In some embodiments, the mammalian cell expresses a membrane-bound CD47 protein, wherein the cell has also been genetically engineered to have decreased or no expression of the B2M gene; the cell has been genetically engineered to have decreased or no expression of the B2M gene F3 gene; the cell has been genetically engineered to have decreased or no expression of the CXCL10 gene; the cell has been genetically engineered to have decreased or no expression of the renalase gene; the cell is ABO blood group type O; the cell has been genetically engineered to have decreased or no expression of the ABO gene; and/or the cell is naturally ABO blood group type O.

In some embodiments, the mammalian cell expresses a membrane-bound CD47 protein, wherein the cell has also been genetically engineered to have decreased or no expression of the B2M gene; the cell has been genetically engineered to have decreased or no expression of the B2M gene F3 gene; the cell has been genetically engineered to have decreased or no expression of the CXCL10 gene; the cell has been genetically engineered to have decreased or no expression of the renalase gene; the cell is ABO blood group type O; the cell has been genetically engineered to have decreased or no expression of the ABO gene; and/or the cell is naturally ABO blood group type O, wherein a transgene encoding the CD47 protein is inserted into a cell's genome such that the expression of the CD47 transgene is tied to the expression of an endogenous target gene in the cell. In some embodiments, the endogenous target gene is a housekeeping gene, such as ACTB, NANOG, or GAPDH. In some embodiments, the transgene is inserted such that the 3′UTR of the housekeeping gene (e.g., the 3′ UTR of the GAPDH gene) is intact.

In some embodiments, the cell's endogenous CD47 gene is mutated such that the cell expresses a CD47 protein that comprises an amino acid sequence that is at least 80%, 85%, 90%, 91%, 92%, 93%, 94%, 95%, 96%, 97%, 98%, 99% or 100% identical to the sequence of SEQ ID NO: 145 or 146, and wherein the CD47 protein comprises at least 3 amino acids added to the N-terminus of the mature CD47 protein, wherein the added 3 amino acids has the formula X₃—X₂—X₁, wherein X₃ is W; X₂ is selected from Q, A and G; and X₁ is selected from R, P, L, T, F, I, and M.

In some embodiments, the CD47 protein comprises an amino acid sequence that is at least 80%, 85%, 90%, 91%, 92%, 93%, 94%, 95%, 96%, 97%, 98%, 99% or 100% identical to the sequence of SEQ ID NO: 245, 162 or 163.

Some aspects of the current disclosure are directed to a genetically modified mammalian cell, wherein the cell is a stem cell. In some embodiments, the modified mammalian cell is a pluripotent stem cell (PSC), an embryonic stem cell (ESC), an induced pluripotent stem cell (iPSC), and/or an embryonic germ stem cell (EGSC). In some embodiments, the modified mammalian cell is differentiated from a pluripotent stem cell. In some embodiments, the genetically engineered mammalian cell is a somatic cell. In some embodiments, the modified mammalian cell is a definitive endoderm cell. In some embodiments, the mammalian cell is a primitive gut tube cell. In some embodiments, the cell is a PDX1-positive pancreatic progenitor cell. In some embodiments, the cell is a NKX6.1-positive pancreatic progenitor cell. In some embodiments, the cell is an Ngn3-positive endocrine progenitor cell. In some embodiments, the cell is an insulin-positive endocrine cell. In some embodiments, the mammalian cell is a pancreatic SC-β cell. In some embodiments, the cell is NKX6.1-positive. In some embodiments, the cell is ISL1-negative. In some embodiments, the mammalian cell is NKX6.1-positive and ISL1-positive. In some embodiments, the mammalian cell is NKX6.1-negative and ISL1-negative. In some embodiments, the mammalian cell is ISL1-positive. In some embodiments, the mammalian cell is NKX6.1-negative. In some embodiments, the cell expresses insulin.

Certain aspects of the disclosure are directed to a mammalian cell that has been genetically modified as described herein, wherein the genetic manipulations are performed using using CRISPR/Cas, piggybac transposon, TALEN, zinc finger technology, homing endonucleases, or meganucleases. In some embodiments, at least one genetic modification is made in an intronic region of the gene. In some embodiments, at least one genetic modification is made in an exon of the gene. In some embodiments, at least one genetic modification is made in a promoter of the gene.

Certain aspects of the current disclosure are directed to a mammalian cell, wherein the mammalian cell has been genetically engineered to have decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 3 and/or SEQ ID NO: 5 or a protein comprising an amino acid sequence that is at least 80%, 85%, 90%, 91%, 92%, 93%, 94%, 95%, 96%, 97%, 98%, 99% or 100% identical to SEQ ID NO: 4 and/or SEQ ID NO: 6, and wherein the cell has also been genetically engineered to have decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 1 or a protein comprising an amino acid sequence that is at least 80%, 85%, 90%, 91%, 92%, 93%, 94%, 95%, 96%, 97%, 98%, 99% or 100% identical to SEQ ID NO: 2; decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 7 or a protein comprising an amino acid sequence that is at least 80%, 85%, 90%, 91%, 92%, 93%, 94%, 95%, 96%, 97%, 98%, 99% or 100% identical to SEQ ID NO: 8; decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 9 or a protein comprising an amino acid sequence that is at least 80%, 85%, 90%, 91%, 92%, 93%, 94%, 95%, 96%, 97%, 98%, 99% or 100% identical to SEQ ID NO: 10; decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 11 or a protein comprising an amino acid sequence that is at least 80%, 85%, 90%, 91%, 92%, 93%, 94%, 95%, 96%, 97%, 98%, 99% or 100% identical to SEQ ID NO: 12; and/or increased expression of a protein encoded by a nucleic acid that is at least 80%, 85%, 90%, 91%, 92%, 93%, 94%, 95%, 96%, 97%, 98%, 99% or 100% identical to SEQ ID NO: 13 and/or SEQ ID NO: 15 or a protein comprising an amino acid sequence that is at least 80%, 85%, 90%, 91%, 92%, 93%, 94%, 95%, 96%, 97%, 98%, 99% or 100% identical to SEQ ID NO: 14, SEQ ID NO: 16, SEQ ID NO: 145 and/or SEQ ID NO: 146, as compared to the expression level of the same cell type that has not been genetically engineered.

In some embodiments, a mammalian cell has been genetically engineered to have decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 7 or a protein comprising an amino acid sequence that is at least 80%, 85%, 90%, 91%, 92%, 93%, 94%, 95%, 96%, 97%, 98%, 99% or 100% identical to SEQ ID NO: 8, and the cell also has been genetically engineered to have decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 1 gene; and/or SEQ ID NO: 11 or a protein comprising an amino acid sequence that is at least 80%, 85%, 90%, 91%, 92%, 93%, 94%, 95%, 96%, 97%, 98%, 99% or 100% identical to SEQ ID NO: 2 or SEQ ID NO: 12, as compared to the expression level of the same cell type that has not been genetically engineered.

In some embodiments, the mammalian cell has been genetically engineered to have decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 7 or a protein comprising an amino acid sequence that is at least 80%, 85%, 90%, 91%, 92%, 93%, 94%, 95%, 96%, 97%, 98%, 99% or 100% identical to SEQ ID NO: 8, and the cell also has been genetically engineered to have decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 1 and/or SEQ ID NO: 11 or a protein comprising an amino acid sequence that is at least 80%, 85%, 90%, 91%, 92%, 93%, 94%, 95%, 96%, 97%, 98%, 99% or 100% identical to SEQ ID NO: 2 and/or SEQ ID NO: 12 and is further genetically engineered to have decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 9 or a protein comprising an amino acid sequence that is at least 80%, 85%, 90%, 91%, 92%, 93%, 94%, 95%, 96%, 97%, 98%, 99% or 100% identical to SEQ ID NO: 10 as compared to the expression level of the same cell type that has not been genetically engineered.

In some embodiments, the mammalian cell has been genetically engineered to have decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 7 or a protein comprising an amino acid sequence that is at least 80%, 85%, 90%, 91%, 92%, 93%, 94%, 95%, 96%, 97%, 98%, 99% or 100% identical to SEQ ID NO: 8, decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 1 or a protein comprising an amino acid sequence that is at least 80%, 85%, 90%, 91%, 92%, 93%, 94%, 95%, 96%, 97%, 98%, 99% or 100% identical to SEQ ID NO: 2; decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 11 or a protein comprising an amino acid sequence that is at least 80%, 85%, 90%, 91%, 92%, 93%, 94%, 95%, 96%, 97%, 98%, 99% or 100% identical to SEQ ID NO: 12; decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 9 or a protein comprising an amino acid sequence that is at least 80%, 85%, 90%, 91%, 92%, 93%, 94%, 95%, 96%, 97%, 98%, 99% or 100% identical to SEQ ID NO: 10; and is further genetically engineered have decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 3 and/or SEQ ID NO: 5 or a protein comprising an amino acid sequence that is at least 80%, 85%, 90%, 91%, 92%, 93%, 94%, 95%, 96%, 97%, 98%, 99% or 100% identical to SEQ ID NO: 4 and/or SEQ ID NO: 6 as compared to the expression level of the same cell type that has not been genetically engineered.

In some embodiments, the mammalian cell has been genetically engineered to have decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 7 or a protein comprising an amino acid sequence that is at least 80%, 85%, 90%, 91%, 92%, 93%, 94%, 95%, 96%, 97%, 98%, 99% or 100% identical to SEQ ID NO: 8, decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 1 or a protein comprising an amino acid sequence that is at least 80%, 85%, 90%, 91%, 92%, 93%, 94%, 95%, 96%, 97%, 98%, 99% or 100% identical to SEQ ID NO: 2; decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 11 or a protein comprising an amino acid sequence that is at least 80%, 85%, 90%, 91%, 92%, 93%, 94%, 95%, 96%, 97%, 98%, 99% or 100% identical to SEQ ID NO: 12; decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 9 or a protein comprising an amino acid sequence that is at least 80%, 85%, 90%, 91%, 92%, 93%, 94%, 95%, 96%, 97%, 98%, 99% or 100% identical to SEQ ID NO: 10; decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 3 and/or SEQ ID NO: 5 or a protein comprising an amino acid sequence that is at least 80%, 85%, 90%, 91%, 92%, 93%, 94%, 95%, 96%, 97%, 98%, 99% or 100% identical to SEQ ID NO: 4 and/or SEQ ID NO: 6, wherein the cell has been genetically engineered to have increased expression of a protein encoded by a nucleic acid that is at least 80%, 85%, 90%, 91%, 92%, 93%, 94%, 95%, 96%, 97%, 98%, 99% or 100% identical to SEQ ID NO: 13 and/or SEQ ID NO: 15 or a protein comprising an amino acid sequence that is at least 80%, 85%, 90%, 91%, 92%, 93%, 94%, 95%, 96%, 97%, 98%, 99% or 100% identical to SEQ ID NO: 14, SEQ ID NO: 16, SEQ ID NO: 145 and/or SEQ ID NO: 146 as compared to the expression level of the same cell type that has not been genetically engineered.

In some embodiments, the mammalian cell has been genetically engineered to have decreased or no expression of proteins encoded by nucleic acid that are at least 80%, 85%, 90%, 91%, 92%, 93%, 94%, 95%, 96%, 97%, 98%, 99% or 100% identical to SEQ ID NO: 9, SEQ ID NO: 7, SEQ ID NO: 1, and SEQ ID NO: 11 or protein comprising amino acid sequences that are at least 80%, 85%, 90%, 91%, 92%, 93%, 94%, 95%, 96%, 97%, 98%, 99% or 100% identical to SEQ ID NO: 10, SEQ ID NO: 8, SEQ ID NO: 2, and SEQ ID NO: 12; have decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 3 and/or SEQ ID NO: 5 or a protein comprising an amino acid sequence that is at least 80%, 85%, 90%, 91%, 92%, 93%, 94%, 95%, 96%, 97%, 98%, 99% or 100% identical to SEQ ID NO: 4 and/or SEQ ID NO: 6; and have increased expression of a protein encoded by a nucleic acid that is at least 80%, 85%, 90%, 91%, 92%, 93%, 94%, 95%, 96%, 97%, 98%, 99% or 100% identical to SEQ ID NO: 13 and/or SEQ ID NO: 15 or a protein comprising an amino acid sequence that is at least 80%, 85%, 90%, 91%, 92%, 93%, 94%, 95%, 96%, 97%, 98%, 99% or 100% identical to SEQ ID NO: 14, SEQ ID NO: 16, SEQ ID NO: 145 and/or SEQ ID NO: 146, as compared to the expression level of the same cell type that has not been genetically engineered.

In some aspects of the current disclosure, a mammalian cell, wherein the cell is ABO blood group type O, has been genetically engineered to have reduced or no expression of a protein encoded by a nucleic acid that is at least 80%, 85%, 90%, 91%, 92%, 93%, 94%, 95%, 96%, 97%, 98%, 99% or 100% identical to SEQ ID NO: 3, SEQ ID NO: 5, and/or SEQ ID NO: 7 or a protein comprising an amino acid sequence that is at least 80%, 85%, 90%, 91%, 92%, 93%, 94%, 95%, 96%, 97%, 98%, 99% or 100% identical to SEQ ID NO: 4, SEQ ID NO: 6 and/or SEQ ID NO: 8, as compared to the expression level of the same cell type that has not been genetically engineered. In some embodiments, the mammalian ABO blood group type O cell has been genetically engineered to have decreased or no expression of proteins encoded by nucleic acids that are at least 80%, 85%, 90%, 91%, 92%, 93%, 94%, 95%, 96%, 97%, 98%, 99% or 100% identical to SEQ ID NO: 9, SEQ ID NO: 7, and SEQ ID NO: 11 or protein comprising amino acid sequences that are at least 80%, 85%, 90%, 91%, 92%, 93%, 94%, 95%, 96%, 97%, 98%, 99% or 100% identical to SEQ ID NO: 10, SEQ ID NO: 8, and SEQ ID NO: 12; have decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 3 and/or SEQ ID NO: 5 or a protein comprising an amino acid sequence that is at least 80%, 85%, 90%, 91%, 92%, 93%, 94%, 95%, 96%, 97%, 98%, 99% or 100% identical to SEQ ID NO: 4 and/or SEQ ID NO: 6; and have increased expression of a protein encoded by a nucleic acid that is at least 80%, 85%, 90%, 91%, 92%, 93%, 94%, 95%, 96%, 97%, 98%, 99% or 100% identical to SEQ ID NO: 13 and/or SEQ ID NO: 15 or a protein comprising an amino acid sequence that is at least 80%, 85%, 90%, 91%, 92%, 93%, 94%, 95%, 96%, 97%, 98%, 99% or 100% identical to SEQ ID NO: 14, SEQ ID NO: 16, SEQ ID NO: 145 and/or SEQ ID NO: 146, as compared to the expression level of the same cell type that has not been genetically engineered.

Some aspects of the current disclosure describe a mammalian cell that has been genetically engineered to have decreased or no expression of the protein encoded by the renalase gene, and wherein the cell also has been genetically engineered to have decreased or no expression of the protein encoded by the ABO gene; decreased or no expression of the protein encoded by the CXCL 10 gene; decreased or no expression of the protein encoded by the beta-2 microglobulin (B2M) gene; decreased or no expression of the protein encoded by the tissue factor (F3) gene; and/or increased expression of the protein encoded by the CD47 gene, as compared to the protein expression level of the same cell type that has not been genetically engineered.

In some embodiments of the disclosure, the mammalian cell has been genetically engineered to have increased expression of the protein encoded by the CD47 gene as compared to the protein expression level of the same cell type that has not been genetically engineered wherein the CD47 protein comprises an amino acid sequence that is at least 85%, 90%, 91%, 92%, 93%, 94%, 95%, 96%, 97%, 98%, 99%, or 100% identical to SEQ ID NO: 145 or 146, wherein the CD47 protein comprises a substitution at one or more of the amino acids corresponding to amino acid positions Q1, L3, A53, and L54 of SEQ ID NO: 145 or 146. In some embodiments, the CD47 protein comprises a P or an L at the amino acid position corresponding to position 1 of SEQ ID NO: 145 or 146. In some embodiments, the CD47 protein comprises an R, A, K, N, E or V at the amino acid position corresponding to position 3 of SEQ ID NO: 145 or 146. In some embodiments, the CD47 protein comprises a W, Y, D, Q or V at the amino acid position corresponding to position 53 of SEQ ID NO: 145 or 146. In some embodiments, the CD47 protein comprises an A, I, K, M, E, W, S, or V at the amino acid position corresponding to position 54 of SEQ ID NO: 145 or 146. In some embodiments, the CD47 protein comprises a P at the amino acid position corresponding to position 1 of SEQ ID NO: 145 or 146. In some embodiments, the CD47 comprises an amino acid other than a Q at the amino acid position corresponding to position 1 of SEQ ID NO: 145 or 146. In some embodiments, the CD47 comprises an amino acid other than L at the amino acid position corresponding to position 3 of SEQ ID NO: 145 or 146. In some embodiments, the CD47 comprises an amino acid other than an A at the amino acid position corresponding to position 53 of SEQ ID NO: 145 or 146. In some embodiments, the CD47 comprises an amino acid other than a L at the amino acid position corresponding to position 54 of SEQ ID NO: 145 or 146. In some embodiments, the CD47 protein is membrane-bound.

Some aspects of the current disclosure describe a composition comprising one or more of the herein described engineered mammalian cells. In some embodiments, the composition comprises a plurality of non-native cells; wherein:
a) at least 30% of the cells in the composition are NKX6.1-positive, ISL1-positive cells;
b) at least 25% of the cells in the composition are NKX6.1-negative, ISL1-positive cells;
c) there are more NKX6.1-positive, ISL1-positive cells than NKX6.1-negative, ISL1-positive cells in the composition;
d)
   i) less than 12% of the cells in the composition are NKX6.1-negative, ISL1-negative cells; and/or
   ii) between 9-25% of the cells in the composition are NKX6.1-positive, ISL1-negative cells; and
e) less than 40% of the cells in the composition are VMAT1-positive cells.

Some aspects of the current disclosure describe methods of administering the composition described to a subject. In some embodiments, the subject has diabetes.

### BRIEF DESCRIPTION OF THE DRAWINGS

FIGS. 1A-1C show that removal of A-antigen protects SC-islets from immune attack. FIG. 1A is a bar graph showing the percent of Type A-positive SC-islet cells that were differentiated from either unedited wildtype hESC or from an ABO-knockout version of the hESCs. FIG. 1B is a bar graph showing percent cytotoxicity of SC-islets differentiated from wildtype hESCs or ABO-knockout hESCs in an antibody-dependent cellular cytotoxicity assay. FIG. 1C is a bar graph showing percent cytotoxicity of SC-islets differentiated from wildtype hESCs or ABO-knockout hESCs in a complement-dependent cytotoxicity assay. Graphs are representative of 5 independent experiments with the ABO-KO SC-islet clonal cell line.

FIG. 2 shows that removal of HLA-class I prevents T-cell activation by SC-islets. T cell response to SC-islets (percentage of IFNγ-positive CD8 T cells) were measured in three different donors (donors 1-3) under three different conditions: 1) no stimulus (first bar in each donor graph); 2) wildtype SC-islets (second bar in each donor graph); 3) B2M knockout SC-islets (third bar in each donor graph).

FIGS. 3A-3B are graphs comparing wildtype cells to B2M-knockout cells. FIG. 3A is a graph showing the percentage of ISL1-positive cells in wildtype or B2M-knockout (“HLA-I^{KO}”) SC-islets that were removed from grafts at the indicated timepoints. FIG. 3B shows the frequency of hCD69 expression on hCD8-positive cells recovered from wildtype or B2M-knockout (“HLA-I^{KO}”) SC-islet grafts removed at the indicated timepoints. Asterisk indicates unpaired T-test.

FIGS. 4A-4B show that CD142 knockout hESCs have significantly reduced tissue factor pathway activation in vitro. FIG. 4A is a bar graph showing the percentage of tissue factor (CD142)-positive cells in wildtype hESCs or in two different CD142-knockout pools. FIG. 4B is a bar graph showing the percentage of tissue factor in wildtype (WT) cells or in cells from one of two different CD142-knockout pools of cells. “WT+αTF mAb” corresponds to wildtype hESCs pre-treated with a saturating level of anti-tissue factor antibody to block the complement pathway activation. The assay of FIG. 4B includes cells, recombinant FVII, and recombinant FX. FX activation is measured with a colorimetric substrate and this depends on CD142 activating FVII, which in turn activates FX. In the experiments shown, leaving out FVII did not completely eliminate the signal (“No FVII (assay baseline)”).

FIGS. 5A-5C illustrate a gene-editing strategy for generating a high-affinity CD47 mutant. FIG. 5A shows exemplary guide sequences close to the editing site in CD47 to enable HDR mediated repair. FIG. 5B shows an example of template sequences for templated repair. FIG. 5C shows a simplified schematic of a portion of the wildtype CD47 protein sequence and a portion of the high-affinity CD47 protein sequence.

FIG. 6 shows a series of flow plots for different CD47 mutants based on expression of NKX6.1 (x-axis) and ISL1 (y-axis). “WT” corresponds to SC-islets generated from wildtype hESCs (top left panel). SB(CD47)51 corresponds to SC-islets generated from hESCs heterozygous for the CD47-high affinity edit and for a CD47 knockout (top two right panels), SB(CD47)53 corresponds to SC-islets generated from hESCs homozygous for the CD47-high affinity edit (bottom two left panels); and SB(CD47)54 corresponds to SC-islets generated from hESCs homozygous for a CD47 knockout (bottom two right panels).

FIG. 7 is a bar graph showing the percentage of CFSE positive, CD11b positive cells across the different test conditions. The “positive CTRL” corresponds to FITC-dextran. “dKO HI TG1 TG2 #22”, “dKO HI TG1 TG2 #23”, “dKO HI TG1 TG2 #24” and “dKO HI TG1 TG2 #54” correspond to four different clones of endothelial cells differentiated from stem cells in which the genes B2M and CIITA had been knocked out, and in which PDL1 and CD47 genes were knocked in.

FIGS. 8A-8B show a series of flow cytometry plots for expression of the indicated genes in wildtype hESCs or in three different clones of hESCs in which B2M and ABO were knocked out and CD47 was knocked in (clones A3, A5 and A11). FIG. 8A shows plots for expression of A-antigen (top row), HLA-A, HLA-B, HLA-C (middle row), and CD47 (bottom row). In the bottom row, the first inset box in each plot indicates portion for expected endogenous levels of CD47, while the second inset box in each plot indicates portion for expected overexpression of CD47. FIG. 8B shows plots for expression of stem cell markers SOX2 and OCT4 in the indicated cell types.

FIGS. 9A-9B show flow cytometry plots. FIG. 9A shows several flow cytometry plots for the expression of NKX6.1 (x-axis) and ISL1 (y-axis) in stage 5 SC-islet cells differentiated from wildtype hESCs (“WT”) or from hESCs that had been engineered to knock out B2M and ABO and to knock in CD47. The lower panel of FIG. 9A shows a graph illustrating the percentage of different cell types in stage 5 SC-islet cells differentiated from wildtype hESCs (“WT”) or from hESCs that had been engineered to knock out B2M and ABO and to knock in CD47. FIG. 9B shows several flow cytometry plots for the expression of NKX6.1 (x-axis) and ISL1 (y-axis) in stage 6 day 6 SC-islet cells differentiated from wildtype hESCs (“WT”) or from hESCs that had engineered to knock out B2M and ABO and to knock in CD47. The lower panel of FIG. 9B shows a graph illustrating the percentage of different cell types in stage 6 day 6 SC-islet cells differentiated from wildtype hESCs (“WT”) or from hESCs that had been engineered to knock out B2M and ABO and to knock in CD47. For each of the bars in the bar graphs in FIGS. 9A and 9B, the top quadrant of each bar corresponds to the percentage of cells that are double-negative for NKX6.1 and ISL1, the second quadrant down in each bar corresponds to the percentage of cells that are NKX6.1-positive and ISL1-negative, the third quadrant down in each bar corresponds to the percentage of cells that are ISL1-positive and NKX6.1-negative, and the bottom quadrant in each bar corresponds to the percentage of cells that are ISL1-positive and NKX6.1-positive.

FIGS. 10A-10B show flow cytometry plots. FIG. 10A shows flow plots for the expression of NKX6.1 (x-axis) and ISL1 (y-axis) in Stage 5 or Stage 6, day 6 SC-islet cells differentiated from hESCs that had been engineered to knock out B2M, ABO and CD142 and to knock in CD47. FIG. 10B shows a flow plot for the expression of CD142 in Stage 6 SC-islets differentiated from hESCs that had been engineered to knock out B2M, ABO and CD142 and to knock in CD47.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION

The following description and examples illustrate embodiments of the present disclosure in detail. It is to be understood that this disclosure is not limited to the particular embodiments described herein and as such can vary. Those of skill in the art will recognize that there are numerous variations and modifications of this disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure can be described in the context of a single embodiment, the features can also be provided separately or in any suitable combination. Conversely, although the present disclosure can be described herein in the context of separate embodiments for clarity, the present disclosure can also be implemented in a single embodiment.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only and is not intended to be limiting.

In this application, the use of the singular includes the plural unless specifically stated otherwise. It must be noted that, as used in the specification, the singular forms “a,” “an” and “the” include plural referents unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

Furthermore, use of the term “including” as well as other forms, such as “include”, “includes,” and “included,” is not limiting.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” includes 10 and any amounts from 9 to 11. In yet another example, the term “about” in relation to a reference numerical value can also include a range of values plus or minus 10%, 9%, 8%, 7%, 6%, 5%, 4%, 3%, 2%, or 1% from that value. Alternatively, particularly with respect to biological systems or processes, the term “about” can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “diabetes” and its grammatical equivalents as used herein can refer to is a disease characterized by high blood sugar levels over a prolonged period. For example, the term “diabetes” and its grammatical equivalents as used herein can refer to all or any type of diabetes, including, but not limited to, type 1, type 2, cystic fibrosis-related, surgical, gestational diabetes, and mitochondrial diabetes. In some embodiments, diabetes can be a form of hereditary diabetes. In some embodiments, diabetes can be an autoimmune form of diabetes.

The term “endocrine cell(s),” if not particularly specified, can refer to hormone-producing cells present in the pancreas of an organism, such as “islet”, “islet cells”, “islet equivalent”, “islet-like cells”, “pancreatic islets” and their grammatical equivalents. In an embodiment, the endocrine cells can be differentiated from pancreatic progenitor cells or precursors. Islet cells can comprise different types of cells, including, but not limited to, pancreatic α cells, pancreatic β cells, pancreatic δ cells, pancreatic F cells, and/or pancreatic ε cells. Islet cells can also refer to a group of cells, cell clusters, or the like.

“Guide RNA” and simply “guide” are used herein interchangeably to refer to either a crRNA (also known as CRISPR RNA) nucleic acid, or the combination of a crRNA nucleic acid and a trRNA (also known as tracrRNA) nucleic acid. The crRNA and trRNA may be associated as a single RNA molecule (single guide RNA, sgRNA) or in two separate RNA molecules (dual guide RNA, dgRNA). “Guide RNA” refers to each type unless specified otherwise. The trRNA may be a naturally-occurring sequence, or a trRNA sequence with modifications or variations compared to naturally-occurring sequences. For clarity, the terms “guide RNA” or “guide” as used herein, and unless specifically stated otherwise, may refer to an RNA molecule (comprising A, C, G, and U nucleotides) or to a DNA molecule encoding such an RNA molecule (comprising A, C, G, and T nucleotides) or complementary sequences thereof. In general, in the case of a DNA nucleic acid construct encoding a guide RNA, the U residues in any of the RNA sequences described herein may be replaced with T residues, and in the case of a guide RNA construct encoded by any of the DNA sequences described herein, the T residues may be replaced with U residues. In some embodiments, any of the guide RNA sequences comprises a scaffold sequence. In some embodiments, the scaffold sequence comprises a sequence that is at least 90%, 91%, 92%, 93%, 94%, 95%, 96%, 97%, 98%, 99% or 100% identical to the sequence of SEQ ID NO: 143 (GTTTTAGAGCTAGAAATAGCAAGTTAAAATAAGGCTAGTCCGTTATCAACTTGAAA AAGTGGCACCGAGTCGGTGCTTTT). In some embodiments, the guide RNA comprises one or more modified nucleotides. A discussion of modified guide RNAs can be found, for example, in WO2022/056000, which is incorporated herein in its entirety. In some embodiments, the guide RNAs are unmodified.

“Polynucleotide,” “nucleic acid,” and “nucleic acid molecule,” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N4-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O6-methylguanine, 4-thio-pyrimidines, 4-amino-pyrimidines, 4-dimethylhydrazine-pyrimidines, and O4-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA. The disclosure provides a number of exemplary nucleotide sequences herein, and contemplates reverse complements of these nucleotide sequences, as well as RNA and/or DNA equivalents of any of these sequences. For example, an RNA equivalent of any of the DNA sequences disclosed herein would comprise uracils in place of thymines in the sequence, whereas a DNA equivalent of any of the RNA sequences disclosed herein would comprise thymines in place of uracils.

As used herein, “CRISPR” systems and “RNA-targeted endonucleases” or “Cas-nucleases” includes the type II CRISPR systems of S. pyogenes, S. aureus, and other prokaryotes, and modified (e.g., engineered or mutant) versions thereof. See, e.g., US2016/0312198 A1; US 2016/0312199 A1. In particular embodiments, the RNA-targeted endonuclease is a type II CRISPR Cas enzyme. Other examples of Cas nucleases include a Csm or Cmr complex of a type III CRISPR system or the Cas 10, Csm1, or Cmr2 subunit thereof; and a Cascade complex of a type I CRISPR system, or the Cas3 subunit thereof. In some embodiments, the Cas nuclease may be from a Type-IIA, Type-IIB, or Type-IIC system. For discussion of various CRISPR systems and Cas nucleases see, e.g., Makarova et al., Nat. Rev. Microbiol., 9:467-477 (2011); Makarova et al., Nat. Rev. Microbiol., 13:722-36 (2015); Shmakov et al., Molecular Cell, 60:385-397 (2015). Non-limiting exemplary species that the Cas nuclease can be derived from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gammaproteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, and Acaryochloris marina. In some embodiments, the Cas protein is a Cpf1 or Cas12 (e.g., Cas12i2) protein. In some embodiments, the disclosure provides for a cell (e.g., a stem cell or stem cell-derived beta cell) comprising one or more genetic disruptions using a CRISPR system and one or more of guide RNAs comprising any of the sequences disclosed herein. In some embodiments, the CRISPR system disrupts the target gene by introducing one or more insertions/deletions (e.g., indels) into the target gene.

In some embodiments, the Cas protein comprises an amino acid sequence that is at least 80%, 85%, 90%, 91%, 92%, 93%, 94%, 95%, 96%, 97%, 98%, 99% or 100% identical to the sequence of SEQ ID NO: 144 (designated herein as SpCas9):

MDKKYSIGLDIGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVEEDKKHERHPIF
GNIVDEVAYHEKYPTIYHLRKKLVDSTDKADLRLIYLALAHMIKFRGHFLIEGDLNPDNS
DVDKLFIQLVQTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGY
AGYIDGGASQEEFYKFIKPILEKMDGTEELLVKLNREDLLRKQRTFDNGSIPHQIHLGEL
HAILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPA
FLSGEQKKAIVDLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKVMKQLKRRRYTG
WGRLSRKLINGIRDKQSGKTILDFLKSDGFANRNFMQLIHDDSLTFKEDIQKAQVSGQG
DSLHEHIANLAGSPAIKKGILQTVKVVDELVKVMGRHKPENIVIEMARENQTTQKGQKN
SRERMKRIEEGIKELGSQILKEHPVENTQLQNEKLYLYYLQNGRDMYVDQELDINRLSD
YDVDHIVPQSFLKDDSIDNKVLTRSDKNRGKSDNVPSEEVVKKMKNYWRQLLNAKLIT
QRKFDNLTKAERGGLSELDKAGFIKRQLVETRQITKHVAQILDSRMNTKYDENDKLIRE
VKVITLKSKLVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRPLIETNGETGEI
VWDKGRDFATVRKVLSMPQVNIVKKTEVQTGGFSKESILPKRNSDKLIARKKDWDPKK
YGGFDSPTVAYSVLVVAKVEKGKSKKLKSVKELLGITIMERSSFEKNPIDFLEAKGYKE
VKKDLIIKLPKYSLFELENGRKRMLASAGELQKGNELALPSKYVNFLYLASHYEKLKGS
PEDNEQKQLFVEQHKHYLDEIIEQISEFSKRVILADANLDKVLSAYNKHRDKPIREQAENI
IHLFTLTNLGAPAAFKYFDTTIDRKRYTSTKEVLDATLIHQSITGLYETRIDLSQLGGD

The terms “progenitor” and “precursor” cell are used interchangeably herein and refer to cells that have a cellular phenotype that is more primitive (e.g., is at an earlier step along a developmental pathway or progression than is a fully differentiated cell) relative to a cell which it can give rise to by differentiation. Often, progenitor cells can also have significant or very high proliferative potential. Progenitor cells can give rise to multiple distinct differentiated cell types or to a single differentiated cell type, depending on the developmental pathway and on the environment in which the cells develop and differentiate.

A “precursor thereof” as the term related to an insulin-positive endocrine cell can refer to any cell that is capable of differentiating into an insulin-positive endocrine cell, including for example, a pluripotent stem cell, a definitive endoderm cell, a primitive gut tube cell, a pancreatic progenitor cell, or endocrine progenitor cell, that if cultured under suitable conditions will differentiate the precursor cell into the insulin-positive endocrine cell.

The terms “stem cell-derived β cell,” “SC-β cell,” “functional β cell,” “functional pancreatic β cell,” “mature SC-β cell,” “β-like cell” and their grammatical equivalents can refer to cells (e.g., non-native pancreatic β cells) that display at least one marker indicative of a pancreatic β cell (e.g., PDX-1 or NKX6.1), expresses insulin, and display a glucose stimulated insulin secretion (GSIS) response similar or superior to that of an endogenous mature β cell (e.g., a mature β from a healthy functioning pancreas from a healthy adult non-diabetic patient). For simplicity, SC-β cells may be referred to as simply “β cells” in this disclosure. In some embodiments, the terms “SC-β cell” and “non-native β cell” as used herein are interchangeable. In some embodiments, the “SC-β cell” expresses lower levels of MAFA than a pancreatic β cell from a healthy adult human patient. In some embodiments, the “SC-β cell” expresses higher levels of MAFB than a pancreatic β cell from a healthy adult human patient. In some embodiments, the “SC-β cell” expresses higher levels of SIX2, HOPX, IAPP and/or UCN3 than a pancreatic β cell from a healthy adult human patient. In some embodiments, the “SC-β cell” comprises a mature pancreatic cell. It is to be understood that the SC-β cells need not be derived (e.g., directly) from stem cells, as the methods of the disclosure are capable of deriving SC-β cells from any insulin-positive endocrine cell or precursor thereof using any cell as a starting point (e.g., one can use embryonic stem cells, induced-pluripotent stem cells, progenitor cells such as definitive endoderm cells, partially reprogrammed somatic cells (e.g., a somatic cell which has been partially reprogrammed to an intermediate state between an induced pluripotent stem cell and the somatic cell from which it was derived), multipotent cells, totipotent cells, a transdifferentiated version of any of the foregoing cells, etc., as the invention is not intended to be limited in this manner). In some embodiments, the SC-β cells exhibit a response to multiple glucose challenges (e.g., at least one, at least two, or at least three or more sequential glucose challenges). In some embodiments, the response resembles the response of endogenous islets (e.g., human islets) to multiple glucose challenges. In some embodiments, the morphology of the SC-β cell resembles the morphology of an endogenous β cell. In some embodiments, the SC-β cell exhibits an in vitro GSIS response that resembles the GSIS response of an endogenous β cell. In some embodiments, the SC-β cell exhibits an in vivo GSIS response that resembles the GSIS response of an endogenous β cell. In some embodiments, the SC-β cell exhibits both an in vitro and in vivo GSIS response that resembles the GSIS response of an endogenous β cell. In some embodiments, the GSIS response of the SC-β cell can be observed within two weeks of transplantation of the SC-β cell into a host (e.g., a human or animal). In some embodiments, the GSIS response of the SC-β cell can be observed within three weeks of transplantation of the SC-β cell into a host (e.g., a human or animal). In some embodiments, the GSIS response of the SC-β cell can be observed within four weeks of transplantation of the SC-β cell into a host (e.g., a human or animal). In some embodiments, the GSIS response of the SC-cell can be observed between one month and three months of transplantation of the SC-β cell into a host (e.g., a human or animal). In some embodiments, the SC-β cells package insulin into secretory granules. In some embodiments, the SC-β cells exhibit encapsulated crystalline insulin granules when viewed using electron microscopy. In some embodiments, the SC-β cells exhibit a stimulation index of greater than 1. In some embodiments, the SC-β cells exhibit a stimulation index of greater than 1.1. In some embodiments, the SC-β cells exhibit a stimulation index of greater than 2. In some embodiments, the stimulation index of the cell is characterized by the ratio of insulin secreted in response to high glucose concentrations (e.g., 15 mM) compared to low glucose concentrations (e.g., 2.5 mM).

In some embodiments, the SC-β cells exhibit cytokine-induced apoptosis in response to cytokines. In some embodiments, insulin secretion from the SC-β cells is enhanced in response to known antidiabetic drugs (e.g., secretagogues). In some embodiments, the SC-β cells are monohormonal. In some embodiments, the SC-β cells do not abnormally co-express other hormones, such as glucagon, somatostatin or pancreatic polypeptide. In some embodiments, the SC-β cells exhibit a low rate of replication. In some embodiments, the SC-β cells increase intracellular Ca2+ in response to glucose.

The terms “stem cell-derived α cell,” “SC-α cell,” “functional α cell,” “functional pancreatic α cell,” “mature SC-α cell,” “α-like cell” and their grammatical equivalents can refer to cells (e.g., non-native pancreatic α cells) that display at least one marker indicative of a pancreatic α cell (e.g., glucagon, expressing ISL1 but not NKX6.1), expresses glucagon, and is capable of secreting functional glucagon in response to a stimulus that induces an endogenous pancreatic α cell to secrete functional glucagon. In some embodiments, the “SC-α cell” does not express somatostatin. In some embodiments, the “SC-α cell” does not express insulin. In some embodiments, the terms “SC-α cell” and “non-native α cell” as used herein are interchangeable. In some embodiments, the “SC-α cell” comprises a mature pancreatic cell. For short, these cells may be referred to as simply “a cells” in this disclosure.

The terms “stem cell-derived δ cell,” “SC-δ cell,” “functional δ cell,” “functional pancreatic δ cell,” “mature SC-δ cell,” “δ-like cell” and their grammatical equivalents can refer to cells (e.g., non-native pancreatic δ cells) that display at least one marker indicative of a pancreatic δ cell (e.g., somatostatin), expresses and is capable of secreting somatostatin in response to a stimulus that induces an endogenous pancreatic δ cell to secrete functional glucagon. For simplicity, SC-δ cells may be referred to as simply “δ cells” in this disclosure. In some embodiments, “SC-δ cell” does not express glucagon. In some embodiments, “SC-δ cell” does not express insulin. In some embodiments, the terms “SC-δ cell” and “non-native δ cell” as used herein are interchangeable. In some embodiments, the “SC-δ cell” comprises a mature pancreatic cell.

The terms “stem cell-derived enterochromaffin (EC) cell,” “SC-EC cell,” and their grammatical equivalents can refer to cells (e.g., non-native pancreatic EC cells) that display at least one marker indicative of a pancreatic EC cell (e.g., VMAT1 (vesicular monoamine transporter 1), expressing NKX6.1 but not ISL1). In some embodiments, the terms “SC-EC cell” and “non-native EC cell” as used herein are interchangeable.

The term “stem cell-derived islet cell,” “SC-islet cell,” and their grammatical equivalents refers to islet cells or islet-like cells that have been differentiated from stem cells in vitro. Examples of SC-islet cells include SC-β cells, SC-α cells, and SC-δ cells.

It is to be understood that the SC-islet cells need not be derived (e.g., directly) from stem cells, as the methods of the disclosure are capable of deriving SC-islet cells from other precursor cells generated during in vitro differentiation of SC-islet cells as a starting point (e.g., one can use embryonic stem cells, induced-pluripotent stem cells, progenitor cells, partially reprogrammed somatic cells (e.g., a somatic cell which has been partially reprogrammed to an intermediate state between an induced pluripotent stem cell and the somatic cell from which it was derived), multipotent cells, totipotent cells, a transdifferentiated version of any of the foregoing cells, etc., as the invention is not intended to be limited in this manner).

As used herein, the term “insulin producing cell” and its grammatical equivalent refer to a cell differentiated from a pancreatic progenitor, or precursor thereof, which secretes insulin. An insulin-producing cell can include pancreatic β cell as that term is described herein, as well as pancreatic β-like cells (e.g., insulin-positive, endocrine cells) that synthesize (e.g., transcribe the insulin gene, translate the proinsulin mRNA, and modify the proinsulin mRNA into the insulin protein), express (e.g., manifest the phenotypic trait carried by the insulin gene), or secrete (release insulin into the extracellular space) insulin in a constitutive or inducible manner. A population of insulin producing cells e.g., produced by differentiating insulin-positive endocrine cells or a precursor thereof into SC-β cells according to the methods of the present disclosure can be pancreatic β cells or β-like cells (e.g., cells that have at least one, or at least two least characteristics of an endogenous β cell and exhibit a glucose stimulated insulin secretion (GSIS) response that resembles an endogenous adult β cell). The population of insulin-producing cells, e.g., produced by the methods as disclosed herein can comprise mature pancreatic β cell or SC-β cells, and can also contain non-insulin-producing cells (e.g., cells of cell like phenotype with the exception they do not produce or secrete insulin).

The terms “insulin-positive β-like cell,” “insulin-positive endocrine cell,” and their grammatical equivalents can refer to cells (e.g., pancreatic endocrine cells) that display at least one marker indicative of a pancreatic β cell and also expresses insulin but, unless specified otherwise, lack a glucose stimulated insulin secretion (GSIS) response characteristic of an endogenous β cell. Exemplary markers of “insulin-positive endocrine cell” include, but are not limited to, NKX6.1 (NK6 homeobox 1), ISL1 (Islet1), and insulin.

The term “β cell marker” refers to, without limitation, proteins, peptides, nucleic acids, polymorphism of proteins and nucleic acids, splice variants, fragments of proteins or nucleic acids, elements, and other analyte which are expressed or present in pancreatic β cells. Exemplary β cell markers include, but are not limited to, pancreatic and duodenal homeobox 1 (PDX1) polypeptide, insulin, c-peptide, amylin, E-cadherin, Hnf3β, PCI/3, B2, Nkx2.2, GLUT2, PC2, ZnT-8, ISL1, Pax6, Pax4, NeuroD, 1 Inf1b, Hnf-6, Hnf-3beta, VMAT2, NKX6.1, and MafA, and those described in Zhang et al., Diabetes. 50 (10): 2231-6 (2001). In some embodiments, the β cell marker is a nuclear β-cell marker. In some embodiments, the β cell marker is PDX1 or PH3.

The term “pancreatic endocrine marker” can refer to without limitation, proteins, peptides, nucleic acids, polymorphism of proteins and nucleic acids, splice variants, fragments of proteins or nucleic acids, elements, and other analytes which are expressed or present in pancreatic endocrine cells. Exemplary pancreatic endocrine cell markers include, but are not limited to, Ngn-3, NeuroD and Islet-1.

The term “pancreatic progenitor,” “pancreatic endocrine progenitor,” “pancreatic precursor,” “pancreatic endocrine precursor” and their grammatical equivalents are used interchangeably herein and can refer to a stem cell which is capable of becoming a pancreatic hormone expressing cell capable of forming pancreatic endocrine cells, pancreatic exocrine cells or pancreatic duct cells. These cells are committed to differentiating towards at least one type of pancreatic cell, e.g. β cells that produce insulin; α cells that produce glucagon; δ cells (or D cells) that produce somatostatin; and/or F cells that produce pancreatic polypeptide. Such cells can express at least one of the following markers: NGN3, NKX2.2, NeuroD, ISL-1, Pax4, Pax6, or ARX.

The term “PDX1-positive pancreatic progenitor” as used herein can refer to a cell which is a pancreatic endoderm (PE) cell which has the capacity to differentiate into SC-β cells, such as pancreatic β cells. A PDX1-positive pancreatic progenitor expresses the marker PDX1. Other markers include, but are not limited to Cdcp1, or Ptf1a, or HNF6 or NRx2.2. The expression of PDX1 may be assessed by any method known by the skilled person such as immunochemistry using an anti-PDX1 antibody or quantitative RT-PCR. In some embodiments, a PDX1-positive pancreatic progenitor cell lacks expression of NKX6.1. In some embodiments, a PDX1-positive pancreatic progenitor cell can also be referred to as PDX1-positive, NKX6.1-negative pancreatic progenitor cell due to its lack of expression of NKX6.1. In some embodiments, the PDX1-positive pancreatic progenitor cells can also be termed as “pancreatic foregut endoderm cells.”

The terms “PDX1-positive, NKX6.1-positive pancreatic progenitor,” and “NKX6.1-positive pancreatic progenitor” are used interchangeably herein and can refer to a cell which is a pancreatic endoderm (PE) cell which has the capacity to differentiate into insulin-producing cells, such as pancreatic β cells. A PDX1-positive, NKX6.1-positive pancreatic progenitor expresses the markers PDX1 and NKX6-1. Other markers may include, but are not limited to Cdcp1, or Ptf1a, or HNF6 or NRx2.2. The expression of NKX6-1 may be assessed by any method known by the skilled person such as immunochemistry using an anti-NKX6-1 antibody or quantitative RT-PCR. As used herein, the terms “NKX6.1” and “NKX6-1” are equivalent and interchangeable. In some embodiments, the PDX1-positive, NKX6.1-positive pancreatic progenitor cells can also be termed as “pancreatic foregut precursor cells.”

The terms “NeuroD” and “NeuroD1” are used interchangeably and identify a protein expressed in pancreatic endocrine progenitor cells and the gene encoding it.

The term “differentiated cell” or its grammatical equivalents means any primary cell that is not, in its native form, pluripotent as that term is defined herein. Stated another way, the term “differentiated cell” can refer to a cell of a more specialized cell type derived from a cell of a less specialized cell type (e.g., a stem cell such as an induced pluripotent stem cell) in a cellular differentiation process. Without wishing to be limited to theory, a pluripotent stem cell in the course of normal ontogeny can differentiate first to an endoderm cell that is capable of forming pancreas cells and other endoderm cell types. Further differentiation of an endoderm cell may lead to the pancreatic pathway, where ˜98% of the cells become exocrine, ductular, or matrix cells, and ˜2% become endocrine cells. Early endocrine cells are islet progenitors, which can then differentiate further into insulin-producing cells (e.g. functional endocrine cells) which secrete insulin, glucagon, somatostatin, or pancreatic polypeptide. Endoderm cells can also be differentiated into other cells of endodermal origin, e.g. lung, liver, intestine, thymus etc.

As used herein, the term “somatic cell” can refer to any cells forming the body of an organism, as opposed to germline cells. In mammals, germline cells (also known as “gametes”) are the spermatozoa and ova which fuse during fertilization to produce a cell called a zygote, from which the entire mammalian embryo develops. Every other cell type in the mammalian body—apart from the sperm and ova, the cells from which they are made (gametocytes) and undifferentiated stem cells—is a somatic cell: internal organs, skin, bones, blood, and connective tissue are all made up of somatic cells. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro. Unless otherwise indicated the methods for converting at least one insulin-positive endocrine cell or precursor thereof to an insulin-producing, glucose responsive cell can be performed both in vivo and in vitro (where in vivo is practiced when at least one insulin-positive endocrine cell or precursor thereof are present within a subject, and where in vitro is practiced using an isolated at least one insulin-positive endocrine cell or precursor thereof maintained in culture).

As used herein, the term “adult cell” can refer to a cell found throughout the body after embryonic development.

The term “endoderm cell” as used herein can refer to a cell which is from one of the three primary germ cell layers in the very early embryo (the other two germ cell layers are the mesoderm and ectoderm). The endoderm is the innermost of the three layers. An endoderm cell differentiates to give rise first to the embryonic gut and then to the linings of the respiratory and digestive tracts (e.g., the intestine), the liver and the pancreas.

The term “a cell of endoderm origin” as used herein can refer to any cell which has developed or differentiated from an endoderm cell. For example, a cell of endoderm origin includes cells of the liver, lung, pancreas, thymus, intestine, stomach and thyroid. Without wishing to be bound by theory, liver and pancreas progenitors (also referred to as pancreatic progenitors) are developed from endoderm cells in the embryonic foregut. Shortly after their specification, liver and pancreas progenitors rapidly acquire markedly different cellular functions and regenerative capacities. These changes are elicited by inductive signals and genetic regulatory factors that are highly conserved among vertebrates. Interest in the development and regeneration of the organs has been fueled by the intense need for hepatocytes and pancreatic β cells in the therapeutic treatment of liver failure and type I diabetes. Studies in diverse model organisms and humans have revealed evolutionarily conserved inductive signals and transcription factor networks that elicit the differentiation of liver and pancreatic cells and provide guidance for how to promote hepatocyte and β cell differentiation from diverse stem and progenitor cell types.

The term “definitive endoderm” as used herein can refer to a cell differentiated from an endoderm cell and which can be differentiated into a SC-β cell (e.g., a pancreatic β cell). A definitive endoderm cell expresses the marker Sox17. Other markers characteristic of definitive endoderm cells may include, but are not limited to MIXL2, GATA4, HNF3b, GSC, FGF17, VWF, CALCR, FOXQ1, CXCR4, Cerberus, OTX2, goosecoid, C-Kit, CD99, CMKOR1 and CRIP1. In particular, definitive endoderm cells herein express Sox 17 and in some embodiments Sox 17 and HNF3B, and do not express significant levels of GATA4, SPARC, APF or DAB. Definitive endoderm cells are not positive for the marker PDX1 (e.g. they are PDX1-negative). Definitive endoderm cells have the capacity to differentiate into cells including those of the liver, lung, pancreas, thymus, intestine, stomach and thyroid. The expression of Sox 17 and other markers of definitive endoderm may be assessed by any method known by the skilled person such as immunochemistry, e.g., using an anti-Sox 17 antibody, or quantitative RT-PCR.

The term “pancreatic endoderm” can refer to a cell of endoderm origin which is capable of differentiating into multiple pancreatic lineages, including pancreatic β cells, but no longer has the capacity to differentiate into non-pancreatic lineages.

The term “pancreatic islet cells” refers to a population of cells that include different types of pancreatic endocrine cells (β-cells, α-cells, δ-cells, ε-cells) and enterochromaffin (EC) cells, e.g., as described in Xavier et al. (J Clin Med. 2018 March; 7(3): 54), incorporated herein by reference.

The term “primitive gut tube cell” or “gut tube cell” as used herein can refer to a cell differentiated from an endoderm cell and which can be differentiated into a SC-β cell (e.g., a pancreatic β cell). A primitive gut tube cell expresses at least one of the following markers: HNP1-β, HNF3-β or HNF4-α. In some embodiments, a primitive gut tube cell is FOXA2-positive and SOX2-positive, i.e., expresses both FOXA2 (also known as HNF3-β) and SOX2. In some embodiments, a primitive gut tube cell is FOXA2-positive and PDX1-negative, i.e., expresses FOXA2 but not PDX1. Primitive gut tube cells have the capacity to differentiate into cells including those of the lung, liver, pancreas, stomach, and intestine. The expression of HNF1-β and other markers of primitive gut tube may be assessed by any method known by the skilled person such as immunochemistry, e.g., using an anti-HNF1-β antibody.

The term “phenotype” can refer to one or a number of total biological characteristics that define the cell or organism under a particular set of environmental conditions and factors, regardless of the actual genotype.

The terms “patient,” “subject,” and “individual” may be used interchangeably and refer to either a human or a non-human animal. The “non-human animals” and “non-human mammals” as used interchangeably herein, includes mammals such as rats, mice, rabbits, sheep, cats, dogs, cows, pigs, and non-human primates. The term “subject” also encompasses any vertebrate including but not limited to mammals, reptiles, amphibians and fish. However, advantageously, the subject is a mammal such as a human, or other mammals such as a domesticated mammal, e.g., dog, cat, horse, and the like, or production mammal, e.g. cow, sheep, pig, and the like. “Patient in need thereof” or “subject in need thereof” is referred to herein as a patient diagnosed with or suspected of having a disease or disorder, for instance, but not restricted to diabetes.

“Administering” as used herein can refer to providing one or more compositions described herein to a patient or a subject. By way of example and not limitation, composition administration, e.g., injection, can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. Alternatively, or concurrently, administration can be by the oral route. Additionally, administration can also be by surgical deposition of a bolus or pellet of cells, or positioning of a medical device. In an embodiment, a composition of the present disclosure can comprise engineered cells or host cells expressing nucleic acid sequences described herein, or a vector comprising at least one nucleic acid sequence described herein, in an amount that is effective to treat or prevent proliferative disorders. A pharmaceutical composition can comprise the cell population as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

The term “genetically engineered”, “genetically altered”, or “genetically modified” and their grammatical equivalents as used herein refer to a non-naturally occurring genetic modification. Examples of genetic engineering include use of gene editing systems such as the CRISPR/Cas, the piggybac, the TALEN, and/or zinc finger systems for disrupting the expression (e.g., to reduce or eliminate expression) of one or more gene targets in a cell, or for increasing the expression (e.g., by inserting a gene of interest) into a cell. A “genetically engineered”, “genetically altered”, or “genetically modified” cell, as used herein, means a cell that was genetically engineered, or a cell that was derived and/or descended from a cell that was genetically engineered. For example, an SC-islet cell that was derived from a stem cell that was genetically engineered would be considered a genetically engineered SC-islet.

In some embodiments, “ABO” as used herein, is a protein encoded by a nucleic acid that is at least 80%, 85%, 90%, 91%, 92%, 93%, 94%, 95%, 96%, 97%, 98%, 99% or 100% identical to SEQ ID NO: 1. In some embodiments, “ABO” is a protein comprising an amino acid sequence that is at least 80%, 85%, 90%, 91%, 92%, 93%, 94%, 95%, 96%, 97%, 98%, 99% or 100% identical to SEQ ID NO: 2.

In some embodiments, “renalase” as used herein, is a protein encoded by a nucleic acid that is at least 80%, 85%, 90%, 91%, 92%, 93%, 94%, 95%, 96%, 97%, 98%, 99% or 100% identical to SEQ ID NO: 3 or 5. In some embodiments, “renalase” is a protein comprising an amino acid sequence that is at least 80%, 85%, 90%, 91%, 92%, 93%, 94%, 95%, 96%, 97%, 98%, 99% or 100% identical to SEQ ID NO: 4 and/or 6.

In some embodiments, “CXCL10” as used herein, is a protein encoded by a nucleic acid that is at least 80%, 85%, 90%, 91%, 92%, 93%, 94%, 95%, 96%, 97%, 98%, 99% or 100% identical to SEQ ID NO: 7. In some embodiments, “CXCL10” is a protein comprising an amino acid sequence that is at least 80%, 85%, 90%, 91%, 92%, 93%, 94%, 95%, 96%, 97%, 98%, 99% or 100% identical to SEQ ID NO: 8.

In some embodiments, “beta-2 microglobulin” or “B2M” as used herein, is a protein encoded by a nucleic acid that is at least 80%, 85%, 90%, 91%, 92%, 93%, 94%, 95%, 96%, 97%, 98%, 99% or 100% identical to SEQ ID NO: 9. In some embodiments, “B2M” is a protein comprising an amino acid sequence that is at least 80%, 85%, 90%, 91%, 92%, 93%, 94%, 95%, 96%, 97%, 98%, 99% or 100% identical to SEQ ID NO: 10.

In some embodiments, “tissue factor” as used herein, is a protein encoded by a nucleic acid that is at least 80%, 85%, 90%, 91%, 92%, 93%, 94%, 95%, 96%, 97%, 98%, 99% or 100% identical to SEQ ID NO: 11. In some embodiments, “tissue factor” is a protein comprising an amino acid sequence that is at least 80%, 85%, 90%, 91%, 92%, 93%, 94%, 95%, 96%, 97%, 98%, 99% or 100% identical to SEQ ID NO: 12.

In some embodiments, “CD47” as used herein, is a protein encoded by a nucleic acid that is at least 80%, 85%, 90%, 91%, 92%, 93%, 94%, 95%, 96%, 97%, 98%, 99% or 100% identical to SEQ ID NO: 13 or 15. In some embodiments, “CD47” is a protein comprising an amino acid sequence that is at least 80%, 85%, 90%, 91%, 92%, 93%, 94%, 95%, 96%, 97%, 98%, 99% or 100% identical to SEQ ID NO: 14, SEQ ID NO: 16, SEQ ID NO: 145 and/or SEQ ID NO: 146.

### Stem Cells

The term “stem cell” is used herein to refer to a mammalian cell that has the ability both to self-renew and to generate a differentiated cell type (Morrison et al. (1997) Cell 88:287-298). In the context of cell ontogeny, the adjective “differentiated,” or “differentiating” is a relative term. A “differentiated cell” is a cell that has progressed further down the developmental pathway than the cell it is being compared with. Thus, pluripotent stem cells can differentiate into lineage-restricted progenitor cells (e.g., mesodermal stem cells), which in turn can differentiate into cells that are further restricted (e.g., neuron progenitors), which can differentiate into end-stage cells (i.e., terminally differentiated cells, e.g., neurons, cardiomyocytes, etc.), which play a characteristic role in a certain tissue type, and can or cannot retain the capacity to proliferate further. Stem cells can be characterized by both the presence of specific markers (e.g., proteins, RNAs, etc.) and the absence of specific markers. Stem cells can also be identified by functional assays both in vitro and in vivo, particularly assays relating to the ability of stem cells to give rise to multiple differentiated progeny. In an embodiment, the host cell is an adult stem cell, a somatic stem cell, a non-embryonic stem cell, an embryonic stem cell, hematopoietic stem cell, an include pluripotent stem cells, and a trophoblast stem cell. In some embodiments, the stem cell line is naturally a Type O blood type cell line (i.e., the cell line is not genetically engineered to be a Type O blood type cell line). In some embodiments, the stem cell line is naturally a Rh⁻ (rhesus factor-negative) cell line. In some embodiments, the cell comprises a genetic disruption in the RHD gene. In some embodiments, the cell does not comprise a genetic disruption in the RHD gene. In some embodiments, the cell comprises a genetic disruption in the RHCE gene. In some embodiments, the cell does not comprise a genetic disruption in the RHCE gene.

Stem cells of interest include pluripotent stem cells (PSCs). The term “pluripotent stem cell” or “PSC” is used herein to mean a stem cell capable of producing all cell types of the organism. Therefore, a PSC can give rise to cells of all germ layers of the organism (e.g., the endoderm, mesoderm, and ectoderm of a vertebrate). Pluripotent cells are capable of forming teratomas and of contributing to ectoderm, mesoderm, or endoderm tissues in a living organism. Pluripotent stem cells of plants are capable of giving rise to all cell types of the plant (e.g., cells of the root, stem, leaves, etc.).

PSCs of animals can be derived in a number of different ways. For example, embryonic stem cells (ESCs) are derived from the inner cell mass of an embryo (Thomson et. Al, Science. 1998 Nov. 6; 282(5391):1145-7) whereas induced pluripotent stem cells (iPSCs) are derived from somatic cells (Takahashi et. Al, Cell. 2007 Nov. 30; 131(5):861-72; Takahashi et. Al, Nat Protoc. 2007; 2(12):3081-9; Yu et. Al, Science. 2007 Dec. 21; 318(5858):1917-20. Epub 2007 Nov. 20). Because the term PSC refers to pluripotent stem cells regardless of their derivation, the term PSC encompasses the terms ESC and iPSC, as well as the term embryonic germ stem cells (EGSC), which are another example of a PSC. PSCs can be in the form of an established cell line, they can be obtained directly from primary embryonic tissue, or they can be derived from a somatic cell.

By “embryonic stem cell” (ESC) is meant a PSC that is isolated from an embryo, typically from the inner cell mass of the blastocyst. ESC lines are listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hESI (MizMedi Hospital-Seoul National University); HSF-1, HSF-6 (University of California at San Francisco); and H1, H7, H9, H13, H14 (Wisconsin Alumni Research Foundation (WiCell Research Institute)). In some embodiments, the ESC is the Cyt49 (CVCL_B850) cell line. Stem cells of interest also include embryonic stem cells from other primates, such as Rhesus stem cells and marmoset stem cells. The stem cells can be obtained from any mammalian species, e.g. human, equine, bovine, porcine, canine, feline, rodent, e.g. mice, rats, hamster, primate, etc. (Thomson et al. (1998) Science 282:1145; Thomson et al. (1995) Proc. Natl. Acad. Sci USA 92:7844; Thomson et al. (1996) Biol. Reprod. 55:254; Shamblott et al., Proc. Natl. Acad. Sci. USA 95:13726, 1998). In preferred embodiments, the stem cells are human stem cells. In culture, ESCs typically grow as flat colonies with large nucleo-cytoplasmic ratios, defined borders and prominent nucleoli. In addition, ESCs express SSEA-3, SSEA-4, TRA-1-60, TRA-1-81, and Alkaline Phosphatase, but not SSEA-1. Examples of methods of generating and characterizing ESCs may be found in, for example, U.S. Pat. Nos. 7,029,913, 5,843,780, and 6,200,806, each of which is incorporated herein by its entirety. Methods for proliferating hESCs in the undifferentiated form are described in WO 99/20741, WO 01/51616, and WO 03/020920, each of which is incorporated herein by its entirety. In some embodiments, the ESC cell line is naturally a Type O blood type cell line (i.e., the cell line is not genetically engineered to be a Type O blood type cell line). In some embodiments, the ESC cell line is naturally a Rh (rhesus factor-negative) cell line. In some embodiments, the cell comprises a genetic disruption in the RHD gene. In some embodiments, the cell does not comprise a genetic disruption in the RHD gene. In some embodiments, the cell comprises a genetic disruption in the RHCE gene. In some embodiments, the cell does not comprise a genetic disruption in the RHCE gene.

By “embryonic germ stem cell” (EGSC) or “embryonic germ cell” or “EG cell,” it is meant a PSC that is derived from germ cells and/or germ cell progenitors, e.g. primordial germ cells, i.e. those that can become sperm and eggs. Embryonic germ cells (EG cells) are thought to have properties similar to embryonic stem cells as described above. Examples of methods of generating and characterizing EG cells may be found in, for example, U.S. Pat. No. 7,153,684; Matsui, Y., et al., (1992) Cell 70:841; Shamblott, M., et al. (2001) Proc. Natl. Acad. Sci. USA 98: 113; Shamblott, M., et al. (1998) Proc. Natl. Acad. Sci. USA, 95:13726; and Koshimizu, U., et al. (1996) Development, 122:1235, each of which are incorporated herein by its entirety.

By “induced pluripotent stem cell” or “iPSC,” it is meant a PSC that is derived from a cell that is not a PSC (i.e., from a cell this is differentiated relative to a PSC). iPSCs can be derived from multiple different cell types, including terminally differentiated cells. iPSCs have an ES cell-like morphology, growing as flat colonies with large nucleo-cytoplasmic ratios, defined borders and prominent nuclei. In addition, iPSCs express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. Examples of methods of generating and characterizing iPSCs can be found in, for example, U.S. Patent Publication Nos. US20090047263, US20090068742, US20090191159, US20090227032, US20090246875, and US20090304646, each of which are incorporated herein by its entirety. Generally, to generate iPSCs, somatic cells are provided with reprogramming factors (e.g. Oct4, SOX2, KLF4, MYC, Nanog, Lin28, etc.) known in the art to reprogram the somatic cells to become pluripotent stem cells.

By “somatic cell,” it is meant any cell in an organism that, in the absence of experimental manipulation, does not ordinarily give rise to all types of cells in an organism. In other words, somatic cells are cells that have differentiated sufficiently that they do not naturally generate cells of all three germ layers of the body, i.e. ectoderm, mesoderm and endoderm. For example, somatic cells can include both neurons and neural progenitors, the latter of which is able to naturally give rise to all or some cell types of the central nervous system but cannot give rise to cells of the mesoderm or endoderm lineages.

In certain examples, the stem cells can be undifferentiated (e.g. a cell not committed to a specific lineage) prior to exposure to at least one β cell maturation factor according to the methods as disclosed herein, whereas in other examples it may be desirable to differentiate the stem cells to one or more intermediate cell types prior to exposure of the at least one cell maturation factor(s) described herein. For example, the stems cells may display morphological, biological or physical characteristics of undifferentiated cells that can be used to distinguish them from differentiated cells of embryo or adult origin. In some examples, undifferentiated cells may appear in the two dimensions of a microscopic view in colonies of cells with high nuclear/cytoplasmic ratios and prominent nucleoli. The stem cells may be themselves (for example, without substantially any undifferentiated cells being present) or may be used in the presence of differentiated cells. In certain examples, the stem cells may be cultured in the presence of) suitable nutrients and optionally other cells such that the stem cells can grow and optionally differentiate. For example, embryonic fibroblasts or fibroblast-like cells may be present in the culture to assist in the growth of the stem cells. The fibroblast may be present during one stage of stem cell growth but not necessarily at all stages. For example, the fibroblast may be added to stem cell cultures in a first culturing stage and not added to the stem cell cultures in one or more subsequent culturing stages.

Stem cells used in all aspects of the present disclosure can be any cells derived from any kind of tissue (for example embryonic tissue such as fetal or pre-fetal tissue, or adult tissue), which stem cells have the characteristic of being capable under appropriate conditions of producing progeny of different cell types, e.g. derivatives of all of at least one of the 3 germinal layers (endoderm, mesoderm, and ectoderm). These cell types may be provided in the form of an established cell line, or they may be obtained directly from primary embryonic tissue and used immediately for differentiation. Included are cells listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hESI (MizMedi Hospital-Seoul National University); HSF-1, FISF-6 (University of California at San Francisco); and H1, H7, H9, H13, H14 (Wisconsin Alumni Research Foundation (WiCell Research Institute)). In some embodiments, the source of human stem cells or pluripotent stem cells used for chemically-induced differentiation into mature, insulin positive cells did not involve destroying a human embryo.

In another embodiment, the stem cells can be isolated from tissue including solid tissue. In some embodiments, the tissue is skin, fat tissue (e.g. adipose tissue), muscle tissue, heart or cardiac tissue. In other embodiments, the tissue is for example but not limited to, umbilical cord blood, placenta, bone marrow, or chondral.

Stem cells of interest also include embryonic cells of various types, exemplified by human embryonic stem (hES) cells, described by Thomson et al, (1998) Science 282:1145; embryonic stem cells from other primates, such as Rhesus stem cells (Thomson et al. (1995) Proc. Natl. Acad. Sci. USA 92:7844); marmoset stem cells (Thomson et al. (1996) Biol. Reprod. 55:254); and human embryonic germ (hEG) cells (Shambloft et al., Proc. Natl. Acad. Sci. USA 95:13726, 1998). Also of interest are lineage committed stem cells, such as mesodermal stem cells and other early cardiogenic cells (see Reyes et al, (2001) Blood 98:2615-2625; Eisenberg & Bader (1996) Circ Res. 78(2):205-16; etc.). The stem cells may be obtained from any mammalian species, e.g. human, equine, bovine, porcine, canine, feline, rodent, e.g. mice, rats, hamster, primate, etc. In some embodiments, a human embryo was not destroyed for the source of pluripotent cell used on the methods and compositions as disclosed herein.

A mixture of cells from a suitable source of endothelial, muscle, and/or neural stem cells can be harvested from a mammalian donor by methods known in the art. A suitable source is the hematopoietic microenvironment. For example, circulating peripheral blood, preferably mobilized (i.e., recruited), may be removed from a subject. In an embodiment, the stem cells can be reprogrammed stem cells, such as stem cells derived from somatic or differentiated cells. In such an embodiment, the de-differentiated stem cells can be for example, but not limited to, neoplastic cells, tumor cells and cancer cells or alternatively induced reprogrammed cells such as induced pluripotent stem cells or iPS cells.

In some embodiments, the SC-β cell can be derived from one or more of trichocytes, keratinocytes, gonadotropes, corticotropes, thyrotropes, somatotropes, lactotrophs, chromaffin cells, parafollicular cells, glomus cells melanocytes, nevus cells, Merkel cells, odontoblasts, cementoblasts corneal keratocytes, retina Muller cells, retinal pigment epithelium cells, neurons, glias (e.g., oligodendrocyte astrocytes), ependymocytes, pinealocytes, pneumocytes (e.g., type I pneumocytes, and type II pneumocytes), clara cells, goblet cells, G cells, β cells, ECL cells, gastric chief cells, parietal cells, foveolar cells, K cells, β cells, I cells, goblet cells, paneth cells, enterocytes, microfold cells, hepatocytes, hepatic stellate cells (e.g., Kupffer cells from mesoderm), cholecystocytes, centroacinar cells, pancreatic stellate cells, pancreatic α cells, pancreatic β cells, pancreatic δ cells, pancreatic F cells (e.g., PP cells), pancreatic ε cells, thyroid (e.g., follicular cells), parathyroid (e.g., parathyroid chief cells), oxyphil cells, urothelial cells, osteoblasts, osteocytes, chondroblasts, chondrocytes, fibroblasts, fibrocytes, myoblasts, myocytes, myosatellite cells, tendon cells, cardiac muscle cells, lipoblasts, adipocytes, interstitial cells of cajal, angioblasts, endothelial cells, mesangial cells (e.g., intraglomerular mesangial cells and extraglomerular mesangial cells), juxtaglomerular cells, macula densa cells, stromal cells, interstitial cells, telocytes simple epithelial cells, podocytes, kidney proximal tubule brush border cells, sertoli cells, leydig cells, granulosa cells, peg cells, germ cells, spermatozoon ovums, lymphocytes, myeloid cells, endothelial progenitor cells, endothelial stem cells, angioblasts, mesoangioblasts, pericyte mural cells, splenocytes (e.g., T lymphocytes, B lymphocytes, dendritic cells, microphages, leukocytes), trophoblast stem cells, or any combination thereof.

### SC-Islets

In some embodiments, any of the genetically engineered cells disclosed herein is an SC-islet cell. In some embodiments, the SC-islet cell is a NKX6.1⁺/ISL1⁺ cell. In some embodiments, the SC-islet cell is a NKX6.1/ISL1⁺ cell. In some embodiments, the SC-islet cell is a NKX6.1⁺/ISL1⁺ cell. In some embodiments, the SC-islet cell expresses insulin. In some embodiments, the SC-islet cell expresses glucagon. In some embodiments, the SC-islet cell expresses somatostatin. In some embodiments, the SC-islet cell is naturally a Type O blood type cell (i.e., the SC-islet cell or its precursor cells were not genetically engineered to be a Type O blood type cell line). In some embodiments, the SC-islet cell is naturally a Rh (rhesus factor-negative) cell. In some embodiments, the cell comprises a genetic disruption in the RHD gene. In some embodiments, the cell does not comprise a genetic disruption in the RHD gene. In some embodiments, the cell comprises a genetic disruption in the RHCE gene. In some embodiments, the cell does not comprise a genetic disruption in the RHCE gene.

In some embodiments, the disclosure provides for a composition comprising a population of genetically engineered SC-islet cells. In some embodiments, the compositions comprise no less than 50%, 40%, 30%, or 20% NKX6.1⁺/ISL1⁺ cells (e.g., as determined by flow cytometry). In some embodiments, no less than 30% of the cells in the composition are NKX6.1-positive, ISL1-positive cells, no less than 25% of the cells in the composition are NKX6.1-negative, ISL1-positive cells, less than 12% of the cells in the composition are NKX6.1-negative, ISL1-negative cells or between 9-25% of the cells in the composition are NKX6.1-positive, ISL1-negative cells (e.g., as determined by flow cytometry). In some embodiments, no less than 40%, 35%, 30%, 26%, 25%, or 20% of the cells in the composition are NKX6.1⁻/ISL1⁺ cells (e.g., as determined by flow cytometry). In some embodiments, no less than 26% of the cells in the composition are NKX6.1⁻/ISL1⁺ cells (e.g., as determined by flow cytometry). In some embodiments, between 5-25%, 5-40%, 5-35%, or 8-20% of the cells in the composition are NKX6.1⁻/ISL1⁺ cells (e.g., as determined by flow cytometry). In some embodiments, no more than 50%, 45%, 40%, 35%, 30%, or 25% of the cells in the composition are NKX6.1⁺/ISL1⁻ cells (e.g., as determined by flow cytometry). In some embodiments, no more than 50% of the cells in the composition are NKX6.1⁺/ISL1⁻ cells (e.g., as determined by flow cytometry).

In some embodiments, less than 12% of the cells (e.g., about 11%, about 10%, about 9%, about 8%, about 7%, about 6%, about 5%, about 4%, about 3%, about 2%, about 1%, or less) in the population are NKX6.1-negative, ISL1-negative cells. In some embodiments, less than 10%, less than 8%, less than 6%, less than 4%, or 1%-11%, 2%-10%, 2%-12%, 4%-12%, 6%-12%, 8%-12%, 2%-8%, 4%-8%, 3%-6% or 3%-5% of the cells in the population are NKX6.1-negative, ISL1-negative cells. In some embodiments, 2%-12%, 4%-12%, 6%-12%, 8%-12%, 2%-8%, 4%-8%, 3%-6% or 3%-5% of the cells in the population are NKX6.1-negative, ISL1-negative cells.

In some embodiments, at least 15% of the cells (e.g., about 15%, about 20%, about 25%, about 30%, about 35%, about 40%, about 45%, about 50%, about 55%, about 60% or more) in the population are NKX6.1-negative, ISL1-positive cells. In some embodiments, at least 15%, at least 20%, at least 25%, at least 30%, at least 35%, at least 40%, at least 45%, at least 50%, at least 55%, at least 60%, or 15%-60%, 15%-45%, 15%-30%, 30%-60%, 30%-45%, 45%-60% of the cells in the population are NKX6.1-negative, ISL1-positive cells. In some embodiments, 20%-60%, 20%-50%, 20%-40%, 20%-30%, 30%-60%, 30%-50%, 30%-40%, 40%-60%, 40%-50%, or 50%-60% of the cells in the population are NKX6.1-negative, ISL1-positive cells.

In some embodiments, at least 15% (e.g., 20%-60%, 20%-50%, 20%-40%, 20%-30%, 30%-60%, 30%-50%, 30%-40%, 40%-60%, 40%-50%, or 50%-60%) of the cells in the population are NKX6.1-negative, ISL1-positive cells and less than 12% (e.g., 2%-12%, 4%-12%, 6%-12%, 8%-12%, 2%-8%, 4%-8%, 3%-6% or 3%-5%) of the cells in the population are NKX6.1-negative, ISL1-negative cells.

In some embodiments, at least 60%, at least 65%, at least 70%, at least 73%, at least 74%, at least 75%, at least 80%, at least 85%, at least 90%, about 85-95%, or about 90-95% of the cells in the population are ISL1-positive cells. In some embodiments, 50-90%, 50-85%, 50-80%, 50-75%, 50-70%, 50-60%, 60-90%, 60-85%, 60-80%, 60-75%, 60-70%, 65-90%, 65-85%, 65-80%, 65-75%, 65-70%, 70-90%, 70-85%, 70-80%, 70-75%, 75-90%, 75-85%, 75-80%, 80-90%, 80-85%, or 85-90% of the cells in the population are ISL1-positive cells. In some embodiments, at least 74%, at least 75%, at least 80%, at least 85%, at least 90%, about 85-95%, or about 90-95% of the cells in the population are ISL1-positive cells. In some embodiments, about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or about 99% of the cells in the population are ISL1-positive cells.

In some embodiments, a population of in vitro differentiated cells described herein comprises more NKX6.1-negative, ISL1-positive cells than NKX6.1-positive, ISL1-positive cells. In some embodiments, at least 40% of the cells in the population are NKX6.1-negative, ISL1-positive cells. In some embodiments, at least 45%, at least 50%, about 40-50%, about 45-55%, or about 50-55% of the cells in the population are NKX6.1-negative, ISL1-positive cells. In some embodiments, about 40%, 41%, 42%, 43%, 44%, 45%, 46%, 47%, 48%, 49%, 50%, 51%, 52%, 53%, 54%, or about 55% of the cells in the population are NKX6.1-negative, ISL1-positive cells.

In some embodiments, at least 20% (e.g., at least 20%, at least 30%, at least 40%, at least 50%, at least 50%, at least 60% or more) of the ISL1-positive cells are NKX6.1-negative. In some embodiments, about 20%-60%, 20%-50%, 20%-40%, 20%-30%, 30%-60%, 30%-50%, 30%-40%, 40%-60%, 40%-50%, or 50%-60% of the ISL1-positive cells are NKX6.1-negative. In some embodiments, about 20%, 25%, 30%, 35%, 40%, 45%, 50%, 55%, 60% or more of the ISL1-positive cells are NKX6.1-negative.

In some embodiments, at least 20% (e.g., at least 20%, at least 30%, at least 40%, at least 50%, at least 50%, at least 60% or more) of the cells in the composition are ISL1-positive and NKX6.1-positive. In some embodiments, about 20%-60%, 20%-50%, 20%-40%, 20%-30%, 30%-60%, 30%-50%, 30%-40%, 40%-60%, 40%-50%, or 50%-60% of the cells in the composition are ISL1-positive and NKX6.1-positive. In some embodiments, about 20%, 25%, 30%, 35%, 40%, 45%, 50%, 55%, 60% or more of the cells in the composition are ISL1-positive and NKX6.1-positive.

In some embodiments, at least 20% (e.g., at least 20%, at least 30%, at least 40%, at least 50%, at least 50%, at least 60% or more) of the cells in the composition are ISL1-positive and NKX6.1-negative. In some embodiments, about 20%-60%, 20%-50%, 20%-40%, 20%-30%, 30%-60%, 30%-50%, 30%-40%, 40%-60%, 40%-50%, or 50%-60% of the cells in the composition are ISL1-positive and NKX6.1-negative. In some embodiments, about 20%, 25%, 30%, 35%, 40%, 45%, 50%, 55%, 60% or more of the cells in the composition are ISL1-positive and NKX6.1-negative.

In some embodiments, a population of in vitro differentiated cells described herein comprises up to 20% (e.g., up to 20%, up to 30%, up to 40% or up to 50%) of NKX6.1-positive, ISL1-positive cells. In some embodiments, a population of in vitro differentiated cells described herein comprises about 20%-50%, 20%-40%, 20%-30%, 30%-50%, 30%-40%, or 40%-50% of NKX6.1-positive, ISL1-positive cells. In some embodiments, a population of in vitro differentiated cells described herein comprises about 20%-50%, 20%-40%, 20%-30%, 30%-50%, 30%-40%, or 40%-50% of NKX6.1-positive, ISL1-positive cells.

In some embodiments, the NKX6.1-positive, ISL1-positive cells also express PDX1. In some embodiments, the NKX6.1-positive, ISL1-positive cells also express insulin. The NKX6.1-positive, ISL1-positive cells also express C-peptide. In some embodiments, the NKX6.1-positive, ISL1-positive cells also express chromogranin A.

In some embodiments, the disclosure provides for a composition comprising a plurality of genetically engineered cells (e.g., a composition comprising a cluster of cells or multiple clusters of cells); wherein 30-60%, 30-55%, 30-50%, 30-45%, 30-40%, 30-35%, 35-60%, 35-55%, 35-50%, 35-45%, 35-40%, 40-60%, 40-55%, 40-50%, 40-45%, 45-60%, 45-55%, 45-50%, 50-60%, or 50-55% of the cells in the composition are NKX6.1-positive, ISL1-positive cells; wherein 20-50%, 20-45%, 20-40%, 20-35%, 20-30%, 20-25%, 25-50%, 25-45%, 25-40%, 25-35%, 25-30%, 30-50%, 30-45%, 30-40%, 30-35%, 35-50%, 35-35%, 35-40%, 40-50%, 40-45%, or 45-50% of the cells in the composition are NKX6.1-negative, ISL1-positive cells; and wherein 1-12%, 1-10%, 1-8%, 1-6%, 1-4%, 3-5%, 1-2%, 2-12%, 2-10%, 2-8%, 2-6%, 2-4%, 4- 12%, 4-10%, 4-8%, 4-6%, 6-12%, 6-10%, 6-8%, 8-12%, 8-10%, or 10-12% of the cells in the composition are NKX6.1-negative, ISL1-negative cells. In some embodiments, the disclosure provides for a composition comprising a plurality of genetically engineered cells (e.g., a composition comprising a cluster of cells or multiple clusters of cells); wherein 35-50% of the cells in the composition are NKX6.1-positive, ISL1-positive cells; wherein 30-45% of the cells in the composition are NKX6.1-negative, ISL1-positive cells; and wherein 2-12% of the cells in the composition are NKX6.1-negative, ISL1-negative cells. In some embodiments, between 3-25%, 3-20%, 3-15%, 3-10%, 3-5%, 5-25%, 5-20%, 5-15%, 5-10%, 10-25%, 10-20%, 10-15%, 15-25%, 15-20% or 20-25% of the cells in the composition are NKX6.1-positive, ISL1-negative cells.

In some embodiments, the disclosure provides for a composition comprising a plurality of genetically engineered cells (e.g., a composition comprising a cluster of cells or multiple clusters of cells); wherein at least 30% of the cells in the composition are NKX6.1-positive, ISL 1-positive cells; wherein at least 25% of the cells in the composition are NKX6.1-negative, ISL1-positive cells; and wherein between 9-25% of the cells in the composition are NKX6.1-positive, ISL1-negative cells. In some embodiments, the disclosure provides for a composition comprising a plurality of genetically engineered cells (e.g., a composition comprising a cluster of cells or multiple clusters of cells); wherein 30-60%, 30-55%, 30-50%, 30-45%, 30-40%, 30-35%, 35-60%, 35-55%, 35-50%, 35-45%, 35-40%, 40-60%, 40-55%, 40-50%, 40-45%, 45-60%, 45-55%, 45-50%, 50-60%, or 50-55% of the cells in the composition are NKX6.1-positive, ISL1-positive cells; wherein 20-50%, 20-45%, 20-40%, 20-35%, 20-30%, 20-25%, 25-50%, 25-45%, 25-40%, 25-35%, 25-30%, 30-50%, 30-45%, 30-40%, 30-35%, 35-50%, 35-35%, 35-40%, 40-50%, 40-45%, or 45-50% of the cells in the composition are NKX6.1-negative, ISL1-positive cells; and wherein 9-30%, 9-25%, 9-20%, 9-15%, 9-12%, 12-30%, 12-25%, 12-20%, 12-15%, 15-30%, 15-25%, 15-20%, 20-30%, 20-25% or 25-30% of the cells in the composition are NKX6.1-positive ISL-negative cells. In some embodiments, 1-12%, 1-10%, 1-8%, 1-6%, 1-4%, 3-5%, 1-2%, 2-12%, 2-10%, 2-8%, 2-6%, 2-4%, 4-12%, 4-10%, 4-8%, 4-6%, 6-12%, 6- 10%, 6-8%, 8-12%, 8-10%, or 10-12% of the cells in the composition are NKX6.1-negative, ISL1-negative cells. In some embodiments, the disclosure provides for a composition comprising a plurality of genetically engineered cells (e.g., a composition comprising a cluster of cells or multiple clusters of cells); wherein 35-50% of the cells in the composition are NKX6.1-positive, ISL1-positive cells; wherein 30-45% of the cells in the composition are NKX6.1-negative, ISL1-positive cells; and wherein 9-25% of the cells in the composition are NKX6.1-positive, ISL1-negative cells.

In some embodiments, less than 12% of the cells (e.g., about 11%, about 10%, about 9%, about 8%, about 7%, about 6%, about 5%, about 4%, about 3%, about 2%, about 1%, or less) in the composition are NKX6.1-negative, ISL1-negative cells. In some embodiments, less than 10%, less than 8%, less than 6%, less than 4%, 1%-11%, 2%-10%, 2%-12%, 4%-12%, 6%-12%, 8%-12%, 2%-8%, 4%-8%, 3%-6% or 3%-5% of the cells in the composition are NKX6.1-negative, ISL1-negative cells. In some embodiments, 2%-12%, 4%-12%, 6%-12%, 8%-12%, 2%-8%, 4%-8%, 3%-6% or 3%-5% of the cells in the population are NKX6.1-negative, ISL1-negative cells.

In some embodiments, at least 15% of the cells (e.g., about 15%, about 20%, about 25%, about 30%, about 35%, about 40%, about 45%, about 50%, about 55%, about 60% or more) in the composition are NKX6.1-negative, ISL1-positive cells. In some embodiments, at least 15%, at least 20%, at least 25%, at least 30%, at least 35%, at least 40%, at least 45%, at least 50%, at least 55%, at least 60%, 15%-60%, 15%-45%, 15%-30%, 30%-60%, 30%-45%, 45%-60% of the cells in the composition are NKX6.1-negative, ISL1-positive cells. In some embodiments, 20%-60%, 20%-50%, 20%-40%, 20%-30%, 30%-60%, 30%-50%, 30%-40%, 40%-60%, 40%-50%, or 50%-60% of the cells in the composition are NKX6.1-negative, ISL1-positive cells.

In some embodiments, at least 15% (e.g., 20%-60%, 20%-50%, 20%-40%, 20%-30%, 30%-60%, 30%-50%, 30%-40%, 40%-60%, 40%-50%, or 50%-60%) of the cells in the composition are NKX6.1-negative, ISL1-positive cells and less than 12% (e.g., 2%-12%, 4%-12%, 6%-12%, 8%-12%, 2%-8%, 4%-8%, 3%-6% or 3%-5%) of the cells in the composition are NKX6.1-negative, ISL1-negative cells.

In some embodiments, at least 60%, at least 65%, at least 70%, at least 73%, at least 74%, at least 75%, at least 80%, at least 85%, at least 90%, about 85-95%, or about 90-95% of the cells in the composition are ISL1-positive cells. In some embodiments, 50-90%, 50-85%, 50-80%, 50-75%, 50-70%, 50-60%, 60-90%, 60-85%, 60-80%, 60-75%, 60-70%, 65-90%, 65-85%, 65-80%, 65-75%, 65-70%, 70-90%, 70-85%, 70-80%, 70-75%, 75-90%, 75-85%, 75-80%, 80-90%, 80-85%, or 85-90% of the cells in the composition are ISL1-positive cells. In some embodiments, at least 74%, at least 75%, at least 80%, at least 85%, at least 90%, about 85-95%, or about 90-95% of the cells in the composition are ISL1-positive cells. In some embodiments, about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or about 99% of the cells in the composition are ISL1-positive cells.

In some embodiments, the composition comprises more NKX6.1-positive, ISL1-positive cells that NKX6.1-negative, ISL1-positive cells. In some embodiments, at least 40% of the cells in the composition are NKX6.1-negative, ISL1-positive cells. In some embodiments, at least 45%, at least 50%, about 40-50%, about 45-55%, or about 50-55% of the cells in the composition are NKX6.1-negative, ISL1-positive cells. In some embodiments, about 40%, 41%, 42%, 43%, 44%, 45%, 46%, 47%, 48%, 49%, 50%, 51%, 52%, 53%, 54%, or about 55% of the cells in the composition are NKX6.1-negative, ISL1-positive cells.

In some embodiments, at least 20% (e.g., at least 20%, at least 30%, at least 40%, at least 50%, at least 50%, at least 60% or more) of the ISL1-positive cells are NKX6.1-negative. In some embodiments, about 20%-60%, 20%-50%, 20%-40%, 20%-30%, 30%-60%, 30%-50%, 30%-40%, 40%-60%, 40%-50%, or 50%-60% of the ISL1-positive cells are NKX6.1-negative. In some embodiments, about 20%, 25%, 30%, 35%, 40%, 45%, 50%, 55%, 60% or more of the ISL1-positive cells are NKX6.1-negative.

In some embodiments, the composition comprises at least 20% (e.g., at least 20%, 30%, 40%, 50% or 60%) of NKX6.1-positive, ISL1-positive cells. In some embodiments, the composition comprises about 20%-50%, 20%-40%, 20%-30%, 30%-50%, 30%-40%, 40%-50%, 40%-60%, or 50-60% of NKX6.1-positive, ISL1-positive cells. In some embodiments, the composition comprises about 20%-50%, 20%-40%, 20%-30%, 30%-50%, 30%-40%, or 40%-50% of NKX6.1-positive, ISL1-positive cells.

In some embodiments, the composition comprises less than 25% (e.g., less than 25%, less than 20%, less than 15%, less than 10%, less than 5%, or less) of NKX6.1-positive, ISL1-negative cells. In some embodiments, the composition comprises about 2%-25%, 2%-20%, 2%-15%, 2%-10%, 2%-5%, 5%-25%, 5%-20%, 5%-15%, 5%-10%, 10%-25%, 10%-20%, 10%-15%, 15%-25%, 15%-20%, or 20%-25% of NKX6.1-positive, ISL1-negative cells. In some embodiments, the composition comprises about 2%-10%, 2%-8%, 2%-6%, 2%-4%, 4%-10%, 4%-8%, 4%-6%, 6%-10%, 6%-8%, or 8%-10% of NKX6.1-positive, ISL1-negative cells. In some embodiments, the composition comprises about 2%, 4%, 6%, 8%, or 10% of NKX6.1-positive, ISL1-negative cells.

In some embodiments, the composition comprises less than 10% SOX9-positive cells. In some embodiments, the composition comprises less than 10%, 9%, 8%, 7%, 6%, 5%, 4%, 3%, 2% or 1% SOX9-positive cells. In some embodiments, the composition comprises 0.1-10%, 0.1-7%, 0.1-3%, 0.1-1%, 0.5-10%, 0.5-7%, 0.5-3%, 0.5-1%, 1-10%, 1-5%, 1-3%, 3-10%, 3-5%, or 5-10% SOX9-positive cells.

In some embodiments, the composition comprises less than 5% Ki67-positive cells. In some embodiments, the composition comprises less than 5%, 4%, 3%, 2% or 1% Ki67-positive cells. In some embodiments, the composition comprises 0.01-0.1%, 0.1-5%, 0.1-3%, 0.1-1%, 0.5-5%, 0.5-3%, 0.5-1%, 1-5%, 1-3%, or 1-2% Ki67-positive cells.

In some embodiments, at least 80%, 85%, 90%, 91%, 92%, 93%, 94%, 95%, 96%, 97%, 98%, or 99% of the cells in the composition are CHGA-positive cells. In some embodiments, 80-100%, 85-100%, 90-100%, 90-99%, 90-98%, 95-99%, or 95-99% of the cells in the composition are CHGA-positive cells.

In some embodiments, the percentage of cells expressing a marker provided herein is measured by flow cytometry. The skilled worker is aware of representative methods for testing whether a cell or collection of cells is positive or negative for expression of a specific gene marker (e.g., NKX6.1, ISL1, INS, GCG, somatostatin, chromogranin A, SOX9, C-peptide, Ki67) by flow cytometry. In some embodiments, a cell is considered positive for expression of a particular gene (e.g., NKX6.1, ISL1, INS, GCG, somatostatin, chromogranin A, SOX9, C-peptide, Ki67) based on median fluorescence intensity (rMFI). As used herein, the term “rMFI” or relative median fluorescence intensity is the ratio between the fluorescence intensity measured by use of an antibody to a specific target (e.g., NKX6.1, ISL1, INS, GCG, somatostatin, chromogranin A, SOX9, C-peptide, Ki67) versus the intensity obtained from a control antibody (isotype control). In some embodiments, an anti-(human) NKX6.1, ISL1, INS, GCG, somatostatin, chromogranin A, or SOX9 antibody is used. Examples of suitable antibodies for use in flow cytometry are any of the antibodies disclosed in Table 1. An example of a suitable flow cytometer is the Accuri 6 flow cytometer. In some embodiments, the target-expressing cells (e.g., cells expressing NKX6.1 and/or ISL1), if tested, exhibit a target relative medium fluorescence intensity (rMFI) of at least 6, 6.5, 7, 8, 9 or 10 as measured by flow cytometry. In another embodiment, said rMFI is between 6.5 and 15, between 6.5 and 14, between 6.5 and 13, between 6.5 and 13, between 6.5 and 12, or between 6.5 and 10.

In some embodiments, the percentage of cells expressing a marker provided herein is measured by qRT-PCR. In some embodiments, the percentage of cells expressing a marker provided herein is measured by single cell RNA sequencing analysis. The skilled worker is aware of methods for testing whether a cell or collection of cells is positive for expression of a specific gene marker (e.g., NKX6.1, ISL1, INS, GCG, ARX, or ghrelin) by single cell RNA sequencing analysis.

In some embodiments, a population of genetically engineered cells described herein comprises less than 25% (e.g., less than 25%, less than 20%, less than 15%, less than 10%, less than 5%, or less) of NKX6.1-positive, ISL1-negative cells. In some embodiments, a population of genetically engineered cells described herein comprises about 2%-25%, 2%-20%, 2%-15%, 2%-10%, 2%-5%, 5%-25%, 5%-20%, 5%-15%, 5%-10%, 10%-25%, 10%-20%, 10%-15%, 15%-25%, 15%-20%, or 20%-25% of NKX6.1-positive, ISL1-negative cells. In some embodiments, a population of genetically engineered cells described herein comprises about 2%-10%, 2%-8%, 2%-6%, 2%-4%, 4%-10%, 4%-8%, 4%-6%, 6%-10%, 6%-8%, or 8%-10% of NKX6.1-positive, ISL1-negative cells. In some embodiments, a population of genetically engineered cells described herein comprises about 2%, 4%, 6%, 8%, or 10% of NKX6.1-positive, ISL1-negative cells.

TABLE 1

| | | | Primary | Secondary | |
|---|---|---|---|---|---|
| Primary | | | Antibody | Antibody | |
| Antibody | Company | Cat# | Species | Species | Cat# |
| NKX6.1 | DSHB | FSSA12 | Mouse | anti-Mouse | A21202 |
| | | | | 488 | |
| IsL1 | abcam | ab178400 | Rabbit | anti-Rabbit- | A31573 |
| | | | | 647 | |
| SST | Santa | SC- | Anti-Somatostatin | AlexaFluor ® | — |
| | Cruz | 55565AF647 | Antibody (G-10) Alexa | 647 | |
| | Biotechnology | | Fluor ® 647 | | |
| Glu | R&D | IC 1249G | Human/Mouse | AlexaFluor ®488 | — |
| | | | Glucagon Alexa Fluor ® | | |
| | | | 488-conjugated | | |
| Sox9 | Epitomics | AC- | Rabbit | anti-Rabbit- | A31573 |
| | | 0284RUOC | | 647 | |
| Ki67 | Thermo | | Ki-67 Monoclonal | PE | — |
| | Fisher | | Antibody (SolA15), PE | | |
| C-pep | DSHB | GN-1D4-S | Rat | anti-Rat-488 | A21208 |
| CHGA | abcam | ab15160 | Rabbit | anti-Rabbit- | A31573 |
| | | | | 647 | |

In some embodiments, a population of genetically engineered cells described herein comprises more NKX6.1-negative, ISL1-positive cells than NKX6.1-positive, ISL1-positive cells. In some embodiments, the population comprises more NKX6.1-positive, ISL1-positive cells that NKX6.1-negative, ISL1-positive cells. In some embodiments, at least 30% or 40% of the cells in the population are NKX6.1-negative, ISL1-positive cells. In some embodiments, at least 45%, at least 50%, about 25-50%, 20-50%, 20-40%, 20-55%, 25-40%, 30-45%, 30-40%, about 40-50%, about 45-55%, or about 50-55% of the cells in the population are NKX6.1-negative, ISL1-positive cells. In some embodiments, about 40%, 41%, 42%, 43%, 44%, 45%, 46%, 47%, 48%, 49%, 50%, 51%, 52%, 53%, 54%, or about 55% of the cells in the population are NKX6.1-negative, ISL1-positive cells.

In some embodiments, at least 20% (e.g., at least 20%, at least 30%, at least 40%, at least 50%, at least 50%, at least 60% or more) of the ISL1-positive cells are NKX6.1-negative. In some embodiments, about 20%-60%, 20%-50%, 20%-40%, 20%-30%, 30%-60%, 30%-50%, 30%-40%, 40%-60%, 40%-50%, or 50%-60% of the ISL1-positive cells are NKX6.1-negative. In some embodiments, about 20%, 25%, 30%, 35%, 40%, 45%, 50%, 55%, 60% or more of the ISL1-positive cells are NKX6.1-negative.

In some embodiments, a population of genetically engineered cells described herein comprises at least 20% (e.g., at least 20%, 30%, 40%, 50% or 60%) of NKX6.1-positive, ISL1-positive cells. In some embodiments, a population of genetically engineered cells described herein comprises about 20%-50%, 20%-40%, 20%-30%, 30%-50%, 30%-40%, 40%-50%, 40%-60%, or 50-60% of NKX6.1-positive, ISL1-positive cells. In some embodiments, a population of genetically engineered cells described herein comprises about 20%-50%, 20%-40%, 20%-30%, 30%-50%, 30%-40%, or 40%-50% of NKX6.1-positive, ISL1-positive cells.

In some embodiments, a population of genetically engineered cells described herein comprises less than 25% (e.g., less than 25%, less than 20%, less than 15%, less than 10%, less than 5%, or less) of NKX6.1-positive, ISL1-negative cells. In some embodiments, a population of genetically engineered cells described herein comprises about 2%-25%, 2%-20%, 2%-15%, 2%-10%, 2%-5%, 5%-25%, 5%-20%, 5%-15%, 5%-10%, 10%-25%, 10%-20%, 10%-15%, 15%-25%, 15%-20%, or 20%-25% of NKX6.1-positive, ISL1-negative cells. In some embodiments, a population of genetically engineered cells described herein comprises about 2%-10%, 2%-8%, 2%-6%, 2%-4%, 4%-10%, 4%-8%, 4%-6%, 6%-10%, 6%-8%, or 8%-10% of NKX6.1-positive, ISL1-negative cells. In some embodiments, a population of genetically engineered cells described herein comprises about 2%, 4%, 6%, 8%, or 10% of NKX6.1-positive, ISL1-negative cells.

In some embodiments, a population of genetically engineered cells described herein comprises ghrelin-positive cells. In some embodiments, a population of genetically engineered cells described herein comprises less than 5% (e.g., less than 5%, less than 3%, less than 2%, less than 1% or less than 0.5%) ghrelin-positive cells. In some embodiments, a population of genetically engineered cells described herein comprises at least 0.05% (e.g., at least 0.05%, 0.1%, 0.2%, 0.3%, 0.4%, 0.5%, 0.6%, 0.8%, 1%, 1.2%, 1.5%, 1.8%, 2%, 3%, 4%, or 5%) ghrelin-positive cells. In some embodiments, a population of genetically engineered cells described herein comprises 1-5%, 2-5%, 3-5%, 0.1-5%, 0.1-3%, 0.1-2%, 0.1-1%, 0.5-5%, 0.5-3%, 0.5-2%, 0.5-1%, 0.5-0.8%, 0.05-1%, 0.05-0.7%, or 0.05-2% ghrelin-positive cells.

In some embodiments, the disclosure provides for a composition comprising genetically engineered NKX6.1-positive, ISL1-positive cells that express lower levels of MAFA than NKX6.1-positive, ISL1-positive cells from the pancreas of a healthy control adult subject or from a cadaveric pancreas. In some embodiments, the pharmaceutical composition comprises genetically engineered NKX6.1-positive, ISL1-positive cells that express higher levels of MAFB than NKX6.1-positive, ISL1-positive cells from the pancreas of a healthy control adult subject or from a cadaveric pancreas. In some embodiments, the pharmaceutical composition comprises genetically engineered NKX6.1-positive, ISL1-positive cells that express higher levels of SIX2, HOPX, IAPP and/or UCN3 than NKX6.1-positive, ISL1-positive cells from the pancreas of a healthy control adult subject or from a cadaveric pancreas. In some embodiments, the pharmaceutical composition comprises genetically engineered NKX6.1-positive, ISL1-positive cells that do not express MAFA. In some embodiments, the pharmaceutical composition comprises genetically engineered NKX6.1-positive, ISL1-positive cells that express MAFB. In some embodiments, the pharmaceutical composition comprises cells that are genetically modified (e.g., using a gene editing technology such as CRISPR). In some embodiments, the pharmaceutical composition comprises genetically engineered NKX6.1-positive, ISL1-positive cells that express lower levels of beta-2 microglobulin, CIITA, HLA-A, HLA-B, HLA-C, HLA-DP, HLA-DQ, and HLADR than NKX6.1-positive, ISL1-positive cells from the pancreas of a healthy control adult subject or from a cadaveric pancreas. In some embodiments, the pharmaceutical composition comprises genetically engineered NKX6.1-positive, ISL1-positive cells that express increased levels of CD47, PDL1, HLA-G, CD46, CD55, CD59 and CTLA than NKX6.1-positive, ISL1-positive cells from the pancreas of a healthy control adult subject or from a cadaveric pancreas. In some embodiments, any of the cell markers disclosed herein (e.g., NKX6.1, PDX1, MAFA, MAFB, SIX2, HOPX, IAPP and/or UCN3) are detected by flow cytometry.

In some embodiments, any of the cells disclosed herein have not been genetically modified to have reduced expression of PDL1. In some embodiments, any of the cells disclosed herein have not been genetically modified to have reduced MHC Class II protein expression. In some embodiments, any of the cells disclosed herein have not been genetically modified to have reduced CIITA protein expression. In some embodiments, any of the genetically modified cells disclosed herein do not have lower MHC Class II protein expression as compared to the same type of cell that has not been genetically modified. In some embodiments, any of the genetically modified cells disclosed herein do not have lower CIITA protein expression as compared to the same type of cell that has not been genetically modified. In some embodiments, any of the cells disclosed herein do not comprise a genetic alteration in any of the HLA-DR, HLA-DP, or HLA-DQ genes. In some embodiments, any of the cells disclosed herein do not comprise a genetic alteration in the CIITA gene.

In some embodiments, any of the genetically modified cells disclosed herein do not comprise reduced expression of Rh protein antigen expression selected from the group consisting of Rh C antigen, Rh E antigen, Kell K antigen (KEL), Duffy (FY) Fya antigen, Duffy Fy3 antigen, Kidd (JK) Jkb antigen, MNS antigen U, and MNS antigen S as compared to a cell of the same type that is not genetically modified. In some embodiments, any of the cells disclosed herein do not have a genetic alteration in the RHD and/or RHCE genes.

In some cases, cell populations or cell clusters disclosed herein are unsorted, e.g., isolated cell populations or cell clusters that have not been through cell sorting process. In some embodiments, the cell cluster disclosed herein can refer to a cell cluster formed by self-aggregation of cells cultured in a given environment, for instance, in a 3D suspension culture. Cell sorting as described herein can refer to a process of isolating a group of cells from a plurality of cells by relying on differences in cell size, shape (morphology), surface protein expression, endogenous signal protein expression, or any combination thereof. In some cases, cell sorting comprises subjecting the cells to flow cytometry. Flow cytometry can be a laser- or impedance-based, biophysical technology. During flow cytometry, one can suspend cells in a stream of fluid and pass them through an electronic detection apparatus. In one type of flow cytometry, fluorescent-activated cell sorting (FACS), based on one or more parameters of the cells' optical properties (e.g., emission wavelength upon laser excitation), one can physically separate and thereby purify cells of interest using flow cytometry. As described herein, an unsorted cell cluster can be cell cluster that formed by a plurality of cells that have not been subject to an active cell sorting process, e.g., flow cytometry. In some cases, flow cytometry as discussed herein can be based on one or more signal peptides expressed in the cells. For example, a cell cluster can comprise cells that express a signal peptide (e.g., a fluorescent protein, e.g., green fluorescent protein (GFP) or tdTomato). In some cases, the signal peptide is expressed as an indicator of insulin expression in the cells. For instance, a cell cluster can comprise cell harboring an exogenous nucleic acid sequence coding for GFP under the control of an insulin promoter. The insulin promoter can be an endogenous or exogenous promoter. In some cases, the expression of GFP in these cells can be indicative of insulin expression in the cells. The GFP signal can thus be a marker of a pancreatic β cell. In some cases, cell sorting as described herein can comprise subjecting cells to magnetic-activated sorting process, where magnetic antibody or other ligand is used to label cells of different types, and the differences in magnetic properties can be used for cell sorting.

The percentage of cells expressing one or more particular markers, like PDX1, NKX6.1, insulin, NGN3, or CHGA, described herein can be the percentage value detected using techniques like flow cytometry assay. In some cases, during a flow cytometry assay, cell population or cell cluster discussed herein are dispersed into single-cell suspension by incubation in digesting enzyme like trypsin or TrypLE™ Express. Dispersed cell can be washed in suitable buffer like PBS, centrifuged and then re-suspended in fixation buffer like 4% PFA. Incubation with primary antibodies against the cell markers of interest can then be conducted, which can be followed by incubation with the secondary antibodies. After antibody incubation, the cells can be washed and the subject to segregation by flow cytometry. Techniques other than flow cytometry can also be used to characterize the cells described herein, e.g., determine the cell percentages. Non-limiting examples of cell characterization methods include gene sequencing, microscopic techniques (fluorescence microscopy, atomic force microscopy), karyotyping, isoenzyme analysis, DNA properties, viral susceptibility.

In some embodiments, in any of the composition disclosed herein, at least a portion of the genetically engineered cells in the population of genetically engineered cells are present in plurality of cell clusters. In some cases, the cell clusters are about 50 μm to about 500 μm, about 50 μm to about 400 μm, about 50 μm to about 300 μm, about 60 μm to about 400 μm, about 60 μm to about 300 μm, about 60 μm to about 250 μm, about 75 μm to about 400 μm, about 75 μm to about 300 μm, about 75 μm to about 250 μm, about 125 μm to about 225 μm, about 130 μm to about 160 μm, about 170 μm to about 225 μm, about 140 μm to about 200 μm, about 140 μm to about 170 μm, about 160 μm to about 220 μm, about 170 μm to about 215 μm, or about 170 μm to about 200 μm in diameter. In some cases, in the pharmaceutical compositions disclosed herein, the population of cells are present as a single cell suspension. In some embodiments, in the pharmaceutical compositions disclosed herein, at least 50%, at least 60%, at least 70%, at least 75%, at least 80%, at least 85%, at least 90%, at least 92%, at least 94%, at least 95%, at least 96%, at least 98%, or at least 99% of the cells are present in cell clusters. In some embodiments, in the pharmaceutical compositions disclosed herein, substantially all of the cells are present in cell clusters, e.g., at least 99.5%, at least 99.6%, at least 99.7%, at least 99.8%, at least 99.9%, at least 99.9%, at least 99.99%, at least 99.999%, or at least 99.9999% of the cells.

In some embodiments, a cell cluster is between about 80 and 270 microns in diameter. In some embodiments, a cell cluster is between about 100 and about 250 microns in diameter (e.g., about 125, about 140, about 150, about 160, about 170, about 180, about 190, about 200, about 200, about 210, about 215, about 220, or about 225, microns in diameter). For example, in some embodiments, the cell cluster is between about 125 and about 225, between about 130 and about 160, between about 170 and about 225, between about 140 and about 200, between about 140 and about 170, between about 160 and about 220, between about 170 and about 215, or between about 170 and about 200, microns in diameter.

In some embodiments, the disclosure provides for a composition comprising one or more cell clusters. In some embodiments, the composition comprises 500-20000, 500-15000, 500-10000, 500-5000, 500-2000, 500-1000, 1000-20000, 1000-15000, 1000-10000, 1000-5000, 1000-2000, 2000-20000, 2000-15000, 2000-10000, 2000-5000, 5000-20000, 5000-15000, 5000-10000, 10000-20000, 10000-15000, 15000-20000, or 3000-9000 cell clusters.

### Methods of Producing Pancreatic Islet Cells

In certain aspects, the present disclosure relates to compositions and methods of generating endocrine cells from genetically engineered pancreatic progenitor cells or genetically engineered precursors. Certain exemplary detailed protocols of generating endocrine cells to provide at least one SC-β cell are described in U.S. Patent Application Publication Nos. US20150240212, US20150218522, US20210238553, and US 2022-0090020 each of which is herein incorporated by reference in its entirety.

In some embodiments, a method of generating a population of endocrine cells leads to increased percentage of pancreatic α and/or δ cells and decreased percentage of pancreatic EC cells when generating pancreatic β cells. In some embodiments, a method described herein may be used to obtain an enriched population of α cells. In some embodiments, a method described herein may be used to obtain an enriched population of β cells. In some embodiments, a method described herein may be used to obtain an enriched population of α cells and β cells. In some embodiments, a method described herein may be used to obtain an increased yield of pancreatic endocrine cells.

The successful differentiation to pancreatic β cells should require that differentiated cells synthesize and secrete physiologically appropriate amounts of insulin. The differentiation of hPSC cells to hormone-expressing pancreatic endocrine cells is conducted by transiting hPSC cells through major stages of embryonic development; differentiation to mesendoderm and definitive endoderm, establishment of the primitive gut endoderm, patterning of the posterior foregut, and specification and maturation of pancreatic endoderm and endocrine precursors. Through these stages, hPSC cells can obtain pancreatic endocrine phenotype and ability of glucose responsive insulin secretion in vitro.

Generally, the at least one pancreatic α, β and/or δ cell or precursor thereof, e.g., pancreatic progenitors produced according to the methods disclosed herein can comprise a mixture or combination of different cells, e.g., for example a mixture of cells such as a PDX1-positive pancreatic progenitors, pancreatic progenitors co-expressing PDX1 and NKX6-1, a Ngn3-positive endocrine progenitor cell, an insulin-positive endocrine cell (e.g., NKX6.1-positive, ISL1-positive cells, or β-like cells), and/or other pluripotent or stem cells.

The at least one pancreatic α, β and/or δ cell or precursor thereof can be produced according to any suitable culturing protocol to differentiate a stem cell or pluripotent cell to a desired stage of differentiation. In some embodiments, the at least one pancreatic α, β and/or δ cell or the precursor thereof are produced by culturing at least one pluripotent cell for a period of time and under conditions suitable for the at least one pluripotent cell to differentiate into the at least one pancreatic α, β and/or δ cell or the precursor thereof.

In some embodiments, the at least one pancreatic α, β and/or δ cell or precursor thereof is a substantially pure population of pancreatic α, β and/or δ cells or precursors thereof. In some embodiments, a population of pancreatic α, β and/or δ cells or precursors thereof comprises a mixture of pluripotent cells or differentiated cells. In some embodiments, a population pancreatic α, β and/or δ cells or precursors thereof are substantially free or devoid of embryonic stem cells or pluripotent cells or iPS cells. In some embodiments, a method described herein produces a population of cells comprising pancreatic α, β and/or δ cells at a ratio that resembles that of a natural pancreatic islet.

In some embodiments, a method described herein comprises (i) culturing a first population of cells comprising pancreatic progenitor cells (e.g., cells that are PDX1-positive, NKX6.1-negative; or a mixture of cells that are PDX1-positive, NKX6.1-negative and cells that are PDX1-positive, NKX6.1-positive) in a first medium comprising a Forkhead Box O1 (FoxO1) inhibitor and a notch signaling pathway inhibitor for a period of time to obtain a second population of cells (e.g., a population of cells that comprises more PDX1-positive, NKX6.1-positive cells than the first population); and (ii) culturing the second population of cells in a second medium comprising a PKC activator and a Wnt signaling pathway inhibitor. In some embodiments, the method generates a population of cells comprising cells that are PDX1-positive, NKX6.1-positive, and insulin-positive.

In some embodiments, a method described herein comprises culturing a first population of cells in a first medium, wherein the first population of cells comprises pancreatic progenitor cells that are PDX1-positive and NKX6.1 negative, and pancreatic progenitor cells that are PDX1-positive and NKX6.1 positive; and the first medium comprises a Forkhead Box O1 (FoxO1) inhibitor (e.g., AS1842856 or a derivative thereof). In some embodiments, the first medium further comprises a notch signaling pathway inhibitor. In some embodiments, the notch signaling pathway inhibitor is a γ-secretase inhibitor (e.g., XXI, DAPT or derivatives thereof). In some embodiments, the γ-secretase inhibitor is XXI. In some embodiments, the first medium does not comprise a Wnt signaling pathway inhibitor.

In some embodiments, the first population of cells comprises pancreatic progenitor cells that are PDX1-positive and NKX6.1 positive. In some embodiments, the first population of cells comprises more pancreatic progenitor cells that are PDX1-positive and NKX6.1 negative than pancreatic progenitor cells that are PDX1-positive and NKX6.1 positive. In some embodiments, the first population of cells comprises more pancreatic progenitor cells that are PDX1-positive and NKX6.1 positive than pancreatic progenitor cells that are PDX1-positive and NKX6.1 negative.

In some embodiments, first medium further comprises a PKC activator (e.g., PdBU, TPB, phorbol 12-myristate 13-acetate, bryostatin 1, or derivatives thereof). In some embodiments, the PKC activator is PdBU. In some embodiments, the first medium further comprises one or more (e.g., 1, 2, 3, 4, 5) agents selected from a fibroblast growth factor (e.g., KGF), a sonic hedgehog (SHH) signaling pathway inhibitor (e.g., SANT-1), retinoic acid, a Rho-associated, coiled-coil containing protein kinase (ROCK) inhibitor (e.g., triazovivin), and a TGF-β ligand (e.g., activin A). In some embodiments, the first medium further comprises a water-soluble synthetic polymer (e.g., PVA such as PVA80%). In some embodiments, the first medium comprises a FoxO1 inhibitor (e.g., AS1842856 or a derivative thereof), a notch signaling pathway inhibitor (e.g., γ-secretase inhibitor such as XXI), a PKC activator (e.g., PdBU), a fibroblast growth factor (e.g., KGF), a sonic hedgehog (SHH) signaling pathway inhibitor (e.g., SANT-1), retinoic acid, a Rho-associated, coiled-coil containing protein kinase (ROCK) inhibitor (e.g., triazovivin), and a TGF-β ligand (e.g., activin A), and a water-soluble synthetic polymer (e.g., PVA such as PVA80%).

In some embodiments, the first population of cells are cultured in the first medium for a period of about 12-72 hours (e.g., about 12-72 hours, 12-66 hours, 12-60 hours, 12-54 hours, 12-48 hours, 12-42 hours, 12-36 hours, 12-30 hours, 12-24 hours, 12-18 hours, 18-72 hours, 18-66 hours, 18-60 hours, 18-54 hours, 18-48 hours, 18-42 hours, 18-36 hours, 18-30 hours, 18-24 hours, 24-72 hours, 24-66 hours, 24-60 hours, 24-54 hours, 24-48 hours, 24-42 hours, 24-36 hours, 24-30 hours, 30-72 hours, 30-66 hours, 30-60 hours, 30-54 hours, 30-48 hours, 30-42 hours, 30-36 hours, 36-72 hours, 36-66 hours, 36-60 hours, 36-54 hours, 36-48 hours, 36-42 hours, 42-72 hours, 42-66 hours, 42-60 hours, 42-54 hours, 42-48 hours, 48-72 hours, 48-66 hours, 48-60 hours, 48-54 hours, 54-72 hours, 54-66 hours, 54-60 hours, 60-72 hours, 60-66 hours, or 66-72 hours). In some embodiments, the first population of cells are cultured in the first medium for a period of about 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 hours. In some embodiments, the first population of cells are cultured in the first medium for a period of about 24 hours. In some embodiments, the first population of cells are cultured in the first medium for a period of about 48 hours.

In some embodiments, culturing the first population of cells in the first media for a contacting period described herein (e.g., 24 or 48 hours) results in a second population of cells. In some embodiments, the second population of cells comprises pancreatic progenitor cells that are PDX1-positive and NKX6.1 positive and pancreatic progenitor cells that are PDX1-positive and NKX6.1 negative. In some embodiments, the second population of cells comprises more pancreatic progenitor cells that are PDX1-positive and NKX6.1-positive than the first population of cells. In some embodiments, the second population of cells comprises more pancreatic progenitor cells that are PDX1-positive and NKX6.1 positive than pancreatic progenitor cells that are PDX1-positive and NKX6.1 negative. In some embodiments, the second population of cells comprises trace amounts (e.g., less than 5%, less than 4%, less than 3%, less than 2%, less than 1% of the second population of cells) of pancreatic progenitor cells that are PDX1-positive and NKX6.1 negative.

In some embodiments, a method described herein further comprises culturing the second population of cells with a second medium comprising a Wnt signaling pathway inhibitor (e.g., a tankyrase inhibitor such as NVP-TNKS656). In some embodiments, the second medium comprises a PKC activator (e.g., PdBu). In some embodiments, the second medium further comprises one or more (e.g., 1, 2, 3, 4, 5, 6, 7, 8, 9, 10) agents selected from an epidermal growth factor (e.g., betacellulin), a thyroid hormone (e.g., GC-1), a TGFβ-R1 kinase inhibitor (e.g., ALK5i), a notch signaling pathway inhibitor (e.g., a γ-secretase inhibitor such as XXI), a sonic hedgehog (SHH) signaling pathway inhibitor (e.g., SANT-1), retinoic acid, a Rho-associated, coiled-coil containing protein kinase (ROCK) inhibitor (e.g., triazovivin), a protein kinase inhibitor (e.g., staurosporine), a bone morphogenetic (BMP) signaling pathway inhibitor (e.g., LDN193189), and a histone methyltransferase EZH2 inhibitor (e.g., DZNep). In some embodiments, the second medium further comprises one or more (e.g., 1, 2, 3, 4) agents selected from an acetyl COA related metabolite (e.g., acetate), an HDAC inhibitor (e.g., β-hydroxybutyrate), a redox homeostasis regulator (e.g., taurine), and a one carbon metabolism pathway intermediate (e.g., formate). In some embodiments, the second medium further comprises a vitamin (e.g., biotin). In some embodiments, the second medium further comprises glutamine. In some embodiments, the second medium further comprises a water soluble synthetic polymer (e.g., PVA such as PVA 87-89%). In some embodiments, the second medium does not comprise a FOXO1 inhibitor. In some embodiments, the second medium comprises a Wnt signaling pathway inhibitor (e.g., a tankyrase inhibitor such as NVP-TNKS656), a PKC activator (e.g., PdBu), an epidermal growth factor (e.g., betacellulin), a thyroid hormone (e.g., GC-1), a TGFβ-R1 kinase inhibitor (e.g., ALK5i), a notch signaling pathway inhibitor (e.g., a γ-secretase inhibitor such as XXI), a sonic hedgehog (SHH) signaling pathway inhibitor (e.g., SANT-1), retinoic acid, a Rho-associated, coiled-coil containing protein kinase (ROCK) inhibitor (e.g., triazovivin), a protein kinase inhibitor (e.g., staurosporine), a bone morphogenetic (BMP) signaling pathway inhibitor (e.g., LDN193189), a histone methyltransferase EZH2 inhibitor (e.g., DZNep), an acetyl CoA related metabolite (e.g., acetate), an HDAC inhibitor (e.g., β-hydroxybutyrate), a redox homeostasis regulator (e.g., taurine), a one carbon metabolism pathway intermediate (e.g., formate), a vitamin (e.g., biotin), glutamine and a water soluble synthetic polymer (e.g., PVA such as PVA 87-89%), and does not comprise a FOXO1 inhibitor.

In some embodiments, the second population of cells are cultured in the second medium for a period of about 12-72 hours (e.g., about 12-72 hours, 12-66 hours, 12-60 hours, 12-54 hours, 12-48 hours, 12-42 hours, 12-36 hours, 12-30 hours, 12-24 hours, 12-18 hours, 18-72 hours, 18-66 hours, 18-60 hours, 18-54 hours, 18-48 hours, 18-42 hours, 18-36 hours, 18-30 hours, 18-24 hours, 24-72 hours, 24-66 hours, 24-60 hours, 24-54 hours, 24-48 hours, 24-42 hours, 24-36 hours, 24-30 hours, 30-72 hours, 30-66 hours, 30-60 hours, 30-54 hours, 30-48 hours, 30-42 hours, 30-36 hours, 36-72 hours, 36-66 hours, 36-60 hours, 36-54 hours, 36-48 hours, 36-42 hours, 42-72 hours, 42-66 hours, 42-60 hours, 42-54 hours, 42-48 hours, 48-72 hours, 48-66 hours, 48-60 hours, 48-54 hours, 54-72 hours, 54-66 hours, 54-60 hours, 60-72 hours, 60-66 hours, or 66-72 hours). In some embodiments, the second population of cells are cultured in the second medium for a period of about 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 hours. In some embodiments, the second population of cells are cultured in the second medium for a period of about 48 hours.

In some embodiments, culturing the second population of cells in the second media for a contacting period described herein (e.g., 48 hours) results in a third population of cells. In some embodiments, the third population of cells comprises pancreatic progenitor cells that are PDX1-positive and NKX6.1-positive. In some embodiments, the third population of cells comprises cells that are ISL1-positive. In some embodiments, the third population of cells comprises cells that are ISL1-negative. In some embodiments, the third population of cells comprises cells that are ISL1-positive. In some embodiments, the third population of cells comprises more cells that are ISL1-positive than the first and second population of cells. In some embodiments, the third population of cells comprises more cells that are ISL1-negative than cells that are ISL1-positive. In some embodiments, the third population of cells comprises cells that are insulin-negative. In some embodiments, the third population of cells comprises cells that are insulin-positive. In some embodiments, the third population of cells comprises more cells that are insulin-negative than cells that are insulin-positive. In some embodiments, the third population of cells comprises more cells that are insulin-positive than the first and second population of cells.

In some embodiments, the method further comprises culturing the third population of cells in a third medium comprising one or more agents selected from: a notch signaling pathway inhibitor (e.g., a γ-secretase inhibitor such as XXI), a TGFβ-R1 kinase inhibitor (e.g., ALK5i), a thyroid hormone (e.g., GC-1), a bone morphogenetic (BMP) signaling pathway inhibitor (e.g., LDN193189), a Rho-associated, coiled-coil containing protein kinase (ROCK) inhibitor (e.g., triazovivin), a protein kinase inhibitor (e.g., staurosporine), and a histone methyltransferase EZH2 inhibitor (e.g., DZNep). In some embodiments, the third medium further comprises one or more agents selected from an acetyl COA related metabolite (e.g., acetate), an HDAC inhibitor (e.g., β-hydroxybutyrate), a redox homeostasis regulator (e.g., taurine), and one carbon metabolism pathway intermediate (e.g., formate). In some embodiments, the third medium further comprises a vitamin (e.g., biotin). In some embodiments, the third medium further comprises glutamine. In some embodiments, the third medium further comprises a water soluble synthetic polymer (e.g., PVA such as PVA 87-89%).

In some embodiments, the third medium does not comprise a Wnt signaling pathway inhibitor or a PKC activator. In some embodiments, the third medium comprises a notch signaling pathway inhibitor (e.g., a γ-secretase inhibitor such as XXI), a TGFβ-R1 kinase inhibitor (e.g., ALK5i), a thyroid hormone (e.g., GC-1), a bone morphogenetic (BMP) signaling pathway inhibitor (e.g., LDN193189), a Rho-associated, coiled-coil containing protein kinase (ROCK) inhibitor (e.g., thiazovivin), a protein kinase inhibitor (e.g., staurosporine), and a histone methyltransferase EZH2 inhibitor (e.g., DZNep), an acetyl COA related metabolite (e.g., acetate), an HDAC inhibitor (e.g., β-hydroxybutyrate), a redox homeostasis regulator (e.g., taurine), a one carbon metabolism pathway intermediate (e.g., formate), a vitamin (e.g., biotin), glutamine and a water soluble synthetic polymer (e.g., PVA such as PVA 87-89%), and does not comprise a Wnt signaling pathway inhibitor and a PKC activator. In some embodiments, the third population of cells are cultured in the third medium (e.g., the third medium that does not comprise a Wnt signaling pathway inhibitor or a PKC activator) for a period of about 24-96 hours (e.g., about 24-96 hours, 24-84 hours, 24-72 hours, 24-60 hours, 24-48 hours, 24-36 hours, 36-96 hours, 36-84 hours, 36-72 hours, 36-60 hours, 36-48 hours, 48-96 hours, 48-84 hours, 48-72 hours, 48-60 hours, 60-96 hours, 60-84 hours, 60-72 hours, 72-96 hours, 72-84 hours, or 84-96 hours). In some embodiments, the third population of cells are cultured in the third medium for a period of about 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours. In some embodiments, the third population of cells are cultured in the third medium for a period of about 96 hours.

In some embodiments, the third medium further comprises a Wnt signaling pathway inhibitor but does not comprise a PKC activator. In some embodiments, the third medium comprises Wnt signaling pathway inhibitor (e.g., a tankyrase inhibitor such as NVP-TNKS656), a notch signaling pathway inhibitor (e.g., a γ-secretase inhibitor such as XXI), a TGFβ-R1 kinase inhibitor (e.g., ALK5i), a thyroid hormone (e.g., GC-1), a bone morphogenetic (BMP) signaling pathway inhibitor (e.g., LDN193189), a Rho-associated, coiled-coil containing protein kinase (ROCK) inhibitor (e.g., thiazovivin), a protein kinase inhibitor (e.g., staurosporine), and a histone methyltransferase EZH2 inhibitor (e.g., DZNep), an acetyl COA related metabolite (e.g., acetate), an HDAC inhibitor (e.g., β-hydroxybutyrate), a redox homeostasis regulator (e.g., taurine), a one carbon metabolism pathway intermediate (e.g., formate), a vitamin (e.g., biotin), glutamine and a water soluble synthetic polymer (e.g., PVA such as PVA 87-89%), and does not comprise a PKC activator. In some embodiments, the third population of cells are cultured in the third medium (e.g., the third medium that comprises a Wnt signaling pathway inhibitor but not a PKC activator) for a period of about 24-48 hours (e.g., about 24-48 hours, 24-36 hours, or 36-48 hours), after which the Wnt signaling pathway inhibitor is removed from the third medium and the cells are further cultured for about 24-48 hours ((e.g., about 24-48 hours, 24-36 hours, or 36-48 hours). In some embodiments, the third population of cells are cultured in the third medium (e.g., the third medium that comprises a Wnt signaling pathway inhibitor but not a PKC activator) for a period of about 48 hours, after which the Wnt signaling pathway inhibitor is removed from the third medium and the cells are further cultured for about 48 hours.

In some embodiments, culturing the third population of cells in the third media for a contacting period described herein (e.g., 96 hours) results in a fourth population of cells. In some embodiments, the fourth population of cells comprises cells that are PDX1-positive and NKX6.1 positive. In some embodiments, the fourth population of cells comprises cells that are insulin-positive. In some embodiments, the fourth population of cells comprises cells that are PDX1-positive, NKX6.1 positive, and insulin-positive. In some embodiments, the fourth population of cells comprise cells that are ISL1-positive. In some embodiments, the fourth population of cells comprises cells that are ISL-1 negative. In some embodiments, at least 30% (e.g., at least 30%, at least 40%, at least 50%, or at least 60%)) of the fourth population of cells are insulin-positive. In some embodiments, 30%-50%, 30%-40%, or 40%-50% of the fourth population of cells are insulin-positive.

In some embodiments, the method further comprises culturing the fourth population of cells in a fourth medium comprising one or more agents selected from: a TGFβ-R1 kinase inhibitor (e.g., ALK5i), a thyroid hormone (e.g., GC-1), a bone morphogenetic (BMP) signaling pathway inhibitor (e.g., LDN193189), a Rho-associated, coiled-coil containing protein kinase (ROCK) inhibitor (e.g., triazovivin), a protein kinase inhibitor (e.g., staurosporine), and a histone methyltransferase EZH2 inhibitor (e.g., DZNep). In some embodiments, the fourth medium further comprises one or more agents selected from an acetyl COA related metabolite (e.g., acetate), an HDAC inhibitor (e.g., β-hydroxybutyrate), a redox homeostasis regulator (e.g., taurine), and a one carbon metabolism pathway intermediate (e.g., formate). In some embodiments, the fourth medium further comprises a vitamin (e.g., biotin). In some embodiments, the fourth medium further comprises one or more of glutamine (e.g., L-glutamine), glutamate (e.g., L-glutamate), and carnitine (e.g., L-carnitine). In some embodiments, the fourth medium further comprises albumin (e.g., human serum albumin or HSA). In some embodiments, the fourth medium further comprises ZnSO₄. In some embodiments, the fourth media does not comprise a Wnt signaling pathway inhibitor or a PKC activator. In some embodiments, the fourth medium comprises a TGFβ-R1 kinase inhibitor (e.g., ALK5i), a thyroid hormone (e.g., GC-1), a bone morphogenetic (BMP) signaling pathway inhibitor (e.g., LDN193189), a Rho-associated, coiled-coil containing protein kinase (ROCK) inhibitor (e.g., triazovivin), a protein kinase inhibitor (e.g., staurosporine), and a histone methyltransferase EZH2 inhibitor (e.g., DZNep), an acetyl CoA related metabolite (e.g., acetate), an HDAC inhibitor (e.g., β-hydroxybutyrate), a redox homeostasis regulator (e.g., taurine), a one carbon metabolism pathway intermediate (e.g., formate), a vitamin (e.g., biotin), glutamine, glutamate, carnitine, albumin (e.g., human serum albumin or HSA), and ZnSO₄, and does not comprise a Wnt signaling pathway inhibitor or a PKC activator.

In some embodiments, the fourth population of cells are cultured in the fourth medium for a period of about 24-96 hours (e.g., about 24-96 hours, 24-84 hours, 24-72 hours, 24-60 hours, 24-48 hours, 24-36 hours, 36-96 hours, 36-84 hours, 36-72 hours, 36-60 hours, 36-48 hours, 48-96 hours, 48-84 hours, 48-72 hours, 48-60 hours, 60-96 hours, 60-84 hours, 60-72 hours, 72-96 hours, 72-84 hours, or 84-96 hours). In some embodiments, the fourth population of cells are cultured in the fourth medium for a period of about 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 hours. In some embodiments, the fourth population of cells are cultured in the fourth medium for a period of about 72 hours.

In some embodiments, culturing the fourth population of cells in the fourth media for a contacting period described herein (e.g., 96 hours) results in a fifth population of cells. In some embodiments, a method described herein further comprises culturing the fifth population of cells in a fifth medium comprising glutamine, albumin (e.g., human serum albumin or HSA), and ZnSO₄. In some embodiments, the fifth medium comprises glutamine, albumin (e.g., human serum albumin or HSA) and ZnSO₄, and does not comprise any one of the agents selected from: a TGFβ-R1 kinase inhibitor (e.g., ALK5i), a thyroid hormone (e.g., GC-1), a bone morphogenetic (BMP) signaling pathway inhibitor (e.g., LDN193189), a Rho-associated, coiled-coil containing protein kinase (ROCK) inhibitor (e.g., triazovivin), a protein kinase inhibitor (e.g., staurosporine), and a histone methyltransferase EZH2 inhibitor (e.g., DZNep). In some embodiments, the fifth media further comprises a histone methyltransferase EZH2 inhibitor (e.g., DZNep), an acetyl COA related metabolite (e.g., acetate), an HDAC inhibitor (e.g., β-hydroxybutyrate), a redox homeostasis regulator (e.g., taurine), a one carbon metabolism pathway intermediate (e.g., formate), a vitamin (e.g., biotin), glutamine, glutamate, and carnitine. In some embodiments, the fifth medium comprises a histone methyltransferase EZH2 inhibitor (e.g., DZNep), an acetyl CoA related metabolite (e.g., acetate), an HDAC inhibitor (e.g., β-hydroxybutyrate), a redox homeostasis regulator (e.g., taurine), an one carbon metabolism pathway intermediate (e.g., formate), a vitamin (e.g., biotin), glutamate, glutamine, carnitine, albumin (e.g., human serum albumin or HSA), and ZnSO₄, and does not comprise any one of the agents selected from: a TGFβ-R1 kinase inhibitor (e.g., ALK5i), a thyroid hormone (e.g., GC-1), a bone morphogenetic (BMP) signaling pathway inhibitor (e.g., LDN193189), a Rho-associated, coiled-coil containing protein kinase (ROCK) inhibitor (e.g., thiazovivin), a protein kinase inhibitor (e.g., staurosporine), and a histone methyltransferase EZH2 inhibitor (e.g., DZNep). In some embodiments, the fifth medium comprises albumin (e.g., human serum albumin or HSA), and ZnSO₄, and does not comprise any one of the agents selected from: a TGFβ-R1 kinase inhibitor (e.g., ALK5i), a thyroid hormone (e.g., GC-1), a bone morphogenetic (BMP) signaling pathway inhibitor (e.g., LDN193189), a Rho-associated, coiled-coil containing protein kinase (ROCK) inhibitor (e.g., triazovivin), a protein kinase inhibitor (e.g., staurosporine), a histone methyltransferase EZH2 inhibitor (e.g., DZNep), an acetyl COA related metabolite (e.g., acetate), an HDAC inhibitor (e.g., β-hydroxybutyrate), a redox homeostasis regulator (e.g., taurine), an one carbon metabolism pathway intermediate (e.g., formate), a vitamin (e.g., biotin), carnitine, glutamate, and glutamine.

In some embodiments, the fifth population of cells are cultured in the fifth medium for a period of about 96-240 hours (e.g., about 96-240 hours, 96-216 hours, 96-192 hours, 96-168 hours, 96-144 hours, 96-120 hours; 120-240 hours, 120-216 hours, 120-192 hours, 120-168 hours, 120-144 hours, 144-240 hours, 144-216 hours, 144-192 hours, 144-168 hours, 168-240 hours, 168-216 hours, 168-192 hours, 192-240 hours, 192-216 hours, or 192-240 hours). In some embodiments, the fifth population of cells are cultured in the fifth medium for a period of about 24, 48, 72, 96, 120, 144, 168, 192, 216, or 240 hours. In some embodiments, the fifth population of cells are cultured in the fifth medium for a period of about 192 hours.

In some embodiments, culturing the fifth population of cells in the fifth media for a contacting period described herein (e.g., 192 hours) results in a sixth population of cells. In some embodiments, at least 15% (e.g., at least 15%, at least 20%, at least 25%, at least 30%, at least 35%, at least 40% or more) of the sixth population of cells are NKX6.1-negative, ISL-positive; and wherein less than 12% (e.g., less than 12%, less than 10%, less than 8%, less than 6%, less than 4%, less than 2% or less) of the sixth population of cells are NKX6.1-negative, ISL-negative.

In some embodiments, a method described herein comprises:
(i) culturing a first population of cells in a first medium to obtain a second population of cells, wherein the first population of cells comprises pancreatic progenitor cells that are PDX1-positive and NKX6.1 negative, and pancreatic progenitor cells that are PDX1-positive and NKX6.1 positive; and the first medium comprises: a FoxO1 inhibitor, a notch signaling pathway inhibitor, a PKC activator, a fibroblast growth factor, a sonic hedgehog (SHH) signaling pathway inhibitor, retinoic acid, a Rho-associated, coiled-coil containing protein kinase (ROCK) inhibitor, a TGF-β ligand, and a water-soluble synthetic polymer;
(ii) culturing the second population of cells obtained in (i) with a second medium to obtain a third population of cells, wherein the second medium comprises: a Wnt signaling pathway inhibitor, a PKC activator, an epidermal growth factor, a thyroid hormone, a TGFβ-R1 kinase inhibitor, a notch signaling pathway inhibitor, a sonic hedgehog (SHH) signaling pathway inhibitor, retinoic acid, a Rho-associated, coiled-coil containing protein kinase (ROCK) inhibitor, a protein kinase inhibitor, a bone morphogenetic (BMP) signaling pathway inhibitor, a histone methyltransferase EZH2 inhibitor, an acetyl CoA related metabolite, an HDAC inhibitor, a redox homeostasis regulator, a one carbon metabolism pathway intermediate, a vitamin, glutamine and a water soluble synthetic polymer (e.g., PVA), and wherein the second medium does not comprise a FOXO1 inhibitor;
(iii) culturing the third population of cells obtained in (ii) with a third medium to obtain a fourth population of cells, wherein the third medium comprises: a notch signaling pathway inhibitor, a TGFβ-R1 kinase inhibitor, a thyroid hormone, a bone morphogenetic (BMP) signaling pathway, a Rho-associated, coiled-coil containing protein kinase (ROCK) inhibitor, a protein kinase inhibitor, and a histone methyltransferase EZH2 inhibitor, an acetyl CoA related metabolite, an HDAC inhibitor, a redox homeostasis regulator, an one carbon metabolism pathway intermediate, a vitamin, glutamine and a water soluble synthetic polymer, and wherein the third medium does not comprise a Wnt signaling pathway inhibitor and a PKC activator;
(iv) culturing the fourth population of cells obtained in (iii) with a fourth medium to obtain a fifth population of cells, wherein the fourth medium comprises a notch signaling pathway inhibitor, a TGFβ-R1 kinase inhibitor, a thyroid hormone, a bone morphogenetic (BMP) signaling pathway inhibitor, a Rho-associated, coiled-coil containing protein kinase (ROCK) inhibitor, a protein kinase inhibitor, a histone methyltransferase EZH2 inhibitor, an acetyl COA related metabolite, an HDAC inhibitor, a redox homeostasis regulator, a one carbon metabolism pathway intermediate, a vitamin, glutamine, glutamate, carnitine, albumin, and ZnSO₄, and wherein the fourth medium does not comprise a Wnt signaling pathway inhibitor and a PKC activator; and
(v) culturing the fifth population of cells obtained in (iv) with a fifth medium to obtain a sixth population of cells, wherein the fifth medium comprises albumin (e.g., human serum albumin or HSA) and ZnSO₄.

In some embodiments, a method described herein further comprises generating the first population of cells comprising pancreatic progenitor cells that are PDX1-positive and NKX6.1 negative and pancreatic progenitor cells that are PDX1-positive and NKX6.1 positive. In some embodiments, the first population of cells are differentiated from stem cells (e.g., embryonic stem cells or pluripotent stem cells). In some embodiments, the stem cells (e.g., embryotic stem cells) are generated from the inner cell mass of blastocyst-stage embryos represent. Stem cells can be maintained in culture, renew for themselves, proliferate unlimitedly as undifferentiated ES cells, and are capable of differentiating into all cell types of the body as the ectoderm, mesoderm, and endoderm lineage cells or tissues.

### Cell Types During Pancreatic Differentiation

Aspects of the present disclosure provide cell types of the pancreatic lineage obtained during differentiation of stem cells to generate pancreatic islet cells. Such cells include any cell that is capable of differentiating into a pancreatic islet cell, including for example, a pluripotent stem cell, a definitive endoderm cell, a primitive gut tube cell, a pancreatic progenitor cell, or endocrine progenitor cell, when cultured under conditions suitable for differentiating the precursor cell into the pancreatic islet cell.

### Stem Cells

In some embodiments, any of the stem cells (e.g., any of the genetically engineered stem cells) disclosed herein may be used in generating genetically engineered SC-islet cells or precursors thereof.

### Definitive Endoderm Cells

The definitive endoderm can be generated in vivo from the inner cell mass by the process of gastrulation of embryogenesis, in which epiblast cells are instructed to form the three germ layers. Definitive endoderm can give rise to diverse cells and tissues that contribute to vital organs as the pancreatic β cells, liver hepatocytes, lung alveolar cells, thyroid, thymus, and the epithelial lining of the alimentary and respiratory tract. It is different from the primitive endoderm of extraembryonic tissues, which can give rise to the visceral and parietal endoderm. The definitive endoderm derived from ES cells is theoretically capable of becoming any endoderm derivatives.

Precise patterning of anterior-posterior axis of the definitive endoderm can eventually form the primitive gut tube. The definitive endoderm-derived primitive gut tube induces the pharynx, esophagus, stomach, duodenum, small and large intestine along the anterior-posterior axis as well as associated organs, including pancreas, lung, thyroid, thymus, parathyroid, and liver. The anterior portion of the foregut of the primitive gut tube becomes lung, thyroid, esophagus, and stomach. The pancreas, liver, and duodenum originate from the posterior portion of the foregut. The midgut and hindgut of primitive gut tube gives rise to the small and large intestine. The anterior foregut expresses developmental markers, NK2 homeobox 1 (NKX2-1) and SRY (sex determining region Y)-box 2 (SOX2); the posterior foregut expresses haematopoietically expressed homeobox (HHEX), pancreatic and duodenal homeobox 1 (PDX1), one cut homeobox 1 (ONECUT1, known as HNF6), and hepatocyte nuclear factor 4 alpha (HNF4A); and the midgut/hindgut expresses caudal type homeobox 1 (CDX1), caudal type homeobox 2 (CDX2), and motor neuron and pancreas homeobox 1 (MNX1) (3, 19, 20).

As described herein definitive endoderm cells of use herein can be derived from any source or generated in accordance with any suitable protocol. In some aspects, pluripotent stem cells, e.g., iPSCs or hESCs, are differentiated to endoderm cells. In some aspects, the endoderm cells (stage 1) are further differentiated, e.g., to primitive gut tube cells (stage 2), PDX1-positive pancreatic progenitor cells (stage 3), NKX6.1-positive pancreatic progenitor cells (stage 4), or Ngn3-positive endocrine progenitor cells or insulin-positive endocrine cells (stage 5), followed by induction or maturation to SC-β cells (stage 6).

In some embodiments, definitive endoderm cells can be obtained by differentiating at least some pluripotent cells in a population into definitive endoderm cells, e.g., by contacting a population of pluripotent cells with i) at least one growth factor from the TGF-β superfamily, and ii) a WNT signaling pathway activator, to induce the differentiation of at least some of the pluripotent cells into definitive endoderm cells, wherein the definitive endoderm cells express at least one marker characteristic of definitive endoderm.

Any growth factor from the TGF-β superfamily capable of inducing the pluripotent stem cells to differentiate into definitive endoderm cells (e.g., alone, or in combination with a WNT signaling pathway activator) can be used in the method provided herein. In some embodiments, the growth factor from the TGF-β superfamily comprises Activin A. In some embodiments, the growth factor from the TGF-β superfamily comprises growth differentiating factor 8 (GDF8). Any WNT signaling pathway activator capable of inducing the pluripotent stem cells to differentiate into definitive endoderm cells (e.g., alone, or in combination with a growth factor from the TGF-β superfamily) can be used in the method provided herein. In some embodiments, the WNT signaling pathway activator comprises CHIR99021. In some embodiments, the WNT signaling pathway activator comprises Wnt3a recombinant protein.

In some embodiments, differentiating at least some pluripotent cells in a population into definitive endoderm cells is achieved by a process of contacting a population of pluripotent cells with i) Activin A, and ii) CHIR99021 for a suitable period of time, e.g., about 2 days, about 3 days, about 4 days, or about 5 days to induce the differentiation of at least some of the pluripotent cells in the population into definitive endoderm cells, wherein the definitive endoderm cells express at least one marker characteristic of definitive endoderm. In some embodiments, the process comprises contacting a population of pluripotent cells with activin A and CHIR99021 for 1 day, and then with activin A (in the absence of CHIR99021) for a further 1 or 2 days.

In some examples, the method comprises differentiating pluripotent cells into definitive endoderm cells by contacting a population of pluripotent cells with a suitable concentration of the growth factor from the TGF-β superfamily (e.g., Activin A), such as, about 10 ng/ml, about 20 ng/ml, about 50 ng/ml, about 75 ng/ml, about 80 ng/mL, about 90 ng/ml, about 95 ng/mL, about 100 ng/ml, about 110 ng/ml, about 120 ng/ml, about 130 ng/ml, about 140 ng/ml, about 150 ng/ml, about 175 ng/mL, about 180 ng/mL, about 200 ng/mL, about 250 ng/ml, or about 300 ng/mL. In some embodiments, the method comprises use of about 70-130 ng·ml, 80-120 ng/ml, or 90-110 ng/ml Activin A for differentiation of pluripotent cells into definitive endoderm cells. In some embodiments, the method comprises use of about 100 ng/ml Activin A for differentiation of pluripotent cells into definitive endoderm cells. In some embodiments, the method comprises use of about 200 ng/mL Activin A for differentiation of pluripotent cells into definitive endoderm cells.

In some examples, the method comprises differentiating pluripotent cells into definitive endoderm cells by contacting a population of pluripotent cells with a suitable concentration of the WNT signaling pathway activator (e.g., CHIR99021), such as, about 0.01 μM, about 0.05 μM, about 0.1 μM, about 0.2 μM, about 0.5 μM, about 0.8 μM, about 1 μM, about 1.5 μM, about 2 μM, about 2.5 μM, about 3 μM, about 3.5 μM, about 4 μM, about 5 μM, about 8 μM, about 10 μM, about 12 μM, about 15 μM, about 20 μM, about 30 μM, about 50 μM, about 100 μM, or about 200 μM. In some embodiments, the method comprises use of about 1-5 μM or 2-4 HM CHIR99021 for differentiation of pluripotent cells into definitive endoderm cells. In some embodiments, the method comprises use of about 2 μM CHIR99021 for differentiation of pluripotent cells into definitive endoderm cells. In some embodiments, the method comprises use of about 3 μM CHIR99021 for differentiation of pluripotent cells into definitive endoderm cells. In some embodiments, the method comprises use of about 5 μM CHIR99021 for differentiation of pluripotent cells into definitive endoderm cells.

In some embodiments, the cells are further contacted with a water-soluble synthetic polymer. In some embodiments, the water-soluble synthetic polymer is polyvinyl alcohol. In some cases, the polyvinyl alcohol is at least 78% hydrolyzed, e.g., 79-81% hydrolyzed, 87-89% hydrolyzed, 87-90% hydrolyzed, or 99% hydrolyzed. In some embodiments, the polyvinyl alcohol is 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% hydrolyzed. In some embodiments, the PVA is 80% hydrolyzed.

In some embodiments, a definitive endoderm cell produced by the methods as disclosed herein expresses at least one marker selected from the group consisting of: Nodal, Tmprss2, Tmem30b, St14, Spink3, Sh3gl2, Ripk4, Rab1S, Npnt, Clic6, Cldn5, Cacna1b, Bnip1, Anxa4, Emb, FoxA1, Sox 17, and Rbm35a, wherein the expression of at least one marker is upregulated to by a statistically significant amount in the definitive endoderm cell relative to the pluripotent stem cell from which it was derived. In some embodiments, a definitive endoderm cell produced by the methods as disclosed herein does not express by a statistically significant amount at least one marker selected the group consisting of: Gata4, SPARC, AFP and Dab2 relative to the pluripotent stem cell from which it was derived. In some embodiments, a definitive endoderm cell produced by the methods as disclosed herein does not express by a statistically significant amount at least one marker selected the group consisting of: Zic1, Pax6, Flk1 and CD31 relative to the pluripotent stem cell from which it was derived. In some embodiments, a definitive endoderm cell produced by the methods as disclosed herein has a higher level of phosphorylation of Smad2 by a statistically significant amount relative to the pluripotent stem cell from which it was derived. In some embodiments, a definitive endoderm cell produced by the methods as disclosed herein has the capacity to form gut tube in vivo. In some embodiments, a definitive endoderm cell produced by the methods as disclosed herein can differentiate into a cell with morphology characteristic of a gut cell, and wherein a cell with morphology characteristic of a gut cell expresses FoxA2 and/or Claudin6. In some embodiments, a definitive endoderm cell produced by the methods as disclosed herein can be further differentiated into a cell of endoderm origin.

In some embodiments, a population of pluripotent stem cells are cultured in the presence of at least one β cell differentiation factor prior to any differentiation or during the first stage of differentiation. One can use any pluripotent stem cell, such as a human pluripotent stem cell, or a human iPS cell or any of pluripotent stem cell as discussed herein or other suitable pluripotent stem cells. In some embodiments, a β cell differentiation factor as described herein can be present in the culture medium of a population of pluripotent stem cells or may be added in bolus or periodically during growth (e.g. replication or propagation) of the population of pluripotent stem cells. In certain examples, a population of pluripotent stem cells can be exposed to at least one β cell differentiation factor prior to any differentiation. In other examples, a population of pluripotent stem cells may be exposed to at least one β cell differentiation factor during the first stage of differentiation.

### Primitive Gut Tube Cells

Aspects of the disclosure involve primitive gut tube cells. Primitive gut tube cells of use herein can be derived from any source or generated in accordance with any suitable protocol. In some aspects, definitive endoderm cells are differentiated to primitive gut tube cells. In some aspects, the primitive gut tube cells are further differentiated, e.g., to PDX1-positive pancreatic progenitor cells, NKX6.1-positive pancreatic progenitor cells, Ngn3-positive endocrine progenitor cells, insulin-positive endocrine cells, followed by induction or maturation to SC-β cells.

In some embodiments, primitive gut tube cells can be obtained by differentiating at least some definitive endoderm cells in a population into primitive gut tube cells, e.g., by contacting definitive endoderm cells with at least one growth factor from the fibroblast growth factor (FGF) family, to induce the differentiation of at least some of the definitive endoderm cells into primitive gut tube cells, wherein the primitive gut tube cells express at least one marker characteristic of primitive gut tube cells.

Any growth factor from the FGF family capable of inducing definitive endoderm cells to differentiate into primitive gut tube cells (e.g., alone, or in combination with other factors) can be used in the method provided herein. In some embodiments, the at least one growth factor from the FGF family comprises keratinocyte growth factor (KGF). In some embodiments, the at least one growth factor from the FGF family comprises FGF2. In some embodiments, the at least one growth factor from the FGF family comprises FGF8B. In some embodiments, the at least one growth factor from the FGF family comprises FGF10. In some embodiments, the at least one growth factor from the FGF family comprises FGF21.

In some embodiments, primitive gut tube cells can be obtained by differentiating at least some definitive endoderm cells in a population into primitive gut tube cells, e.g., by contacting definitive endoderm cells with KGF for a certain period of time, e.g., about 1 day, about 2 days, about 3 days, or about 4 days, to induce the differentiation of at least some of the definitive endoderm cells into primitive gut tube cells.

In some embodiments, the method comprises differentiating definitive endoderm cells into primitive gut tube cells by contacting definitive endoderm cells with a suitable concentration of the growth factor from the FGF family (e.g., KGF), such as, about 10 ng/ml, about 20 ng/mL, about 50 ng/mL, about 75 ng/ml, about 80 ng/ml, about 90 ng/ml, about 95 ng/ml, about 100 ng/ml, about 110 ng/ml, about 120 ng/mL, about 130 ng/ml, about 140 ng/ml, about 150 ng/ml, about 175 ng/ml, about 180 ng/mL, about 200 ng/ml, about 250 ng/ml, or about 300 ng/mL. In some embodiments, the method comprises use of about 20-80 ng/ml, 30-70 ng/ml, or 40-60 ng/mL KGF for differentiation of definitive endoderm cells into primitive gut tube cells. In some embodiments, the method comprises use of about 50 ng/ml KGF for differentiation of definitive endoderm cells into primitive gut tube cells. In some embodiments, the method comprises use of about 100 ng/mL KGF for differentiation of definitive endoderm cells into primitive gut tube cells.

In some embodiments, the cells are further contacted with a water-soluble synthetic polymer. In some embodiments, the water-soluble synthetic polymer is polyvinyl alcohol. In some cases, the polyvinyl alcohol is at least 78% hydrolyzed, e.g., 79-81% hydrolyzed, 87-89% hydrolyzed, 87-90% hydrolyzed, or 99% hydrolyzed. In some embodiments, the polyvinyl alcohol (PVA) is 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% hydrolyzed. In some embodiments, the PVA is 80% hydrolyzed.

### PDX1-Positive Pancreatic Progenitor Cells

Aspects of the disclosure involve PDX1-positive pancreatic progenitor cells. PDX1-positive pancreatic progenitor cells of use herein can be derived from any source or generated in accordance with any suitable protocol. In some aspects, primitive gut tube cells are differentiated to PDX1-positive pancreatic progenitor cells. In some aspects, the PDX1-positive pancreatic progenitor cells are NKX6.1 negative, and can be further differentiated to, e.g., NKX6.1-positive pancreatic progenitor cells, Ngn3-positive endocrine progenitor cells, insulin-positive endocrine cells, followed by induction or maturation to SC-β cells.

In some aspects, PDX1-positive pancreatic progenitor cells can be obtained by differentiating at least some primitive gut tube cells in a population into PDX1-positive pancreatic progenitor cells, e.g., by contacting primitive gut tube cells with i) at least one BMP signaling pathway inhibitor, ii) a growth factor from TGF-β superfamily, iii) at least one growth factor from the FGF family, iv) at least one SHH pathway inhibitor, v) at least one retinoic acid (RA) signaling pathway activator; vi) at least one protein kinase C activator, and vii) a ROCK inhibitor to induce the differentiation of at least some of the primitive gut tube cells into PDX1-positive pancreatic progenitor cells, wherein the PDX1-positive pancreatic progenitor cells express PDX1.

In some aspects, PDX1-positive pancreatic progenitor cells can be obtained by differentiating at least some primitive gut tube cells in a population into PDX1-positive pancreatic progenitor cells, e.g., by contacting primitive gut tube cells with i) at least one BMP signaling pathway inhibitor, ii) a growth factor from TGF-β superfamily, iii) at least one growth factor from the FGF family, iv) at least one SHH pathway inhibitor, v) at least one retinoic acid (RA) signaling pathway activator; and vi) at least one protein kinase C activator, to induce the differentiation of at least some of the primitive gut tube cells into PDX1-positive pancreatic progenitor cells, wherein the PDX1-positive pancreatic progenitor cells express PDX1.

In some embodiments, PDX1-positive pancreatic progenitor cells can be obtained by differentiating at least some primitive gut tube cells in a population into PDX1-positive pancreatic progenitor cells, e.g., by contacting primitive gut tube cells with i) at least one BMP signaling pathway inhibitor, ii) at least one growth factor from the FGF family, iii) at least one SHH pathway inhibitor, iv) at least one retinoic acid (RA) signaling pathway activator; and v) at least one protein kinase C activator, to induce the differentiation of at least some of the primitive gut tube cells into PDX1-positive pancreatic progenitor cells, wherein the PDX1-positive pancreatic progenitor cells express PDX1.

In some embodiments, PDX1-positive pancreatic progenitor cells can be obtained by differentiating at least some primitive gut tube cells in a population into PDX1-positive pancreatic progenitor cells, e.g., by contacting primitive gut tube cells with i) at least one SHH pathway inhibitor, ii) at least one retinoic acid (RA) signaling pathway activator; and iii) at least one protein kinase C activator, wherein the PDX1-positive pancreatic progenitor cells express PDX1.

In some embodiments, PDX1-positive pancreatic progenitor cells can be obtained by differentiating at least some primitive gut tube cells in a population into PDX1-positive pancreatic progenitor cells, e.g., by contacting primitive gut tube cells with i) at least one growth factor from the FGF family, and ii) at least one retinoic acid (RA) signaling pathway activator, to induce the differentiation of at least some of the primitive gut tube cells into PDX1-positive pancreatic progenitor cells, wherein the PDX1-positive pancreatic progenitor cells express PDX1.

Any BMP signaling pathway inhibitor capable of inducing primitive gut tube cells to differentiate into PDX1-positive pancreatic progenitor cells (e.g., alone, or with any combination of a growth factor from TGF-β superfamily, at least one growth factor from the FGF family, at least one SHH pathway inhibitor, at least one retinoic acid signaling pathway activator, at least one protein kinase C activator, and ROCK inhibitor) can be used in the method provided herein. In some embodiments, the BMP signaling pathway inhibitor comprises LDN193189 or DMH-1. In some examples, the method comprises contacting primitive gut tube cells with a concentration of BMP signaling pathway inhibitor (e.g., LDN1931189), such as, about 30 nM, about 40 nM, about 50 nM, about 60 nM, about 70 nM, about 80 nM, about 90 nM, about 100 nM, about 110 nM, about 120 nM, about 130 nM, about 140 nM, about 150 nM, about 160 nM, about 170 nM, about 180 nM, about 190 nM, about 200 nM, about 210 nM, about 220 nM, about 230 nM, about 240 nM, about 250 nM, about 280 nM, about 300 nM, about 400 nM, about 500 nM, or about 1 μM. In some examples, the method comprises contacting primitive gut tube cells with a concentration of BMP signaling pathway inhibitor (e.g., DMH-1), such as, about 0.01 μM, about 0.02 μM, about 0.05 μM, about 0.1 μM, about 0.2 μM, about 0.5 μM, about 0.8 μM, about 1 μM, about 1.2 μM, about 1.5 μM, about 1.75 μM, about 2 μM, about 2.2 μM, about 2.5 μM, about 2.75 μM, about 3 μM, about 3.25 μM, about 3.5 μM, about 3.75 μM, about 4 μM, about 4.5 μM, about 5 μM, about 8 μM, about 10 μM, about 15 μM, about 20 μM, about 30 μM, about 40 μM, about 50 μM, or about 100 μM. In some examples, the method comprises contacting primitive gut tube cells with a concentration of BMP signaling pathway inhibitor (e.g., DMH-1), such as, about 220-280 nM, about 230-270 nM, about 240-260 nM, or about 245-255 nM. In some examples, the method comprises contacting primitive gut tube cells with a concentration of BMP signaling pathway inhibitor (e.g., DMH-1) about 250 nM.

Any growth factor from the TGF-β superfamily capable of inducing primitive gut tube cells to differentiate into PDX1-positive pancreatic progenitor cells (e.g., alone, or with any combination of at least one BMP signaling pathway inhibitor, a growth factor from the FGF family, at least one SHH pathway inhibitor, at least one retinoic acid signaling pathway activator, at least one protein kinase C activator, and ROCK inhibitor) can be used. In some embodiments, the growth factor from TGF-β family comprises Activin A. In some embodiments, the growth factor from TGF-β family comprises GDF8. In some examples, the method comprises contacting primitive gut tube cells with a concentration of a growth factor from TGF-β superfamily (e.g., Activin A), such as, about 5 ng/ml, about 7.5 ng/ml, about 8 ng/ml, about 9 ng/ml, about 10 ng/mL, about 11 ng/ml, about 12 ng/ml, about 13 ng/ml, about 14 ng/ml, about 15 ng/ml, about 16 ng/ml, about 17 ng/ml, about 18 ng/mL, about 19 ng/mL, about 20 ng/mL, about 21 ng/mL, about 22 ng/mL, about 23 ng/ml, about 24 ng/ml, about 25 ng/ml, about 26 ng/ml, about 27 ng/ml, about 28 ng/ml, about 29 ng/ml, about 30 ng/ml, about 35 ng/ml, about 40 ng/mL, about 50 ng/ml, or about 100 ng/mL. In some examples, the method comprises contacting primitive gut tube cells with a concentration of a growth factor from TGF-β superfamily (e.g., Activin A), such as, about 17-23 ng/ml, about 18-22 ng/ml, or about 19-21 ng/ml. In some examples, the method comprises contacting primitive gut tube cells with a concentration of a growth factor from TGF-β superfamily (e.g., Activin A) of about 20 ng/ml.

Any growth factor from the FGF family capable of inducing primitive gut tube cells to differentiate into PDX1-positive pancreatic progenitor cells (e.g., alone, or with any combination of at least one BMP signaling pathway inhibitor, a growth factor from TGF-β superfamily, at least one SHH pathway inhibitor, at least one retinoic acid signaling pathway activator, at least one protein kinase C activator, and ROCK inhibitor) can be used. In some embodiments, the at least one growth factor from the FGF family comprises keratinocyte growth factor (KGF). In some embodiments, the at least one growth factor from the FGF family is selected from the group consisting of FGF2, FGF8B, FGF10, and FGF21. In some examples, the method comprises contacting primitive gut tube cells with a concentration of a growth factor from FGF family (e.g., KGF), such as, about 10 ng/ml, about 20 ng/ml, about 50 ng/ml, about 75 ng/ml, about 80 ng/ml, about 90 ng/ml, about 95 ng/ml, about 100 ng/ml, about 110 ng/ml, about 120 ng/ml, about 130 ng/ml, about 140 ng/mL, about 150 ng/ml, about 175 ng/ml, about 180 ng/ml, about 200 ng/mL, about 250 ng/mL, or about 300 ng/ml. In some examples, the method comprises contacting primitive gut tube cells with a concentration of a growth factor from FGF family (e.g., KGF), such as, about 20-80 ng/ml, about 30-70 ng/ml, about 40-60 ng/ml, or about 45-55 ng/ml. In some examples, the method comprises contacting primitive gut tube cells with a concentration of a growth factor from FGF family (e.g., KGF) of about 50 ng/ml.

Any SHH pathway inhibitor capable of inducing primitive gut tube cells to differentiate into PDX1-positive pancreatic progenitor cells (e.g., alone, or with any combination of at least one BMP signaling pathway inhibitor, at least one growth factor from the FGF family, a growth factor from TGF-β superfamily, at least one retinoic acid signaling pathway activator, at least one protein kinase C activator, and ROCK inhibitor) can be used. In some embodiments, the SHH pathway inhibitor comprises Sant1. In some examples, the method comprises contacting primitive gut tube cells with a concentration of a SHH pathway inhibitor (e.g., Sant1), such as, about 0.001 μM, about 0.002 μM, about 0.005 μM, about 0.01 μM, about 0.02 μM, about 0.03 μM, about 0.05 μM, about 0.08 μM, about 0.1 μM, about 0.12 μM, about 0.13 μM, about 0.14 μM, about 0.15 μM, about 0.16 μM, about 0.17 μM, about 0.18 μM, about 0.19 μM, about 0.2 μM, about 0.21 μM, about 0.22 μM, about 0.23 μM, about 0.24 μM, about 0.25 μM, about 0.26 μM, about 0.27 μM, about 0.28 μM, about 0.29 μM, about 0.3 μM, about 0.31 μM, about 0.32 μM, about 0.33 μM, about 0.34 μM, about 0.35 μM, about 0.4 μM, about 0.45 μM, about 0.5 μM, about 0.6 μM, about 0.8 μM, about 1 μM, about 2 μM, or about 5 μM. In some examples, the method comprises contacting primitive gut tube cells with a concentration of a SHH pathway inhibitor (e.g., Sant1), such as, about 220-280 nM, about 230-270 nM, about 240-260 nM, or about 245-255 nM. In some examples, the method comprises contacting primitive gut tube cells with a concentration of a SHH pathway inhibitor (e.g., Sant1) of about 250 nM.

Any RA signaling pathway activator capable of inducing primitive gut tube cells to differentiate into PDX1-positive pancreatic progenitor cells (e.g., alone, or with any combination of at least one BMP signaling pathway inhibitor, at least one growth factor from the FGF family, at least one SHH pathway inhibitor, at least one protein kinase C activator, and ROCK inhibitor) can be used. In some embodiments, the RA signaling pathway activator comprises retinoic acid. In some examples, the method comprises contacting primitive gut tube cells with a concentration of an RA signaling pathway activator (e.g., retinoic acid), such as, about 0.02 μM, about 0.1 μM, about 0.2 μM, about 0.25 μM, about 0.3 μM, about 0.4 μM, about 0.45 μM, about 0.5 μM, about 0.55 μM, about 0.6 μM, about 0.65 μM, about 0.7 μM, about 0.75 μM, about 0.8 μM, about 0.85 μM, about 0.9 μM, about 1 μM, about 1.1 μM, about 1.2 μM, about 1.3 μM, about 1.4 μM, about 1.5 μM, about 1.6 μM, about 1.7 μM, about 1.8 μM, about 1.9 μM, about 2 μM, about 2.1 μM, about 2.2 μM, about 2.3 μM, about 2.4 μM, about 2.5 μM, about 2.6 μM, about 2.7 μM, about 2.8 μM, about 3 μM, about 3.2 μM, about 3.4 μM, about 3.6 μM, about 3.8 μM, about 4 μM, about 4.2 μM, about 4.4 μM, about 4.6 μM, about 4.8 μM, about 5 μM, about 5.5 μM, about 6 μM, about 6.5 μM, about 7 μM, about 7.5 μM, about 8 μM, about 8.5 μM, about 9 μM, about 9.5 μM, about 10 μM, about 12 μM, about 14 μM, about 15 μM, about 16 μM, about 18 μM, about 20 μM, about 50 μM, or about 100 μM. In some examples, the method comprises contacting primitive gut tube cells with a concentration of an RA signaling pathway activator (e.g., retinoic acid), such as, about 1.7-2.3 μM, about 1.8-2.2 μM, or about 1.9-2.1 μM. In some examples, the method comprises contacting primitive gut tube cells with a concentration of an RA signaling pathway activator (e.g., retinoic acid) of about 2 μM.

Any PKC activator capable of inducing primitive gut tube cells to differentiate into PDX1-positive pancreatic progenitor cells (e.g., alone, or with any combination of at least one BMP signaling pathway inhibitor, at least one growth factor from the FGF family, at least one SHH pathway inhibitor, at least one RA signaling pathway activator, and ROCK inhibitor) can be used. In some embodiments, the PKC activator comprises PdBU. In some embodiments, the PKC activator comprises TPPB. In some examples, the method comprises contacting primitive gut tube cells with a concentration of a PKC activator (e.g., PdBU or TPPB), such as, about 10 nM, 50 nM, 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 μM, 10 μM, about 20 μM, about 50 μM, about 75 μM, about 80 μM, about 100 μM, about 120 μM, about 140 μM, about 150 μM, about 175 μM, about 180 μM, about 200 μM, about 210 μM, about 220 μM, about 240 μM, about 250 μM, about 260 μM, about 280 μM, about 300 μM, about 320 μM, about 340 μM, about 360 μM, about 380 μM, about 400 μM, about 420 μM, about 440 μM, about 460 μM, about 480 μM, about 500 μM, about 520 μM, about 540 μM, about 560 μM, about 580 μM, about 600 μM, about 620 μM, about 640 μM, about 660 μM, about 680 μM, about 700 μM, about 750 μM, about 800 μM, about 850 μM, about 900 μM, about 1 mM, about 2 mM, about 3 mM, about 4 mM, or about 5 mM. In some embodiments, the method comprises contacting primitive gut tube cells with a concentration of a PKC activator (e.g., PdBU or TPPB) of 10 nM-1 mM, 10 nM-500 μM, 10 nM-1 μM, 10-800 nM, 100-900 nM, 300-800 nM, 300-600 nM, 400-600 nM, 450-550 nM, or about 500 nM. In some examples, the method comprises contacting primitive gut tube cells with a concentration of a PKC activator (e.g., PdBU or TPPB), such as, about 450-550 mM, about 475-525 nM, about 490-510 nM, or about 495-505 nM. In some examples, the method comprises contacting primitive gut tube cells with a concentration of a PKC activator (e.g., PdBU or TPPB) of about 500 nM. In some embodiments, primitive gut tube cells are not treated with a PKC activator (e.g., PDBU).

Any ROCK inhibitor capable of inducing primitive gut tube cells to differentiate into PDX1-positive pancreatic progenitor cells (e.g., alone, or with any combination of at least one BMP signaling pathway inhibitor, at least one growth factor from the FGF family, at least one SHH pathway inhibitor, PKC activator, and at least one RA signaling pathway activator) can be used. In some embodiments, the ROCK inhibitor comprises Thiazovivin, Y-27632, Fasudil/HA1077, or H-1152. In some embodiments, the ROCK inhibitor comprises Y-27632. In some embodiments, the ROCK inhibitor comprises Thiazovivin. In some examples, the method comprises contacting primitive gut tube cells with a concentration of a ROCK inhibitor (e.g., Y-27632 or Thiazovivin), such as, about 0.2 μM, about 0.5 μM, about 0.75 μM, about 1 μM, about 2 μM, about 3 μM, about 4 μM, about 5 μM, about 6 μM, about 7 μM, about 7.5 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 50 μM, or about 100 μM. In some examples, the method comprises contacting primitive gut tube cells with a concentration of a ROCK inhibitor (e.g., Y-27632 or Thiazovivin), such as, about 2.2-2.8 μM, about 2.3-2.7 μM, or about 2.4-2.6 μM. In some examples, the method comprises contacting primitive gut tube cells with a concentration of a ROCK inhibitor (e.g., Y-27632 or Thiazovivin) of about 2.5 μM.

In some embodiments, the cells are further contacted with a water-soluble synthetic polymer. In some embodiments, the water-soluble synthetic polymer is polyvinyl alcohol. In some cases, the polyvinyl alcohol is at least 78% hydrolyzed, e.g., 79-81% hydrolyzed, 87-89% hydrolyzed, 87-90% hydrolyzed, or 99% hydrolyzed. In some embodiments, the polyvinyl alcohol (PVA) is 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% hydrolyzed. In some embodiments, the PVA is 80% hydrolyzed.

In some embodiments, PDX1-positive pancreatic progenitor cells can be obtained by differentiating at least some primitive gut tube cells in a population into PDX1-positive pancreatic progenitor cells, e.g., by contacting primitive gut tube cells with retinoic acid, KGF, Sant1, DMH-1, PdBU, thiazovivin, and Activin A, for a suitable period of time, e.g., about 1 day, about 2 days, about 3 days, or about 4 days. In some embodiments, PDX1-positive pancreatic progenitor cells can be obtained by differentiating at least some primitive gut tube cells in a population into PDX1-positive pancreatic progenitor cells, e.g., by contacting primitive gut tube cells with retinoic acid, KGF, Sant1, DMH-1, PdBU, thiazovivin, and Activin A, for about 2 days. In some embodiments, PDX1-positive pancreatic progenitor cells can be obtained by differentiating at least some primitive gut tube cells in a population into PDX1-positive pancreatic progenitor cells, e.g., by contacting primitive gut tube cells with retinoic acid, KGF, Sant1, DMH-1, PdBU, thiazovivin, and Activin A for 1 day, followed by contacting the cells with retinoic acid, KGF, Sant1, PdBU, thiazovivin, and Activin A for 1 day (in the absence of DMH-1).

### NKX6.1-Positive Pancreatic Progenitor Cells

Aspects of the disclosure involve NKX6.1-positive pancreatic progenitor cells. NKX6.1-positive pancreatic progenitor cells of use herein can be derived from any source or generated in accordance with any suitable protocol. In some aspects, PDX1-positive, NKX6.1-negative pancreatic progenitor cells are differentiated to PDX1-positive, NKX6.1-positive pancreatic progenitor cells. In some aspects, the NKX6.1-positive pancreatic progenitor cells are further differentiated, e.g., to Ngn3-positive endocrine progenitor cells, or insulin-positive endocrine cells, followed by induction or maturation to SC-β cells.

In some aspects, a method of producing a NKX6.1-positive pancreatic progenitor cell from a PDX1-positive pancreatic progenitor cell comprises contacting a population of cells (e.g., under conditions that promote cell clustering and/or promoting cell survival) comprising PDX1-positive pancreatic progenitor cells with at least two β cell-differentiation factors comprising a) at least one growth factor from the fibroblast growth factor (FGF) family, b) a sonic hedgehog pathway inhibitor, and optionally c) a low concentration of a retinoic acid (RA) signaling pathway activator, to induce the differentiation of at least one PDX1-positive pancreatic progenitor cell in the population into NKX6.1-positive pancreatic progenitor cells, wherein the NKX6.1-positive pancreatic progenitor cells expresses NKX6.1.

In some embodiments, the PDX1-positive, NKX6.1-positive pancreatic progenitor cells are obtained by contacting PDX1-positive pancreatic progenitor cells with i) at least one growth factor from the FGF family, ii) at least one SHH pathway inhibitor, and optionally iii) a RA signaling pathway activator, to induce the differentiation of at least some of the PDX1-positive pancreatic progenitor cells into PDX1-positive, NKX6.1-positive pancreatic progenitor cells, wherein the PDX1-positive, NKX6.1-positive pancreatic progenitor cells express PDX1 and NKX6.1.

In some embodiments, the PDX1-positive, NKX6.1-positive pancreatic progenitor cells are obtained by contacting PDX1-positive pancreatic progenitor cells with i) at least one growth factor from the FGF family, ii) at least one SHH pathway inhibitor, and optionally iii) a RA signaling pathway activator, iv) ROCK inhibitor, and v) at least one growth factor from the TGF-β superfamily, to induce the differentiation of at least some of the PDX1-positive pancreatic progenitor cells into PDX1-positive, NKX6.1-positive pancreatic progenitor cells. In some embodiments, following 3, 4, or 5 days of contacting the PDX1-positive, NKX6.1-positive pancreatic progenitor cells are obtained by contacting PDX1-positive pancreatic progenitor cells with i) at least one growth factor from the FGF family, ii) at least one SHH pathway inhibitor, and optionally iii) a RA signaling pathway activator, iv) ROCK inhibitor, and v) at least one growth factor from the TGF-β superfamily; the cells are then contacted with i) at least one growth factor from the FGF family, ii) at least one SHH pathway inhibitor, and optionally iii) a RA signaling pathway activator, iv) ROCK inhibitor, and v) at least one growth factor from the TGF-β superfamily, and vi) a PKC activator and optionally a gamma-secretase inhibitor. In some embodiments, the PDX1-positive, NKX6.1-positive pancreatic progenitor cells are obtained by contacting PDX1-positive pancreatic progenitor cells under conditions that promote cell clustering with at least one growth factor from the FGF family. In some embodiments, the growth factor from the FGF family is KGF.

In some embodiments, the disclosure provides for a method in which a first population of cells comprising PDX1-positive, NKX6.1-negative cells is cultured in a media comprising any one or combination of: i) at least one growth factor from the FGF family, ii) at least one SHH pathway inhibitor, iii) a RA signaling pathway activator, iv) a ROCK inhibitor, and v) a growth factor from the TGF-β superfamily for a period of about 1, 2, 3, 4 or 5 days (e.g., 2-4, 3-4, or 4-5 days); thereby generating a second population of cells. In some embodiments, the second population of cells is then incubated in a composition comprising any one or combination of: i) at least one growth factor from the FGF family, ii) at least one SHH pathway inhibitor, iii) a RA signaling pathway activator, iv) a ROCK inhibitor, v) a growth factor from the TGF-β superfamily, vi) a PKC activator, vii) a FoxO1 inhibitor, and optionally viii) a notch signaling inhibitor for about 1, 2, or 3 days (e.g., 1-2, 1-3, or 2-3 days).

In some embodiments, in the media for culturing the first population of cells, the growth factor from the FGF family is present at a concentration of about 45-55 ng/ml, about 46-54 ng/ml, about 47-53 ng/ml, about 48-52 ng/ml, or about 49-51 ng/ml, the SHH pathway inhibitor is present at a concentration of about 200-300 nM, about 220-280 nM, or about 240-260 nM, the RA signaling pathway activator is present at a concentration of about 1.7-2.3 μM, about 1.8-2.2 μM, or about 1.9-2.1 μM, the ROCK inhibitor is present at a concentration of about 2-3 μM, about 2.2-2.8 μM, or about 2.4-2.6 μM, and/or the growth factor from the TGF-β superfamily is present at a concentration of about 2-8 ng/ml, about 3-7 ng/ml or about 4-6 ng/ml.

In some embodiments, in the media for culturing the second population of cells, the growth factor from the FGF family is present at a concentration of about 45-55 ng/ml, about 46-54 ng/ml, about 47-53 ng/ml, about 48-52 ng/ml, or about 49-51 ng/ml, the SHH pathway inhibitor is present at a concentration of about 200-300 nM, about 220-280 nM, or about 240-260 nM, the RA signaling pathway activator is present at a concentration of about 1.7-2.3 μM, about 1.8-2.2 μM, or about 1.9-2.1 μM, the ROCK inhibitor is present at a concentration of about 2-3 μM, about 2.2-2.8 μM, or about 2.4-2.6 μM, the growth factor from the TGF-β superfamily is present at a concentration of 2 about-8 ng/ml, about 3-7 ng/ml or about 4-6 ng/ml, the PKC activator is present at a concentration of about 0.2-0.8 μM, about 0.3-0.7 μM, or about 0.4-0.6 μM, and the FoxO1 inhibitor is present at a concentration of about 0.7-1.3 μM, about 0.8-1.2 μM, or about 0.9-1.1 μM, and optionally the notch signaling inhibitor is present at a concentration of about 1.7-2.3 μM, about 1.8-2.2 μM, or about 1.9-2.1 μM.

In some embodiments, the PDX1-positive pancreatic progenitor cells are produced from a population of pluripotent cells. In some embodiments, the PDX1-positive pancreatic progenitor cells are produced from a population of iPS cells. In some embodiments, the PDX1-positive pancreatic progenitor cells are produced from a population of ESC cells. In some embodiments, the PDX1-positive pancreatic progenitor cells are produced from a population of definitive endoderm cells. In some embodiments, the PDX1-positive pancreatic progenitor cells are produced from a population of primitive gut tube cells.

Any growth factor from the FGF family capable of inducing PDX1-positive pancreatic progenitor cells to differentiate into NKX6.1-positive pancreatic progenitor cells (e.g., alone, or with any combination of at least one SHH pathway inhibitor, a ROCK inhibitor, a growth factor from the TGF-β superfamily, and at least one retinoic acid signaling pathway activator) can be used in the method provided herein. In some embodiments, the at least one growth factor from the FGF family comprises keratinocyte growth factor (KGF). In some embodiments, the at least one growth factor from the FGF family is selected from the group consisting of FGF8B, FGF 10, and FGF21. In some examples, the method comprises contacting PDX1-positive pancreatic progenitor cells with a concentration of a growth factor from FGF family (e.g., KGF), such as, about 10 ng/ml, about 20 ng/ml, about 50 ng/ml, about 75 ng/ml, about 80 ng/ml, about 90 ng/ml, about 95 ng/ml, about 100 ng/ml, about 110 ng/ml, about 120 ng/ml, about 130 ng/ml, about 140 ng/ml, about 150 ng/mL, about 175 ng/ml, about 180 ng/ml, about 200 ng/ml, about 250 ng/mL, or about 300 ng/mL. In some examples, the method comprises contacting PDX1-positive pancreatic progenitor cells with a concentration of a growth factor from FGF family (e.g., KGF), such as, about 20-80 ng/ml, about 30-70 ng/ml, about 40-60 ng/ml, or about 45-55 ng/ml. In some examples, the method comprises contacting PDX1-positive pancreatic progenitor cells with a concentration of a growth factor from FGF family (e.g., KGF) of about 50 ng/ml.

Any SHH pathway inhibitor capable of inducing PDX1-positive pancreatic progenitor cells to differentiate into NKX6.1-positive pancreatic progenitor cells (e.g., alone, or with any combination of at least one growth factor from the FGF family, a retinoic acid signaling pathway activator, ROCK inhibitor, and at least one growth factor from the TGF-β superfamily) can be used in the method provided herein. In some embodiments, the SHH pathway inhibitor comprises Sant1. In some examples, the method comprises contacting PDX1-positive pancreatic progenitor cells with a concentration of a SHH pathway inhibitor (e.g., Sant1), such as, about 0.001 μM, about 0.002 μM, about 0.005 μM, about 0.01 μM, about 0.02 μM, about 0.03 μM, about 0.05 μM, about 0.08 μM, about 0.1 μM, about 0.12 μM, about 0.13 μM, about 0.14 μM, about 0.15 μM, about 0.16 μM, about 0.17 μM, about 0.18 μM, about 0.19 μM, about 0.2 μM, about 0.21 μM, about 0.22 μM, about 0.23 μM, about 0.24 μM, about 0.25 μM, about 0.26 μM, about 0.27 μM, about 0.28 μM, about 0.29 μM, about 0.3 μM, about 0.31 μM, about 0.32 μM, about 0.33 μM, about 0.34 μM, about 0.35 μM, about 0.4 μM, about 0.45 μM, about 0.5 μM, about 0.6 μM, about 0.8 μM, about 1 μM, about 2 μM, or about 5 μM. In some examples, the method comprises contacting PDX1-positive pancreatic progenitor cells with a concentration of a SHH pathway inhibitor (e.g., Sant1), such as, about 220-280 nM, about 230-270 nM, about 240-260 nM, or about 245-255 nM. In some examples, the method comprises contacting PDX1-positive pancreatic progenitor cells with a concentration of a SHH pathway inhibitor (e.g., Sant1) of about 250 nM.

Any RA signaling pathway activator capable of inducing PDX1-positive pancreatic progenitor cells to differentiate into NKX6.1-positive pancreatic progenitor cells (e.g., alone, or with any combination of at least one growth factor from the FGF family, at least one SHH pathway inhibitor, ROCK inhibitor, and at least one growth factor from the TGF-β superfamily) can be used. In some embodiments, the RA signaling pathway activator comprises retinoic acid. In some examples, the method comprises contacting PDX1-positive pancreatic progenitor cells with a concentration of an RA signaling pathway activator (e.g., retinoic acid), such as, about 0.02 μM, about 0.1 μM, about 0.2 μM, about 0.25 μM, about 0.3 μM, about 0.4 μM, about 0.45 μM, about 0.5 μM, about 0.55 μM, about 0.6 μM, about 0.65 μM, about 0.7 μM, about 0.75 μM, about 0.8 μM, about 0.85 μM, about 0.9 μM, about 1 μM, about 1.1 μM, about 1.2 μM, about 1.3 μM, about 1.4 μM, about 1.5 μM, about 1.6 μM, about 1.7 μM, about 1.8 μM, about 1.9 μM, about 2 μM, about 2.1 μM, about 2.2 μM, about 2.3 μM, about 2.4 μM, about 2.5 μM, about 2.6 μM, about 2.7 μM, about 2.8 μM, about 3 μM, about 3.2 μM, about 3.4 μM, about 3.6 μM, about 3.8 μM, about 4 μM, about 4.2 μM, about 4.4 μM, about 4.6 μM, about 4.8 μM, about 5 μM, about 5.5 μM, about 6 μM, about 6.5 μM, about 7 μM, about 7.5 μM, about 8 μM, about 8.5 μM, about 9 μM, about 9.5 μM, about 10 μM, about 12 μM, about 14 μM, about 15 μM, about 16 μM, about 18 μM, about 20 μM, about 50 μM, or about 100 μM. In some examples, the method comprises contacting PDX1-positive pancreatic progenitor cells with a concentration of an RA signaling pathway activator (e.g., retinoic acid), such as, about 70-130 nM, about 80-120 nM, about 90-110 nM, or about 95-105 nM. In some examples, the method comprises contacting PDX1-positive pancreatic progenitor cells with a concentration of an RA signaling pathway activator (e.g., retinoic acid) of about 100 nM.

Any ROCK inhibitor capable of inducing PDX1-positive pancreatic progenitor cells to differentiate into NKX6.1-positive pancreatic progenitor cells (e.g., alone, or with any combination of at least one growth factor from the FGF family, at least one SHH pathway inhibitor, a RA signaling pathway activator, and at least one growth factor from the TGF-β superfamily) can be used. In some embodiments, the ROCK inhibitor comprises Thiazovivin, Y-27632, Fasudil/HA1077, or 14-1152. In some examples, the method comprises contacting PDX1-positive pancreatic progenitor cells with a concentration of a ROCK inhibitor (e.g., Y-27632 or Thiazovivin), such as, about 0.2 μM, about 0.5 μM, about 0.75 μM, about 1 μM, about 2 μM, about 3 μM, about 4 μM, about 5 μM, about 6 μM, about 7 μM, about 7.5 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 50 μM, or about 100 μM. In some examples, the method comprises contacting PDX1-positive pancreatic progenitor cells with a concentration of a ROCK inhibitor (e.g., Y-27632 or Thiazovivin), such as, about 2.2-2.8 μM, about 2.3-2.7 μM, or about 2.4-2.6 μM. In some examples, the method comprises contacting PDX1-positive pancreatic progenitor cells with a concentration of a ROCK inhibitor (e.g., Y-27632 or Thiazovivin) of about 2.5 μM.

Any activator from the TGF-β superfamily capable of inducing PDX1-positive pancreatic progenitor cells to differentiate into NKX6.1-positive pancreatic progenitor cells (e.g., alone, or with any combination of at least one growth factor from the FGF family, at least one SHH pathway inhibitor, a RA signaling pathway activator, and ROCK inhibitor) can be used. In some embodiments, the activator from the TGF-β superfamily comprises Activin A or GDF8. In some examples, the method comprises contacting PDX1-positive pancreatic progenitor cells with a concentration of a growth factor from TGF-β superfamily (e.g., Activin A), such as, about 0.1 ng/ml, about 0.2 ng/ml, about 0.3 ng/ml, about 0.4 ng/ml, about 0.5 ng/ml, about 0.6 ng/ml, about 0.7 ng/ml, about 0.8 ng/ml, about 1 ng/ml, about 1.2 ng/ml, about 1.4 ng/ml, about 1.6 ng/ml, about 1.8 ng/ml, about 2 ng/ml, about 2.2 ng/mL, about 2.4 ng/ml, about 2.6 ng/ml, about 2.8 ng/mL, about 3 ng/mL, about 3.2 ng/ml, about 3.4 ng/ml, about 3.6 ng/ml, about 3.8 ng/mL, about 4 ng/ml, about 4.2 ng/ml, about 4.4 ng/ml, about 4.6 ng/ml, about 4.8 ng/ml, about 5 ng/ml, about 5.2 ng/ml, about 5.4 ng/ml, about 5.6 ng/ml, about 5.8 ng/ml, about 6 ng/ml, about 6.2 ng/ml, about 6.4 ng/ml, about 6.6 ng/ml, about 6.8 ng/mL, about 7 ng/ml, about 8 ng/ml, about 9 ng/mL, about 10 ng/ml, about 20 ng/mL, about 30 ng/mL, or about 50 ng/mL. In some examples, the method comprises contacting PDX1-positive pancreatic progenitor cells with a concentration of a growth factor from TGF-β superfamily (e.g., Activin A), such as, about 2-8 ng/ml, about 3-7 ng/ml, about 4-6 ng/ml, or about 4.5-5.5 ng/ml. In some examples, the method comprises contacting PDX1-positive pancreatic progenitor cells with a concentration of a growth factor from TGF-β superfamily (e.g., Activin A), such as, about 5 ng/mL.

Any FoxO1 inhibitor capable of inducing PDX1-positive pancreatic progenitor cells to differentiate into NKX6.1-positive pancreatic progenitor cells (e.g., alone, or with any combination of at least one growth factor from the FGF family, at least one retinoic acid signaling pathway activator, ROCK inhibitor, at least one growth factor from the TGF-β superfamily, PKC activator, and Notch signaling inhibitor) can be used in the method provided herein. In some embodiments, the FoxO1 inhibitor is AS1842856. In some examples, the method comprises contacting PDX1-positive pancreatic progenitor cells with a concentration of a FoxO1 inhibitor (e.g., AS1842856), such as, about 0.1 μM, about 0.12 μM, about 0.13 μM, about 0.14 μM, about 0.15 μM, about 0.16 μM, about 0.17 μM, about 0.18 μM, about 0.19 μM, about 0.2 μM, about 0.21 μM, about 0.22 μM, about 0.23 μM, about 0.24 μM, about 0.25 μM, about 0.26 μM, about 0.27 μM, about 0.28 μM, about 0.29 μM, about 0.3 μM, about 0.31 μM, about 0.32 μM, about 0.33 μM, about 0.34 μM, about 0.35 μM, about 0.4 μM, about 0.45 μM, about 0.5 μM, about 0.6 μM, about 0.8 μM, about 1 μM, about 2 μM, or about 5 μM. In some examples, the method comprises contacting PDX1-positive pancreatic progenitor cells with a concentration of a FoxO1 inhibitor (e.g., AS1842856), such as, about 0.7-1.3 μM, about 0.8-1.2 μM, about or 0.9-1.1 μM. In some examples, the method comprises contacting PDX1-positive pancreatic progenitor cells with a concentration of a FoxO1 inhibitor (e.g., AS1842856), such as, about 1 μM.

Any PKC activator capable of inducing PDX1-positive pancreatic progenitor cells to differentiate into NKX6.1-positive pancreatic progenitor cells (e.g., alone, or with any combination of at least one growth factor from the FGF family, at least one retinoic acid signaling pathway activator, ROCK inhibitor, at least one growth factor from the TGF-β superfamily, FoxO1 inhibitor, and Notch signaling inhibitor) can be used in the method provided herein. In some embodiments, the PKC activator is PDBU. In some examples, the method comprises contacting PDX1-positive pancreatic progenitor cells with a concentration of a PKC activator (e.g., PDBU), such as, about 0.1 μM, about 0.12 μM, about 0.13 μM, about 0.14 μM, about 0.15 μM, about 0.16 μM, about 0.17 μM, about 0.18 μM, about 0.19 μM, about 0.2 μM, about 0.21 μM, about 0.22 μM, about 0.23 μM, about 0.24 μM, about 0.25 μM, about 0.26 μM, about 0.27 μM, about 0.28 μM, about 0.29 μM, about 0.3 μM, about 0.31 μM, about 0.32 μM, about 0.33 μM, about 0.34 μM, about 0.35 μM, about 0.4 μM, about 0.45 μM, about 0.5 μM, about 0.6 μM, about 0.8 μM, about 1 μM, about 2 μM, or about 5 μM. In some examples, the method comprises contacting PDX1-positive pancreatic progenitor cells with a concentration of a PKC activator (e.g., PDBU), such as, about 0.2-0.8 μM, about 0.3-0.7 μM, about 0.4-0.6 μM. In some examples, the method comprises contacting PDX1-positive pancreatic progenitor cells with a concentration of a PKC activator (e.g., PDBU), such as, about 0.5 μM.

Any Notch signaling inhibitor capable of inducing PDX1-positive pancreatic progenitor cells to differentiate into NKX6.1-positive pancreatic progenitor cells (e.g., alone, or with any combination of at least one growth factor from the FGF family, at least one retinoic acid signaling pathway activator, ROCK inhibitor, at least one growth factor from the TGF-β superfamily, FoxO1 inhibitor, and PKC activator) can be used in the method provided herein. In some embodiments, the Notch signaling inhibitor is XXI. In some examples, the method comprises contacting PDX1-positive pancreatic progenitor cells with a concentration of a Notch signaling inhibitor (e.g., XXI), such as, about 0.1 μM, about 0.12 μM, about 0.13 μM, about 0.14 μM, about 0.15 μM, about 0.16 μM, about 0.17 μM, about 0.18 μM, about 0.19 μM, about 0.2 μM, about 0.21 μM, about 0.22 μM, about 0.23 μM, about 0.24 μM, about 0.25 μM, about 0.26 μM, about 0.27 μM, about 0.28 μM, about 0.29 μM, about 0.3 μM, about 0.31 μM, about 0.32 μM, about 0.33 μM, about 0.34 μM, about 0.35 μM, about 0.4 μM, about 0.45 μM, about 0.5 μM, about 0.6 μM, about 0.8 μM, about 1 μM, about 2 μM, or about 5 μM. In some examples, the method comprises contacting PDX1-positive pancreatic progenitor cells with a concentration of a Notch signaling inhibitor (e.g., XXI), such as, about 1.7-2.3 μM, about 1.8-2.2 μM, or about 1.9-2.1 μM. In some examples, the method comprises contacting PDX1-positive pancreatic progenitor cells with a concentration of a Notch signaling inhibitor (e.g., XXI), such as, about 2 μM.

In some embodiments, the cells are further contacted with a water-soluble synthetic polymer. In some embodiments, the water-soluble synthetic polymer is polyvinyl alcohol. In some cases, the polyvinyl alcohol is at least 78% hydrolyzed, e.g., 79-81% hydrolyzed, 87-89% hydrolyzed, 87-90% hydrolyzed, or 99% hydrolyzed. In some embodiments, the polyvinyl alcohol (PVA) is 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% hydrolyzed. In some embodiments, the PVA is 80% hydrolyzed.

In some embodiments, the PDX1-positive, NKX6.1-positive pancreatic progenitor cells are obtained by contacting PDX1-positive pancreatic progenitor cells under conditions that promote cell clustering with KGF, Sant1, and RA, for a period of 5 days or 6 days. In some embodiments, the PDX1-positive, NKX6.1-positive pancreatic progenitor cells are obtained by contacting PDX1-positive pancreatic progenitor cells under conditions that promote cell clustering with KGF, Sant1, RA, thiazovivin, and Activin A, for a period of 5 or 6 days. In some embodiments, the PDX1-positive, NKX6.1-positive pancreatic progenitor cells are obtained by contacting PDX1-positive pancreatic progenitor cells under conditions that promote cell clustering with KGF for a period of 5 days. In some embodiments, the PDX1-positive, NKX6.1-positive pancreatic progenitor cells are obtained by contacting PDX1-positive pancreatic progenitor cells under conditions that promote cell clustering with KGF for a period of 6 days. In some embodiments, the PDX1-positive, NKX6.1-positive pancreatic progenitor cells are obtained by: a) contacting PDX1-positive pancreatic progenitor cells with KGF, Sant1, RA, thiazovivin, and Activin A, for a period of 3, 4 or 5 days (e.g., 4 days), followed by; b) contacting the cells of a) with PDBU, XXI, KGF, Sant1, RA, thiazovivin, and Activin A and optionally AS1842856 for a period of 1, 2 or 3 days (e.g., 2 days).

### Insulin-Positive Endocrine Cells

Aspects of the disclosure involve insulin-positive endocrine cells (e.g., NKX6.1-positive, ISL1-positive cells, or β-like cells) and additional methods of generating insulin-positive endocrine cells. Insulin-positive endocrine cells of use herein can be derived from any source or generated in accordance with any suitable protocol. In some aspects, NKX6.1-positive pancreatic progenitor cells are differentiated to insulin-positive endocrine cells (e.g., NKX6.1-positive, ISL1-positive cells, or β-like cells). In some aspects, the insulin-positive endocrine cells are further differentiated, e.g., by induction or maturation to SC-β cells.

In some aspects, a method of producing an insulin-positive endocrine cell from an NKX6.1-positive pancreatic progenitor cell comprises contacting a population of cells (e.g., under conditions that promote cell clustering) comprising NKX6-1-positive pancreatic progenitor cells with a) a TGF-β signaling pathway inhibitor, b) a thyroid hormone signaling pathway activator, c) a BMP pathway inhibitor, and/or d) a protein kinase inhibitor to induce the differentiation of at least one NKX6.1-positive pancreatic progenitor cell in the population into an insulin-positive endocrine cell, wherein the insulin-positive endocrine ceil expresses insulin. In some embodiments, insulin-positive endocrine cells express PDX1, NKX6.1, ISL1, NKX2.2, Mafb, glis3, Sur1, Kir6.2, Znt8, SLC2A1, SLC2A3 and/or insulin.

Any TGF-β signaling pathway inhibitor capable of inducing the differentiation of NKX6.1-positive pancreatic progenitor cells to differentiate into insulin-positive endocrine cells (e.g., alone, or in combination with other β cell-differentiation factors, e.g., a thyroid hormone signaling pathway activator) can be used. In some embodiments, the TGF-β signaling pathway comprises TGF-β receptor type I kinase signaling. In some embodiments, the TGF-β signaling pathway inhibitor comprises Alk5 inhibitor II. In some examples, the method comprises contacting NKX6.1-positive pancreatic progenitor cells with a concentration of a TGF-β signaling pathway inhibitor (e.g., Alk5 inhibitor such as Alk5 inhibitor II), such as, about 0.1 μM, about 0.5 μM, about 1 μM, about 1.5 μM, about 2 μM, about 2.5 μM, about 3 μM, about 3.5 μM, about 4 μM, about 4.5 μM, about 5 μM, about 5.5 μM, about 6 μM, about 6.5 μM, about 7 μM, about 7.5 μM, about 8 μM, about 8.5 μM, about 9 μM, about 9.5 μM, about 10 μM, about 10.5 μM, about 11 μM, about 11.5 μM, about 12 μM, about 12.5 μM, about 13 μM, about 13.5 μM, about 14 μM, about 14.5 μM, about 15 μM, about 15.5 μM, about 16 μM, about 16.5 μM, about 17 μM, about 17.5 μM, about 18 μM, about 18.5 μM, about 19 μM, about 19.5 μM, about 20 μM, about 25 μM, about 30 μM, about 35 μM, about 40 μM, about 45 μM, or about 50 μM. In some examples, the method comprises contacting NKX6.1-positive pancreatic progenitor cells with a concentration of a TGF-β signaling pathway inhibitor (e.g., Alk5 inhibitor such as Alk5 inhibitor II), such as, about 7-13 μM, about 8-12 μM, about 9-11 μM. In some examples, the method comprises contacting NKX6.1-positive pancreatic progenitor cells with a concentration of a TGF-β signaling pathway inhibitor (e.g., Alk5 inhibitor such as Alk5 inhibitor II), such as, about 10 μM.

Any thyroid hormone signaling pathway activator capable of inducing the differentiation of NKX6.1-positive pancreatic progenitor cells to differentiate into insulin-positive endocrine cells (e.g., alone, or in combination with other β cell-differentiation factors, e.g., a TGF-β signaling pathway inhibitor) can be used. In some embodiments, the thyroid hormone signaling pathway activator comprises triiodothyronine (T3). In some embodiments, the thyroid hormone signaling pathway activator comprises GC-1. In some examples, the method comprises contacting NKX6.1-positive pancreatic progenitor cells with a concentration of thyroid hormone signaling pathway activator (e.g., GC-1), such as, about 0.1 μM, about 0.12 μM, about 0.13 μM, about 0.14 μM, about 0.15 μM, about 0.16 μM, about 0.17 μM, about 0.18 μM, about 0.19 μM, about 0.2 μM, about 0.21 μM, about 0.22 μM, about 0.23 μM, about 0.24 μM, about 0.25 μM, about 0.26 μM, about 0.27 μM, about 0.28 μM, about 0.29 μM, about 0.3 μM, about 0.31 μM, about 0.32 μM, about 0.33 μM, about 0.34 μM, about 0.35 μM, about 0.4 μM, about 0.45 μM, about 0.5 μM, about 0.6 μM, about 0.8 μM, about 1 μM, about 2 μM, or about 5 μM. In some examples, the method comprises contacting NKX6.1-positive pancreatic progenitor cells with a concentration of thyroid hormone signaling pathway activator (e.g., GC-1), such as, about 0.7-1.3 μM, about 0.8-1.2 μM, or about 0.9-1.1 μM. In some examples, the method comprises contacting NKX6.1-positive pancreatic progenitor cells with a concentration of thyroid hormone signaling pathway activator (e.g., GC-1), such as, about 1 μM.

In some embodiments, the method comprises contacting the population of cells (e.g., NKX6.1-positive pancreatic progenitor cells) with at least one additional factor. In some embodiments, the method comprises contacting the PDX1-positive NKX6.1-positive pancreatic progenitor cells with at least one of i) a SHH pathway inhibitor, ii) a γ-secretase inhibitor, iii) at least one growth factor from the epidermal growth factor (EGF) family, iv) a TGF-β signaling pathway inhibitor, or vii) a thyroid hormone signaling pathway activator. In some embodiments, the method comprises contacting the population of cells (e.g., NKX6.1-positive pancreatic progenitor cells) with at least one additional factor. In some embodiments, the method comprises contacting the PDX1-positive NKX6.1-positive pancreatic progenitor cells with at least one of i) a SHH pathway inhibitor, ii) a RA signaling pathway activator, iii) a γ-secretase inhibitor, iv) at least one growth factor from the epidermal growth factor (EGF) family, v) a protein kinase inhibitor, vi) a TGF-β signaling pathway inhibitor, vii) a thyroid hormone signaling pathway activator, viii) a wnt signaling pathway inhibitor, or ix) a PKC activator.

In some embodiments, the method comprises contacting the PDX1-positive NKX6.1-positive pancreatic progenitor cells with at least one of i) a SHH pathway inhibitor, ii) a RA signaling pathway activator, iii) a γ-secretase inhibitor, iv) at least one growth factor from the epidermal growth factor (EGF) family, v) at least one bone morphogenetic protein (BMP) signaling pathway inhibitor, vi) a TGF-β signaling pathway inhibitor, vii) a thyroid hormone signaling pathway activator, viii) a protein kinase inhibitor, or ix) a ROCK inhibitor.

In some embodiments, the method comprises contacting the PDX1-positive NKX6.1-positive pancreatic progenitor cells with at least one of i) a SHH pathway inhibitor, ii) a RA signaling pathway activator, iii) a γ-secretase inhibitor, iv) at least one growth factor from the epidermal growth factor (EGF) family, v) at least one bone morphogenetic protein (BMP) signaling pathway inhibitor, vi) a TGF-β signaling pathway inhibitor, vii) a thyroid hormone signaling pathway activator, viii) an epigenetic modifying compound, ix) a protein kinase inhibitor, or x) a ROCK inhibitor. In some embodiments, the method comprises contacting the PDX1-positive, NKX6.1-positive pancreatic progenitor cells in a culture with a i) a SHH pathway inhibitor, ii) a RA signaling pathway activator, iii) a γ-secretase inhibitor, iv) at least one growth factor from the epidermal growth factor (EGF) family, v) at least one bone morphogenetic protein (BMP) signaling pathway inhibitor, vi) a TGF-β signaling pathway inhibitor, vii) a thyroid hormone signaling pathway activator, viii) an epigenetic modifying compound, ix) a protein kinase inhibitor, x) a ROCK inhibitor, xi) a PKC activator and xii) a Wnt signaling pathway inhibitor for 1, 2, or 3 days (e.g., 1-2, 1-3, or 2-3 days), and then contacting the cells in the culture with i) a γ-secretase inhibitor, ii) at least one growth factor from the epidermal growth factor (EGF) family, iii) at least one bone morphogenetic protein (BMP) signaling pathway inhibitor, iv) a TGF-β signaling pathway inhibitor, v) a thyroid hormone signaling pathway activator, vi) an epigenetic modifying compound, vii) a protein kinase inhibitor, and viii) a ROCK inhibitor for a period of 1, 2, 3, 4, 5, 6, or 7 days (e.g., 1-7, 1-5, 1-3, 3-7, 3-5, 5-7, or 4-6 days) in the absence of a SHH pathway inhibitor, a RA signaling pathway activator, a Wnt signaling pathway inhibitor, PKC activator, and/or growth factor from the epidermal growth factor (EGF) family.

In some embodiments, in the method of generating the insulin-positive endocrine cells from the PDX1-positive NKX6.1-positive pancreatic progenitor cells, some of the differentiation factors are present only for the first 1, 2, 3, 4, or 5 days during the differentiation step. In some embodiments, some of the differentiation factors, such as the SHH pathway inhibitor, the RA signaling pathway activator, the PKC activator, and the at least one growth factor from the EGF family are removed from the culture medium after the first 1, 2, or 3 days of incubation.

Any γ-secretase inhibitor that is capable of inducing the differentiation of NKX6.1-positive pancreatic progenitor cells in a population into insulin-positive endocrine cells (e.g., alone, or in combination with any of a TGF-β signaling pathway inhibitor and/or a thyroid hormone signaling pathway activator) can be used. In some embodiments, the γ-secretase inhibitor comprises XXI. In some embodiments, the γ-secretase inhibitor comprises DAPT. In some examples, the method comprises contacting NKX6.1-positive pancreatic progenitor cells with a concentration of a γ-secretase inhibitor (e.g., XXI), such as, about 0.01 μM, about 0.02 μM, about 0.05 μM, about 0.075 μM, about 0.1 μM, about 0.2 μM, about 0.3 μM, about 0.4 μM, about 0.5 μM, about 0.6 μM, about 0.7 μM, about 0.8 μM, about 0.9 μM, about 1 μM, about 1.1 μM, about 1.2 μM, about 1.3 μM, about 1.4 μM, about 1.5 μM, about 1.6 μM, about 1.7 μM, about 1.8 μM, about 1.9 μM, about 2 μM, about 2.1 μM, about 2.2 μM, about 2.3 μM, about 2.4 μM, about 2.5 μM, about 2.6 μM, about 2.7 μM, about 2.8 μM, about 2.9 μM, about 3 μM, about 3.2 μM, about 3.4 μM, about 3.6 μM, about 3.8 μM, about 4 μM, about 4.2 μM, about 4.4 μM, about 4.6 μM, about 4.8 μM, about 5 μM, about 5.2 μM, about 5.4 μM, about 5.6 μM, about 5.8 μM, about 6 μM, about 6.2 μM, about 6.4 μM, about 6.6 μM, about 6.8 μM, about 7 μM, about 8 μM, about 9 μM, about 10 μM, about 20 μM, about 30 μM, or about 50 μM. In some examples, the method comprises contacting NKX6.1-positive pancreatic progenitor cells with a concentration of a γ-secretase inhibitor (e.g., XXI), such as, about 1.7-2.3 μM, about 1.8-2.2 μM, or about 1.9-2.1 μM. In some examples, the method comprises contacting NKX6.1-positive pancreatic progenitor cells with a concentration of a γ-secretase inhibitor (e.g., XXI), such as about 2 μM.

Any growth factor from the EGF family capable of inducing the differentiation of NKX6.1-positive pancreatic progenitor cells in a population into insulin-positive endocrine cells (e.g., alone, or in combination with any of a TGF-β signaling pathway inhibitor and/or a thyroid hormone signaling pathway activator) can be used. In some embodiments, the at least one growth factor from the EGF family comprises betacellulin. In some embodiments, at least one growth factor from the EGF family comprises EGF. In some examples, the method comprises contacting NKX6.1-positive pancreatic progenitor cells with a concentration of a growth factor from EGF family (e.g., betacellulin), such as, about 1 ng/mL, about 2 ng/ml, about 4 ng/ml, about 6 ng/mL, about 8 ng/ml, about 10 ng/ml, about 12 ng/ml, about 14 ng/mL, about 16 ng/ml, about 18 ng/mL, about 20 ng/ml, about 22 ng/ml, about 24 ng/ml, about 26 ng/ml, about 28 ng/mL, about 30 ng/ml, about 40 ng/mL, about 50 ng/ml, about 75 ng/ml, about 80 ng/mL, about 90 ng/ml, about 95 ng/mL, about 100 ng/ml, about 150 ng/ml, about 200 ng/ml, about 250 ng/ml, or about 300 ng/mL. In some examples, the method comprises contacting NKX6.1-positive pancreatic progenitor cells with a concentration of a growth factor from EGF family (e.g., betacellulin), such as, about 17-23 ng/ml, about 18-22 ng/ml, or about 19-21 ng/ml. In some examples, the method comprises contacting NKX6.1-positive pancreatic progenitor cells with a concentration of a growth factor from EGF family (e.g., betacellulin), such as, about 20 ng/ml.

Any RA signaling pathway activator capable of inducing the differentiation of NKX6.1-positive pancreatic progenitor cells to differentiate into insulin-positive endocrine cells (e.g., alone, or in combination with any of a TGF-β signaling pathway inhibitor and/or a thyroid hormone signaling pathway activator) can be used. In some embodiments, the RA signaling pathway activator comprises RA. In some examples, the method comprises contacting NKX6.1-positive pancreatic progenitor cells with a concentration of an RA signaling pathway activator (e.g., retinoic acid), such as, about 0.02 μM, about 0.05 μM, about 0.1 μM, about 0.2 μM, about 0.25 μM, about 0.3 μM, about 0.4 μM, about 0.45 μM, about 0.5 μM, about 0.55 μM, about 0.6 μM, about 0.65 μM, about 0.7 μM, about 0.75 μM, about 0.8 μM, about 0.85 μM, about 0.9 μM, about 1 μM, about 1.1 μM, about 1.2 μM, about 1.3 μM, about 1.4 μM, about 1.5 μM, about 1.6 μM, about 1.7 μM, about 1.8 μM, about 1.9 μM, about 2 μM, about 2.1 μM, about 2.2 μM, about 2.3 μM, about 2.4 μM, about 2.5 μM, about 2.6 μM, about 2.7 μM, about 2.8 μM, about 3 μM, about 3.2 μM, about 3.4 μM, about 3.6 μM, about 3.8 μM, about 4 μM, about 4.2 μM, about 4.4 μM, about 4.6 μM, about 4.8 μM, about 5 μM, about 5.5 μM, about 6 μM, about 6.5 μM, about 7 μM, about 7.5 μM, about 8 μM, about 8.5 μM, about 9 μM, about 9.5 μM, about 10 μM, about 12 μM, about 14 μM, about 15 μM, about 16 μM, about 18 μM, about 20 μM, about 50 μM, or about 100 μM. In some examples, the method comprises contacting NKX6.1-positive pancreatic progenitor cells with a concentration of an RA signaling pathway activator (e.g., retinoic acid), such as, about 20-80 nM, about 30-70 nM, or about 40-60 nM. In some examples, the method comprises contacting NKX6.1-positive pancreatic progenitor cells with a concentration of an RA signaling pathway activator (e.g., retinoic acid), such as, about 50 nM.

Any SHH pathway inhibitor capable of inducing the differentiation of NKX6.1-positive pancreatic progenitor cells to differentiate into insulin-positive endocrine cells (e.g., alone, or in combination with any of a TGF-β signaling pathway inhibitor and/or a thyroid hormone signaling pathway activator) can be used in the method provided herein. In some embodiments, the SHH pathway inhibitor comprises Sant1. In some examples, the method comprises contacting NKX6.1-positive pancreatic progenitor cells with a concentration of a SHH pathway inhibitor (e.g., Sant1), such as, about 0.001 μM, about 0.002 μM, about 0.005 μM, about 0.01 μM, about 0.02 μM, about 0.03 μM, about 0.05 μM, about 0.08 μM, about 0.1 μM, about 0.12 μM, about 0.13 μM, about 0.14 μM, about 0.15 μM, about 0.16 μM, about 0.17 μM, about 0.18 μM, about 0.19 μM, about 0.2 μM, about 0.21 μM, about 0.22 μM, about 0.23 μM, about 0.24 μM, about 0.25 μM, about 0.26 μM, about 0.27 μM, about 0.28 μM, about 0.29 μM, about 0.3 μM, about 0.31 μM, about 0.32 μM, about 0.33 μM, about 0.34 μM, about 0.35 μM, about 0.4 μM, about 0.45 μM, about 0.5 μM, about 0.6 μM, about 0.8 μM, about 1 μM, about 2 μM, or about 5 μM. In some examples, the method comprises contacting NKX6.1-positive pancreatic progenitor cells with a concentration of a SHH pathway inhibitor (e.g., Sant1), such as, about 220-280 nM, about 230-270 nM, about 240-260 nM, or about 245-255 nM. In some examples, the method comprises contacting NKX6.1-positive pancreatic progenitor cells with a concentration of a SHH pathway inhibitor (e.g., Sant1), such as, about 250 nM.

Any BMP signaling pathway inhibitor capable of inducing the differentiation of NKX6.1-positive pancreatic progenitor cells to differentiate into insulin-positive endocrine cells (e.g., alone, or in combination with any of a TGF-β signaling pathway inhibitor and/or a thyroid hormone signaling pathway activator) can be used. In some embodiments, the BMP signaling pathway inhibitor comprises LDN193189 or DMH-1. In some examples, the method comprises contacting NKX6.1-positive pancreatic progenitor cells with a concentration of BMP signaling pathway inhibitor (e.g., LDN1931189), such as, about 30 nM, about 40 nM, about 50 nM, about 60 nM, about 70 nM, about 80 nM, about 90 nM, about 100 nM, about 110 nM, about 120 nM, about 130 nM, about 140 nM, about 150 nM, about 160 nM, about 170 nM, about 180 nM, about 190 nM, about 200 nM, about 210 nM, about 220 nM, about 230 nM, about 240 nM, about 250 nM, about 280 nM, about 300 nM, about 400 nM, about 500 nM, or about 1 μM. In some examples, the method comprises contacting NKX6.1-positive pancreatic progenitor cells with a concentration of BMP signaling pathway inhibitor (e.g., LDN1931189), such as, about 70-130 nM, about 80-120 nM, about 90-110 nM. In some examples, the method comprises contacting NKX6.1-positive pancreatic progenitor cells with a concentration of BMP signaling pathway inhibitor (e.g., LDN1931189), such as, about 100 nM.

Any ROCK inhibitor that is capable of inducing the differentiation of NKX6.1-positive pancreatic progenitor cells in a population into insulin-positive endocrine cells (e.g., alone, or in combination with any of a TGF-β signaling pathway inhibitor and/or a thyroid hormone signaling pathway activator) can be used. In some embodiments, the ROCK inhibitor comprises Thiazovivin, Y-27632, Fasudil/HA1077, or H-1152. In some embodiments, the ROCK inhibitor comprises Y-27632. In some embodiments, the ROCK inhibitor comprises Thiazovivin. In some examples, the method comprises contacting PDX1-positive, NKX6.1-positive pancreatic progenitor cells with a concentration of a ROCK inhibitor (e.g., Y-27632 or Thiazovivin), such as, about 0.2 μM, about 0.5 μM, about 0.75 μM, about 1 μM, about 2 μM, about 3 μM, about 4 μM, about 5 μM, about 6 μM, about 7 μM, about 7.5 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 50 μM, or about 100 μM. In some embodiments, the ROCK inhibitor comprises Thiazovivin. In some examples, the method comprises contacting PDX1-positive, NKX6.1-positive pancreatic progenitor cells with a concentration of a ROCK inhibitor (e.g., Y-27632 or Thiazovivin), such as, about 2.2-2.8 μM, about 2.3-2.7 μM, or about 2.4-2.6 μM. In some embodiments, the ROCK inhibitor comprises Thiazovivin. In some examples, the method comprises contacting PDX1-positive, NKX6.1-positive pancreatic progenitor cells with a concentration of a ROCK inhibitor (e.g., Y-27632 or Thiazovivin), such as, about 2.5 μM.

Any epigenetic modifying compound that is capable of inducing the differentiation of NKX6.1-positive pancreatic progenitor cells in a population into insulin-positive endocrine cells (e.g., alone, or in combination with any of a TGF-β signaling pathway inhibitor and/or a thyroid hormone signaling pathway activator) can be used. In some embodiments, the epigenetic modifying compound comprises a histone methyltransferase inhibitor or a HDAC inhibitor. In some embodiments, the epigenetic modifying compound comprises a histone methyltransferase inhibitor, e.g., DZNep. In some embodiments, the epigenetic modifying compound comprises a HDAC inhibitor, e.g., KD5170. In some examples, the method comprises contacting PDX1-positive, NKX6.1-positive pancreatic progenitor cells with a concentration of an epigenetic modifying compound (e.g., DZNep or KD5170), such as, about 0.01 μM, about 0.025 μM, about 0.05 μM, about 0.075 μM, about 0.1 μM, about 0.15 μM, about 0.2 μM, about 0.5 μM, about 0.75 μM, about 1 μM, about 2 μM, about 3 μM, about 4 μM, about 5 μM, about 6 μM, about 7 μM, about 7.5 μM, about 8 μM, about 9 μM, about 10 μM, about 15 μM, about 20 μM, about 25 μM, about 30 μM, about 35 μM, about 40 μM, about 50 μM, or about 100 μM. In some examples, the method comprises contacting PDX1-positive, NKX6.1-positive pancreatic progenitor cells with a concentration of an epigenetic modifying compound (e.g., DZNep or KD5170), such as, about 70-130 nM, about 80-120 nM, or about 90-110 nM. In some examples, the method comprises contacting PDX1-positive, NKX6.1-positive pancreatic progenitor cells with a concentration of an epigenetic modifying compound (e.g., DZNep or KD5170), such as, about 100 nM.

Any Wnt signaling pathway inhibitor that is capable of inducing the differentiation of NKX6.1-positive pancreatic progenitor cells in a population into insulin-positive endocrine cells (e.g., alone, or in combination with any of a TGF-β signaling pathway inhibitor and/or a thyroid hormone signaling pathway activator) can be used. In some embodiments, the Wnt signaling pathway inhibitor comprises a tankyrase inhibitor. In some embodiments, the tankyrase inhibitor is NVP-TNKS656. In some examples, the method comprises contacting PDX1-positive, NKX6.1-positive pancreatic progenitor cells with a concentration of a Wnt signaling pathway inhibitor (e.g., a tankyrase inhibitor such as NVP-TNKS656), such as, about 0.1 μM, about 0.15 μM, about 0.2 μM, about 0.25 μM, about 0.3 μM, about 0.35 μM, about 0.4 μM, about 0.45 μM, about 0.5 μM, about 0.55 μM, about 0.6 μM, about 0.65 μM, about 0.7 μM, about 0.75 μM, about 0.8 μM, about 0.85 μM, about 0.9 μM, about 0.95 μM, about 1 μM, about 1.5 μM, about 2 μM, about 2.5 μM, about 3 μM, about 3.5 μM, about 4 μM, about 4.5 μM, or about 5 μM. In some examples, the method comprises contacting PDX1-positive, NKX6.1-positive pancreatic progenitor cells with a concentration of a Wnt signaling pathway inhibitor (e.g., a tankyrase inhibitor such as NVP-TNKS656), such as, about 1.7-2.3 μM, about 1.8-2.2 μM, or about 1.9-2.1 μM. In some examples, the method comprises contacting PDX1-positive, NKX6.1-positive pancreatic progenitor cells with a concentration of a Wnt signaling pathway inhibitor (e.g., a tankyrase inhibitor such as NVP-TNKS656), such as, about 2 μM.

Any PKC activator that is capable of inducing the differentiation of NKX6.1-positive pancreatic progenitor cells in a population into insulin-positive endocrine cells (e.g., alone, or in combination with any of a TGF-β signaling pathway inhibitor and/or a thyroid hormone signaling pathway activator) can be used. In some embodiments, the PKC activator is TPB or PDBU. In some examples, the method comprises contacting PDX1-positive, NKX6.1-positive pancreatic progenitor cells with a concentration of a PKC activator (TPB or PDBU), such as, about 0.01 μM, about 0.025 μM, about 0.05 μM, about 0.075 μM, about 0.1 μM, about 0.15 μM, about 0.2 μM, about 0.25 μM, about 0.3 μM, about 0.35 μM, about 0.4 μM, about 0.45 μM, about 0.5 μM, about 0.55 μM, about 0.6 μM, about 0.65 μM, about 0.7 μM, about 0.75 μM, about 0.8 μM, about 0.85 μM, about 0.9 μM, about 0.95 μM, about 1 μM, about 2 μM, about 3 μM, about 4 μM, about 5 μM, about 6 μM, about 7 μM, about 7.5 μM, about 8 μM, about 9 μM, about 10 μM, about 15 μM, or about 20 μM. In some examples, the method comprises contacting PDX1-positive, NKX6.1-positive pancreatic progenitor cells with a concentration of a PKC activator (TPB or PDBU), such as, about 450-550 mM, about 475-525 nM, about 490-510 nM, or about 495-505 nM. In some examples, the method comprises contacting PDX1-positive, NKX6.1-positive pancreatic progenitor cells with a concentration of a PKC activator (TPB or PDBU), such as, about 500 nM.

In some embodiments, the population of cells is optionally contacted with a protein kinase inhibitor. In some embodiments, the population of cells is not contacted with the protein kinase inhibitor. In some embodiments, the population of cells is contacted with the protein kinase inhibitor. Any protein kinase inhibitor that is capable of inducing the differentiation of NKX6.1-positive pancreatic progenitor cells in a population into insulin-positive endocrine cells (e.g., alone, or in combination with any of a TGF-β signaling pathway inhibitor and/or a thyroid hormone signaling pathway activator). In some embodiments, the protein kinase inhibitor comprises staurosporine. In some examples, the method comprises contacting NKX6.1-positive pancreatic progenitor cells with a concentration of a protein kinase inhibitor (e.g., staurosporine), such as, about 0.1 nM, about 0.2 nM, about 0.3 nM, about 0.4 nM, about 0.5 nM, about 0.6 nM, about 0.7 nM, about 0.8 nM, about 0.9 nM, about 1 nM, about 1.1 nM, about 1.2 nM, about 1.3 nM, about 1.4 nM, about 1.5 nM, about 1.6 nM, about 1.7 nM, about 1.8 nM, about 1.9 nM, about 2.0 nM, about 2.1 nM, about 2.2 nM, about 2.3 nM, about 2.4 nM, about 2.5 nM, about 2.6 nM, about 2.7 nM, about 2.8 μM, about 2.9 nM, about 3 nM, about 3.1 nM, about 3.2 nM, about 3.3 nM, about 3.4 nM, about 3.5 nM, about 3.6 nM, about 3.7 nM, about 3.8 nM, about 3.9 nM, about 4.0 nM, about 4.1 nM, about 4.2 nM, about 4.3 nM, about 4.4 nM, about 4.5 nM, about 4.6 nM, about 4.7 nM, about 4.8 μM, about 4.9 nM, or about 5 nM. In some examples, the method comprises contacting NKX6.1-positive pancreatic progenitor cells with a concentration of a protein kinase inhibitor (e.g., staurosporine), such as, about 1-5 nM, about 2-4 nM, or about 2.5-3.5 nM. In some examples, the method comprises contacting NKX6.1-positive pancreatic progenitor cells with a concentration of a protein kinase inhibitor (e.g., staurosporine), such as, about 3 nM.

In some embodiments, the cells are further contacted with a water-soluble synthetic polymer. In some embodiments, the water-soluble synthetic polymer is polyvinyl alcohol. In some cases, the polyvinyl alcohol is at least 78% hydrolyzed, e.g., 79-81% hydrolyzed, 87-89% hydrolyzed, 87-90% hydrolyzed, or 99% hydrolyzed. In some embodiments, the polyvinyl alcohol (PVA) is 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% hydrolyzed. In some embodiments, the PVA is 89% hydrolyzed.

In some embodiments, the method comprises contacting the population of cells (e.g., NKX6.1-positive pancreatic progenitor cells) with XXI, Alk5i, T3 or GC-1, RA, Sant1, and betacellulin, PDBU, and NVP-TNKS656 for a period of 7 days, to induce the differentiation of at least one NKX6.1-positive pancreatic progenitor cell in the population into an insulin-positive endocrine cell, wherein the insulin-positive endocrine cell expresses insulin. In some embodiments, the method comprises contacting the population of cells (e.g., NKX6.1-positive pancreatic progenitor cells) with XXI, Alk5i, T3 or GC-1, RA, Sant1, betacellulin, and LDN193189 for a period of 7 days, to induce the differentiation of at least one NKX6.1-positive pancreatic progenitor cell in the population into an insulin-positive endocrine cell, wherein the insulin-positive endocrine cell expresses insulin. In some embodiments, one or more differentiation factors are added in a portion of the Stage 5, for instance, only the first 1, 2, 3, 4, 5, or 6 days of the period of time for Stage 5, or the last 1, 2, 3, 4, 5, or 6 days of the period of time for Stage 5. In one example, the cells are contacted with SHH signaling pathway inhibitor the PKC activator, the retinoic acid, and/or the wnt signaling pathway inhibitor for only the first 2, 3, 4, or 5 days during Stage 5, after which the SHH signaling pathway inhibitor, the PKC activator, the retinoic acid, and/or the wnt signaling pathway inhibitor are not included in or removed from the culture medium. In another example, the cells are contacted with BMP signaling pathway inhibitor for only the first 1, 2, or 3 days during Stage 5, after which the BMP signaling pathway inhibitor is removed from the culture medium.

In some embodiments, the method comprises contacting the population of cells (e.g., NKX6.1-positive pancreatic progenitor cells) with one or more metabolites. In some embodiments, the method comprises contacting the population of cells (e.g., NKX6.1-positive pancreatic progenitor cells) with one or more of an acetyl CoA-related metabolite, a vitamin, histone deacetylase inhibitor (HDACi), a redox homeostasis regulator, a one carbon metabolism pathway intermediate, and/or glutamine. Examples of metabolites include glutamine, taurine, acetate, beta-hydroxybutyrate, biotin, and formate.

In some embodiments, a composition (e.g., medium) of the disclosure comprises anacetyl CoA-related metabolite. Exemplary acetyl COA-related metabolites include, but are not limited to acetate, pyruvate, ketogenic amino acids, valine, leucine, isoleucine, phenylalanine, tyrosine, lysine, tryptophan, fatty acids, CoA, Isovaleryl-CoA, and β-hydroxybutyrate. In some embodiments, the acetyl COA-related metabolite is acetate. In some embodiments, the acetyl CoA-related metabolite is present in or is added to a composition of the disclosure at a concentration of about 10 nM, about 50 nM, about 80 nM, about 100 nM, about 120 nM, about 140 nM, about 150 nM, about 200 nM, about 300 nM, about 500 nM, about 800 nM, about 1 μM, about 10 μM, about 100 μM, about 500 μM, about 800 μM, about 900 μM, about 1 mM, about 2 mM, about 3 mM, about 5 mM, or about 10 mM. In some embodiments, the acetyl CoA-related metabolite is present in or is added to a composition of the disclosure at a concentration of about 0.01-50 mM, 0.1-50 mM, 0.5-50 mM, 0.01-20 mM, 0.1-20 mM, 0.5-20 mM, 0.01-10 mM, 0.1-10 mM, 0.5-10 mM, 0.8-25 mM, 0.8-10 mM, 0.8-5 mM, 0.8-2 mM, 0.8-1.5 mM, 0.8-1.2 mM, 0.9-1.1 mM, or 0.95-1.05 mM. In some embodiments, the acetyl CoA-related metabolite is acetate present at a concentration of about 1 mM. In some embodiments, the acetyl COA-related metabolite is acetate present at a concentration of about 50-1000 nM, 50-800 nM, 50-500 nM, 50-300 nM, 50-250 nM, 100-200 nM, or 125-175 nM. In some embodiments, the acetyl CoA-related metabolite is acetate present at a concentration of about 160 nM.

In some embodiments, a composition (e.g., medium) of the disclosure comprises one or more vitamins. Exemplary vitamins include, but are not limited to biotin, vitamin B1 (thiamine), vitamin B2 (riboflavin), vitamin B3 (niacin), vitamin B6 (pyridoxine) and vitamin B12 (cyanocobalamin). In some embodiments the vitamin modulates fatty acid synthesis. In some embodiments the vitamin modulates branched-chain amino acid metabolism. In some embodiments the vitamin modulates or participates as a co-factor in the TCA cycle, e.g., as a cofactor for pyruvate carboxylase. In some embodiments, the vitamin is biotin. In some embodiments, the vitamin is present in or is added to a composition of the disclosure at a concentration of about 100 nM, about 300 nM, about 500 nM, about 600 nM, about 700 nM, about 800 nM, about 900 nM, about 1 μM, about 1.5 μM, about 3 μM, about 5 μM, about 10 μM, or about 100 μM. In some embodiments, the vitamin is biotin present at a concentration of about 800 nM. In some embodiments, the vitamin is present in or is added to a composition of the disclosure at a concentration of about 1 nM to 500 μM, 1 nM to 100 μM, 1 nM to 10 μM, 1 nM to 1 μM, 1 nM to 800 nM, 1 nM to 600 nM, 1 nM to 400 nM, 1 nM to 300 nM, 1 nM to 200 nM, 25 nM to 500 μM, 25 nM to 100 μM, 25 nM to 10 μM, 25 nM to 1 μM, 25 nM to 800 nM, 25 nM to 600 nM, 25 nM to 400 nM, 25 nM to 300 nM, 25 nM to 200 nM, 50 nM to 500 μM, 50 nM to 100 μM, 50 nM to 10 μM, 50 nM to 1 μM, 50 nM to 800 nM, 50 nM to 600 nM, 50 nM to 400 nM, 50 nM to 300 nM, 50 nM to 200 nM, 100 nM to 500 μM, 100 nM to 100 μM, 100 nM to 10 μM, 100 nM to 1 μM, 100 nM to 800 nM, 100 nM to 600 nM, 100 nM to 400 nM, 100 nM to 300 nM, or 100 nM to 200 nM.

In some embodiments, a composition (e.g., medium) of the disclosure comprises a histone deacetylase inhibitor (HDACi). Exemplary histone deacetylase inhibitors (HDACi) include, but are not limited to β-Hydroxybutyrate, butyric acid, class I HDACi, class IIA HDACi, class IIB HDACi, class III HDACi, class IV HDACi, HDAC-1, HDAC-2, HDAC-3, HDAC-4, HDAC-5, HDAC-6, HDAC-7, HDAC-8, HDAC-9, HDAC-10, HDAC-11, sirtuins, SIRT1, SIRT2, SIRT3, SIRT4, SIRT5, SIRT6, SIRT7, Vorinostat (suberoylanilide hydroxamic acid, SAHA, MK0683), Entinostat (MS-275, SNDX-275), Panobinostat (LBH589, NVP-LBH589), Trichostatin A (TSA), Mocetinostat (MGCD0103, MG0103), GSK3117391 (GSK3117391A, HDAC-IN-3), BRD3308, BRD3308, Tubastatin A TFA (Tubastatin A trifluoroacetate salt), Tubastatin A, SIS17, NKL 22, BML-210 (CAY10433), TC-H 106, SR-4370, Belinostat (PXD101, NSC726630, PX-105684), Romidepsin (FK228, Depsipeptide, FR 901228, NSC 630176), MC1568, Givinostat (ITF2357), Dacinostat (LAQ824, NVP-LAQ824), CUDC-101, Quisinostat (JNJ-26481585), Pracinostat (SB939), PCI-34051, Droxinostat (NS 41080), Abcxinostat (PCI-24781), Abexinostat (PCI-24781, CRA-024781), RGFP966, AR-42 (HDAC-42), Ricolinostat (ACY-1215, Rocilinostat), Valproic acid sodium salt (Sodium valproate), Tacedinaline (CI994, PD-123654, GOE-5549, Acetyldinaline), Fimepinostat (CUDC-907), Sodium butyrate (NaB), Curcumin, Diferuloylmethane, M344, Tubacin, RG2833 (RGFP109), RG2833 (RGFP109), Resminostat (RAS2410), Divalproex Sodium, Scriptaid (GCK 1026), Sodium Phenylbutyrate, Sinapinic acid (Sinapic acid), TMP269, Santacruzamate A (CAY10683), TMP195 (TFMO 2), Valproic acid (VPA), UF010, Tasquinimod (ABR-215050), SKLB-23bb, Isoguanosine, Sulforaphane, BRD73954, Citarinostat (ACY-241, HDAC-IN-2), Suberohydroxamic acid, Splitomicin, HPOB, LMK-235, Biphenyl-4-sulfonyl chloride (p-Phenylbenzenesulfonyl, 4-Phenylbenzenesulfonyl, p-Biphenylsulfonyl), Nexturastat A, TH34, Tucidinostat (Chidamide, HBI-8000, CS-055), (−)-Parthenolide, WT161, CAY10603, CAY10603, ACY-738, Raddeanin A, Tinostamustine (EDO-S101), Domatinostat (4SC-202), and BG45. In some embodiments, the HDACi is β-Hydroxybutyrate. In some embodiments, the HDACi is present in or is added to a composition of the disclosure at a concentration of about 100 nM, about 300 nM, about 500 nM, about 600 nM, about 700 nM, about 800 nM, about 900 nM, about 1 μM, about 1.5 μM, about 3 μM, about 5 μM, about 10 μM, or about 100 μM. In some embodiments, the HDACi is β-Hydroxybutyrate present at a concentration of about 200 nM. In some embodiments, the HDACi is present in or is added to a composition of the disclosure at a concentration of about 1 nM to 500 μM, 1 nM to 100 μM, 1 nM to 10 μM, 1 nM to 1 μM, 1 nM to 800 nM, 1 nM to 600 nM, 1 nM to 400 nM, 1 nM to 300 nM, 1 nM to 200 nM, 25 nM to 500 μM, 25 nM to 100 μM, 25 nM to 10 μM, 25 nM to 1 μM, 25 nM to 800 nM, 25 nM to 600 nM, 25 nM to 400 nM, 25 nM to 300 nM, 25 nM to 200 nM, 50 nM to 500 μM, 50 nM to 100 μM, 50 nM to 10 μM, 50 nM to 1 μM, 50 nM to 800 nM, 50 nM to 600 nM, 50 nM to 400 nM, 50 nM to 300 nM, 50 nM to 200 nM, 100 nM to 500 μM, 100 nM to 100 μM, 100 nM to 10 μM, 100 nM to 1 μM, 100 nM to 800 nM, 100 nM to 600 nM, 100 nM to 400 nM, 100 nM to 300 nM, or 100 nM to 200 nM.

In some embodiments, a composition (e.g., medium) of the disclosure comprises a redox homeostasis regulator. Exemplary redox homeostasis regulators include, but are not limited to taurine, respiratory chain regulators, free radical scavengers, regulators of mitochondrial protein synthesis, allium sulphur compounds, anthocyanins, beta-carotene, catechins, copper, cryptoxanthins, flavonoids, indoles, isoflavonoids, lignans, lutein, lycopene, alpha lipoic acid, ellagic acid, manganese, polyphenols, selenium, glutathione, vitamin A, vitamin C, vitamin E, zinc, superoxide disutases, GSHPx, Prx-I, catalase, and co-enzyme Q10. In some embodiments, the redox homeostasis regulator is taurine. In some embodiments, the redox homeostasis regulator is present in or is added to a composition of the disclosure at a concentration of about 100 nM, about 500 nM, 1 μM, about 10 μM, about 20 μM, about 30 μM, about 40 μM, about 50 μM, about 60 μM, about 70 μM, about 80 μM, about 90 μM, about 100 μM, about 110 μM, about 110 μM, about 150 μM, or about 200 μM. In some embodiments, the redox homeostasis regulator is taurine. In some embodiments, the redox homeostasis regulator is taurine present at a concentration of about 90 μM. In some embodiments, the redox homeostasis regulator intermediate is present or is added at a concentration of about 100 nM to 1 mM, 500 nM to 1 mM, 1 μM to 1 mM, 10 μM to 1 mM, 20 μM to 1 mM, 30 μM to 1 mM, 30 μM to 1 mM, 40 μM to 1 mM, 50 μM to 1 mM, 60 μM to 1 mM, 70 μM to 1 mM, 80 μM to 1 mM, 100 nM to 250 μM, 500 nM to 250 μM, 1 μM to 250 μM, 10 μM to 250 μM, 20 μM to 250 μM, 30 μM to 250 μM, 30 μM to 250 μM, 40 μM to 250 μM, 50 μM to 250 μM, 60 μM to 250 μM, 70 μM to 250 μM, 100 nM to 100 μM, 500 nM to 100 μM, 1 μM to 100 μM, 10 μM to 100 μM, 20 μM to 100 μM, 30 μM to 100 μM, 40 μM to 100 μM, 50 μM to 100 μM, 60 μM to 100 μM, 70 μM to 100 μM, or 80 μM to 100 μM.

In some embodiments, a composition (e.g., medium) of the disclosure comprises a one carbon metabolism pathway intermediate. Exemplary one carbon metabolism pathway intermediates include, but are not limited to formate, tetrahydrofolate (THF), 10-formylTHF; 5,10-meTHF; 5,10-meTHF; and 10-formylTHF. In some embodiments, the one carbon metabolism pathway intermediate is formate present at a concentration of about 50 μM. In some embodiments, the one carbon metabolism pathway intermediate is present or is added at a concentration of about 100 nM to 1 mM, 500 nM to 1 mM, 1 μM to 1 mM, 10 μM to 1 mM, 20 μM to 1 mM, 30 μM to 1 mM, 100 nM to 250 μM, 500 nM to 250 μM, 1 μM to 250 μM, 10 μM to 250 μM, 20 μM to 250 μM, 30 μM to 250 μM, 100 nM to 100 μM, 500 nM to 100 μM, 1 μM to 100 μM, 10 μM to 100 μM, 20 μM to 100 μM, 30 μM to 100 μM, 100 nM to 60 μM, 500 nM to 60 μM, 1 μM to 60 μM, 10 μM to 60 μM, 20 μM to 60 μM, 30 μM to 60 μM, 40 μM to 60 μM, or 45 μM to 55 μM.

In some embodiments, a composition (e.g., medium) of the disclosure comprises glutamine. Thus in some embodiments, compositions and methods of the disclosure utilize glutamine in a form with increased bioavailability, such as a free glutamine form, such as a non-dipeptide form, a non-alanine-glutamine dipeptide form (e.g., a non-alanyl-l-glutamine form), a non-glycine-glutamine dipeptide form (e.g., a non-glycyl-1-glutamine form), a form that in which glutamine is not conjugated to another amino acid or stabilizing moiety, a monomeric form, a free form, or a combination thereof. In some embodiments, glutamine is provided as a protein hydrolysate. In some embodiments, glutamine is present or is added to a composition of the disclosure at a concentration of from 0.5-20 mM, 0.5-10 mM, 0.5-5 mM, 1-5 mM, 2-5 mM, or 1 mM to 10 mM. In some embodiments, glutamine is present or is added to a composition of the disclosure at a concentration of 3.8-4.2 mM. In some embodiments, glutamine is present or is added to a composition of the disclosure at a concentration of 1-10, 1-7, 1-8, 1-6, 1-5, 1-4, 2-10, 2-7, 2-8, 2-6, 2-5, 2-4, 3-10, 3-7, 3-8, 3-6, 3-5, 3-4, 3.5-4.5, 3.8-4.2, or 3.9-4.1 mM. In some embodiments, glutamine is present or is added to a composition of the disclosure at a concentration of about 4 mM. In some embodiments, at least 0.5 mM, 0.6 mM, 0.7 mM, 0.8 mM, 0.9 mM, 1 mM, 1.5 mM, 2 mM, 2.5 mM, 3 mM, 3.5 mM, 4 mM, 4.5 mM, or 5 mM of the glutamine is not in a dipeptide form. In some embodiments, at least 500 μM, at least 750 μM, at least 1 mM, at least 1.5 mM, at least 2 mM, at least 2.5 mM, at least 2.6 mM, at least 2.7 mM, at least 2.8 mM, at least 2.9 mM, at least 3 mM, at least 3.1 mM, at least 3.2 mM, at least 3.3 mM, at least 3.4 mM, at least 3.5 mM, at least 3.6 mM, at least 3.7 mM, at least 3.8 mM, at least 3.9 mM, at least 4 mM, at least 5 mM, at least 5.5 mM, at least 6 mM, at least 6.5 mM, at least 7 mM, at least 7.5 mM, at least 8 mM, at least 8.5 mM, at least 9 mM, at least 9.5 mM, or at least 10 mM of the glutamine is in a free form.

In some embodiments, the method comprises culturing the population of cells (e.g., NKX6.1-positive pancreatic progenitor cells) in a medium, to induce the differentiation of at least one NKX6.1-positive pancreatic progenitor cell in the population into an insulin-positive endocrine cell, wherein the insulin-positive endocrine cell expresses insulin.

Aspects of the disclosure involve treatment of cell population comprising PDX1-positive, NKX6.1-positive pancreatic progenitor cells with PKC activator and/or wnt signaling pathway inhibitor, which can lead to increase in percentage of pancreatic α cells, increase in percentage of pancreatic δ cells, increase in percentage of pancreatic β cells, reduction in percentage of EC cells, or any combination thereof, in the cell population of pancreatic endocrine cells generated according to the method disclosed herein.

In some embodiments, the method comprises contacting a population of cells comprising PDX1-positive, NKX6.1-positive pancreatic progenitor cells with a first composition comprising a FOXO1 inhibitor, notch signaling inhibitor, a PKC activator, a ROCK inhibitor, a growth factor from TGFβ superfamily, a growth factor from FGF family, a RA signaling pathway activator, and a SHH pathway inhibitor, for one to two days, thereby obtaining a first transformation cell population comprising PDX1-positive, NKX6.1-positive pancreatic progenitor cells; and contacting the first transformation cell population comprising PDX1-positive, NKX6.1-positive pancreatic progenitor cells with a second composition comprising the PKC activator, notch signaling inhibitor, a TGF-β signaling pathway inhibitor, a TH signaling pathway activator, BMP pathway inhibitor, ROCK inhibitor, retinoic acid, and EGF-family growth factor, wnt signaling pathway inhibitor, and/or an epigenetic modifying compound, for one to two days, thereby obtaining a second transformation cell population comprising NKX6.1-positive, ISL1-positive endocrine cells.

### Pancreatic β Cells

Aspects of the disclosure involve generating pancreatic β cells (e.g., non-native pancreatic β cells/SC-β cells) and additional methods of generating them. Non-native pancreatic β cells. In some embodiments, resemble endogenous mature β cells in form and function, but nevertheless are distinct from native β cells.

In some embodiments, the insulin-positive pancreatic endocrine cells generated using the method provided herein can form a cell cluster, alone or together with other types of cells, e.g., precursors thereof, e.g., stem cell, definitive endoderm cells, primitive gut tube cell, PDX1-positive pancreatic progenitor cells, or NKX6.1-positive pancreatic progenitor cells.

In some embodiments, any of the cells or populations of cells disclosed herein are in a cell cluster. In some embodiments, the disclosure provides for a composition comprising one or more cell clusters. In some embodiments, the composition comprises 500-20000, 500-15000, 500-10000, 500-5000, 500-2000, 500-1000, 1000-20000, 1000-15000, 1000-10000, 1000-5000, 1000-2000, 2000-20000, 2000-15000, 2000-10000, 2000-5000, 5000-20000, 5000-15000, 5000-10000, 10000-20000, 10000-15000, 15000-20000, or 3000-9000 cell clusters. In some aspects, provided herein are cell clusters that resemble the functions and characteristics of endogenous pancreatic islets. Such cell clusters can mimic the function of endogenous pancreatic islets in regulating metabolism, e.g., glucose metabolism in a subject.

In some embodiments, a composition or cell population of the present disclosure comprises NKX6.1-positive, ISL-positive cells that express lower levels of MAFA than NKX6.1-positive, ISL-positive cells from the pancreas of a healthy control adult subject. In some embodiments, the composition or cell population comprises NKX6.1-positive, ISL-positive cells that express higher levels of MAFB than NKX6.1-positive, ISL-positive cells from the pancreas of a healthy control adult subject. In some embodiments, the composition or cell population comprises NKX6.1-positive, ISL-positive cells that express higher levels of SIX2, HOPX, IAPP and/or UCN3 than NKX6.1-positive, ISL-positive cells from the pancreas of a healthy control adult subject.

In some embodiments, a composition or cell population of the present disclosure comprises NKX6.1-positive, ISL-positive cells that do not express MAFA. In some embodiments, the composition or cell population comprises NKX6.1-positive, ISL-positive cells that express MAFB.

In some embodiments, the cell population comprising the insulin-positive endocrine cells can be directly induced to mature into SC-β cells without addition of any exogenous differentiation factors (such as inhibitor of TGF-β signaling pathway, thyroid hormone signaling pathway activator, PKC activator, growth factors from TGF-β superfamily, FGF family, or EGF family, SHH signaling pathway inhibitor, γ-secretase inhibitor, ROCK inhibitor, or BMP signaling pathway inhibitor). In some embodiments, the method provided herein comprises contacting a cell population comprising NKX6.1-positive, ISL1-positive endocrine cells with a serum albumin protein, a TGF-β signaling pathway inhibitor, a SHH pathway inhibitor, a TH signaling pathway activator, a protein kinase inhibitor, a ROCK inhibitor, a BMP signaling pathway inhibitor, and/or an epigenetic modifying compound. In some embodiments, the method provided herein comprises contacting a cell population comprising NKX6.1-positive, ISL1-positive endocrine cells with human serum albumin protein. In some embodiments, the method provided herein comprises contacting a cell population comprising NKX6.1-positive, ISL1-positive endocrine cells with a PKC activator.

In some embodiments, the cell population comprising the insulin-positive endocrine cells can be induced to mature into SC-β cells by contacting the insulin-positive endocrine cells with differentiation factors. The differentiation factors can comprise at least one inhibitor of TGF-β signaling pathway and thyroid hormone signaling pathway activator as described herein. In some embodiments, SC-β cells can be obtained by contacting a population of cells comprising insulin-positive endocrine cells with Alk5i and T3 or GC-1.

In some embodiments, the method provided herein comprises contacting a cell population comprising NKX6.1-positive, ISL1-positive endocrine cells with (i) a growth factor from the FGF family, (ii) a TGF-β signaling pathway inhibitor, (iii) a thyroid hormone signaling pathway activator, (iv) an epigenetic modifying compound, (v) a protein kinase inhibitor, (vi) a ROCK inhibitor, (vii) a BMP signaling pathway inhibitor, and (viii) a lipase inhibitor for about one two five days. In some embodiments, the contacting is for about three days.

Any TGF-β signaling pathway inhibitor capable of inducing the differentiation of insulin-positive endocrine cells to mature into SC-β cells (e.g., alone, or in combination with other β cell-differentiation factors, e.g., a thyroid hormone signaling pathway activator) can be used. In some embodiments, the TGF-β signaling pathway comprises TGF-β receptor type I kinase signaling. In some embodiments, the TGF-β signaling pathway inhibitor comprises Alk5 inhibitor II. In some examples, the method comprises contacting insulin-positive endocrine cells with a concentration of a TGF-β signaling pathway inhibitor (e.g., Alk5 inhibitor such as Alk5 inhibitor II), such as, about 0.1 μM, about 0.5 μM, about 1 μM, about 1.5 μM, about 2 μM, about 2.5 μM, about 3 μM, about 3.5 μM, about 4 μM, about 4.5 μM, about 5 μM, about 5.5 μM, about 6 μM, about 6.5 μM, about 7 μM, about 7.5 μM, about 8 μM, about 8.5 μM, about 9 μM, about 9.5 μM, about 10 μM, about 10.5 μM, about 11 μM, about 11.5 μM, about 12 μM, about 12.5 μM, about 13 μM, about 13.5 μM, about 14 μM, about 14.5 μM, about 15 μM, about 15.5 μM, about 16 μM, about 16.5 μM, about 17 μM, about 17.5 μM, about 18 μM, about 18.5 μM, about 19 μM, about 19.5 μM, about 20 μM, about 25 μM, about 30 μM, about 35 μM, about 40 μM, about 45 μM, or about 50 μM. In some examples, the method comprises contacting insulin-positive endocrine cells with a concentration of a TGF-β signaling pathway inhibitor (e.g., Alk5 inhibitor such as Alk5 inhibitor II), such as, about 7-13 μM, about 8-12 μM, or about 9-11 μM. In some examples, the method comprises contacting insulin-positive endocrine cells with a concentration of a TGF-β signaling pathway inhibitor (e.g., Alk5 inhibitor such as Alk5 inhibitor II), such as, about 10 μM.

Any thyroid hormone signaling pathway activator capable of inducing the differentiation of insulin-positive endocrine cells to mature into SC-β cells (e.g., alone, or in combination with other β cell-differentiation factors, e.g., a TGF-β signaling pathway inhibitor) can be used. In some embodiments, the thyroid hormone signaling pathway activator comprises triiodothyronine (T3). In some embodiments, the thyroid hormone signaling pathway activator comprises GC-1. In some examples, the method comprises contacting insulin-positive endocrine cells with a concentration of thyroid hormone signaling pathway activator (e.g., GC-1), such as, about 0.1 μM, about 0.12 μM, about 0.13 μM, about 0.14 μM, about 0.15 μM, about 0.16 μM, about 0.17 μM, about 0.18 μM, about 0.19 μM, about 0.2 μM, about 0.21 μM, about 0.22 μM, about 0.23 μM, about 0.24 μM, about 0.25 μM, about 0.26 μM, about 0.27 μM, about 0.28 μM, about 0.29 μM, about 0.3 μM, about 0.31 μM, about 0.32 μM, about 0.33 μM, about 0.34 μM, about 0.35 μM, about 0.4 μM, about 0.45 μM, about 0.5 μM, about 0.6 μM, about 0.8 μM, about 1 μM, about 2 μM, or about 5 μM. In some examples, the method comprises contacting insulin-positive endocrine cells with a concentration of thyroid hormone signaling pathway activator (e.g., GC-1), such as, about 0.7-1.3 μM, about 0.8-1.2 μM, or about 0.9-1.1 μM. In some examples, the method comprises contacting insulin-positive endocrine cells with a concentration of thyroid hormone signaling pathway activator (e.g., GC-1), such as, about 1 μM.

Any BMP signaling pathway inhibitor capable of inducing the differentiation of insulin-positive endocrine cells to mature into SC-β cells (e.g., alone, or in combination with any of a TGF-β signaling pathway inhibitor and/or a thyroid hormone signaling pathway activator) can be used. In some embodiments, the BMP signaling pathway inhibitor comprises LDN193189 or DMH-1. In some examples, the method comprises contacting insulin-positive endocrine cells with a concentration of BMP signaling pathway inhibitor (e.g., LDN1931189), such as, about 30 nM, about 40 nM, about 50 nM, about 60 nM, about 70 nM, about 80 nM, about 90 nM, about 100 nM, about 110 nM, about 120 nM, about 130 nM, about 140 nM, about 150 nM, about 160 nM, about 170 nM, about 180 nM, about 190 nM, about 200 nM, about 210 nM, about 220 nM, about 230 nM, about 240 nM, about 250 nM, about 280 nM, about 300 nM, about 400 nM, about 500 nM, or about 1 μM. In some examples, the method comprises contacting insulin-positive endocrine cells with a concentration of BMP signaling pathway inhibitor (e.g., LDN1931189), such as, about 70-130 nM, about 80-120 nM, about 90-110 nM. In some examples, the method comprises contacting NKX6.1-positive pancreatic progenitor cells with a concentration of BMP signaling pathway inhibitor (e.g., LDN1931189), such as, about 100 nM.

Any ROCK inhibitor that is capable of inducing the differentiation of insulin-positive endocrine cells to mature into SC-β cells (e.g., alone, or in combination with any of a TGF-β signaling pathway inhibitor and/or a thyroid hormone signaling pathway activator) can be used. In some embodiments, the ROCK inhibitor comprises Thiazovivin, Y-27632, Fasudil/HA1077, or H-1152. In some embodiments, the ROCK inhibitor comprises Y-27632. In some embodiments, the ROCK inhibitor comprises Thiazovivin. In some examples, the method comprises contacting insulin-positive endocrine cells with a concentration of a ROCK inhibitor (e.g., Y-27632 or Thiazovivin), such as, about 0.2 μM, about 0.5 μM, about 0.75 μM, about 1 μM, about 2 μM, about 3 μM, about 4 μM, about 5 μM, about 6 μM, about 7 μM, about 7.5 μM, about 8 μM, about 9 μM, about 10 μM, about 11 μM, about 12 μM, about 13 μM, about 14 μM, about 15 μM, about 16 μM, about 17 μM, about 18 μM, about 19 μM, about 20 μM, about 21 μM, about 22 μM, about 23 μM, about 24 μM, about 25 μM, about 26 μM, about 27 μM, about 28 μM, about 29 μM, about 30 μM, about 35 μM, about 40 μM, about 50 μM, or about 100 μM. In some embodiments, the ROCK inhibitor comprises Thiazovivin. In some examples, the method comprises contacting insulin-positive endocrine cells with a concentration of a ROCK inhibitor (e.g., Y-27632 or Thiazovivin), such as, about 2.2-2.8 μM, about 2.3-2.7 μM, or about 2.4-2.6 μM. In some embodiments, the ROCK inhibitor comprises Thiazovivin. In some examples, the method comprises contacting insulin-positive endocrine cells with a concentration of a ROCK inhibitor (e.g., Y-27632 or Thiazovivin), such as, about 2.5 μM.

Any epigenetic modifying compound that is capable of inducing the differentiation of insulin-positive endocrine cells to mature into SC-β cells (e.g., alone, or in combination with any of a TGF-β signaling pathway inhibitor and/or a thyroid hormone signaling pathway activator) can be used. In some embodiments, the epigenetic modifying compound comprises a histone methyltransferase inhibitor or a HDAC inhibitor. In some embodiments, the epigenetic modifying compound comprises a histone methyltransferase inhibitor, e.g., DZNep. In some embodiments, the epigenetic modifying compound comprises a HDAC inhibitor, e.g., KD5170. In some examples, the method comprises contacting insulin-positive endocrine cells to mature into SC-β cells with a concentration of an epigenetic modifying compound (e.g., DZNep or KD5170), such as, about 0.01 μM, about 0.025 μM, about 0.05 μM, about 0.075 μM, about 0.1 μM, about 0.15 μM, about 0.2 μM, about 0.5 μM, about 0.75 μM, about 1 μM, about 2 μM, about 3 μM, about 4 μM, about 5 μM, about 6 μM, about 7 μM, about 7.5 μM, about 8 μM, about 9 μM, about 10 μM, about 15 μM, about 20 μM, about 25 μM, about 30 μM, about 35 μM, about 40 μM, about 50 μM, or about 100 μM. In some examples, the method comprises contacting insulin-positive endocrine cells to mature into SC-β cells with a concentration of an epigenetic modifying compound (e.g., DZNep or KD5170), such as, about 70-130 nM, about 80-120 nM, or about 90-110 nM. In some examples, the method comprises contacting insulin-positive endocrine cells to mature into SC-β cells with a concentration of an epigenetic modifying compound (e.g., DZNep or KD5170), such as, about 100 nM.

Any protein kinase inhibitor that is capable of inducing the differentiation insulin-positive endocrine cells to mature into SC-β cells (e.g., alone, or in combination with any of a TGF-β signaling pathway inhibitor and/or a thyroid hormone signaling pathway activator). In some embodiments, the protein kinase inhibitor comprises staurosporine. In some examples, the method comprises contacting insulin-positive endocrine cells with a concentration of a protein kinase inhibitor (e.g., staurosporine), such as, about 0.1 nM, about 0.2 nM, about 0.3 nM, about 0.4 nM, about 0.5 nM, about 0.6 nM, about 0.7 nM, about 0.8 nM, about 0.9 nM, about 1 nM, about 1.1 nM, about 1.2 nM, about 1.3 nM, about 1.4 nM, about 1.5 nM, about 1.6 nM, about 1.7 nM, about 1.8 nM, about 1.9 nM, about 2.0 nM, about 2.1 nM, about 2.2 nM, about 2.3 nM, about 2.4 nM, about 2.5 nM, about 2.6 nM, about 2.7 nM, about 2.8 μM, about 2.9 nM, about 3 nM, about 3.1 nM, about 3.2 nM, about 3.3 nM, about 3.4 nM, about 3.5 nM, about 3.6 nM, about 3.7 nM, about 3.8 nM, about 3.9 nM, about 4.0 nM, about 4.1 nM, about 4.2 nM, about 4.3 nM, about 4.4 nM, about 4.5 nM, about 4.6 nM, about 4.7 nM, about 4.8 μM, about 4.9 nM, or about 5 nM. In some examples, the method comprises contacting insulin-positive endocrine cells with a concentration of a protein kinase inhibitor (e.g., staurosporine), such as, about 1-5 nM, about 2-4 nM, or about 2.5-3.5 nM. In some examples, the method comprises contacting insulin-positive endocrine cells with a concentration of a protein kinase inhibitor (e.g., staurosporine), such as, about 3 nM.

In some embodiments, the method comprises contacting the population of cells (e.g., NKX6.1-positive, ISL1-positive, insulin-positive cells) with one or more metabolites. In some embodiments, the method comprises contacting the population of cells (e.g., NKX6.1-positive, ISL1-positive, insulin-positive cells) with one or more of an acetyl CoA-related metabolite, a vitamin, histone deacetylase inhibitor (HDACi), a redox homeostasis regulator, a one carbon metabolism pathway intermediate, glutamate, and/or carnitine. Examples of metabolites include taurine, acetate, beta-hydroxybutyrate, biotin, carnitine, glutamate, and formate.

In some embodiments, a composition (e.g., medium) of the disclosure comprises an acetyl CoA-related metabolite. Exemplary acetyl COA-related metabolites include, but are not limited to acetate, pyruvate, ketogenic amino acids, valine, leucine, isoleucine, phenylalanine, tyrosine, lysine, tryptophan, fatty acids, CoA, Isovaleryl-CoA, and β-hydroxybutyrate. In some embodiments, the acetyl COA-related metabolite is acetate. In some embodiments, the acetyl CoA-related metabolite is present in or is added to a composition of the disclosure at a concentration of about 10 nM, about 50 nM, about 80 nM, about 100 nM, about 120 nM, about 140 nM, about 150 nM, about 200 nM, about 300 nM, about 500 nM, about 800 nM, about 1 μM, about 10 μM, about 100 μM, about 500 μM, about 800 μM, about 900 μM, about 1 mM, about 2 mM, about 3 mM, about 5 mM, or about 10 mM. In some embodiments, the acetyl CoA-related metabolite is present in or is added to a composition of the disclosure at a concentration of about 0.01-50 mM, 0.1-50 mM, 0.5-50 mM, 0.01-20 mM, 0.1-20 mM, 0.5-20 mM, 0.01-10 mM, 0.1-10 mM, 0.5-10 mM, 0.8-25 mM, 0.8-10 mM, 0.8-5 mM, 0.8-2 mM, 0.8-1.5 mM, 0.8-1.2 mM, 0.9-1.1 mM, or 0.95-1.05 mM. In some embodiments, the acetyl CoA-related metabolite is acetate present at a concentration of about 1 mM. In some embodiments, the acetyl CoA-related metabolite is acetate present at a concentration of about 50-1000 nM, 50-800 nM, 50-500 nM, 50-300 nM, 50-250 nM, 100-200 nM, or 125-175 nM. In some embodiments, the acetyl COA-related metabolite is acetate present at a concentration of about 160 nM.

In some embodiments, a composition (e.g., medium) of the disclosure comprises one or more vitamins. Exemplary vitamins include, but are not limited to biotin, vitamin B1 (thiamine), vitamin B2 (riboflavin), vitamin B3 (niacin), vitamin B6 (pyridoxine) and vitamin B12 (cyanocobalamin). In some embodiments the vitamin modulates fatty acid synthesis. In some embodiments the vitamin modulates branched-chain amino acid metabolism. In some embodiments the vitamin modulates or participates as a co-factor in the TCA cycle, e.g., as a cofactor for pyruvate carboxylase. In some embodiments, the vitamin is biotin. In some embodiments, the vitamin is present in or is added to a composition of the disclosure at a concentration of about 100 nM, about 300 nM, about 500 nM, about 600 nM, about 700 nM, about 800 nM, about 900 nM, about 1 μM, about 1.5 μM, about 3 μM, about 5 μM, about 10 μM, or about 100 μM. In some embodiments, the vitamin is biotin present at a concentration of about 800 nM. In some embodiments, the vitamin is present in or is added to a composition of the disclosure at a concentration of about 1 nM to 500 μM, 1 nM to 100 μM, 1 nM to 10 μM, 1 nM to 1 μM, 1 nM to 800 nM, 1 nM to 600 nM, 1 nM to 400 nM, 1 nM to 300 nM, 1 nM to 200 nM, 25 nM to 500 μM, 25 nM to 100 μM, 25 nM to 10 μM, 25 nM to 1 μM, 25 nM to 800 nM, 25 nM to 600 nM, 25 nM to 400 nM, 25 nM to 300 nM, 25 nM to 200 nM, 50 nM to 500 μM, 50 nM to 100 μM, 50 nM to 10 μM, 50 nM to 1 μM, 50 nM to 800 nM, 50 nM to 600 nM, 50 nM to 400 nM, 50 nM to 300 nM, 50 nM to 200 nM, 100 nM to 500 μM, 100 nM to 100 μM, 100 nM to 10 μM, 100 nM to 1 μM, 100 nM to 800 nM, 100 nM to 600 nM, 100 nM to 400 nM, 100 nM to 300 nM, or 100 nM to 200 nM.

In some embodiments, a composition (e.g., medium) of the disclosure comprises a histone deacetylase inhibitor (HDACi). Exemplary histone deacetylase inhibitors (HDACi) include, but are not limited to β-Hydroxybutyrate, butyric acid, class I HDACi, class IIA HDACi, class IIB HDACi, class III HDACi, class IV HDACi, HDAC-1, HDAC-2, HDAC-3, HDAC-4, HDAC-5, HDAC-6, HDAC-7, HDAC-8, HDAC-9, HDAC-10, HDAC-11, sirtuins, SIRT1, SIRT2, SIRT3, SIRT4, SIRT5, SIRT6, SIRT7, Vorinostat (suberoylanilide hydroxamic acid, SAHA, MK0683), Entinostat (MS-275, SNDX-275), Panobinostat (LBH589, NVP-LBH589), Trichostatin A (TSA), Mocetinostat (MGCD0103, MG0103), GSK3117391 (GSK3117391A, HDAC-IN-3), BRD3308, BRD3308, Tubastatin A TFA (Tubastatin A trifluoroacetate salt), Tubastatin A, SIS17, NKL 22, BML-210 (CAY10433), TC-H 106, SR-4370, Belinostat (PXD101, NSC726630, PX-105684), Romidepsin (FK228, Depsipeptide, FR 901228, NSC 630176), MC1568, Givinostat (ITF2357), Dacinostat (LAQ824, NVP-LAQ824), CUDC-101, Quisinostat (JNJ-26481585), Pracinostat (SB939), PCI-34051, Droxinostat (NS 41080), Abexinostat (PCI-24781), Abexinostat (PCI-24781, CRA-024781), RGFP966, AR-42 (HDAC-42), Ricolinostat (ACY-1215, Rocilinostat), Valproic acid sodium salt (Sodium valproate), Tacedinaline (CI994, PD-123654, GOE-5549, Acetyldinaline), Fimepinostat (CUDC-907), Sodium butyrate (NaB), Curcumin, Diferuloylmethane, M344, Tubacin, RG2833 (RGFP109), RG2833 (RGFP109), Resminostat (RAS2410), Divalproex Sodium, Scriptaid (GCK 1026), Sodium Phenylbutyrate, Sinapinic acid (Sinapic acid), TMP269, Santacruzamate A (CAY10683), TMP195 (TFMO 2), Valproic acid (VPA), UF010, Tasquinimod (ABR-215050), SKLB-23bb, Isoguanosine, Sulforaphane, BRD73954, Citarinostat (ACY-241, HDAC-IN-2), Suberohydroxamic acid, Splitomicin, HPOB, LMK-235, Biphenyl-4-sulfonyl chloride (p-Phenylbenzenesulfonyl, 4-Phenylbenzenesulfonyl, p-Biphenylsulfonyl), Nexturastat A, TH34, Tucidinostat (Chidamide, HBI-8000, CS-055), (−)-Parthenolide, WT161, CAY10603, CAY10603, ACY-738, Raddeanin A, Tinostamustine (EDO-S101), Domatinostat (4SC-202), and BG45. In some embodiments, the HDACi is β-Hydroxybutyrate. In some embodiments, the HDACi is present in or is added to a composition of the disclosure at a concentration of about 100 nM, about 300 nM, about 500 nM, about 600 nM, about 700 nM, about 800 nM, about 900 nM, about 1 μM, about 1.5 μM, about 3 μM, about 5 μM, about 10 μM, or about 100 μM. In some embodiments, the HDACi is β-Hydroxybutyrate present at a concentration of about 200 nM. In some embodiments, the HDACi is present in or is added to a composition of the disclosure at a concentration of about 1 nM to 500 μM, 1 nM to 100 μM, 1 nM to 10 μM, 1 nM to 1 μM, 1 nM to 800 nM, 1 nM to 600 nM, 1 nM to 400 nM, 1 nM to 300 nM, 1 nM to 200 nM, 25 nM to 500 μM, 25 nM to 100 μM, 25 nM to 10 μM, 25 nM to 1 μM, 25 nM to 800 nM, 25 nM to 600 nM, 25 nM to 400 nM, 25 nM to 300 nM, 25 nM to 200 nM, 50 nM to 500 μM, 50 nM to 100 μM, 50 nM to 10 μM, 50 nM to 1 μM, 50 nM to 800 nM, 50 nM to 600 nM, 50 nM to 400 nM, 50 nM to 300 nM, 50 nM to 200 nM, 100 nM to 500 μM, 100 nM to 100 μM, 100 nM to 10 μM, 100 nM to 1 μM, 100 nM to 800 nM, 100 nM to 600 nM, 100 nM to 400 nM, 100 nM to 300 nM, or 100 nM to 200 nM.

In some embodiments, a composition (e.g., medium) of the disclosure comprises a redox homeostasis regulator. Exemplary redox homeostasis regulators include, but are not limited to taurine, respiratory chain regulators, free radical scavengers, regulators of mitochondrial protein synthesis, allium sulphur compounds, anthocyanins, beta-carotene, catechins, copper, cryptoxanthins, flavonoids, indoles, isoflavonoids, lignans, lutein, lycopene, alpha lipoic acid, ellagic acid, manganese, polyphenols, selenium, glutathione, vitamin A, vitamin C, vitamin E, zinc, superoxide disutases, GSHPx, Prx-I, catalase, and co-enzyme Q10. In some embodiments, the redox homeostasis regulator is taurine. In some embodiments, the redox homeostasis regulator is present in or is added to a composition of the disclosure at a concentration of about 100 nM, about 500 nM, 1 μM, about 10 μM, about 20 μM, about 30 μM, about 40 μM, about 50 μM, about 60 μM, about 70 μM, about 80 μM, about 90 μM, about 100 μM, about 110 μM, about 110 μM, about 150 μM, or about 200 μM. In some embodiments, the redox homeostasis regulator is taurine. In some embodiments, the redox homeostasis regulator is taurine present at a concentration of about 90 μM. In some embodiments, the redox homeostasis regulator intermediate is present or is added at a concentration of about 100 nM to 1 mM, 500 nM to 1 mM, 1 μM to 1 mM, 10 μM to 1 mM, 20 μM to 1 mM, 30 μM to 1 mM, 30 μM to 1 mM, 40 μM to 1 mM, 50 μM to 1 mM, 60 μM to 1 mM, 70 μM to 1 mM, 80 μM to 1 mM, 100 nM to 250 μM, 500 nM to 250 μM, 1 μM to 250 μM, 10 μM to 250 μM, 20 μM to 250 μM, 30 μM to 250 μM, 30 μM to 250 μM, 40 μM to 250 μM, 50 μM to 250 μM, 60 μM to 250 μM, 70 μM to 250 μM, 100 nM to 100 μM, 500 nM to 100 μM, 1 μM to 100 μM, 10 μM to 100 μM, 20 μM to 100 μM, 30 μM to 100 μM, 40 μM to 100 μM, 50 μM to 100 μM, 60 μM to 100 μM, 70 μM to 100 UM, or 80 μM to 100 μM.

In some embodiments, a composition (e.g., medium) of the disclosure comprises a one carbon metabolism pathway intermediate. Exemplary one carbon metabolism pathway intermediates include, but are not limited to formate, tetrahydrofolate (THF), 10-formylTHF; 5,10-meTHF; 5,10-meTHF; and 10-formylTHF. In some embodiments, the one carbon metabolism pathway intermediate is formate present at a concentration of about 50 μM. In some embodiments, the one carbon metabolism pathway intermediate is present or is added at a concentration of about 100 nM to 1 mM, 500 nM to 1 mM, 1 μM to 1 mM, 10 μM to 1 mM, 20 μM to 1 mM, 30 μM to 1 mM, 100 nM to 250 μM, 500 nM to 250 μM, 1 μM to 250 μM, 10 μM to 250 μM, 20 μM to 250 μM, 30 μM to 250 μM, 100 nM to 100 μM, 500 nM to 100 μM, 1 μM to 100 μM, 10 μM to 100 μM, 20 μM to 100 μM, 30 μM to 100 μM, 100 nM to 60 μM, 500 nM to 60 μM, 1 μM to 60 μM, 10 μM to 60 μM, 20 μM to 60 μM, 30 μM to 60 μM, 40 μM to 60 μM, or 45 μM to 55 μM.

In some embodiments, a composition (e.g., medium) of the disclosure comprises glutamate (e.g., L-glutamate). In some embodiments, glutamate can be present in a composition of the disclosure at a concentration of about 100 μM, about 200 μM, about 300 μM, about 400 μM, about 450 μM, about 500 μM, about 550 μM, about 600 μM, about 700 μM, about 800 μM, about 900 μM, about 1 mM, about 1.5 mM, about 2 mM, about 2.5 mM, about 3 mM, about 4 mM, or about 5 mM. In some embodiments, glutamate is present or is added to a composition of the disclosure at a concentration of about 500 μM. In some embodiments, glutamate is present or is added to a composition of the disclosure at a concentration of from about 100 μM to 5 mM, 200 μM to 5 mM, 300 μM to 5 mM, 400 μM to 5 mM, 100 μM to 3 mM, 200 μM to 3 mM, 300 μM to 3 mM, 400 μM to 3 mM, 100 μM to 2 mM, 200 μM to 2 mM, 300 μM to 2 mM, 400 μM to 2 mM, 100 μM to 1 mM, 200 μM to 1 mM, 300 μM to 1 mM, 400 μM to 1 mM, 100 μM to 700 μM, 200 μM to 700 μM, 300 μM to 700 μM, 400 μM to 700 μM, 100 μM to 600 μM, 200 μM to 600 μM, 300 μM to 600 μM, or 400 μM to 600 μM.

In some embodiments, a composition (e.g., medium) of the disclosure comprises carnitine. In some embodiments, carnitine is present in or is added to a composition of the disclosure at a concentration of about 100 nM, about 500 nM, about 1 μM, about 10 μM, about 15 μM, about 20 μM, about 25 μM, about 30 μM, about 35 μM, about 40 μM, about 45 μM, about 50 μM, about 55 μM, about 60 μM, about 75 μM, or about 100 μM. In some embodiments, carnitine is present or is added at a concentration of about 40 μM. In some embodiments, carnitine is present in or is added to a composition of the disclosure at a concentration of about 100 nM to 1 mM, 500 nM to 1 mM, 1 μM to 1 mM, 10 μM to 1 mM, 20 μM to 1 mM, 30 μM to 1 mM, 100 nM to 250 μM, 500 nM to 250 μM, 1 μM to 250 μM, 10 μM to 250 μM, 20 μM to 250 μM, 30 μM to 250 μM, 100 nM to 100 μM, 500 nM to 100 μM, 1 μM to 100 μM, 10 μM to 100 μM, 20 μM to 100 μM, 30 μM to 100 μM, 100 nM to 60 μM, 500 nM to 60 μM, 1 μM to 60 μM, 10 μM to 60 μM, 20 μM to 60 μM, 30 μM to 60 μM, 35 μM to 60 μM, or 30 μM to 50 μM.

In some embodiments, the method comprises contacting the population of cells (e.g., NKX6.1-positive, ISL1-positive, insulin-positive cells) with a serum albumin protein (e.g., HSA). In some embodiments, the serum albumin is present at a concentration of 0.01-2% HSA. In some embodiments, the serum albumin is present at a concentration of 0.03-0.1%, 0.03-0.07%, or 0.04-0.05%. In some embodiments, the serum albumin is present at a concentration of 0.05%. In some embodiments, the serum albumin is present at a concentration of 0.7-1.3%, 0.8-1.2%, 0.9-1.1% or at 1%. In some embodiments, the serum albumin is present at a concentration of 1%.

In some embodiments, the method comprises contacting the population of cells (e.g., NKX6.1-positive, ISL1-positive, insulin-positive cells) with ZnSO₄. In some embodiments, the method comprises contacting the cells with 1-100 μM, 1-50 μM, 1-20 μM, 1-12 μM, 5-15 μM, 8-12 μM or 9-11 μM of ZnSO₄. In some embodiments, the method comprising contacting the cells with about 10 μM of ZnSO₄.

In some embodiments, the method comprises contacting the population of cells (e.g., NKX6.1-positive, ISL1-positive, insulin-positive cells) with one or more of an a serum albumin protein, a TGF-β signaling pathway inhibitor, a TH signaling pathway activator, a protein kinase inhibitor, a ROCK inhibitor, a BMP signaling pathway inhibitor, an epigenetic modifying compound, acetyl CoA-related metabolite, a vitamin, histone deacetylase inhibitor (HDACi), a redox homeostasis regulator, a one carbon metabolism pathway intermediate, glutamate, and/or carnitine for a first period of 1, 2, 3, 4, 5, 6, or 7 days (e.g., 4 days). In some embodiments, the method further comprises contacting the population of cells following the first period with one or more of a serum albumin protein, an acetyl CoA-related metabolite, a vitamin, histone deacetylase inhibitor (HDACi), a redox homeostasis regulator, a one carbon metabolism pathway intermediate, glutamate, and/or carnitine for a second period of 1, 2, 3, 4, 5, 6, or 7 days (e.g., 3 days) or more in the absence of a TGF-β signaling pathway inhibitor, a TH signaling pathway activator, a protein kinase inhibitor, a ROCK inhibitor, a BMP signaling pathway inhibitor, and/or an epigenetic modifying compound. In some embodiments, the cells are contacted with a higher concentration of the serum albumin in the second period as compared to the first period. In some embodiments, the compositions further comprise ZnSO₄.

In some embodiments, the method comprises contacting the population of cells (e.g., NKX6.1-positive, ISL1-positive, insulin-positive cells) with one or more of HSA, Alk5 inhibitor II, GC-1, staurosporine, thiazovivin, LDN193189, DZNEP, taurine, acetate, beta-hydroxybutyrate, biotin, carnitine, glutamate, and formate for a first period of 1, 2, 3, 4, 5, 6, or 7 days (e.g., 4 days). In some embodiments, the method further comprises contacting the population of cells following the first period with one or more of HSA, taurine, acetate, beta-hydroxybutyrate, biotin, carnitine, glutamate, and formate for a second period of 1, 2, 3, 4, 5, 6, or 7 days (e.g., 3 days) or more in the absence of an Alk5 inhibitor II, GC-1, staurosporine, thiazovivin, LDN193189, DZNEP. In some embodiments, the compositions further comprise ZnSO₄. In some embodiments, the cells are contacted with a higher concentration of the HSA (e.g., about 1.0%) in the second period as compared to the first period (e.g., about 0.05%).

In some examples, insulin-positive endocrine cells can be matured in a NS-GFs medium, MCDB131 medium, DMEM medium, or CMRL medium. In some embodiments, the insulin-positive endocrine cells can be matured in a CMRL medium supplemented with 10% FBS. In some embodiments, the insulin-positive endocrine cells can be matured in a DMEM/F12 medium supplemented with 1% HSA. In other cases, SC-β cells can be obtained by culturing the population of cells containing the insulin-positive endocrine cells in a MCDB131 medium that can be supplemented by 2% BSA. In some embodiments, the MCDB 131 medium with 2% BSA for maturation of insulin-positive endocrine cells into SC-β cells can be comprise no small molecule factors as described herein. In some case, the MCDB 131 medium with 2% BSA for maturation of insulin-positive endocrine cells into SC-β cells can comprise no serum (e.g., no FBS). In other cases, SC-β cells can be obtained by culturing the population of cells containing the insulin-positive endocrine cells in a MCDB131 medium that can be supplemented by 0.05% HSA and vitamin C. In some embodiments, SC-β cells can be obtained by culturing the population of cells containing the insulin-positive endocrine cells in a MCDB131 medium that can be supplemented by 0.05% HSA, ITS-X, vitamin C, and glutamine (Gln, e.g., 4 mM). In some embodiments, the type of culture medium may be changed during S6. For instance, the S6 cells are cultured in a MCDB131 medium that can be supplemented by 0.05% HSA and vitamin C for the first two to four days, and then followed by a DMEM/F12 medium supplemented with 1% HSA. In some embodiments, additional factors are introduced into the culture medium. For instance, S6 cells can be cultured in a MCDB131 medium that can be supplemented by 0.05% HSA, ITS-X, vitamin C, and glutamine (Gln, e.g., 4 mM) throughout the 10-12 days, during which ZnSO4 is introduced from day 4 of S6.

In some embodiments, the medium used to culture the cells as described herein can be xeno-free. A xeno-free medium for culturing cells and/or cell clusters of originated from an animal can have no product from other animals. In some embodiments, a xeno-free medium for culturing human cells and/or cell clusters can have no products from any non-human animals. For example, a xeno-free medium for culturing human cells and/or cell clusters can comprise human platelet lysate (PLT) instead of fetal bovine serum (FBS). For example, a medium can comprise from about 1% to about 20%, from about 5% to about 15%, from about 8% to about 12%, from about 9 to about 11% serum. In some embodiments, medium can comprise about 10% of serum. In some embodiments, the medium can be free of small molecules and/or FBS. For example, a medium can comprise MCDB131 basal medium supplemented with 2% BSA. In some embodiments, the medium is serum-free. In some examples, a medium can comprise no exogenous small molecules or signaling pathway agonists or antagonists, such as, growth factor from fibroblast growth factor family (FGF, such as FGF2, FGF8B, FGF 10, or FGF21), Sonic Hedgehog Antagonist (such as Sant1, Sant2, Sant4, Sant4, Cur61414, forskolin, tomatidine, AY9944, triparanol, cyclopamine, or derivatives thereof), Retinoic Acid Signaling agonist (e.g., retinoic acid, CD1530, AM580, TTHPB, CD437, Ch55, BMS961, AC261066, AC55649, AM80, BMS753, tazarotene, adapalene, or CD2314), inhibitor of Rho-associated, coiled-coil containing protein kinase (ROCK) (e.g., Thiazovivin, Y-27632, Fasudil/HA1077, or 14-1152), activator of protein kinase C (PKC) (e.g., phorbol 12,13-dibutyrate (PDBU), TPB, phorbol 12-myristate 13-acetate, bryostatin 1, or derivatives thereof), antagonist of TGF β super family (e.g., Alk5 inhibitor II (CAS 446859-33-2), A83-01, SB431542, D4476, GW788388, LY364947, LY580276, SB505124, GW6604, SB-525334, SD-208, SB-505124, or derivatives thereof), inhibitor of Bone Morphogenetic Protein (BMP) type 1 receptor (e.g., LDN193189 or derivatives thereof), thyroid hormone signaling pathway activator (e.g., T3, GC-1 or derivatives thereof), gamma-secretase inhibitor (e.g., XXI, DAPT, or derivatives thereof), activator of TGF-β signaling pathway (e.g., WNT3a or Activin A) growth factor from epidermal growth factor (EGF) family (e.g., betacellulin or EGF), broad kinase (e.g., staurosporine or derivatives thereof), non-essential amino acids, vitamins or antioxidants (e.g., cyclopamine, vitamin D, vitamin C, vitamin A, or derivatives thereof), or other additions like N-acetyl cysteine, zinc sulfate, or heparin. In some embodiments, the reaggregation medium can comprise no exogenous extracellular matrix molecule. In some embodiments, the reaggregation medium does not comprise Matrigel™. In some embodiments, the reaggregation medium does not comprise other extracellular matrix molecules or materials, such as, collagen, gelatin, poly-L-lysine, poly-D-lysine, vitronectin, laminin, fibronectin, PLO laminin, fibrin, thrombin, and RetroNectin and mixtures thereof, for example, or lysed cell membrane preparations.

A person of ordinary skill in the art will appreciate that the concentration of serum albumin supplemented into the medium may vary. For example, a medium (e.g., MCDB131) can comprise about 0.01%, 0.05%, 0.1%, 1%, about 2%, about 3%, about 4%, about 5%, about 10%, or about 15% BSA. In other cases, a medium can comprise about 0.01%, 0.05%, 0.1%, 1%, about 2%, about 3%, about 4%, about 5%, about 10%, or about 15% HSA. The medium used (e.g., MCDB131 medium) can contain components not found in traditional basal media, such as trace elements, putrescine, adenine, thymidine, and higher levels of some amino acids and vitamins. These additions can allow the medium to be supplemented with very low levels of serum or defined components. The medium can be free of proteins and/or growth factors, and may be supplemented with EGF, hydrocortisone, and/or glutamine. The medium can comprise one or more extracellular matrix molecules (e.g., extracellular proteins). Non-limiting exemplary extracellular matrix molecules used in the medium can include collagen, placental matrix, fibronectin, laminin, merosin, tenascin, heparin, heparin sulfate, chondroitin sulfate, dermatan sulfate, aggrecan, biglycan, thrombospondin, vitronectin, and decorin. In some embodiments, the medium comprises laminin, such as LN-332. In some embodiments, the medium comprises heparin.

The medium can be changed periodically in the culture, e.g., to provide optimal environment for the cells in the medium. When culturing the cells dissociated from the first cell cluster for re-aggregation, the medium can be changed at least or about every 4 hours, 12 hours, 24 hours, 48 hours, 3 days or 4 days. For example, the medium can be changed about every 48 hours.

In some embodiments, cells can be cultured under dynamic conditions (e.g., under conditions in which the cells are subject to constant movement or stirring while in the suspension culture). For dynamic culturing of cells, the cells can be cultured in a container (e.g., a non-adhesive container such as a spinner flask (e.g., of 200 ml to 3000 ml, for example 250 ml; of 100 ml; or in 125 ml Erlenmeyer), which can be connected to a control unit and thus present a controlled culturing system. Alternatively, the cells can be cultured in a bioreactor. In some embodiments, cells can be cultured under non-dynamic conditions (e.g., a static culture) while preserving their proliferative capacity. For non-dynamic culturing of cells, the cells can be cultured in an adherent culture vessel. An adhesive culture vessel can be coated with any of substrates for cell adhesion such as extracellular matrix (ECM) to improve the adhesiveness of the vessel surface to the cells. The substrate for cell adhesion can be any material intended to attach stem cells or feeder cells (if used). The substrate for cell adhesion includes collagen, gelatin, poly-L-lysine, poly-D-lysine, vitronectin, laminin, fibronectin, PLO laminin, fibrin, thrombin, and RetroNectin and mixtures thereof, for example, Matrigel™, and lysed cell membrane preparations.

Medium in a dynamic cell culture vessel (e.g., a spinner flask or bioreactor) can be stirred (e.g., by a stirrer). The spinning speed can correlate with the size of the re-aggregated second cell cluster. The spinning speed can be controlled so that the size of the second cell cluster can be similar to an endogenous pancreatic islet. In some embodiments, the spinning speed is controlled so that the size of the second cell cluster can be from about 75 μm to about 250 μm. The spinning speed of a dynamic cell culture vessel (e.g., a spinner flask or bioreactor) can be about 20 rounds per minute (rpm) to about 100 rpm, e.g., from about 30 rpm to about 90 rpm, from about 40 rpm to about 60 rpm, from about 45 rpm to about 50 rpm. In some embodiments, the spinning speed can be about 50 rpm.

Stage 6 cells as provided herein may or may not be subject to the dissociation and reaggregation process as described herein. In some embodiments, the cell cluster comprising the insulin-positive endocrine cells can be reaggregated. The reaggregation of the cell cluster can enrich the insulin-positive endocrine cells. In some embodiments, the insulin-positive endocrine cells in the cell cluster can be further matured into pancreatic β cells. For example, after reaggregation, the second cell cluster can exhibit in vitro GSIS, resembling native pancreatic islet. For example, after reaggregation, the second cell cluster can comprise non-native pancreatic β cell that exhibits in vitro GSIS. In some embodiments, the reaggregation process can be performed according to the disclosure of PCT application PCT/US2018/043179, which is incorporated herein by reference in its entirety.

Stage 6 cells obtained according to methods provided herein can have high recovery yield after cryopreservation and reaggregation procedures. In some embodiments, stage 6 cells that are obtained in a differentiation process that involves treatment of a BMP signaling pathway inhibitor (e.g., DMH-1 or LDN) and a growth factor from TGF-β superfamily (e.g., Activin A) at stage 3 and treatment of an epigenetic modifying compound (e.g., histone methyltransferase inhibitor, e.g., EZH2 inhibitor, e.g., DZNep) at stage 5 can have a higher recovery yield after cryopreservation post stage 5, as compared to a corresponding cell population without such treatment. In some embodiments, stage 6 cells that are obtained in a differentiation process that involves treatment of a BMP signaling pathway inhibitor (e.g., DMH-1 or LDN) and a growth factor from TGF-β superfamily (e.g., Activin A) at stage 3 and treatment of an epigenetic modifying compound (e.g., histone methyltransferase inhibitor, e.g., EZH2 inhibitor, e.g., DZNep) at stage 5 can have a higher recovery yield after cryopreservation post stage 5, as compared to a corresponding cell population without treatment of a BMP signaling pathway inhibitor (e.g., DMH-1 or LDN) and a growth factor from TGF-β superfamily (e.g., Activin A) at stage 3. In some embodiments, stage 6 cells that are obtained in a differentiation process that involves treatment of a BMP signaling pathway inhibitor (e.g., DMH-1 or LDN) and a growth factor from TGF-β superfamily (e.g., Activin A) at stage 3 and treatment of an epigenetic modifying compound (e.g., histone methyltransferase inhibitor, e.g., EZH2 inhibitor, e.g., DZNep) at stage 5 can have a recovery yield after cryopreservation post stage 5 that is at least about 35%, 37.5%, 40%, 42.5%, 45%, 47.5%, 48%, 49%, or 50%. The recovery yield can be calculated as a percentage of cells that survive and form reaggregated cell clusters after cryopreservation, thawing and recovery, and reaggregation procedures, as compared to the cells before the cryopreservation.

In some embodiments, the present disclosure relates to cryopreservation of the non-native pancreatic β cells or precursors thereof obtained using the methods provided herein. In particular embodiments, the cells are cryopreserved following stage 5 and before stage 6. In some embodiments, the cell population comprising non-native pancreatic β cells can be stored via cryopreservation. For instances, the cell population comprising non-native β cells, e.g., Stage 6 cells. In some embodiments, the cells can be dissociated into cell suspension, e.g., single cell suspension, and the cell suspension can be cryopreserved, e.g., frozen in a cryopreservation solution. The dissociation of the cells can be conducted by any of the technique provided herein, for example, by enzymatic treatment. The cells can be frozen at a temperature of at highest −20° C., at highest −30° C., at highest −40° C., at highest −50° C., at highest −60° C., at highest −70° C., at highest −80° C., at highest −90° C., at highest −100° C., at highest −110° C., at highest −120° C., at highest −130° C., at highest −140° C., at highest −150° C., at highest −160° C., at highest −170° C., at highest −180° C., at highest −190° C., or at highest −200° C. In some embodiments, the cells are frozen at a temperature of about −80° C. In some embodiments, the cells are frozen at a temperature of about −195° C. Any cooling methods can be used for providing the low temperature needed for cryopreservation, such as, but not limited to, electric freezer, solid carbon dioxide, and liquid nitrogen. In some embodiments, any cryopreservation solution available to one skilled in the art can be used for incubating the cells for storage at low temperature, including both custom made and commercial solutions. For example, a solution containing a cryoprotectant can be used. The cryoprotectant can be an agent that is configured to protect the cell from freezing damage. For instance, a cryoprotectant can be a substance that can lower the glass transition temperature of the cryopreservation solution. Exemplary cryoprotectants that can be used include DMSO (dimethyl sulfoxide), glycols (e.g., ethylene glycol, propylene glycol and glycerol), dextran (e.g., dextran-40), and trehalose. Additional agents can be added into the cryopreservation solution for other effects. In some embodiments, commercially available cryopreservation solutions can be used in the method provided herein, for instance, FrostaLife™, pZerve™, Prime-XV®, Gibco Synth-a-Freeze Cryopreservation Medium, STEM-CELLBANKER®, CryoStor® Freezing Media, HypoThermosol® FRS Preservation Media, and CryoDefend® Stem Cells Media.

During the differentiation process, the cells can be subject to irradiation treatment as provided herein. In some embodiments, the cell population at Stage 6, e.g., the cell population or cell cluster that has cells being differentiated from insulin-positive endocrine cells into pancreatic β cells, is irradiated for a period of time. In some embodiments, the cell population at Stage 6 after reaggregation following the recovery from cryopreservation is irradiated for a period of time. In some embodiments, the cryopreserved cells (e.g., the cells that are cryopreserved at the end of Stage 5) are irradiated for a certain period of time prior to thawing and recovery for subsequent differentiation process.

In some embodiments, the stage 6 cells comprise NKX6.1-positive, insulin-positive cells. In some embodiments, the stage 6 cells comprise NKX6.1-positive, insulin-negative cells. In some embodiments, the stage 6 cells comprise C-peptide positive cells. In some embodiments, Stage 6 cells or cells that have characteristics of stage 6 cells are incubated in NS-GFs medium, MCDB131 medium, DMEM medium, or CMRL medium. In some embodiments, the stage 6 cells or cells that have characteristics of stage 6 cells are contacted with any one or more of a vitamin or anti-oxidant (e.g., vitamin C), an albumin protein (e.g., a human serum albumin protein), a TGF-beta pathway inhibitor (e.g., an ALK5 inhibitor II), a bone morphogenic protein (BMP) type 1 receptor inhibitor (e.g., LDN193189), a Rho-associated coiled-coil containing protein kinase (ROCK) inhibitor (e.g., thiazovivin), a histone methyltransferase inhibitor (e.g., DZNEP), and a protein kinase inhibitor (e.g., staurosporine). See, e.g., WO2020264072. In some embodiments, the stage 6 cells are contacted with a PKC activator (see, e.g., WO2019217487, which is incorporated by reference herein in its entirety).

### Differentiation Factors

Aspects of the disclosure relate to contacting progenitor cells (e.g., stem cells, e.g., iPS cells, definitive endoderm cells, primitive gut tube cells, PDX1-positive pancreatic progenitor cells, NKX6.1-positive pancreatic progenitor cells, insulin-positive endocrine cells) with β cell differentiation factors, for example, to induce the maturation of the insulin-positive endocrine cells or differentiation of other progenitor cells into SC-β cells (e.g., mature pancreatic β cells). In some embodiments, the differentiation factor can induce the differentiation of pluripotent cells (e.g., iPSCs or hESCs) into definitive endoderm cells, e.g., in accordance with a method described herein. In some embodiments, the differentiation factor can induce the differentiation of definitive endoderm cells into primitive gut tube cells, e.g., in accordance with a method described herein. In some embodiments, the differentiation factor(s) can induce the differentiation of primitive gut tube cells into PDX1-positive pancreatic progenitor cells, e.g., in accordance with a method described herein. In some embodiments, the differentiation factor(s) can induce the differentiation of PDX1-positive pancreatic progenitor cells into NKX6-1-positive pancreatic progenitor cells, e.g., in accordance with a method described herein. In some embodiments, the differentiation factor(s) can induce the differentiation of NKX6-1-positive pancreatic progenitor cells into insulin-positive endocrine cells, e.g., in accordance with a method described herein. In some embodiments, the differentiation factor(s) can induce the maturation of insulin-positive endocrine cells into pancreatic islet cells, e.g., in accordance with a method described herein.

At least one differentiation factor described herein can be used alone, or in combination with other differentiation actors, to generate pancreatic islet cells (e.g., SC-beta cells) according to the methods as disclosed herein. In some embodiments, at least two, at least three, at least four, at least five, at least six, at least seven, at least eight, at least nine, or at least ten differentiation factors described herein are used in the methods of generating pancreatic islet cells.

In some embodiments, a composition described herein does not comprise one or more of the differentiation factors provided herein.

### Forkhead Box O1 (FoxO1) Inhibitor

Aspects of the disclosure relate to the use of Forkhead Box 01 (FoxO1) inhibitors as differentiation factors. In some embodiments, the FoxO1 inhibitor used in the compositions and methods described herein is a compound of Formula (I):

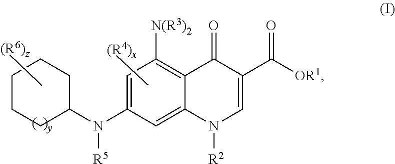

or a pharmaceutically acceptable salt, solvate, hydrate, polymorph, co-crystal, tautomer, stereoisomer, isotopically labeled derivative, prodrug, composition, or mixture thereof, wherein:
R¹ is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or an oxygen protecting group;
R² is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;
each instance of R³ is independently optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R³ are taken together with their intervening atoms to form a substituted or unsubstituted heterocyclic or substituted or unsubstituted heteroaryl ring;
each instance of R⁴ is independently halogen, optionally substituted acyl, optionally substituted alkyl, optionally substituted heteroalkyl, optionally substituted alkenyl, optionally substituted alkynyl, optionally substituted carbocyclyl, optionally substituted heterocyclyl, optionally substituted aryl, optionally substituted heteroaryl, —OR^{c1}, —NO₂, —N(R^{c2})₂, —SR^{c1}, —CN, or —SCN;
R⁵ is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group;
each instance of R⁶ is independently halogen, optionally substituted acyl, optionally substituted alkyl, optionally substituted heteroalkyl, optionally substituted alkenyl, optionally substituted alkynyl, optionally substituted carbocyclyl, optionally substituted heterocyclyl, optionally substituted aryl, optionally substituted heteroaryl, —OR^{c1}, —NO₂, —N(R^{c2})₂, —SR^{c1}, —CN, or —SCN;
wherein R^{c1} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom;
wherein each instance of R^{c2} is independently hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{c2} are taken together with their intervening atoms to form a substituted or unsubstituted heterocyclic or substituted or unsubstituted heteroaryl ring;
x is 0, 1, or 2;
y is 0 or 1; and
z is 0, 1, 2, 3, 4, 5, 6, 7, 8, 9, or 10, as valency permits.

In some embodiments, the compound is of Formula (I-A):

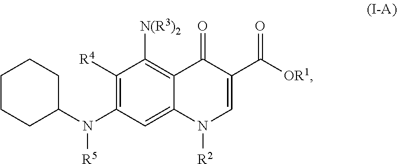

or a pharmaceutically acceptable salt, solvate, hydrate, polymorph, co-crystal, tautomer, stereoisomer, isotopically labeled derivative, prodrug, composition, or mixture thereof, wherein:
R¹ is hydrogen, optionally substituted alkyl, optionally substituted alkenyl, or optionally substituted alkynyl;
R² is hydrogen, optionally substituted alkyl, optionally substituted alkenyl, or optionally substituted alkynyl;
each instance of R³ is independently hydrogen, optionally substituted alkyl, optionally substituted alkenyl, or optionally substituted alkynyl;
R⁴ is halogen, optionally substituted acyl, optionally substituted alkyl, optionally substituted heteroalkyl, optionally substituted alkenyl; and
R⁵ is hydrogen, optionally substituted alkyl, optionally substituted alkenyl, or optionally substituted alkynyl.

In some embodiments, R¹ is hydrogen. In some embodiments, R² is optionally substituted alkyl. In some embodiments, R² is ethyl. In some embodiments, at least one instance of R³ is hydrogen. In some embodiments, both instances of R³ are hydrogen. In some embodiments, at least one instance of R⁴ is halogen. In some embodiments, at least one instance of R⁴ is fluorine. In some embodiments, x is 1. In some embodiments, R⁵ is hydrogen. In some embodiments, y is 1. In some embodiments, z is 0.

In some embodiments, the compound is of formula:

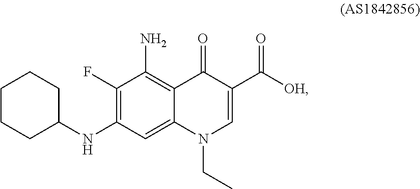 [SMILES: CCn1cc(C(=O)O)c(=O)c2c(N)c(F)c(NC3CCCCC3)cc21]

or a pharmaceutically acceptable salt, solvate, hydrate, polymorph, co-crystal, tautomer, stereoisomer, isotopically labeled derivative, prodrug, composition, or mixture thereof.

In some embodiments, the compound is AS1842856.

In some embodiments, a medium described herein does not comprise a FoxO1 inhibitor.

### Transforming Growth Factor-β (TGF-β) Superfamily

Aspects of the disclosure relate to the use of growth factors from the transforming growth factor-β (TGF-β) superfamily as differentiation factors. The “TGF-β superfamily” means proteins having structural and functional characteristics of known TGFβ family members. The TGFβ family of proteins can include the TGFβ series of proteins, the Inhibins (including Inhibin A and Inhibin B), the Activins (including Activin A, Activin B, and Activin AB), MIS (Müllerian inhibiting substance), BMP (bone morphogenetic proteins), dpp (decapentaplegic), Vg-1, MNSF (monoclonal nonspecific suppressor factor), and others. Activity of this family of proteins can be based on specific binding to certain receptors on various cell types. Members of this family can share regions of sequence identity, particularly at the C-terminus, that correlate to their function. The TGFβ family can include more than one hundred distinct proteins, all sharing at least one region of amino acid sequence identity. Members of the family that can be used in the method disclosed herein can include, but are not limited to, the following proteins, as identified by their GenBank accession numbers: P07995, P18331, P08476, Q04998, P03970, P43032, P55102, P27092, P42917, P09529, P27093, P04088, Q04999, P17491, P55104, Q9WUK5, P55103, O88959, O08717, P58166, 061643, P35621, P09534, P48970, Q9NR23, P25703, P30884, P12643, P49001, P21274, O46564, O19006, P22004, P20722, Q04906, Q07104, P30886, P18075, P23359, P22003, P34821, P49003, Q90751, P21275, Q06826, P30885, P34820, Q29607, P12644, Q90752, O46576, P27539, P48969, Q26974, P07713, P91706, P91699, P27091, O42222, Q24735, P20863, O18828, P55106, Q9PTQ2, O14793, O08689, O42221, O18830, O18831, O18836, O35312, O42220, P43026, P43027, P43029, O95390, Q9R229, O93449, Q9Z1W4, Q9BDW8, P43028, Q7Z4P5, P50414, P17246, P54831, P04202, P01137, P09533, P18341, O19011, Q9Z1Y6, P07200, Q9Z217, O95393, P55105, P30371, Q9MZE2, Q07258, Q96S42, P97737, AAA97415.1, NP-776788.1, NP-058824.1, EAL24001.1, 1 S4Y, NP-001009856.1, NP-1-032406.1, NP-999193.1, XP-519063.1, AAG17260.1, CAA40806.1, NP-1-001009458.1, AAQ55808.1, AAK40341.1, AAP33019.1, AAK21265.1, AAC59738.1, CAI46003.1, B40905, AAQ55811.1, AAK40342.1, XP-540364.1, P55102, AAQ55810.1, NP-990727.1, CAA51163.1, AAD50448.1, JC4862, PN0504, BAB17600.1, AAH56742.1, BAB17596.1, CAG06183.1, CAG05339.1, BAB17601.1, CAB43091.1, A36192, AAA49162.1, AAT42200.1, NP-789822.1, AAA59451.1, AAA59169.1, XP-541000.1, NP-990537.1, NP-1-002184.1, AAC14187.1, AAP83319.1, AAA59170.1, BAB16973.1, AAM66766.1, WFPGBB, 1201278C, AAH30029.1, CAA49326.1, XP-344131.1, AA-148845.1, XP-1-148966.3, 148235, B41398, AAH77857.1, AAB26863.1, 1706327A, BAA83804.1, NP-571143.1, CAG00858.1, BAB17599.1, BAB17602.1, AAB61468.1, PN0505, PN0506, CAB43092.1, BAB17598.1, BAA22570.1, BAB 16972.1, BAC81672.1, BAA12694.1, BAA08494.1, B36192, C36192, BAB16971.1, NP-034695.1, AAA49160.1, CAA62347.1, AAA49161.1, AAD30132.1, CAA58290.1, NP-005529.1, XP-522443.1, AAM27448.1, XP-538247.1, AAD30133. I, AAC36741.1, AAH10404.1, NP-032408.1, AAN03682.1, XP-509161.1, AAC32311.1, NP-651942.2, AAL51005.1, AAC39083.1, AAH85547.1, NP-571023.1, CAF94113.1, EAL29247.1, AAW30007.1, AAH90232.1, A29619, NP-001007905.1, AAH73508.1, AADO2201.1, NP-999793.1, NP-990542.1, AAF19841.1, AAC97488.1, AAC60038.1, NP 989197.1, NP-571434.1, EAL41229.1, AAT07302.1, CAI19472.1, NP-031582.1, AAA40548.1, XP-535880.1, NP-1-037239.1, AAT72007.1, XP-418956.1, CAA41634.1, BAC30864.1, CAA38850.1, CAB81657.2, CAA45018.1, CAA45019.1, BAC28247.1, NP-031581.1, NP-990479.1, NP-999820.1, AAB27335.1, S45355, CAB82007.1, XP-534351.1, NP-058874.1, NP-031579.1, 1REW, AAB96785.1, AAB46367.1, CAA05033.1, BAA89012.1, IES7, AAP20870.1, BAC24087.1, AAG09784.1, BAC06352.1, AAQ89234.1, AAM27000.1, AAH30959.1, CAGO1491.1, NP-571435.1, 1REU, AAC60286.1, BAA24406.1, A36193, AAH55959.1, AAH54647.1, AAH90689.1, CAG09422.1, BAD16743.1, NP-032134.1, XP-532179.1, AAB24876.1, AAH57702.1, AAA82616.1, CAA40222.1, CAB90273.2, XP-342592.1, XP-534896.1, XP-534462.1, 1LXI, XP-417496.1, AAF34179.1, AAL73188.1, CAF96266.1, AAB34226.1, AAB33846.1, AAT12415.1, AA033819.1, AAT72008.1, AAD38402.1, BAB68396.1, CAA45021.1, AAB27337.1, AAP69917.1, AATI2416.1, NP-571396.1, CAA53513.1, AA033820.1, AAA48568.1, BAC02605.1, BAC02604.1, BAC02603.1, BAC02602.1, BAC02601.1, BAC02599.1, BAC02598.1, BAC02597.1, BAC02595.1, BAC02593.1, BAC02592.1, BAC02590.1, AAD28039.1, AAP74560.1, AAB94786.1, NP-001483.2, XP-528195.1, NP-571417.1, NP-001001557. I, AAH43222.1, AAM33143.1, CAG10381.1, BAA31132.1, EAL39680.1, EAA12482.2, P34820, AAP88972.1, AAP74559.1, CAI16418.1, AAD30538.1, XP-345502.1, NP-1-038554.1, CAG04089.1, CAD60936.2, NP-031584.1, B55452, AAC60285.1, BAA06410.1, AAH52846.1, NP-031580.1, NP-1-036959.1, CAA45836.1, CAA45020.1, Q29607, AAB27336.1, XP-547817.1, AAT12414.1, AAM54049.1, AAH78901.1, AA025745.1, NP-570912.1, XP-392194.1, AAD20829.1, AAC97113.1, AAC61694.1, AAH60340.1, AAR97906.1, BAA32227.1, BAB68395.1, BAC02895.1, AAWS 1451.1, AAF82188.1, XP-544189.1, NP-990568.1, BAC80211.1, AAW82620.1, AAF99597.1, NP-571062.1, CAC44179.1, AAB97467.1, AAT99303.1, AAD28038.1, AAH52168.1, NP-001004122.1, CAA72733.1, NP-032133.2, XP-394252.1, XP-224733.2, JH0801, AAP97721.1, NP-989669.1, S43296, P43029, A55452, AAH32495.1, XP-542974.1, NP-032135.1, AAK30842.1, AAK27794.1, BAC30847.1, EAA12064.2, AAP97720.1, XP-525704.1, AAT07301.1, BAD07014.1, CAF94356.1, AAR27581.1, AAG13400.1, AAC60127.1, CAF92055.1, XP-540103.1, AA020895.1, CAF97447.1, AAS01764.1, BAD08319.1, CAA10268.1, NP-998140.1, AAR03824.1, AAS48405.1, AAS48403.1, AAK53545.1, AAK84666.1, XP-395420.1, AAK56941.1, AAC47555.1, AAR88255.1, EAL33036.1, AAW47740.1, AAW29442.1, NP-722813.1, AARO8901.1, AAO 15420.2, CAC59700.1, AAL26886.1, AAK71708.1, AAK71707.1, CAC51427.2, AAK67984.1, AAK67983.1, AAK28706.1, P07713, P91706, P91699, CAG02450.1, AAC47552.1, NP-005802.1, XP-343149.1, AW34055.1, XP-538221.1, AAR27580.1, XP-125935.3, AAF21633.1, AAF21630.1, AAD05267.1, Q9Z1 W4, NP-1-031585.2, NP-571094.1, CAD43439.1, CAF99217.1, CAB63584.1, NP-722840.1, CAE46407.1, XP-1-417667.1, BAC53989.1, BAB19659.1, AAM46922.1, AAA81169.1, AAK28707.1, AAL05943.1, AAB17573.1, CAH25443.1, CAG10269.1, BAD16731.1, EAA00276.2, AAT07320.1, AAT07300.1, AAN15037.1, CAH25442.1, AAK08152.2, 2009388A, AAR12161.1, CAGO1961.1, CAB63656.1, CAD67714.1, CAF94162.1, NP-477340.1, EAL24792.1, NP-1-001009428.1, AAB86686.1, AAT40572.1, AAT40571.1, AAT40569.1, NP-033886.1, AAB49985.1, AAG39266.1, Q26974, AAC77461.1, AAC47262.1, BAC05509.1, NP-055297.1, XP-546146.1, XP-525772.1, NP-060525.2, AAH33585.1, AAH69080.1, CAG12751.1, AAH74757.2, NP-034964.1, NP-038639.1, 042221, AAF02773.1, NP-062024.1, AAR18244.1, AAR14343.1, XP-228285.2, AAT40573.1, AAT94456.1, AAL35278.1, AAL35277.1, AAL17640.1, AAC08035.1, AAB86692.1, CAB40844.1, BAC38637.1, BAB 16046.1, AAN63522.1, NP-571041.1, AAB04986.2, AAC26791.1, AAB95254.1, BAA11835.1, AAR18246.1, XP-538528.1, BAA31853.1, AAK18000.1, XP-1-420540.1, AAL35276.1, AAQ98602.1, CAE71944.1, AAW50585.1, AAV63982.1, AAW29941.1, AAN87890.1, AAT40568.1, CAD57730.1, AAB81508.1, AAS00534.1, AAC59736.1, BAB79498.1, AAA97392.1, AAP85526.1, NP-999600.2, NP-878293.1, BAC82629.1, CAC60268.1, CAG04919.1, AAN10123.1, CAA07707.1 AAK20912.1, AAR88254.1, CAC34629.1, AAL35275.1, AAD46997. I, AAN03842.1, NP-571951.2, CAC50881.1, AAL99367.1, AAL49502.1, AAB71839.1, AAB65415.1, NP-624359.1, NP-990153.1, AAF78069.1, AAK49790.1, NP-919367.2, NP-001192.1, XP-544948.1, AAQ18013.1, AAV38739.1, NP-851298.1, CAA67685.1, AAT67171.1, AAT37502.1, AAD27804.1, AAN76665.1, BAC11909.1, XP-1-421648.1, CAB63704.1, NP-037306.1, A55706, AAF02780.1, CAG09623.1, NP-067589.1, NP-035707.1, AAV30547.1, AAP49817.1, BAC77407.1, AAL87199.1, CAG07172.1, B36193, CAA33024.1, NP-1-001009400.1, AAP36538.1, XP-512687.1, XP-510080.1, AAH05513.1, 1KTZ, AAH14690.1, AAA31526.1.

The growth factor from the TGF-β superfamily in the methods and compositions provided herein can be naturally obtained or recombinant. In some embodiments, the growth factor from the TGF-β superfamily comprises Activin A. The term “Activin A” can include fragments and derivatives of Activin A. The sequence of an exemplary Activin A is provided as SEQ ID NO: 17. Other non-limiting examples of Activin A are provided in SEQ ID NO: 19-32, and non-limiting examples of nucleic acids encoding Activin A are provided in SEQ ID NO: 18, SEQ ID NO: 33, and SEQ ID NO: 34. In some embodiments, the growth factor from the TGF-β superfamily comprises a polypeptide comprising an amino acid sequence that is at least 30%, at least 40%, at least 50%, at least 60%, at least 70%, at least 75%, at least 80%, at least 85%, at least 90%, at least 95%, at least 98% or at least 99% identical to the amino acid sequence of any one of SEQ ID NOs: 17 and 19-32, or functional fragments thereof. In some embodiments, the growth factor from the TGF-β superfamily comprises a polypeptide comprising the amino acid sequence of—any one of SEQ ID NOs: 17 and 19-32.

Homo sapiens Inhibin beta A subunit
(Activin A) amino acid sequence:
SEQ ID NO: 17
GLECDGKVNICCKKQFFVSFKDIGWNDWIIAPSGYHANYCEGECPSHIAGTSGSSLSFHS
TVINHYRMRGHSPFANLKSCCVPTKLRPMSMLYYDDGQNIIKKDIQNMIVEECGCS
Homo sapiens Inhibin beta A chain (Activin A)
nucleic acid sequence:
SEQ ID NO: 18
GGCTTGGAGTGTGATGGCAAGGTCAACATCTGCTGTAAGAAACAGTTCTTTGTCAGT
TTCAAGGACATCGGCTGGAATGACTGGATCATTGCTCCCTCTGGCTATCATGCCAAC
TACTGCGAGGGTGAGTGCCCGAGCCATATAGCAGGCACGTCCGGGTCCTCACTGTCC
TTCCACTCAACAGTCATCAACCACTACCGCATGCGGGGCCATAGCCCCTTTGCCAAC
CTCAAATCGTGCTGTGTGCCCACCAAGCTGAGACCCATGTCCATGTTGTACTATGAT
GATGGTCAAAACATCATCAAAAAGGACATTCAGAACATGATCGTGGAGGAGTGTGG
GTGCTCATAG
Homo sapiens Erythroid differentiation
protein (EDF) ovarian amino acid sequence:
SEQ ID NO: 19
MPLLWLRGFLLASCWIIVRSSPTPGSEGHSAAPDCPSCALAALPKDVPNSQPEMVEAVK
KHILNMLHLKKRPDVTQPVPKAALLNAIRKLHVGKVGENGYVEIEDDIGRRAEMNELM
EQTSEIITFAESGTARKTLHFEISKEGSDLSVVERAEVWLFLKVPKANRTRTKVTIRLFQQ
QKHPQGSLDTGEEAEEVGLKGERSELLLSEKVVDARKSTWHVFPVSSSIQRLLDQGKSS
LDVRIACEQCQESGASLVLLGKKKKKEEEGEGKKKGGGEGGAGADEEKEQSHRPFLML
QARQSEDHPHRRRRRGLECDGKVNICCKKQFFVSFKDIGWNDWIIAPSGYHANYCEGE
CPSHIAGTSGSSLSFHSTVINHYRMRGHSPFANLKSCCVPTKLRPMSMLYYDDGQNIIKK
DIQNMIVEECGCS
Homo sapiens Inhibin B subunit amino acid sequence:
SEQ ID NO: 20
ARQSEDHPHRRRRRGLECDGKVNICCKKQFFVSFKDIGWNDWIIAPSGYHANYCEGECP
SHIAGTSGSSLSFHSTVINHYRMRGHSPFANLKSCCVPTKLRPMSMLYYDDGQNIIKKDI
QNMIVEECGCS
Homo sapiens Inhibin B subunit in testis Homo sapiens
amino acid sequence:
SEQ ID NO: 21
GLECDGKVNICCKKQFFVSFKDIGWNDWIIAPSGYHANYCEGECPSHIAGTSGSSLSFHS
TVINHYACGHSPFANLKSCCVPTKLRPMSMLYYDDGQNIIKKDIQNMIVEECGCS
Homo sapiens Inhibin B subunit erythroid differentiation
protein (EDF), amino acid sequence:
SEQ ID NO: 22
MPLLWLRGFLLASCWIIVRSSPTPGSEGHSAAPDCPSCALAALPKDVPNSQPEMVEAVK
KHILNMLHLKKRPDVTQPVPKAALLNAIRKLHVGKVGENGYVEIEDDIGRRAEMNELM
EQTSEIITFAESGTARKTLHFEISKEGSDLSVVERAEVWLFLKVPKANRTRTKVTIRLFQQ
QKHPQGSLDTGEEAEEVGLKGERSELLLSEKVVDARKSTWHVFPVSSSIQRLLDQGKSS
LDVRIACEQCQESGASLVLLGKKKKKEEEGEGKKKGGGEGGAGADEEKEQSHRPFLML
QARQSEDHPHRRRRRGLECDGKVNICCKKQFFVSFKDIGWNDWIIAPSGYHANYCEGE
CPSHIAGTSGSSLSFHSTVINHYRMRGHSPFANLKSCCVPTKLRPMSMLYYDDGQNIIKK
DIQNMIVEECGCS
Mus musculus (Mouse) Inhibin beta A chain (Activin beta-A chain)
amino acid sequence:
SEQ ID NO: 23
MPLLWLRGFLLASCWIIVRSSPTPGSEGHGSAPDCPSCALATLPKDGPNSQPEMVEAVK
KHILNMLHLKKRPDVTQPVPKAALLNAIRKLHVGKVGENGYVEIEDDIGRRAEMNELM
EQTSEIITFAESGTARKTLHFEISKEGSDLSVVERAEVWLFLKVPKANRTRTKVTIRLFQQ
QKHPQGSLDTGDEAEEMGLKGERSELLLSEKVVDARKSTWHIFPVSSSIQRLLDQGKSS
LDVRIACEQCQESGASLVLLGKKKKKEVDGDGKKKDGSDGGLEEEKEQSHRPFLMLQA
RQSEDHPHRRRRRGLECDGKVNICCKKQFFVSFKDIGWNDWIIAPSGYHANYCEGECPS
HIAGTSGSSLSFHSTVINHYRMRGHSPFANLKSCCVPTKLRPMSMLYYDDGQNIIKKDIQ
NMIVEECGCS
Rattus norvegicus (Rat) Inhibin beta A chain (Activin beta-A
chain) amino acid sequence:
SEQ ID NO: 24
MPLLWLRGFLLASCWIIVRSSPTPGSEGHGAAPDCPSCALATLPKDGPNSQPEMVEAVK
KHILNMLHLKKRPDVTQPVPKAALLNAIRKLHVGKVGENGYVEIEDDIGRRAEMNELM
EQTSEIITFAESGTARKTLHFEISKEGSDLSVVERAEVWLFLKVPKANRTRTKVTIRLFQQ
QKHPQGSLDMGDEAEEMGLKGERSELLLSEKVVDARKSTWHIFPVSSSIQRLLDQGKSS
LDVRIACEQCQESGASLVLLGKKKKKEVDGDGKKKDGSDGGLEEEKEQSHRPFLMLQA
RQSEDHPHRRRRRGLECDGKVNICCKKQFFVSFKDIGWNDWIIAPSGYHANYCEGECPS
HIAGTSGSSLSFHSTVINHYRMRGHSPFANLKSCCVPTKLRPMSMLYYDDGQNIIKKDIQ
NMIVEECGCS
Gallus gallus (Chicken) Inhibin beta A chain (Activin beta-A
chain) amino acid sequence:
SEQ ID NO: 25
MPLLWKRGFLLVICWIIVRSSPTPGSEGHSSVADCPSCALTTLSKDVPSSQPEMVEAVKK
HILNMLHLRDRPNITQPVPKAALLNATKKLHVGKVGDDGYVEIEDDVGRRAEMNEVVE
QTSEIITFAESGTPKKTLHFEISKEGSELSVVEHAEVWLFLKVSKANRSRTKVTIRLFQQQ
RQPKGNSEAAEDMEDMGLKGERSETLISEKAVDARKSTWHIFPISSSVQRLLDQGQSSL
DVRIACDLCQETGASLVLLGKKKKKEDDGEGKEKDGGELTGEEEKEQSHRPFLMMLAR
HSEDRQHRRRERGLECDGKVNICCKKQFFVSFKDIGWSDWIIAPTGYHANYCEEECPSHI
AGTSGSSLSFHSTVINHYRMRGHSPFANLKSCCVPTKLRPMSMLYYDDGQNIIKKDIQN
MIVEECGCS
Bos taurus (Bovine) Inhibin beta A chain (Activin beta-A chain)
amino acid sequence:
SEQ ID NO: 26
MPLLWLRGFLLASCWIIVRSSPTPGSEGHSAAPDCPSCALATLPKDVPNSQPEMVEAVK
KHILNMLHLKKRPDVTQPVPKAALLNAIRKLHVGKVGENGYVEIEDDIGRRAEMNELM
EQTSEIITFAESGTARKTLHFEISKEGSDLSVVERAEIWLFLKVPKANRTRSKVTIRLFQQQ
KHLQGSLDAGEEAEEVGLKGEKSEMLISEKVVDARKSTWHIFPVSSCIQRLLDQGKSSL
DIRIACEQCQETGASLVLLGKKKKKEEEGEGKKRDGEGGAGGDEEKEQSHRPFLMLQA
RQSEDHPHRRRRRGLECDGKVNICCKKQFFVSFKDIGWNDWIIAPSGYHANYCEGECPS
HIAGTSGSSLSFHSTVINHYRMRGHSPFANLKSCCVPTKLRPMSMLYYDDGQNIIKKDIQ
NMIVEECGCS
Equus caballus (Horse) Inhibin beta A chain (Activin beta-A
chain) amino acid sequence:
SEQ ID NO: 27
MPLLWLRGFLLASCWIIVKSSPTPGSEGHSAAPNCPSCALATLPKDVPNAQPEMVEAVK
KHILNMLHLKKRPDVTQPVPKAALLNAIRKLHVGKVGENGYVEIEDDIGRRAEMNELM
EQTSEIITFAESGTARKTLHFEISKEGSDLSVVERAEVWLFLKVPKANRTRSKVTIRLLQQ
QKHPQGSSDTREEAEEADLMEERSEQLISEKVVDARKSTWHIFPVSSSIQRLLDQGKSSL
DIRIACDQCHETGASLVLLGKKKKKEEEGEGKKKDGGEAGAGVDEEKEQSHRPFLMLQ
ARQSEDHPHRRRRRGLECDGKVNICCKKQFFVSFKDIGWNDWIIAPSGYHANYCEGECP
SHIAGTSGSSLSFHSTVINQYRLRGHNPFANLKSCCVPTKLRPMSMLYYDDGQNIIKKDI
QNMIVEECGCS
Sus scrofa (Pig) Inhibin beta A chain (Activin beta-A chain)
amino acid sequence:
SEQ ID NO: 28
MPLLWLRGFLLASCWIIVRSSPTPGSGGHSAAPDCPSCALATLPKDVPNSQPEMVEAVK
KHILNMLHLKKRPDVTQPVPKAALLNAIRKLHVGKVGENGYVELEDDIGRRAEMNELM
EQTSEIITFAEAGTARKTLRFEISKEGSDLSVVERAEIWLFLKVPKANRTRTKVSIRLFQQ
QRRPQGSADAGEEAEDVGFPEEKSEVLISEKVVDARKSTWHIFPVSSSIQRLLDQGKSAL
DIRTACEQCHETGASLVLLGKKKKKEEEAEGRKRDGEGAGVDEEKEQSHRPFLMLQAR
QSEEHPHRRRRRGLECDGKVNICCKKQFFVSFKDIGWNDWIIAPSGYHANYCEGECPSHI
AGTSGSSLSFHSTVINHYRMRGHSPFANLKSCCVPTKLRPMSMLYYDDGQNIIKKDIQN
MIVEECGCS
Ovis aries (Sheep) Inhibin beta A chain (Activin beta-A chain)
amino acid sequence:
SEQ ID NO: 29
MPLLWLRGFLLASCWIIVRSSPTPGSEGHSAAPDCPSCALATLPKDVPNSQPEMVEAVK
KHILNMLHLKKRPDVTQPVPKAALLNAIRKLHVGKVGENGYVEIEDDIGRRAEMNELM
EQTSEIITFAESGTARKTLHFEISQEGSDLSVVERAEIWLFLKVPKANRTRSKVTIRLFQQQ
KHLQGSLDAGEEAEEVGLKGEKSEMLISEKVVDARKSTWHIFPVSSCIQRLLDQGKSSL
DIRIACEQCQETGASLVLLGKKKRKEEEGEGKKRDGEGGAGGDEEKEQSHRPFLMLQA
RQSEDHPHRRRRRGLECDGKVNICCKKQFYVSFKDIGWNDWIIAPSGYHANYCEGECPS
HIAGTSGSSLSFHSTVINHYRMRGHSPFANLKSCCVPTKLRPMSMLYYDDGQNIIKKDIQ
NMIVEECGCS
Felis catus (cat) Inhibin beta A chain (Activin beta-A chain)
amino acid sequence:
SEQ ID NO: 30
MPLLWLRGFLLASCWIIVRSSPTPGSEGPGAAPDCPSCALATLPKDVPNSQPEMVEAVK
KHILNMLHLKKRPEVTQPVPKAALLNAIRKLHVGKVGENGYVEIEDDIGRRAEMNELM
EQTSEIITFAESGTARKTLHFEISKEGSDLSVVERAEVWLFLKVPKANRTRTKVTIQLLQK
QPQGGVDAGEEAEEMGLMEERNEVLISEKVVDARKSTWHIFPVSSSIQRLLDQGKSSLD
VRIACEQCHETGASLVLLGKKKKKEEEGEGKKKDGGDGGAGADEDKEQSHRPFLMLQ
ARQSEDHPHRRRRRGLECDGKVNICCKKQFFVSFKDIGWNDWIIAPSGYHANYCEGECP
SHIAGTSGSSLSFHSTVINHYRMRGHSPFANLKSCCVPTKLRPMSMLYYDDGQNIIKKDI
QNMIVEECGCS
Danio rerio (zebrafish) Inhibin beta A chain (Activin beta-A
chain) amino acid sequence:
SEQ ID NO: 31
MSPLPLLSGILLLLIRSCSLSAMVTKGSLPMSEQQAGATVCPSCALARFRKGVSESEDEG
AQQDVVEAVKRHILNMLHLQERPNITHPVPRAALLNAIRKVHVGRVAKDGSVLIEDEAS
NRAETEQAEQTEIITFAETGEAPGIVNFLISKEGGEMSVVDQANVWIFLRLPKGNRTRAN
VNIRLLLQQGAGEKILAEKSVDTRRSGWHTFPASESVQSLLQRGGSTLSLRVSCPLCADA
RATPVLVSPGGSEREQSHRPFLMAVVRQMDELSLRRRRKRGLECDGKARVCCKRQFYV
NFKDIGWNDWIIAPSGYHANYCEGDCASNVASITGNSLSFHSTVISHYRIRGYSPFTNIKS
CCVPTRLRAMSMLYYNEEQKIVKKDIQNMIVEECGCS
Carassius auratus (goldfish) Inhibin beta A chain
(Activin beta-A chain) amino acid sequence:
SEQ ID NO: 32
MSSLTLVNRGTAALRLFVRGLLTHSSREWLSGDGEPDDPVTPCPSCALAQRQKDSEEQT
DMVEAVKRHILNMLHLNTRPNVTHPVPRAALLNAIRRLHVGRVGEDGTVEMEEDGGG
LGEHREQSEEQPFEIITFAEPGDAPDIMKFDISMEGNTLSVVEQANVWLLLKVAKGSRGK
GKVSVQLLQHGKADPGSADGPQEAVVSEKTVDTRRSGWHTLPVSRTVQTLLDGDSSM
LSLRVSCPMCAEAGAVPILVPTESNKGKEREQSHRPFLMVVLKPAEEHPHRRSKRGLEC
DGKIRVCCKRQFYVNFKDIGWSDWIIAPSGYHANYCEGDCPSHVASITGSALSFHSTVIN
HYRMRGYSPFNNIKSCCVPTRLRAMSMLYYNEEQKIIKKDIQNMIVEECGCS
Recombinant Inhibin B subunit nucleic acid sequence
SEQ ID NO: 33
GCCCGGCAGTCTGAAGACCACCCTCATCGCCGGCGTCGGCGGGGCTTGGAGTGTGA
TGGCAAGGTCAACATCTGCTGTAAGAAACAGTTCTTTGTCAGTTTCAAGGACATCGG
CTGGAATGACTGGATCATTGCTCCCTCTGGCTATCATGCCAACTACTGCGAGGGTGA
GTGCCCGAGCCATATAGCAGGCACGTCCGGGTCCTCACTGTCCTTCCACTCAACAGT
CATCAACCACTACCGCATGCGGGGCCATAGCCCCTTTGCCAACCTCAAATCGTGCTG
TGTGCCCACCAAGCTGAGACCCATGTCCATGTTGTACTATGATGATGGTCAAAACAT
CATCAAAAAGGACATTCAGAACATGATCGTGGAGGAGTGTGGGTGCTCATAGAGTT
GCCCAGCCCAGGGGGAAAGGGAGCAAGA
Homo sapiens mature subunit beta(A) inhibin in testis
nucleic acid sequence
SEQ ID NO: 34
GGCCTGGAGTGCGACGGCAAGGTCAACATCTGCTGTAAGAAACAGTTCTTTGTCAGT
TTCAAGGACATCGGCTGGAATGACTGGATCATTGCTCCCTCTGGCTATCATGCCAAC
TACTGCGAGGGTGAGTGCCCGAGCCATATAGCAGGCACGTCCGGGTCCTCACTGTCC
TTCCACTCAACAGTCATCAACCACTACGCATGCGGCCATAGCCCCTTTGCCAACCTC
AAATCGTGCTGTGTGCCCACCAAGCTGAGACCCATGTCCATGTTGTACTATGATGAT
GGTCAAAACATCATCAAAAAGGACATTCAGAACATGATCGTGGAGGAGTGCGGGTG
CTCCTAA

In some embodiments, the growth factor from the TGF-β superfamily comprises growth differentiation factor 8 (GDF8). The term “GDF8” can include fragments and derivatives of GDF8. The sequences of GDF8 polypeptides are available to the skilled artisan. In some embodiments, the growth factor from the TGF-β superfamily comprises a polypeptide having an amino acid sequence at least 30%, at least 40%, at least 50%, at least 60%, at least 70%, at least 80%, at least 90%, at least 95%, or at least 99%, or greater identical to the human GDF8 polypeptide sequence (GenBank Accession EAX10880).

In some embodiments, the growth factor from the TGF-β superfamily comprises a growth factor that is closely related to GDF8, e.g., growth differentiation factor 11 (GDF11). In some embodiments, the growth factor from the TGF-β superfamily comprises a polypeptide having an amino acid sequence at least 30%, at least 40%, at least 50%, at least 60%, at least 70%, at least 80%, at least 90%, at least 95%, or at least 99%, or greater identical to the human GDF11 polypeptide sequence (GenBank Accession AAF21630).

In some embodiments, the growth factor from the TGF-β superfamily can be replaced with an agent mimics the at least one growth factor from the TGF-β superfamily. Exemplary agents that mimic the at least one growth factor from the TGF-β superfamily, include, without limitation, IDE1 and IDE2.

In some embodiments, a medium described herein does not comprise a TGF-β superfamily protein.

### Bone Morphogenetic Protein (BMP) Signaling Pathway Inhibitors

Aspects of the disclosure relate to the use of BMP signaling pathway inhibitors (which also may be referred to as “BMP inhibitors” herein) as cell differentiation factors. The BMP signaling family is a diverse subset of the TGF-β superfamily (Sebald et al. Biol. Chem. 385:697-710, 2004). Over twenty known BMP ligands are recognized by three distinct type II (BMPRII, ActRIIa, and ActRIIb) and at least three type I (ALK2, ALK3, and ALK6) receptors. Dimeric ligands facilitate assembly of receptor heteromers, allowing the constitutively-active type II receptor serine/threonine kinases to phosphorylate type I receptor serine/threonine kinases. Activated type I receptors phosphorylate BMP-responsive (BR-) SMAD effectors (SMADs 1, 5, and 8) to facilitate nuclear translocation in complex with SMAD4, a co-SMAD that also facilitates TGF signaling. In addition, BMP signals can activate intracellular effectors such as MAPK p38 in a SMAD-independent manner (Nohe et al. Cell Signal 16:291-299, 2004). Soluble BMP antagonists such as noggin, chordin, gremlin, and follistatin limit BMP signaling by ligand sequestration.

In some embodiments, the BMP signaling pathway inhibitor in the methods and composition provided herein comprises DMH-1, or a derivative, analogue, or variant thereof. In some embodiments, the BMP signaling pathway inhibitor in the methods and composition provided herein comprises the following compound or a derivative, analogue, or variant of the following compound:

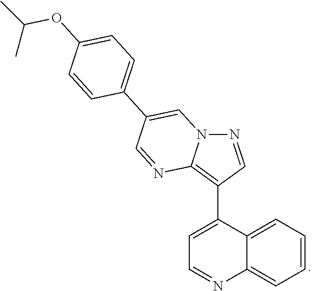 [SMILES: CC(C)Oc1ccc(-c2cnc3c(-c4ccnc5ccccc45)cnn3c2)cc1]

In some embodiments, the BMP signaling pathway inhibitor in the methods and composition provided herein comprises LDN193189 (also known as LDN193189, 1062368-24-4, LDN-193189, DM 3189, DM-3189, IUPAC Name: 4-[6-(4-piperazin-1-ylphenyl)pyrazolo[1,5-a]pyrimidin-3-yl]quinolone). In some embodiments, the BMP signaling pathway inhibitor in the methods and composition provided herein comprises the following compound or a derivative, analogue, or variant of the following compound:

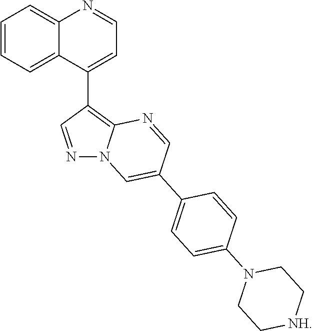 [SMILES: c1ccc2c(-c3cnn4cc(-c5ccc(N6CCNCC6)cc5)cnc34)ccnc2c1]

In some embodiments, DMH-1 can be more selective as compared to LDN193189. In some embodiments of the present disclosure, DMH-1 can be particularly useful for the methods provided herein. In some embodiments, the methods and compositions provided herein, or specific stages of the methods disclosed herein (e.g., stage 3), exclude use of LDN193189. In some embodiments, the methods and compositions provided herein exclude use of LDN193189, or a derivative, analogue, or variant thereof for generating PDX1-positive pancreatic progenitor cells from primitive gut tube cells. In some embodiments, the methods and compositions provided herein relate to use of DMH-1, or a derivative, analogue, or variant thereof for generating PDX1-positive pancreatic progenitor cells from primitive gut tube cells.

In some embodiments, the BMP signaling pathway inhibitor in the methods and composition provided herein comprise an analog or derivative of LDN193189, e.g., a salt, hydrate, solvent, ester, or prodrug of LDN193189. In some embodiments, a derivative (e.g., salt) of LDN193189 comprises LDN193189 hydrochloride.

In some embodiments, the BMP signaling pathway inhibitor in the methods and composition provided herein comprises a compound of Formula I from U.S. Patent Publication No. 2011/0053930.

In some embodiments, a medium described herein does not comprise a BMP signaling pathway inhibitor.

### TGF-β Signaling Pathway Inhibitors

Aspects of the disclosure relate to the use of TGF-β signaling pathway inhibitors as cell differentiation factors.

In some embodiments, the TGF-β signaling pathway comprises TGF-β receptor type I kinase (TGF-β RI) signaling. In some embodiments, the TGF-β signaling pathway inhibitor comprises ALK5 inhibitor II (CAS 446859-33-2, an ATP-competitive inhibitor of TGF-β RI kinase, also known as RepSox, IUPAC Name: 2-[5-(6-methylpyridin-2-yl)-1H-pyrazol-4-yl]-1,5-naphthyridine. In some embodiments, the TGF-β signaling pathway inhibitor is an analog or derivative of ALK5 inhibitor II.

In some embodiments, the analog or derivative of ALK5 inhibitor II (also named “ALK5i”) is a compound of Formula I as described in U.S. Patent Publication No. 2012/0021519, incorporated by reference herein in its entirety.

In some embodiments, the TGF-β signaling pathway inhibitor in the methods and compositions provided herein is a TGF-β receptor inhibitor described in U.S. Patent Publication No. 2010/0267731. In some embodiments, the TGF-β signaling pathway inhibitor in the methods and compositions provided herein comprises an ALK5 inhibitor described in U.S. Patent Publication Nos. 2009/0186076 and 2007/0142376. In some embodiments, the TGF-β signaling pathway inhibitor in the methods and compositions provided herein is A 83-01. In some embodiments, the TGF-β signaling pathway inhibitor in the methods and compositions provided herein is not A 83-01. In some embodiments, the compositions and methods described herein exclude A 83-01. In some embodiments, the TGF-β signaling pathway inhibitor in the methods and compositions provided herein is SB 431542. In some embodiments, the TGF-β signaling pathway inhibitor is not SB 431542. In some embodiments, the compositions and methods described herein exclude SB 431542. In some embodiments, the TGF-β signaling pathway inhibitor in the methods and compositions provided herein is D 4476. In some embodiments, the TGF-β signaling pathway inhibitor is not D 4476. In some embodiments, the compositions and methods described herein exclude D 4476. In some embodiments, the TGF-β signaling pathway inhibitor in the methods and compositions provided herein is GW 788388. In some embodiments, the TGF-β signaling pathway inhibitor is not GW 788388. In some embodiments, the compositions and methods described herein exclude GW 788388. In some embodiments, the TGF-β signaling pathway inhibitor in the methods and compositions provided herein is LY 364947. In some embodiments, the TGF-β signaling pathway inhibitor is not LY 364947. In some embodiments, the compositions and methods described herein exclude LY 364947. In some embodiments, the TGF-β signaling pathway inhibitor in the methods and compositions provided herein is LY 580276. In some embodiments, the TGF-β signaling pathway inhibitor is not LY 580276. In some embodiments, the compositions and methods described herein exclude LY 580276. In some embodiments, the TGF-β signaling pathway inhibitor in the methods and compositions provided herein is SB 525334. In some embodiments, the TGF-β signaling pathway inhibitor is not SB 525334. In some embodiments, the compositions and methods described herein exclude SB 525334. In some embodiments, the TGF-β signaling pathway inhibitor in the methods and compositions provided herein is SB 505124. In some embodiments, the TGF-β signaling pathway inhibitor is not SB 505124. In some embodiments, the compositions and methods described herein exclude SB 505124. In some embodiments, the TGF-β signaling pathway inhibitor in the methods and compositions provided herein is SD 208. In some embodiments, the TGF-β signaling pathway inhibitor is not SD 208. In some embodiments, the compositions and methods described herein exclude SD 208. In some embodiments, the TGF-β signaling pathway inhibitor in the methods and compositions provided herein is GW 6604. In some embodiments, the TGF-β signaling pathway inhibitor is not GW 6604. In some embodiments, the compositions and methods described herein exclude GW 6604. In some embodiments, the TGF-β signaling pathway inhibitor in the methods and compositions provided herein is GW 788388. In some embodiments, the TGF-β signaling pathway inhibitor in the methods and compositions provided herein is not GW 788388. In some embodiments, the compositions and methods described herein exclude GW 788388.

From the collection of compounds described above, the following can be obtained from various sources: LY-364947, SB-525334, SD-208, and SB-505124 available from Sigma, P.O. Box 14508, St. Louis, Mo., 63178-9916; 616452 and 616453 available from Calbiochem (EMD Chemicals, Inc.), 480 S. Democrat Road, Gibbstown, N.J., 08027; GW788388 and GW6604 available from GlaxoSmithKline, 980 Great West Road, Brentford, Middlesex, TW8 9GS, United Kingdom; LY580276 available from Lilly Research, Indianapolis, Ind. 46285; and SM16 available from Biogen Idec, P.O. Box 14627, 5000 Davis Drive, Research Triangle Park, N.C., 27709-4627.

In some embodiments, a medium described herein does not comprise a TGF-β signaling pathway inhibitor.

### WNT Signaling Pathway

Aspects of the disclosure relate to the use of activators of the WNT signaling pathway as cell differentiation factors.

In some embodiments, the WNT signaling pathway activator in the methods and compositions provided herein comprises CHIR99021. In some embodiments, the WNT signaling pathway activator in the methods and compositions provided herein comprises a derivative of CHIR99021, e.g., a salt of CHIR99021, e.g., trihydrochloride, a hydrochloride salt of CHIR99021. In some embodiments, the WNT signaling pathway activator in the methods and compositions provided herein comprises Wnt3a recombinant protein. In some embodiments, the WNT signaling pathway activator in the methods and compositions provided herein comprises a glycogen synthase kinase 3 (GSK3) inhibitor. Exemplary GSK3 inhibitors include, without limitation, 3F8, A 1070722, AR-A 014418, BIO, BIO-acetoxime, FRATide, 10Z-Hymenialdisine, Indirubin-3′oxime, kenpaullone, L803, L803-mts, lithium carbonate, NSC 693868, SB 216763, SB 415286, TC-G 24, TCS 2002, TCS 21311, TWS 119, and analogs or derivatives of any of these. In certain embodiments, the methods, compositions, and kits disclosed herein exclude a WNT signaling pathway activator.

In some embodiments, a medium described herein does not comprise a Wnt signaling pathway activator.

Aspects of the disclosure relate to the use of inhibitors of the WNT signaling pathway as β cell differentiation factors.

In some embodiments, the WNT signaling inhibitor is a tankyrase inhibitor that inhibits expression or activity of at least one tankyrase (TNKS) protein. In some embodiments, the at least one tankyrase protein is tankyrase 1 or tankyrase 2. In some embodiments, the WNT signaling inhibitor inhibits binding of a substrate to a nicotinamide subsite or an adenosine subsite, or both, of a tankyrase protein. In some embodiments, the tankyrase inhibitor is AZ 6102, JW55, MN64, IWR-1-endo, TC-E5001, WIKI4, TNKS 22, TNKS 49, 2X-121 (E7449), XAV-939 (XAV), G007-LK, NVP-TNKS656, decernotinib, (VX-509), vismodegib (GDC-0449), IM-12, GSK429286A, INO-1001, Ofloxacin, TG101209, FG-4592, 1-BET-762, LY2157299, MK-0752, Wnt-C59 (C59), MC1568, Pacritinib (SB 1518), SB415286, Drocinostat, IWR-1-endo, Norfloxacin, SH-4-54, Nexturastat A, SB216763, UNCO 79, dephnetin, GF109203X, RepSox, Sotrastaurin, SB431542, tofacitinib (CP-690550, Tasocitinib), AG-14361, CI994 (tacedinaline), Ro 31-8220 mesylate, resveratrol, NVP-TNKS656, or YO-01027. In some embodiments, said tankyrase inhibitor is AZ 6102, NVP-TNKS656, or IWR-1-endo. In some embodiments, the tankyrase inhibitor is NVP-TNKS656 (NVP). In some embodiments, the tankyrase inhibitor selectively inhibits tankyrase 1 over tankyrase 2. In some embodiments, the tankyrase inhibitor selectively inhibits tankyrase 2 over tankyrase 1.

In some embodiments, a medium described herein does not comprise a Wnt signaling pathway inhibitor.

### Fibroblast Growth Factor (FGF) Family

Aspects of the disclosure relate to the use of growth factors from the FGF family as cell differentiation factors.

In some embodiments, the growth factor from the FGF family in the methods and compositions provided herein comprises keratinocyte growth factor (KGF). The polypeptide sequences of KGF are available to the skilled artisan. An example of human KGF amino acid sequence is provided in GenBank Accession No. AAB21431, provided as SEQ ID NO: 37).

In some embodiments, the growth factor from the FGF family comprises a polypeptide comprises an amino acid sequence that is at least 30%, at least 40%, at least 50%, at least 60%, at least 70%, at least 75%, at least 80%, at least 85%, at least 90%, at least 95%, at least 98% or at least 99% identical to the amino acid sequence of SEQ ID NO: 37, or a functional fragment thereof. In some embodiments, the growth factor from the FGF family comprises a polypeptide comprising the amino acid sequence of SEQ ID NO: 37.

Human KGF amino acid sequence
(GenBank Accession AAB21431; SEQ ID NO: 37)
MHKWILTWILPTLLYRSCFHIICLVGTISLACNDMTPEQMATNVNCSSPE
RHTRSYDYMEGGDIRVRRLFCRTQWYLRIDKRGKVKGTQEMKNNYNIMEI
RTVAVGIVAIKGVESEFYLAMNKEGKLYAKKECNEDCNFKELILENHYNT
YASAKWTHNGGEMFVALNQKGIPVRGKKTKKEQKTAHFLPMAIT

In some embodiments, the growth factor from the FGF family in the methods and composition provided herein comprises FGF2. The polypeptide sequences of FGF2 are available to the skilled artisan. In some embodiments, the growth factor from the FGF family comprises a polypeptide having an amino acid sequence at least 30%, at least 40%, at least 50%, at least 60%, at least 70%, at least 80%, at least 90%, at least 95%, or at least 99%, or greater identical to the human FGF2 polypeptide sequence (GenBank Accession NP 001997).

In some embodiments, the at least one growth factor from the FGF family in the methods and composition provided herein comprises FGF8B. The polypeptide sequences of FGF8B are available to the skilled artisan. In some embodiments, the growth factor from the FGF family comprises a polypeptide having an amino acid sequence at least 30%, at least 40%, at least 50%, at least 60%, at least 70%, at least 80%, at least 90%, at least 95%, or at least 99%, or greater identical to the human FGF8B polypeptide sequence (GenBank Accession AAB40954).

In some embodiments, the at least one growth factor from the FGF family in the methods and composition provided herein comprises FGF10. The polypeptide sequences of FGF10 are available to the skilled artisan. In some embodiments, the growth factor from the FGF family comprises a polypeptide having an amino acid sequence at least 30%, at least 40%, at least 50%, at least 60%, at least 70%, at least 80%, at least 90%, at least 95%, or at least 99%, or greater identical to the human FGF10 polypeptide sequence (GenBank Accession CAG46489).

In some embodiments, the at least one growth factor from the FGF family in the methods and composition provided herein comprises FGF21. The polypeptide sequences of FGF21 are available to the skilled artisan. In some embodiments, the growth factor from the FGF family comprises a polypeptide having an amino acid sequence at least 30%, at least 40%, at least 50%, at least 60%, at least 70%, at least 80%, at least 90%, at least 95%, or at least 99%, or greater identical to the human FGF21 polypeptide sequence (GenBank Accession AAQ89444.1).

In some embodiments, a medium described herein does not comprise a FGF family protein.

### Sonic Hedgehog (SHH) Signaling Pathway

Aspects of the disclosure relate to the use of SHH signaling pathway inhibitors as cell differentiation factors.

In some embodiments, the SHH signaling pathway inhibitor in the methods and composition provided herein comprises Sant1. In some embodiments, the SHH signaling pathway inhibitor in the methods and composition provided herein comprises SANT2. In some embodiments, the SHH signaling pathway inhibitor in the methods and composition provided herein comprises SANT3. In some embodiments, the SHH signaling pathway inhibitor in the methods and composition provided herein comprises SANT4. In some embodiments, the SHH signaling pathway inhibitor comprises Cur61414. In some embodiments, the SHH signaling pathway inhibitor in the methods and composition provided herein comprises forskolin. In some embodiments, the SHH signaling pathway inhibitor in the methods and composition provided herein comprises tomatidine. In some embodiments, the SHH signaling pathway inhibitor in the methods and composition provided herein comprises AY9944. In some embodiments, the SHH signaling pathway inhibitor in the methods and composition provided herein comprises triparanol. In some embodiments, the SHH signaling pathway inhibitor in the methods and composition provided herein comprises compound A or compound B (as disclosed in U.S. Pub. No. 2004/0060568). In some embodiments, the SHH signaling pathway inhibitor in the methods and composition provided herein comprises a steroidal alkaloid that antagonizes hedgehog signaling (e.g., cyclopamine or a derivative thereof) as disclosed in U.S. Pub. No. 2006/0276391. In certain embodiments, the methods, compositions, and kits disclosed herein exclude a SHH signaling pathway inhibitor.

### Rho Kinase (ROCK) Signaling Pathway

Aspects of the disclosure relate to the use of ROCK signaling pathway inhibitors (ROCK inhibitors) as cell differentiation factors.

In some embodiments, the ROCK inhibitor in the methods and composition provided herein comprises Y-27632 or Thiazovivin. In some embodiments, the ROCK inhibitor in the methods and composition provided herein comprises Thiazovivin. In some embodiments, the ROCK inhibitor in the methods and composition provided herein comprises Y-27632. In some embodiments, the ROCK inhibitor in the methods and composition provided herein comprises the following compound or a derivative thereof:

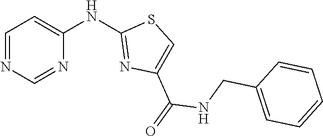 [SMILES: O=C(NCc1ccccc1)c1csc(Nc2ccncn2)n1]

In some embodiments, the ROCK inhibitor in the methods and composition provided herein comprises the following compound or a derivative thereof:

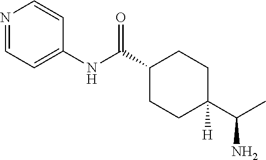 [SMILES: C[C@@H](N)[C@H]1CC[C@H](C(=O)Nc2ccncc2)CC1]

Non-limiting examples of ROCK inhibitor that can be used in the methods and compositions provided herein include Thiazovivin, Y-27632, Fasudil/HA1077, H-1152, Ripasudil, Y39983, Wf-536, SLx-2119, Azabenzimidazole-aminofurazans, DE-104, Olefins, Isoquinolines, Indazoles, and pyridinealkene derivatives, ROKα inhibitor, XD-4000, HMN-1152, 4-(1-aminoalkyl)-N-(4-pyridyl)cyclohexane-carboxamides, Rhostatin, BA-210, BA-207, BA-215, BA-285, BA-1037, Ki-23095, VAS-012, and quinazoline.

In some embodiments, a medium described herein does not comprise a SHH signaling pathway inhibitor.

### Retinoic Acid Signaling Pathway

Aspects of the disclosure relate to the use of modulators of retinoic acid signaling as cell differentiation factors.

In some embodiments, the modulator of retinoic acid signaling in the methods and composition provided herein comprises an activator of retinoic acid signaling. In some embodiments, the RA signaling pathway activator in the methods and composition provided herein comprises retinoic acid. In some embodiments, the RA signaling pathway activator in the methods and composition provided herein comprises a retinoic acid receptor agonist. Exemplary retinoic acid receptor agonists in the methods and composition provided herein include, without limitation, CD 1530, AM 580, TTNPB, CD 437, Ch 55, BMS 961, AC 261066, AC 55649, AM 80, BMS 753, tazarotene, adapalene, and CD 2314.

In some embodiments, the modulator of retinoic acid signaling in the methods and composition provided herein comprises an inhibitor of retinoic acid signaling. In some embodiments, the retinoic acid signaling pathway inhibitor comprises DEAB (IUPAC Name: 2-[2-(diethylamino) ethoxy]-3-prop-2-enylbenzaldehyde). In some embodiments, the retinoic acid signaling pathway inhibitor comprises an analog or derivative of DEAB.

In some embodiments, the retinoic acid signaling pathway inhibitor in the methods and composition provided herein comprises a retinoic acid receptor antagonist. In some embodiments, the retinoic acid receptor antagonist in the methods and composition provided herein comprises (E)-4-[2-(5,6-dihydro-5,5-dimethyl-8-phenyl-2-naphthalenyl)ethenyl]benzoic acid, (E)-4-[[(5,6-dihydro-5,5-dimethyl-8-phenylethynyl)-2-naphthalenyl]ethenyl]benzoic acid, (E)-4-[2-[5,6-dihydro-5,5-dimethyl-8-(2-naphthalenyl)-2-naphthalenyl]ethenyl]-benzoic acid, and (E)-4-[2-[5,6-dihydro-5,5-dimethyl-8-(4-methoxyphenyl)-2-naphthalenyl]ethenyl]benzoic acid. In some embodiments, the retinoic acid receptor antagonist comprises BMS 195614 (CAS #253310-42-8), ER 50891 (CAS #187400-85-7), BMS 493 (CAS #170355-78-9), CD 2665 (CAS #170355-78-9), LE 135 (CAS #155877-83-1), BMS 453 (CAS #166977-43-1), or MM 11253 (CAS #345952-44-5).

In certain embodiments, the methods, compositions, and kits disclosed herein exclude a modulator of retinoic acid signaling. In certain embodiments, the methods, compositions, and kits disclosed herein exclude a retinoic acid signaling pathway activator. In certain embodiments, the methods, compositions, and kits disclosed herein exclude a retinoic acid signaling pathway inhibitor.

In some embodiments, a medium described herein does not comprise retinoic acid.

### Protein Kinase C

Aspects of the disclosure relate to the use of protein kinase C activators as cell differentiation factors. Protein kinase C is one of the largest families of protein kinase enzymes and is composed of a variety of isoforms. Conventional isoforms include a, βI, βII, γ; novel isoforms include δ, ε, η, Θ; and atypical isoforms include ε, and ι/λ. PKC enzymes are primarily cytosolic but translocate to the membrane when activated. In the cytoplasm, PKC is phosphorylated by other kinases or autophosphorylated. In order to be activated, some PKC isoforms (e.g., PKC-ε) require a molecule to bind to the diacylglycerol (“DAG”) binding site or the phosphatidylserine (“PS”) binding site. Others are able to be activated without any secondary binding messengers at all. PKC activators that bind to the DAG site include, but are not limited to, bryostatin, picologues, phorbol esters, aplysiatoxin, and gnidimacrin. PKC activators that bind to the PS site include, but are not limited to, polyunsaturated fatty acids and their derivatives. It is contemplated that any protein kinase C activator that is capable, either alone or in combination with one or more other β cell differentiation factors, of inducing the differentiation of at least one insulin-producing, endocrine cell or precursor thereof into a SC-β cell can be used in the methods, compositions, and kits described herein.

In some embodiments, any of the PKC activators disclosed herein is a PKC activator capable of binding to a DAG binding site on a PKC. In some embodiments, the PKC activator is capable of binding to a CI domain of a PKC. In some embodiments, the PKC activator is a benzolactam-derivative. In some embodiments, the benzolactam-derivative is ((2S,5S)-(E,E)-8-(5-(4-(Trifluoromethyl)phenyl)-2,4-pentadienoylamino)benzolactam), which may be referred to herein as TPPB or TPB. In some embodiments, contacting a population of cells with a benzolactam-derivative PKC activator (e.g., TPPB) increases cell yield as compared to a population of cells not treated with the benzolactam-derivative PKC activator. In some embodiments, the PKC activator is a phorbol ester. In some embodiments, the phorbol ester is Phorbol 12,13-dibutyrate, which may be referred to herein as PDBU or PdbU. In some embodiments, contacting a population of cells with a benzolactam-derivative PKC activator (e.g., TPPB) increases cell yield as compared to a population of cells treated with a phorbol ester PKC activator (e.g., PdbU). In some embodiments, the PKC activator in the methods and composition provided herein comprises PdbU. In some embodiments, the PKC activator in the methods and composition provided herein comprises TPB. In some embodiments, the PKC activator in the methods and composition provided herein comprises cyclopropanated polyunsaturated fatty acids, cyclopropanated monounsaturated fatty acids, cyclopropanated polyunsaturated fatty alcohols, cyclopropanated monounsaturated fatty alcohols, cyclopropanated polyunsaturated fatty acid esters, cyclopropanated monounsaturated fatty acid esters, cyclopropanated polyunsaturated fatty acid sulfates, cyclopropanated monounsaturated fatty acid sulfates, cyclopropanated polyunsaturated fatty acid phosphates, cyclopropanated monounsaturated fatty acid phosphates, macrocyclic lactones, DAG derivatives, isoprenoids, octylindolactam V, gnidimacrin, iripallidal, ingenol, napthalenesulfonamides, diacylglycerol kinase inhibitors, fibroblast growth factor 18 (FGF-18), insulin growth factor, hormones, and growth factor activators, as described in WIPO Pub. No. WO/2013/071282. In some embodiments, the bryostain comprises bryostatin-1, bryostatin-2, bryostatin-3, bryostatin-4, bryostatin-5, bryostatin-6, bryostatin-7, bryostatin-8, bryostatin-9, bryostatin-10, bryostatin-11, bryostatin-12, bryostatin-13, bryostatin-14, bryostatin-15, bryostatin-16, bryostatin-17, or bryostatin-18. In certain embodiments, the methods, compositions, and kits disclosed herein exclude a protein kinase C activator.

In some embodiments, a medium Described herein does not comprise a protein kinase C activator.

### γ-Secretase Inhibitors

Aspects of the disclosure relate to the use of γ-secretase inhibitors as cell differentiation factors.

In some embodiments, the γ-secretase inhibitor in the methods and composition provided herein comprises XXI. In some embodiments, the γ-secretase inhibitor in the methods and composition provided herein comprises DAPT. Additional exemplary γ-secretase inhibitors in the methods and composition provided herein include, without limitation, the γ-secretase inhibitors described in U.S. Pat. Nos. 7,049,296, 8,481,499, 8,501,813, and WIPO Pub. No. WO/2013/052700. In certain embodiments, the methods, compositions, and kits disclosed herein exclude a γ-secretase inhibitor.

In some embodiments, a medium described herein does not comprise a γ-secretase inhibitor.

### Thyroid Hormone Signaling Pathway Activators

Aspects of the disclosure relate to the use of thyroid hormone signaling pathway activators as cell differentiation factors.

In some embodiments, the thyroid hormone signaling pathway activator in the methods and composition provided herein comprises triiodothyronine (T3). In some embodiments, the thyroid hormone signaling pathway activator in the methods and composition provided herein comprises GC-1. In some embodiments, the thyroid hormone signaling pathway activator in the methods and composition provided herein comprises an analog or derivative of T3 or GC-1. Exemplary analogs of T3 in the methods and composition provided herein include, but are not limited to, selective and non-selective thyromimetics, TRβ selective agonist-GC-1, GC-24,4-Hydroxy-PCB 106, MB07811, MB07344,3,5-diiodothyropropionic acid (DITPA); the selective TR-β agonist GC-1; 3-Iodothyronamine (T(1)AM) and 3,3′,5-triiodothyroacetic acid (Triac) (bioactive metabolites of the hormone thyroxine (T(4)); KB-2115 and KB-141; thyronamines; SKF L-94901; DIBIT; 3′-AC-T2; tetraiodothyroacetic acid (Tetrac) and triiodothyroacetic acid (Triac) (via oxidative deamination and decarboxylation of thyroxine [T4] and triiodothyronine [T3] alanine chain), 3,3′,5′-triiodothyronine (rT3) (via T4 and T3 deiodination), 3,3′-diiodothyronine (3,3′-T2) and 3,5-diiodothyronine (T2) (via T4, T3, and rT3 deiodination), and 3-iodothyronamine (TIAM) and thyronamine (TOAM) (via T4 and T3 deiodination and amino acid decarboxylation), as well as for TH structural analogs, such as 3,5,3′-triiodothyropropionic acid (Triprop), 3,5-dibromo-3-pyridazinone-1-thyronine (L-940901), N-[3,5-dimethyl-4-(4′-hydroxy-3′-isopropylphenoxy)-phenyl]-oxamic acid (CGS 23425), 3,5-dimethyl-4-[(4′-hydroxy-3′-isopropylbenzyl)-phenoxy]acetic acid (GC-1), 3,5-dichloro-4-[(4-hydroxy-3-isopropylphenoxy)phenyl]acetic acid (KB-141), and 3,5-diiodothyropropionic acid (DITPA).

In some embodiments, the thyroid hormone signaling pathway activator in the methods and composition provided herein comprises a prodrug or prohormone of T3, such as T4 thyroid hormone (e.g., thyroxine or L-3,5,3′,5′-tetraiodothyronine).

In some embodiments, the thyroid hormone signaling pathway activator in the methods and composition provided herein is an iodothyronine composition described in U.S. Pat. No. 7,163,918.

In some embodiments, a medium described herein does not comprise a thyroid hormone.

### Epidermal Growth Factor (EGF) Family

Aspects of the disclosure relate to the use of growth factors from the EGF family as cell differentiation factors.

In some embodiments, the at least one growth factor from the EGF family in the methods and composition provided herein comprises betacellulin. An example of human betacellulin amino acid sequence is provided in GenBank Accession No.: AAB25452.1 (SEQ ID NO: 38). In some embodiments, the growth factor from the EGF family used in the compositions and methods described herein comprises an amino acid sequence that is at least 30%, at least 40%, at least 50%, at least 60%, at least 70%, at least 75%, at least 80%, at least 85%, at least 90%, at least 95%, at least 98% or at least 99% identical to the amino acid sequence of SEQ ID NO: 38, or a functional fragment thereof. In some embodiments, the growth factor from the EGF family used in the compositions and methods described herein comprises the amino acid sequence of SEQ ID NO: 38.

Human betacellulin amino acid sequence
(GenBank: AAB25452.1; SEQ ID NO: 38)
MDRAARCSGASSLPLLLALALGLVILHCVVADGNSTRSPETNGLLCGDPE
ENCAATTTQSKRKGHFSRCPKQYKHYCIKGRCRFVVAEQTPSCVCDEGYI
GARCERVDLFYLRGDRGQILVICLIAVMVVFIILVIGVCTCCHPLRKRRK
RKKKEEEMETLGKDITPINEDIEETN

In some embodiments, at least one growth factor from the EGF family in the methods and composition provided herein comprises EGF. Epidermal growth factor (EGF) is a 53 amino acid cytokine which is proteolytically cleaved from a large integral membrane protein precursor. In some embodiments, the growth factor from the EGF family in the methods and composition provided herein comprises a variant EGF polypeptide, for example an isolated epidermal growth factor polypeptide having at least 90% amino acid identity to the human wild-type EGF polypeptide sequence, as disclosed in U.S. Pat. No. 7,084,246. In some embodiments, the growth factor from the EGF family in the methods and composition provided herein comprises an engineered EGF mutant that binds to and agonizes the EGF receptor, as is disclosed in U.S. Pat. No. 8,247,531. Non-limiting examples of amino acid sequences of growth factors from the EGF family that may be used in the compositions and methods described are provided below. In some embodiments, the growth factor from the EGF family used in the compositions and methods described herein comprises an amino acid sequence that is at least 30%, at least 40%, at least 50%, at least 60%, at least 70%, at least 75%, at least 80%, at least 85%, at least 90%, at least 95%, at least 98% or at least 99% identical to the amino acid sequence of any one of SEQ ID NOs: 39-50, or a functional fragment thereof. In some embodiments, the growth factor from the EGF family used in the compositions and methods described herein comprises the amino acid sequence of any one of SEQ ID NO: 39-50.

Homo sapiens epidermal growth factor (WT)
(Genbank: AAS83395.1; SEQ ID NO: 39)
NSDSECPLSHDGYCLHDGVCMYIEALDKYACNCVVGYIGERCQYRDLKWW
ELR
Homo sapiens epidermal growth factor (mutant)
(SEQ ID NO: 40)
NSDSECPLSHDGYCLHGGVCMYIKAVDRYACNCVVGYIGERCQYRDLTWW
GPR
Homo sapiens epidermal growth factor (mutant)
(SEQ ID NO: 41)
NSDSECPLSHDGYCLHDGVCMYIKALDKYACNCVVGYTGERCQYRDLRWW
GRR
Homo sapiens epidermal growth factor (mutant)
(SEQ ID NO: 42)
NSNSECPLSHDGYCLHDGVCRYIEALDRYACNCVVGYIGERCQYGDLRWW
GRR
Homo sapiens epidermal growth factor (mutant)
(SEQ ID NO: 43)
NSDSGCPLSHSGYCLHDGVCMYIKALDRYACNCVVGYAGERCQYRDLRWW
ARR
Homo sapiens epidermal growth factor (mutant)
(SEQ ID NO: 44)
TRGSECPLSHDGYCLHDGVCMYIGALDRYACNCVVGYTGERCQYRDLRWW
ARR
Homo sapiens epidermal growth factor (mutant)
(SEQ ID NO: 45)
NSDFGCPLSYDGYCLHDGVCMYIKALDKYACNCVVGYAGERCQYRDLRWW
GRR
Homo sapiens epidermal growth factor (mutant)
(SEQ ID NO: 46)
SRGSKCPPSHDGYCLHDGVCMYIEALDRYACNCVVGYAGERCQYRDLRWW
ARR
Homo sapiens epidermal growth factor (mutant)
(SEQ ID NO: 47)
SSGSECPSSHDGYCLHDGACMYIEALDRYACNCAVGYAGERCQYRDLRWW
GRR
Homo sapiens epidermal growth factor (mutant)
(SEQ ID NO: 48)
SSNSECPPSHDGYCLHDGVCMYIEALDRYACNCVVGYAGERCQYRDLRWW
ARR
Homo sapiens epidermal growth factor (mutant)
(SEQ ID NO: 49)
NSYSECPPSYDGYCLHDGVCRYIEALDSYACNCVVGYAGERCQYRDLRWW
GRR
Homo sapiens epidermal growth factor (mutant)
(SEQ ID NO: 50)
SSGSECPLSHDGYCLNDGVCMYIEALDKYACNCVVGYVGERCQYRDLRWW
ARR

In some embodiments, the at least one growth factor from the EGF family in the methods and composition provided herein is replaced with an agent that activates a signaling pathway in the EGF family. In some embodiments, the growth factor from the EGF family in the methods and composition provided herein comprises a compound that mimics EGF. In certain embodiments, the methods, compositions, and kits disclosed herein exclude a growth factor from the EGF family.

In some embodiments, a medium described herein does not comprise a EGF family growth factor.

### Epigenetic Modifying Compounds

Aspects of the disclosure relate to the use of epigenetic modifying compound as cell differentiation factors.

The term “epigenetic modifying compound” can refer to a chemical compound that can make epigenetic changes genes, i.e., change gene expression(s) without changing DNA sequences. Epigenetic changes can help determine whether genes are turned on or off and can influence the production of proteins in certain cells, e.g., beta-cells. Epigenetic modifications, such as DNA methylation and histone modification, can alter DNA accessibility and chromatin structure, thereby regulating patterns of gene expression. These processes can be crucial to normal development and differentiation of distinct cell lineages in the adult organism. They can be modified by exogenous influences, and, as such, can contribute to or be the result of environmental alterations of phenotype or pathophenotype. Importantly, epigenetic modification can have a crucial role in the regulation of pluripotency genes, which become inactivated during differentiation. Non-limiting exemplary epigenetic modifying compound include a DNA methylation inhibitor, a histone acetyltransferase inhibitor, a histone deacetylase inhibitor, a histone methyltransferase inhibitor, a bromodomain inhibitor, or any combination thereof.

In an embodiment, the histone methyltransferase inhibitor is an inhibitor of enhancer of zeste homolog 2 (EZH2). EZH2 is a histone-lysine N-methyltransferase enzyme. Non-limiting examples of an EZH2 inhibitor that can be used in the methods provided herein include 3-deazaneplanocin A (DZNep), EPZ6438, EPZ005687 (an S-adenosylmethionine (SAM) competitive inhibitor), EII, GSK126, and UNC1999. DZNep can inhibit the hydrolysis of S-adenosyl-L-homocysteine (SAH), which is a product-based inhibitor of all protein methyltransferases, leading to increased cellular concentrations of SAH which in turn inhibits EZH2. DZNep may not be specific to EZH2 and can also inhibit other DNA methyltransferases. GSK126 is a SAM-competitive EZH2 inhibitor that has 150-fold selectivity over EZH1. UNC1999 is an analogue of GSK126, and it is less selective than its counterpart GSK126.

In an embodiment, the histone methyltransferase inhibitor is DZNep. In an embodiment, the HDAC inhibitor is a class I HDAC inhibitor, a class II HDAC inhibitor, or a combination thereof. In an embodiment, the HDAC inhibitor is KD5170 (mercaptoketone-based HdAC inhibitor), MC1568 (class Ila HDAC inhibitor), TMP195 (class Ila HDAC inhibitor), or any combination thereof. In some embodiments, HDAC inhibitor is vorinostat, romidepsin (Istodax), chidamide, panobinostat (farydak), belinostat (PXD101), panobinostat (LBH589), valproic acid, mocctinostat (MGCD0103), abexinostat (PCI-24781), entinostat (MS-275), SB939, resminostat (4SC-201), givinostat (ITF2357), quisinostat (JNJ-26481585), HBI-8000, (a benzamide HDI), kevetrin, CUDC-101, AR-42, CHR-2845, CHR-3996, 4SC-202, CG200745, ACY-1215, ME-344, sulforaphane, or any variant thereof.

In some embodiments, a medium described herein does not comprise an epigenetic modifying compound.

### Protein Kinase Inhibitors

Aspects of the disclosure relate to the use of protein kinase inhibitors as cell differentiation factors.

In some embodiments, the protein kinase inhibitor in the methods and composition provided herein comprises staurosporine. In some embodiments, the protein kinase inhibitor in the methods and composition provided herein comprises an analog of staurosporine. Exemplary analogs of staurosporine in the methods and composition provided herein include, without limitation, Ro-31-8220, a bisindolylmaleimide (Bis) compound, 10′-{5″-[(methoxycarbonyl)amino]-2″-methyl}-phenylaminocarbonylstaurosporine, a staralog (see, e.g., Lopez et al., “Staurosporine-derived inhibitors broaden the scope of analog-sensitive kinase technology”, J. Am. Chem. Soc. 2013; 135(48):18153-18159), and, cgp41251.

In some embodiments, the protein kinase inhibitor in the methods and composition provided herein is an inhibitor of PKCβ. In some embodiments, the protein kinase inhibitor in the methods and composition provided herein is an inhibitor of PKCβ with the following structure or a derivative, analogue or variant of the compound as follows:

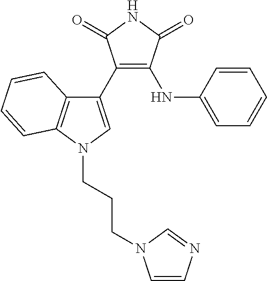 [SMILES: O=C1NC(=O)C(c2cn(CCCn3ccnc3)c3ccccc23)=C1Nc1ccccc1]

In some embodiments, the inhibitor of PKCβ is a GSK-2 compound with the following structure or a derivative, analogue or variant of the compound as follows:

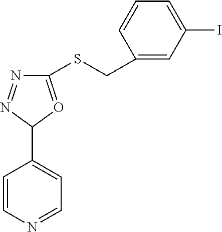

In some embodiments, the inhibitor of PKC in the methods and composition provided herein is a bisindolylmaleimide. Exemplary bisindolylmaleimides include, without limitation, bisindolylmaleimide I, bisindolylmaleimide II, bisindolylmaleimide Ill, hydrochloride, or a derivative, analogue or variant thereof.

In some embodiments, the PKC inhibitor in the methods and composition provided herein is a pseudohypericin, or a derivative, analogue, or variant thereof. In some embodiments, the PKC inhibitor in the methods and composition provided herein is indorublin-3-monoxime, 5-Iodo or a derivative, analogue or variant thereof. In certain embodiments, the methods, compositions, and kits disclosed herein exclude a protein kinase inhibitor.

In some embodiments, a medium described herein does not comprise a protein kinase inhibitor.

### Acetyl CoA-Related Metabolite

In some embodiments, a composition (e.g., medium) of the disclosure comprises an acetyl CoA-related metabolite. Metabolism of acetyl-coenzyme A (acetyl-CoA) can confer numerous metabolic functions, including energy production, lipid synthesis, and protein acetylation.

Exemplary acetyl COA-related metabolites include, but are not limited to acetate, pyruvate, ketogenic amino acids, valine, leucine, isoleucine, phenylalanine, tyrosine, lysine, tryptophan, fatty acids, CoA, Isovaleryl-CoA, and β-hydroxybutyrate. In some embodiments, the acetyl COA-related metabolite is acetate. In some embodiments, a composition of the disclosure contains two or more different acetyl COA related metabolites, for example, 2, 3, 4, 5, 6, 7, 8, 9, 10, or more different acetyl COA-related metabolites. In some embodiments, the acetyl CoA-related metabolite is acetate.

In some embodiments, a medium described herein does not include an acetyl COA-related metabolite (e.g., does not include acetate).

### Histone Deacetylase Inhibitor (HDACi)

In some embodiments, a composition (e.g., medium) of the disclosure comprises a histone deacetylase inhibitor (HDACi). Histone deacetylase inhibitors (HDACi) are a class of compounds that increase acetylation of lysine residues on histone proteins as well as other, nonhistone, proteins by inhibiting the activity of HDAC enzymes.

Exemplary histone deacetylase inhibitors (HDACi) include, but are not limited to β-Hydroxybutyrate, butyric acid, class I HDACi, class IIA HDACi, class IIB HDACi, class III HDACi, class IV HDACi, HDAC-1, HDAC-2, HDAC-3, HDAC-4, HDAC-5, HDAC-6, HDAC-7, HDAC-8, HDAC-9, HDAC-10, HDAC-11, sirtuins, SIRT1, SIRT2, SIRT3, SIRT4, SIRT5, SIRT6, SIRT7, Vorinostat (suberoylanilide hydroxamic acid, SAHA, MK0683), Entinostat (MS-275, SNDX-275), Panobinostat (LBH589, NVP-LBH589), Trichostatin A (TSA), Mocetinostat (MGCD0103, MG0103), GSK3117391 (GSK3117391A, HDAC-IN-3), BRD3308, BRD3308, Tubastatin A TFA (Tubastatin A trifluoroacetate salt), Tubastatin A, SIS17, NKL 22, BML-210 (CAY10433), TC-H 106, SR-4370, Belinostat (PXD101, NSC726630, PX-105684), Romidepsin (FK228, Depsipeptide, FR 901228, NSC 630176), MC1568, Givinostat (ITF2357), Dacinostat (LAQ824, NVP-LAQ824), CUDC-101, Quisinostat (JNJ-26481585), Pracinostat (SB939), PCI-34051, Droxinostat (NS 41080), Abexinostat (PCI-24781), Abexinostat (PCI-24781, CRA-024781), RGFP966, AR-42 (HDAC-42), Ricolinostat (ACY-1215, Rocilinostat), Valproic acid sodium salt (Sodium valproate), Tacedinaline (C1994, PD-123654, GOE-5549, Acetyldinaline), Fimepinostat (CUDC-907), Sodium butyrate (NaB), Curcumin, Diferuloylmethane, M344, Tubacin, RG2833 (RGFP109), RG2833 (RGFP109), Resminostat (RAS2410), Divalproex Sodium, Scriptaid (GCK 1026), Sodium Phenylbutyrate, Sinapinic acid (Sinapic acid), TMP269, Santacruzamate A (CAY10683), TMP195 (TFMO 2), Valproic acid (VPA), UF010, Tasquinimod (ABR-215050), SKLB-23bb, Isoguanosine, Iforaphane, BRD73954, Citarinostat (ACY-241, HDAC-IN-2), Suberohydroxamic acid, plitomicin, HPOB, LMK-235, Biphenyl-4-sulfonyl chloride (p-Phenylbenzenesulfonyl, 4-phenylbenzenesulfonyl, p-Biphenylsulfonyl), Nexturastat A, TH34, Tucidinostat (Chidamide, HBI-8000, CS-055), (−)-Parthenolide, WT161, CAY10603, CAY10603, ACY-738, RaddeaninA, Tinostamustine (EDO-S101), Domatinostat (4SC-202), and BG45.

In some embodiments, the HDACi is β-Hydroxybutyrate. β-Hydroxybutyric acid is a ketone body that, along with butyric acid, is an agonist of hydroxycarboxylic acid receptor 2 (HCA2), a Gi/o-coupled GPCR. In some embodiments, an HDACi inhibitor is an agonist of hydroxycarboxylic acid receptor 2.

In some embodiments, a medium described herein does not comprise an HDACi (e.g., does not include β-Hydroxybutyrate).

### Redox Homeostasis Regulator

In some embodiments, a composition (e.g., medium) of the disclosure comprises a redox homeostasis regulator.

Exemplary redox homeostasis regulators include, but are not limited to taurine, respiratory chain regulators, free radical scavengers, regulators of mitochondrial protein synthesis, allium sulphur compounds, anthocyanins, beta-carotene, catechins, copper, cryptoxanthins, flavonoids, indoles, isoflavonoids, lignans, lutein, lycopene, alpha lipoic acid, ellagic acid, manganese, polyphenols, selenium, glutathione, vitamin A, vitamin C, vitamin E, zinc, superoxide disutases, GSHPx, Prx-I, catalase, and co-enzyme Q10.

In some embodiments, the redox homeostasis regulator is taurine.

In some embodiments, a medium described herein does not comprise a redox homeostasis regulator.

Taurine is a non-proteinogenic β-aminosulfonic acid that can be derived from methionine and cysteine metabolism. In some embodiments, taurine can inhibit ROS generation within the respiratory chain.

In some embodiments, a medium described herein does not comprise a redox homeostasis regulator (e.g., does not include taurine).

### One Carbon Metabolism Pathway Intermediate

In some embodiments, a composition (e.g., medium) of the disclosure comprises a one carbon metabolism pathway intermediate. One-carbon metabolism mediated by folate cofactors, supports multiple physiological processes including amino acid homeostasis (methionine, glycine and serine), biosynthesis of nucleotides (purines, thymidine), epigenetic maintenance, and redox defense.

Exemplary one carbon metabolism pathway intermediates include, but are not limited to formate, tetrahydrofolate (THF), 10-formylTHF; 5,10-meTHF; 5,10-meTHF; and 10-formylTHF.

In some embodiments, a medium described herein does not comprise a one carbon metabolism pathway intermediate (e.g., does not include formate).

### Glutamine

In some embodiments, a composition (e.g., medium) of the disclosure comprises glutamine. Glutamine (Gln or Q) is an alpha-amino acid. Glutamine can be an essential amino acid within in vitro cell cultures. Glutamine supports the growth of cells, including cells that have high energy demands and synthesize large amounts of proteins and nucleic acids. It is an alternative energy source for rapidly dividing cells and cells that use glucose inefficiently.

In some embodiments, compositions and methods of the disclosure utilize glutamine in a form with increased bioavailability. Because of its chemical instability and importance for cell growth and function, it is important that delivery of L-glutamine be tailored to each unique cell culture process. Glutamine (e.g., L-glutamine) in a free form can be unstable at physiological pH in liquid media, breaking down to ammonium and pyroglutamate at rates that make it a problem in many cell culture and biomanufacturing applications. Therefore, many cell culture media contain stabilized forms of glutamine, including dipeptide forms, such as alanyl-l-glutamine and glycyl-1-glutamine. However, these more stable forms of L-glutamine can also have limited bioavailability, for example, due to a requirement for processing by enzymes, such as cell surface peptidases. Thus in some embodiments, compositions and methods of the disclosure utilize glutamine in a form with increased bioavailability, such as a free glutamine form, such as a non-dipeptide form, a non-alanine-glutamine dipeptide form (e.g., a non-alanyl-l-glutamine form), a non-glycine-glutamine dipeptide form (e.g., a non-glycyl-l-glutamine form), a form that in which glutamine is not conjugated to another amino acid or stabilizing moiety, a monomeric form, a free form, or a combination thereof. In some embodiments, glutamine is provided as a protein hydrolysate.

In some embodiments, a basal media contains glutamine. In some embodiments, glutamine in a form as disclosed herein is added to a media that already contains glutamine. In some embodiments, glutamine in a form as disclosed herein is added to a basal media that contains no glutamine or only low levels of glutamine to increase the bioavailability of glutamine.

In some embodiments, a medium described herein does not comprise glutamine.

### Glutamate

In some embodiments, a composition (e.g., medium) of the disclosure comprises glutamate (e.g., L-glutamate). Glutamate can be converted into, for example, g-amino butyric acid (GABA), ornithine, 2-oxoglutarate, glucose or glutathione. Glutamate and metabolites generated therefrom can contribute to, for example, redox homeostasis, cell signaling, nitrogen assimilation, amine catabolism, amino acid biosynthesis, nucleoside biosynthesis, and cofactor production.

In some embodiments, contacting cells with glutamate can improve production of SC-β cells in vitro, for example, providing higher cell yields and recoveries, increased numbers and relative percentages of SC-β cells, enhanced stability and shelf-life of SC-β cells, SC-islet clusters with advantageous characteristics such as reduced size and increased uniformity, improved function of the SC-β cells in vitro, improved cell viability, improved cell function, reduced immunogenicity after transplantation, or a combination thereof, e.g., relative to a composition that lacks glutamate, or contains a lower concentration of glutamate.

In some embodiments, a medium described herein does not comprise glutamate.

### Vitamins

In some embodiments, a composition (e.g., medium) of the disclosure comprises one or more vitamins.

Exemplary vitamins include, but are not limited to biotin, vitamin B1 (thiamine), vitamin B2 (riboflavin), vitamin B3 (niacin), vitamin B6 (pyridoxine) and vitamin B12 (cyanocobalamin). In some embodiments the vitamin modulates fatty acid synthesis. In some embodiments the vitamin modulates branched-chain amino acid metabolism. In some embodiments the vitamin modulates or participates as a co-factor in the TCA cycle, e.g., as a cofactor for pyruvate carboxylase. In some embodiments, the vitamin is biotin. In some embodiments, a composition of the disclosure contains two or more different vitamins, for example, 2, 3, 4, 5, 6, 7, 8, 9, 10, or more different vitamins.

In some embodiments, a medium described herein does not comprise a vitamin.

### Water-Soluble Synthetic Polymer

Water-soluble polymer described herein can refer to any polymer that has hydrophilic property and is soluble in aqueous solution at room temperature. The water-soluble polymer can be either naturally occurring or synthetic. In some embodiments, a water-soluble polymer is an albumin protein (e.g., human serum albumin or bovine serum albumin). In some embodiments, the water-soluble polymer is a water-soluble synthetic polymer. Water-soluble synthetic polymers described herein can refer to any synthetic polymer that has hydrophilic property and is soluble in aqueous solution at room temperature. Water-soluble synthetic polymers applicable in the subject methods and compositions include, but not limited to, poloxamer, polyvinyl alcohol, polyvinylpyrrolidone, polyethylene glycol (PEG), PEG copolymers, poly(Nisopropylacrylamide), and polyacrylamide. The water-soluble synthetic polymer can refer to a polymer compound or a mixture of polymer compounds that may have an idealized chemical formula but a variety of derivatives and/or precursors of the idealized formula, depending on the applicable manufacturing method. In some embodiments, the water-soluble synthetic polymer is used to replace at least partially serum or serum albumin, e.g., BSA or HSA, that is typically utilized in cell differentiation, e.g., differentiation of pancreatic β cells or precursor cells thereof. In some embodiments, the water-soluble synthetic polymer replaces 100% of serum albumin, e.g., BSA or HSA, that is typically utilized in cell differentiation, e.g., differentiation of pancreatic β cells or precursor cells thereof. In some embodiments, the water-soluble synthetic polymer reduces the amount of serum albumin, e.g., BSA or HSA, by at least 20%, 30%, 40%, 50%, 60%, 80%, 90%, 95%, or 99% of that is typically utilized in cell differentiation, e.g., differentiation of pancreatic β cells or precursor cells thereof. In some embodiments, the disclosure provides for a composition comprising a population of any of the cells disclosed herein (e.g., pluripotent stem cells; endoderm cells; primitive gut cells; PDX1-positive, NKX6.1-negative pancreatic progenitor cells; PDX1-positive, NKX6.1-positive pancreatic progenitor cells; insulin-positive cells; and/or pancreatic beta cells) and water soluble polymers, wherein at least 20%, 30%, 40%, 50%, 60%, 80%, 90%, 95%, or 99% of the water soluble polymers in the composition are water-soluble synthetic polymers (e.g., any of the PVA molecules disclosed herein) and wherein the remainder of the water soluble polymers are human serum albumin polypeptides. In some embodiments, the disclosure provides for a composition comprising a population of any of the cells disclosed herein (e.g., pluripotent stem cells; endoderm cells; primitive gut cells; PDX1-positive, NKX6.1-negative pancreatic progenitor cells; PDX1-positive, NKX6.1-positive pancreatic progenitor cells; insulin-positive cells; and/or pancreatic beta cells) and water soluble polymers, wherein no more than 1%, 2%, 3%, 4%, 5%, 6%, 7%, 8%, 9%, 10%, 15%, 20%, 30%, 40%, 50%, 60%, 80%, 90%, 95%, or 99% of the water soluble polymers are naturally occurring water-soluble polymers (e.g., HSA or BSA). In some embodiments, more than 90%, 95%, 99%, and up to 100% of the water-soluble polymers in the composition are water-soluble synthetic polymers (e.g., PVA).

In some embodiments, the water-soluble synthetic polymer applicable to the subject compositions and methods includes polyvinyl alcohol (PVA). Polyvinyl alcohol described herein can refer to a water-soluble synthetic polymer that has an idealized formula [CH2CH(OH)]n, which can be either partially or completed hydrolyzed. In some embodiments, the polyvinyl alcohol is manufactured by either partial or complete hydrolysis of polyvinyl acetate to remove acetate groups. In some embodiments, the polyvinyl alcohol is at most 85% hydrolyzed, e.g., 80% hydrolyzed. The percentage of hydrolyzation measures the approximate percentage (e.g., average percentage) of acetate residue that is hydrolyzed in the polyvinyl acetate precursor polymer. In some embodiments, the polyvinyl alcohol is at least 85% hydrolyzed, e.g., 87-89% hydrolyzed, 87-90% hydrolyzed, or 99% hydrolyzed. In some embodiments, the polyvinyl alcohol is 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% hydrolyzed. Without wishing to be bound by a certain theory, the polyvinyl alcohol can assume a function of carrier-molecule in the culture medium, which is typically carried out by serum or serum albumin, e.g., HSA. The percentage of hydrolyzation of polyvinyl alcohol can be determined by the manufacturing method utilized to produce the polyvinyl alcohol, e.g., how polyvinyl acetate precursor polymer is converted into polyvinyl alcohol, e.g., conversion by base-catalyzed transesterification with ethanol. In some embodiments, the water-soluble synthetic polymer preparation, e.g., polyvinyl alcohol, that is used in the subject method or present in the subject composition has purity of at least 90%, such as at least 92%, at least 95%, at least 98%, at least 99%, at least 99.5%, at least 99.9%, or nearly 100%. Purity of polyvinyl alcohol measures the percentage of synthetic polymer that has the idealized formula [CH2CH(OH)]n in the preparation, which includes polyvinyl alcohol of any percentage of hydrolyzation. Impurity of polyvinyl alcohol preparation can include other polymer materials that do not have the idealized formula [CH2CH(OH)]n, or other organic inorganic materials.

In some embodiments, a medium described herein does not comprise a water-soluble synthetic polymer.

Pharmaceutically Acceptable Excipients and/or Carriers

In some aspects, any of the compositions disclosed herein includes a population of genetically engineered cells in a liquid suspension. The liquid suspension can contain an aqueous solution that comprises pharmaceutically acceptable excipient(s) and/or carrier(s). For instance, the pharmaceutical compositions can further comprise a physiologically compatible solution including, for example, phosphate-buffered saline.

In some cases, the present disclosure provides compositions that can utilize genetically engineered cell populations and cell components and products in various methods for treatment of a disease (e.g., diabetes). Certain cases encompass pharmaceutical compositions comprising live cells (e.g., non-native pancreatic β cells alone or admixed with other cell types). Other cases encompass pharmaceutical compositions comprising non-native pancreatic β cell components (e.g., cell lysates, soluble cell fractions, conditioned medium, ECM, or components of any of the foregoing) or products (e.g., trophic and other biological factors produced by non-native pancreatic β cells or through genetic modification, conditioned medium from non-native pancreatic β cell culture). In either case, the pharmaceutical composition may further comprise other active agents, such as anti-inflammatory agents, exogenous small molecule agonists, exogenous small molecule antagonists, anti-apoptotic agents, antioxidants, and/or growth factors known to a person having skill in the art.

Pharmaceutical compositions of the present disclosure can comprise genetically engineered cells, or components or products thereof, formulated with a pharmaceutically acceptable carrier (e.g. a medium or an excipient). The term pharmaceutically acceptable carrier (or medium or excipient), which may be used interchangeably with the term biologically compatible carrier or medium, can refer to reagents, cells, compounds, materials, compositions, and/or dosage forms that are not only compatible with the cells and other agents to be administered therapeutically, but also are suitable for use in contact with the tissues of human beings and animals without excessive toxicity, irritation, allergic response, or other complication. Suitable pharmaceutically acceptable carriers can include water, salt solution (such as Ringer's solution), alcohols, oils, gelatins, and carbohydrates, such as lactose, amylose, or starch, fatty acid esters, hydroxymethylcellulose, and polyvinyl pyrolidine. Such preparations can be sterilized, and if desired, mixed with auxiliary agents such as lubricants, preservatives, stabilizers, wetting agents, emulsifiers, salts for influencing osmotic pressure, buffers, and coloring. Pharmaceutical compositions comprising cellular components or products, but not live cells, can be formulated as a liquid suspension.

Pharmaceutical compositions may comprise auxiliary components as would be familiar to a person having skill in the art. For example, they may contain antioxidants in ranges that vary depending on the kind of antioxidant used. Reasonable ranges for commonly used antioxidants are about 0.01% to about 0.15% weight by volume of EDTA, about 0.01% to about 2.0% weight volume of sodium sulfite, and about 0.01% to about 2.0% weight by volume of sodium metabisulfite. One skilled in the art may use a concentration of about 0.1% weight by volume for each of the above. Other representative compounds include mercaptopropionyl glycine, N-acetyl cysteine, beta-mercaptoethylamine, glutathione and similar species, although other anti-oxidant agents suitable for renal administration, e.g. ascorbic acid and its salts or sulfite or sodium metabisulfite may also be employed.

A buffering agent may be used to maintain the pH of formulations in the range of about 4.0 to about 8.0; so as to minimize irritation in the target tissue. For direct intraperitoneal injection, formulations should be at pH 7.2 to 7.5, preferably at pH 7.35-7.45. The compositions may also include tonicity agents suitable for administration to the kidney. Among those suitable is sodium chloride to make formulations approximately isotonic with blood.

In certain cases, pharmaceutical compositions are formulated with viscosity enhancing agents. Exemplary agents are hydroxyethylcellulose, hydroxypropylcellulose, methylcellulose, and polyvinylpyrrolidone. The pharmaceutical compositions may have cosolvents added if needed. Suitable cosolvents may include glycerin, polyethylene glycol (PEG), polysorbate, propylene glycol, and polyvinyl alcohol. Preservatives may also be included, e.g., benzalkonium chloride, benzethonium chloride, chlorobutanol, phenylmercuric acetate or nitrate, thimerosal, or methyl or propylparabens.

Pharmaceutical compositions comprising genetically engineered cells, cell components or cell products may be delivered to the liver of a patient in one or more of several methods of delivery known in the art. In some cases, the compositions are delivered to the liver. In another embodiment, the compositions may be delivered to the liver via intra-portal injection.

In some embodiments, any of the genetically engineered cell compositions disclosed herein (e.g., a composition comprising in vitro differentiated genetically engineered islet cells) further comprises a liquid solution (or a medium). In some embodiments, the liquid solution comprises a sugar. In some embodiments, the sugar is sucrose or glucose. In some embodiments, the liquid solution comprises the sugar at a concentration of between about 0.05% and about 1.5%. In some embodiments, the liquid solution is a CMRL medium. In some embodiments, the composition comprises HypoThermosol® FRS Preservation Media. In some cases, the liquid solution is serum free. In some cases, the liquid solution that the cells are suspended in is free of proteins. In some cases, the liquid solution that the cells are suspended in is free of animal components. In some cases, the liquid solution that the cells are suspended in is free of proteins and animal components. In some cases, the liquid solution has physiological osmolality. In some cases, the liquid solution is pH buffered.

In some instances, pharmaceutical compositions of the genetically engineered cells are formulated in a conventional manner using one or more physiologically acceptable carriers including excipients and auxiliaries which facilitate processing of the active compounds into preparations which can be used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N. Y., 1980; and Pharmaceutical Dosage Forms and Drug Delivery Systems, Seventh Ed. (Lippincott Williams & Wilkins 1999).

Pharmaceutical compositions are optionally manufactured in a conventional manner, such as, by way of example only, by means of conventional mixing, dissolving, granulating, dragee-making, levigating, emulsifying, encapsulating, entrapping or compression processes.

In certain embodiments, compositions may also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In other embodiments, compositions can also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The pharmaceutical compositions described herein are administered by any suitable administration route, including but not limited to, oral, parenteral (e.g., intravenous, subcutaneous, intramuscular, intracerebral, intracerebroventricular, intra-articular, intraperitoneal, or intracranial), intranasal, buccal, sublingual, or rectal administration routes. In some instances, the pharmaceutical composition is formulated for parenteral (e.g., intravenous, subcutaneous, intramuscular, intracerebral, intracerebroventricular, intra-articular, intraperitoneal, or intracranial) administration. In some embodiments, any of the pharmaceutical compositions disclosed herein is administered to a subject by infusion via the hepatic portal vein.

The pharmaceutical compositions described herein are formulated into any suitable dosage form, including but not limited to, aqueous oral dispersions, liquids, gels, syrups, elixirs, slurries, suspensions and the like, for oral ingestion by an individual to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations. In some embodiments, the pharmaceutical compositions are formulated into capsules. In some embodiments, the pharmaceutical compositions are formulated into solutions (for example, for IV administration). In some cases, the pharmaceutical composition is formulated as an infusion. In some cases, the pharmaceutical composition is formulated as an injection.

The pharmaceutical solid dosage forms described herein optionally include a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

In still other aspects, using standard coating procedures, such as those described in Remington's Pharmaceutical Sciences, 20th Edition (2000), a film coating is provided around the compositions. In some embodiments, the compositions are formulated into particles (for example for administration by capsule) and some or all of the particles are coated. In some embodiments, the compositions are formulated into particles (for example for administration by capsule) and some or all of the particles are microencapsulated. In some embodiments, the compositions are formulated into particles (for example for administration by capsule) and some or all of the particles are not microencapsulated and are uncoated.

In certain embodiments, compositions provided herein may also include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

In some embodiments, a composition of the present disclosure can comprise the genetically engineered stem cell derived islet cells, in an amount that is effective to treat or prevent e.g., diabetes. A pharmaceutical composition can comprise the genetically engineered stem cell derived islet cells as described herein, in combination with one or more pharmaceutically or physiologically acceptable carriers, diluents or excipients. Such compositions can comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Pharmaceutical compositions can comprise auxiliary components as would be familiar to a person having skill in the art. For example, they can contain antioxidants in ranges that vary depending on the kind of antioxidant used. Reasonable ranges for commonly used antioxidants are about 0.01% to about 0.15% weight by volume of EDTA, about 0.01% to about 2.0% weight volume of sodium sulfite, and about 0.01% to about 2.0% weight by volume of sodium metabisulfite. One skilled in the art may use a concentration of about 0.1% weight by volume for each of the above. Other representative compounds include mercaptopropionyl glycine, N-acetyl cysteine, β-mercaptoethylamine, glutathione and similar species, although other anti-oxidant agents suitable for renal administration, e.g. ascorbic acid and its salts or sulfite or sodium metabisulfite may also be employed.

A buffering agent can be used to maintain the pH of formulations in the range of about 4.0 to about 8.0; as to minimize irritation in the target tissue. For direct intraperitoneal injection, formulations should be at pH 7.2 to 7.5, preferably at pH 7.35-7.45. The compositions may also include tonicity agents suitable for administration to the kidney. Among those suitable is sodium chloride to make formulations approximately isotonic with blood.

In certain cases, pharmaceutical compositions are formulated with viscosity enhancing agents. Exemplary agents are hydroxyethylcellulose, hydroxypropylcellulose, methylcellulose, and polyvinylpyrrolidone. The pharmaceutical compositions may have cosolvents added if needed. Suitable cosolvents may include glycerin, polyethylene glycol (PEG), polysorbate, propylene glycol, and polyvinyl alcohol. Preservatives may also be included, e.g., benzalkonium chloride, benzethonium chloride, chlorobutanol, phenylmercuric acetate or nitrate, thimerosal, or methyl or propylparabens.

Formulations described herein may benefit from antioxidants, metal chelating agents, thiol containing compounds and other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (c) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

A “carrier” or “carrier materials” include any commonly used excipients in pharmaceutics and should be selected on the basis of compatibility with compounds disclosed herein, such as, compounds of ibrutinib and an anticancer agent, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” may include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

“Dispersing agents,” and/or “viscosity modulating agents” include materials that control the diffusion and homogeneity of a drug through liquid media or a granulation method or blend method. In some embodiments, these agents also facilitate the effectiveness of a coating or eroding matrix. Exemplary diffusion facilitators/dispersing agents include, e.g., hydrophilic polymers, electrolytes, Tween® 60 or 80, PEG, polyvinylpyrrolidone (PVP; commercially known as Plasdone®), and the carbohydrate-based dispersing agents such as, for example, hydroxypropyl celluloses (e.g., HPC, HPC-SL, and HPC-L), hydroxypropyl methylcelluloses (e.g., HPMC K100, HPMC K4M, HPMC K15M, and HPMC K100M), carboxymethylcellulose sodium, methylcellulose, hydroxyethylcellulose, hydroxypropylcellulose, hydroxypropylmethylcellulose phthalate, hydroxypropylmethylcellulose acetate stearate (HPMCAS), noncrystalline cellulose, magnesium aluminum silicate, triethanolamine, polyvinyl alcohol (PVA), vinyl pyrrolidone/vinyl acetate copolymer (S630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 908®, also known as Poloxamine 908®, which is a tetrafunctional block copolymer derived from sequential addition of propylene oxide and ethylene oxide to ethylenediamine (BASF Corporation, Parsippany, N.J.)), polyvinylpyrrolidone K12, polyvinylpyrrolidone K17, polyvinylpyrrolidone K25, or polyvinylpyrrolidone K30, polyvinylpyrrolidone/vinyl acetate copolymer (S-630), polyethylene glycol, e.g., the polyethylene glycol can have a molecular weight of about 300 to about 6000, or about 3350 to about 4000, or about 7000 to about 5400, sodium carboxymethylcellulose, methylcellulose, polysorbate-80, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, polysorbate-80, sodium alginate, polyethoxylated sorbitan monolaurate, polyethoxylated sorbitan monolaurate, povidone, carbomers, polyvinyl alcohol (PVA), alginates, chitosans and combinations thereof. Plasticizers such as cellulose or triethyl cellulose can also be used as dispersing agents. Dispersing agents particularly useful in liposomal dispersions and self-emulsifying dispersions are dimyristoyl phosphatidyl choline, natural phosphatidyl choline from eggs, natural phosphatidyl glycerol from eggs, cholesterol and isopropyl myristate.

Combinations of one or more erosion facilitator with one or more diffusion facilitator can also be used in the present compositions.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution. In certain embodiments, diluents increase bulk of the composition to facilitate compression or create sufficient bulk for homogenous blend for capsule filling. Such compounds include e.g., lactose, starch, mannitol, sorbitol, dextrose, microcrystalline cellulose such as Avicel®; dibasic calcium phosphate, dicalcium phosphate dihydrate; tricalcium phosphate, calcium phosphate; anhydrous lactose, spray-dried lactose; pregelatinized starch, compressible sugar, such as Di-Pac® (Amstar); mannitol, hydroxypropylmethylcellulose, hydroxypropylmethylcellulose acetate stearate, sucrose-based diluents, confectioner's sugar; monobasic calcium sulfate monohydrate, calcium sulfate dihydrate; calcium lactate trihydrate, dextrates; hydrolyzed cereal solids, amylose; powdered cellulose, calcium carbonate; glycine, kaolin; mannitol, sodium chloride; inositol, bentonite, and the like.

“Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Lubricants” and “glidants” are compounds that prevent, reduce or inhibit adhesion or friction of materials. Exemplary lubricants include, e.g., stearic acid, calcium hydroxide, talc, sodium stearyl fumerate, a hydrocarbon such as mineral oil, or hydrogenated vegetable oil such as hydrogenated soybean oil (Sterotex®), higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol (e.g., PEG-4000) or a methoxypolyethylene glycol such as Carbowax™, sodium oleate, sodium benzoate, glyceryl behenate, polyethylene glycol, magnesium or sodium lauryl sulfate, colloidal silica such as Syloid™, Cab-O-Sil®, a starch such as corn starch, silicone oil, a surfactant, and the like.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. In some embodiments, plasticizers can also function as dispersing agents or wetting agents.

“Solubilizers” include compounds such as triacetin, triethylcitrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

“Stabilizers” include compounds such as any antioxidation agents, buffers, acids, preservatives and the like.

“Suspending agents” include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

“Surfactants” include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF), and the like. Some other surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40. In some embodiments, surfactants may be included to enhance physical stability or for other purposes.

“Viscosity enhancing agents” include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

In some embodiments, any of the compositions disclosed herein are encapsulated in any of the devices disclosed herein.

### Improved Survivability

In some embodiments, any one or more of the cells disclosed herein is genetically modified to improve survivability of the cell. In some embodiments, the cell is genetically modified such that the cell has improved survivability following implantation into a subject (e.g., a human) as compared to a cell that has not been genetically modified. In some embodiments, the cell is genetically modified such that it triggers less (e.g., at least 5%, 10%, 15%, 20%, 25%, 30%, 40%, 50%, 60%, 70%, 80%, or 90% less) immediate blood mediated inflammatory reaction (IBMIR) following implantation in a subject as compared to a cell that is not genetically modified. In some embodiments, the cell is genetically modified such that it triggers a lower (e.g., at least 5%, 10%, 15%, 20%, 25%, 30%, 40%, 50%, 60%, 70%, 80%, or 90% less) antibody response (e.g., lower antibody generation or release) following implantation in a subject as compared to a cell that is not genetically modified. In some embodiments, the cell is genetically modified such that it recruits fewer (e.g., at least 5%, 10%, 15%, 20%, 25%, 30%, 40%, 50%, 60%, 70%, 80%, or 90% less) T cells following implantation in a subject as compared to a cell that is not genetically modified. In some embodiments, the cell is genetically modified such that it is less likely (e.g., at least 5%, 10%, 15%, 20%, 25%, 30%, 40%, 50%, 60%, 70%, 80%, or 90% less) to die following implantation in a subject as compared to a cell that is not genetically modified. In some embodiments, the disclosure provides for a population of genetically modified cells, wherein at least 50%, 55%, 60%, 65%, 70%, 75%, 80%, 85%, 90% or 95% of the cells in the population of genetically modified cells survive at least 1 hour, 5 hours, 12 hours, 24 hours, 2 days, 10 days, 15 days, 30 days, 60 days or 90 days following implantation into a subject.

### Genes of Interest

As described, some aspects of the current disclosure are directed to mammalian cells genetically modified to increase or decrease the expression of one or more genes of interest. In some embodiments, “genes of interest” include ABO, renalase, CXCL10, B2M, tissue factor (F3) and CD47.

The ABO gene determines the blood group antigen. In some embodiments, a cell (e.g., an isolated stem cell or a NKX6.1-positive, ISL1-positive cell) described herein is negative for A antigen and negative for B antigen. In some embodiments, the cell described herein is negative for A antigen. In some embodiments, the cell described herein is negative for B antigen. In some embodiments, a cell (e.g., an isolated stem cell or a NKX6.1-positive, ISL1-positive cell) described herein is negative for Rh antigen. In some embodiments, a cell (e.g., an isolated stem cell or a NKX6.1-positive, ISL1-positive cell) described herein is negative for A antigen, negative for B antigen, and negative for Rh antigen. An “A antigen,” as used herein, refers to a histo-blood group antigen produced by 3α-N-acetylgalactosaminyltransferase and expressed as a cell-surface antigen. A “B antigen,” as used herein, refers to a histo-blood group antigen produced by 3α-galactosaminyltransferase and expressed as a cell-surface antigen. In some embodiments, the cell comprises a disruption in the ABO gene. In some embodiments, the cell comprises a disruption in the ABO gene such that the cell has reduced or absent levels of A and B antigens. In the absence of the ABO gene, the blood group is designated O and has been referred to as the universal donor blood group due to the lack of blood group antigen.

In particular embodiments, the ABO gene is the human ABO gene identified as NCBI Gene No. 28. The human ABO Genbank cDNA Accession No. is NM_020469 and is set out as SEQ ID NO: 1. The human ABO Genbank protein Accession No. is NP_065202 and is set out as SEQ ID NO: 2.

In some embodiments, the ABO gene is disrupted using a CRISPR/Cas system. In some embodiments, the CRISPR/Cas system utilizes Cas9 (e.g., SpCas9) and one or more guide RNAs, e.g., one or more single guide (sgRNAs). In some embodiments, the one or more sgRNAs target an ABO gene and comprise the nucleotide sequence of any one of the sequences in Table 2. In some embodiments, the one or more sgRNAs target an ABO gene and comprise the nucleotide sequence of any one of SEQ ID NOs: 59-89.

TABLE 2

| Guide | Sequence | SEQ ID NO: |
|---|---|---|
| C9_ABO_ex2_gRNA005 | GGATCATAGGTCGAAGTGCG | 59 |
| C9_ABO_ex5_gRNA008 | AGCCAAAGGTGCTGACACCG | 60 |
| C9_ABO_ex1_gRNA004 | GGCCAGCGTCCGCAACACCT | 61 |
| C9_ABO_ex1_gRNA023 | GAGGTGTTGCGGACGCTGGC | 62 |
| C9_ABO_ex3_gRNA021 | ACTTCCTTTCTGCAGTTACG | 63 |
| C9_ABO_ex4_gRNA005 | GAGACGCGCTGCAGATGGTC | 64 |
| C9_ABO_ex3_gRNA003 | GCTTCCTGGCATTAGACTTC | 65 |
| C9_ABO_ex5_gRNA004 | CGGTGTCAGCACCTTTGGCT | 66 |
| C9_ABO_ex5_gRNA006 | GTGTCAGCACCTTTGGCTGG | 67 |
| C9_ABO_ex6_gRNA006 | GGAGCCTGAACTGCTCGTTG | 68 |
| C9_ABO_ex3_gRNA002 | AACCCCCGTTCCAGGCTTCC | 69 |
| C9_ABO_ex1_gRNA026 | CAGACGCGGAGCCATGGCCG | 70 |
| C9_ABO_ex6_gRNA032 | CAGTAGGAAGGATGTCCTCG | 71 |
| C9_ABO_ex6_gRNA025 | ACATCCTCAACGAGCAGTTC | 72 |
| C9_ABO_ex5_gRNA005 | GGTGTCAGCACCTTTGGCTG | 73 |
| C9_ABO_ex1_gRNA005 | CGTCCGCAACACCTCGGCCA | 74 |
| C9_ABO_ex6_gRNA008 | AATGTGCCCTCCCAGACAAT | 75 |
| C9_ABO_ex4_gRNA009 | TGCAGCGCGTCTCGTTGCCA | 76 |
| C9_ABO_ex3_gRNA020 | AGCCCCAGAAGTCTAATGCC | 77 |
| C9_ABO_ex6_gRNA030 | TCCTCGTGGTACCCCTTGGC | 78 |
| C9_ABO_ex5_gRNA003 | ACGGTGTCAGCACCTTTGGC | 79 |
| C9_ABO_ex3_gRNA006 | GCATTAGACTTCTGGGGCTT | 80 |
| C9_ABO_ex1_gRNA024 | GGCCGAGGTGTTGCGGACGC | 81 |
| C9_ABO_ex5_gRNA002 | CACCACGGTGTCAGCACCTT | 82 |
| C9_ABO_ex6_gRNA005 | TTAACCCAATGGTGGTGTTC | 83 |
| C9_ABO_ex3_gRNA019 | AAGTCTAATGCCAGGAAGCC | 84 |
| C9_ABO_ex4_gRNA004 | GCAACGAGACGCGCTGCAGA | 85 |
| C9_ABO_ex2_gRNA006 | GGTCGAAGTGCGTGGCATTT | 86 |
| C9_ABO_ex5_gRNA009 | GATGGTCTACCCCCAGCCAA | 87 |
| C9_ABO_ex6_gRNA012 | GCCAGCCAAGGGGTACCACG | 88 |
| C9_ABO_ex6_gRNA031 | GATGTCCTCGTGGTACCCCT | 89 |

When SC-islets are transplanted into individuals with type 1 diabetes caused by beta cell autoimmunity, it is possible that the islets could be detected and destroyed by multiple effector arms of the immune system. Such effector arms include CD4+ T cells that recognize autoantigens presented by the recipient's own immune cells with no requirement of HLA expression on transplanted cells. Renalase is also designated monoamine oxidase C (MAO-C). In some embodiments, deletion of RNLS protects transplanted beta cells.

Renalase has been described as a flavin adenine dinucleotide-containing monoamine oxidase with an activity that selectively deaminates the catecholamines epinephrine, norepinephrine and dopamine. The renalase gene contains 9 exons spanning 310188 bp in chromosome 10 of human genome. As used herein, the term “renalase” also includes renalase isoforms. The renalase gene contains 9 exons spanning 310,188 bp in chromosome 10 of the human genome. In some embodiments, the renalase is a human renalase identified as Gene ID 55328.

The renalase clone (SEQ ID NO:35, GenBank accession number: BC005364) is a gene containing exons 1, 2, 3, 4, 5, 6, 8. There are at least two alternatively-spliced forms of renalase protein as shown in the human genome database. One alternatively spliced form of human renalase contains exons 1, 2, 3, 4, 5, 6, 9, identified by clones in the human genome database as GenBank accession number AK002080 (SEQ ID NO: 36) and NM_018363 (SEQ ID NO: 5). Other alternatively spliced form contains exons 5, 6, 7, 8, identified by clones in the human genome database as GenBank accession number. In summary, there is one renalase gene with at least 4 different transcripts (i.e., 948 bp, 1447 bp, 1029 bp, and 3385 bp) encoding 2 different renalase isoforms (342 aa and 315 aa). One transcript variant has 2409 bp and is identified with Accession No. NM_001031709 and is shown in SEQ ID NO: 3. This transcript variant encodes the 342 aa length isoform has Accession No. NP_001026879 and is shown in SEQ ID NO: 4. The 315 aa length isoform is encoded by the 2175 bp mRNA with Accession No. NM_018363 shown in SEQ ID NO: 5. The transcribed protein has Accession No. NP_060833 and is shown in SEQ ID NO: 6. Unless otherwise indicated, “renalase” encompasses all known mammalian renalases and renalases to be discovered, including but not limited to, mouse renalase and chimpanzee renalase, having the characteristics and/or physical features of the renalase disclosed herein.

In some embodiments, the renalase gene is disrupted using a CRISPR/Cas system. In some embodiments, the CRISPR/Cas system utilizes Cas9 (e.g., SpCas9) and one or more guide RNAs, e.g., one or more single guide (sgRNAs). In some embodiments, the one or more sgRNAs target the renalase gene and comprise the nucleotide sequence of any one of the sequences in Table 3. In some embodiments, the one or more sgRNAs target the renalase gene and comprise the nucleotide sequence of any one of the sequences of SEQ ID Nos: 164-195.

TABLE 3

| Sequence | SEQ ID NO |
|---|---|
| TGTGCGCTGCGCTGCTGAGG | 164 |
| GCTTGTGCGCTGCGCTGCTG | 165 |
| ATCGTGGGCGCCGGGATGAC | 166 |
| AGGTGCTGATCGTGGGCGCC | 167 |
| ATGGCGCAGGTGCTGATCGT | 168 |
| CATGGCGCAGGTGCTGATCG | 169 |
| GCUCCCUCUCGCCAUGGCGC | 170 |
| CACGATCAGCACCTGCGCCA | 171 |
| GATAATAAGAAGCGCAATAT | 172 |
| AGATTGATGTCCCTTGGGCT | 173 |
| AAGATTGATGTCCCTTGGGC | 174 |
| GTGATGTACTGCCCAGCCCA | 175 |
| TCGTACCAGCTTCATAAAAG | 176 |
| UCGUACCAGCUUCAUAAAAG | 177 |
| GAGGCCCAGAGCATATCGAG | 178 |
| CAGCCUCCAGUUGCUGCCUU | 179 |
| GATAATAAGAAGCGCAATAT | 180 |
| AGAUUGAUGUCCCUUGGGCU | 181 |
| AAGAUUGAUGUCCCUUGGGC | 182 |
| TACGAAGATTGATGTCCCTT | 183 |
| GTACGAAGATTGATGTCCCT | 184 |
| CUGGGCCUCUUUUAUGAAGC | 185 |
| TACTCCTCTCGATATGCTCT | 186 |
| CTACTCCTCTCGATATGCTC | 187 |
| AUGCCAAAGGCAGCAACUGG | 188 |
| UGAAUGCCAAAGGCAGCAAC | 189 |
| CAGUAAUUAGUGAAUGCCAA | 190 |
| ATTGCGCTTCTTATTATCAA | 191 |
| AAUGGAGACGAAGCGUAUGC | 192 |
| ATGGAGACGAAGCGTATGCA | 193 |
| TGAATGCCAAAGGCAGCAAC | 194 |
| UGAUGUACUGCCCAGCCCAA | 195 |

Chemokines play a key role in the recruitment of immune cells to sites of inflammation. In some embodiments, following transplantation, SC-islets may experience cellular stress caused by multiple factors that include hypoxia and inflammation. C—X—C motif chemokine ligand 10 (CXCL10) is a key driver of T cell recruitment in autoimmune diabetes and in islet transplantation. The human CXCL10 Genbank cDNA accession no is NM_001565 and shown in SEQ ID NO: 7. The human CXCL10 Genbank protein Accession No. is NP_001556 and shown in SEQ ID NO: 8.

In some embodiments, the CXCL10 gene is disrupted using a CRISPR/Cas system. In some embodiments, the CRISPR/Cas system utilizes Cas9 (e.g., SpCas9) and one or more guide RNAs, e.g., one or more single guide (sgRNAs). In some embodiments, the one or more sgRNAs target the CXCL10 gene and comprise the nucleotide sequence of any one of the sequences in Table 4. In some embodiments, the one or more sgRNAs target the CXCL10 gene and comprise the nucleotide sequence of any one of the sequences of SEQ ID Nos: 155-161 and 196-204.

TABLE 4

| Sequence | SEQ ID NO |
|---|---|
| GTAATCAACCTGTTAATCCA | 155 |
| CAGCGTACAGTTCTAGAGAG | 156 |
| TTGATTACTAATGCTGATGC | 157 |
| CTGACTCTAAGTGGCATTCA | 158 |
| CAGAATGGCAGTTTGATTCA | 159 |
| TCTAAAGACCTTGGATTAAC | 160 |
| GATAAGGCAGCAAATCAGAA | 161 |
| CTTATCTTTCTGACTCTAAG | 196 |
| ACTCACATGATCTCAACAGC | 197 |
| TCTCAACACGTGGACAAAAT | 198 |
| TTCTTGATGGCCTTCGATTC | 199 |
| TGCTTTCAGTAAATTCTTGA | 200 |
| AGCAGTTAGCAAGGAAAGGT | 201 |
| TGAAAGCAGTTAGCAAGGAA | 202 |
| ATGTCTGAATCCAGAATCGA | 203 |
| CGTGGACAAAATTGGCTTGC | 204 |

In some embodiments, a barrier to transplantation is the disparity between donor and recipient HLA molecules. These include class I HLA-A/B/C, and class II HLA DP/DQ/DR. HLA-disparity may cause acute T cell-dependent graft rejection. HLA mismatch can also elicit a damaging antibody response. The expression of HLA class I molecules is strictly dependent on its non-polymorphic component, the beta 2 microglobulin chain encoded by the B2M gene. HLA class II molecules are only expressed on select cell populations and have not been convincingly detected on SC-islets. The β chain of MHC class I molecules consists of beta-2 microglobulin, which is encoded by the non-polymorphic beta-2 microglobulin (B2M) gene on chromosome 15. Beta-2 microglobulin is non-covalently linked to α3 subunit and is common to all MHC class I molecules. Furthermore, expression of MHC class I molecules at the cell surface requires its association with beta-2 microglobulin. The human B2M gene is identified by NCBI Gene ID NO. 567 (Accession No. NM_004048), which is set forth in SEQ ID NO: 9. The human NCBI protein Accession No. is NP_004039 and is set forth in SEQ ID NO: 10.

In some embodiments, the beta-2 microglobulin gene is disrupted using a CRISPR/Cas system. In some embodiments, the CRISPR/Cas system utilizes Cas9 (e.g., SpCas9) and one or more guide RNAs, e.g., one or more single guide (sgRNAs). In some embodiments, the one or more sgRNAs target the beta-2 microglobulin gene and comprise the nucleotide sequence of any one of the sequences in Table 5. In some embodiments, the one or more sgRNAs target the beta-2 microglobulin gene and comprise the nucleotide sequence of any one of the sequences of SEQ ID Nos: 117-127 and 205-225.

TABLE 5

| Sequence | SEQ ID NO |
|---|---|
| AGTCACATGGTTCACACGGC | 117 |
| CGTGAGTAAACCTGAATCTT | 118 |
| CAGTAAGTCAACTTCAATGT | 119 |
| TCACGTCATCCAGCAGAGAA | 120 |
| CACAGCCCAAGATAGTTAAG | 121 |
| CAGCCCAAGATAGTTAAGTG | 122 |
| ACAGCCCAAGATAGTTAAGT | 123 |
| CTTACCCCACTTAACTATCT | 124 |
| TCCTGAATTGCTATGTGTCT | 125 |
| CATACTCATCTTTTTCAGTG | 126 |
| ACAAAGTCACATGGTTCACA | 127 |
| ACTCTCTCTTTCTGGCCTGG | 205 |
| GCCCGAATGCTGTCAGCTTC | 206 |
| GGCCACGGAGCGAGACATCT | 207 |
| CGCGAGCACAGCTAAGGCCA | 208 |
| GAGTAGCGCGAGCACAGCTA | 209 |
| ACTCACGCTGGATAGCCTCC | 210 |
| GGCCGAGATGTCTCGCTCCG | 211 |
| CTCGCGCTACTCTCTCTTTC | 212 |
| GCTACTCTCTCTTTCTGGCC | 213 |
| TTTGACTTTCCATTCTCTGC | 214 |
| ACCCAGACACATAGCAATTC | 215 |
| AAGTCAACTTCAATGTCGGA | 216 |
| ATACTCATCTTTTTCAGTGG | 217 |
| GCATACTCATCTTTTTCAGT | 218 |
| GGCATACTCATCTTTTTCAG | 219 |
| TGGGCTGTGACAAAGTCACA | 220 |
| TTACCCCACTTAACTATCTT | 221 |
| TTCCTGAATTGCTATGTGTC | 222 |
| GAAGTTGACTTACTGAAGAA | 223 |
| TTCAGACTTGTCTTTCAGCA | 224 |
| ACTTGTCTTTCAGCAAGGAC | 225 |

The F3 gene encodes a surface molecule on transplanted cells known as tissue factor (aka factor III). Factor III may trigger the immediate blood mediated inflammatory reaction (IBMIR) which could cause the loss of islet cells within minutes post infusion into the liver. IBMIR is characterized by coagulation and complement activation. Factor III, (also called CD142, coagulation factor III, or tissue factor) may enable cells to initiate the blood coagulation cascades, and it may function as the high-affinity receptor for the coagulation factor VII. The resulting complex may provide a catalytic event that is responsible for initiation of the coagulation protease cascades by specific limited proteolysis. Unlike the other cofactors of these protease cascades, which circulate as nonfunctional precursors, this factor may be a potent initiator that is fully functional when expressed on cell surfaces, for example, on monocytes. There are 3 distinct domains of this factor: extracellular, transmembrane, and cytoplasmic. In some embodiments, F3 is human F3 identified by NCBI gene ID 2152 and human F3 GenBank cDNA accession no is NM_001993 and disclosed in SEQ ID NO: 11. The corresponding human F3 GenBank protein Accession No. is NP_001984.1 and disclosed in SEQ ID NO: 12.

In some embodiments, the tissue factor gene is disrupted using a CRISPR/Cas system. In some embodiments, the CRISPR/Cas system utilizes Cas9 (e.g., SpCas9) and one or more guide RNAs, e.g., one or more single guide (sgRNAs). In some embodiments, the one or more sgRNAs target the tissue factor gene and comprise the nucleotide sequence of any one of the sequences in Table 6. In some embodiments, the one or more sgRNAs target the tissue factor gene and comprise the nucleotide sequence of any one of the sequences of SEQ ID Nos: 128-142 and 226-241.

TABLE 6

| Sequence | SEQ ID NO |
|---|---|
| GTCGCTCGGACGCTCCTGCT | 128 |
| CCTTCACAATCTCGTCGGTG | 129 |
| CCTCACCGACGAGATTGTGA | 130 |
| GGCACGGGTCTTCTCCTACC | 131 |
| GGATGTGAAGCAGACGTACT | 132 |
| TCGGACGCTCCTGCTCGGCT | 133 |
| GCCCAGGTGGCCGGCGCTTC | 134 |
| CACATCCTTCACAATCTCGT | 135 |
| GAAGCAGACGTACTTGGCAC | 136 |
| GGGCGAAGACCCAGCCGAGC | 137 |
| ACCTGAAGCGCCGGCCACCT | 138 |
| GTGCCACTCACCTGAAGCGC | 139 |
| GCTCGGCTGGGTCTTCGCCC | 140 |
| TAAAGGCACTACAAATACTG | 141 |
| TAGACTTGATTGACGGGTTT | 142 |
| GAGCAGGAGCGTCCGAGCGA | 226 |
| CAGGAGCGTCCGAGCGACGG | 227 |
| CGTCCGAGCGACGGCGGTCT | 228 |
| GTCCGAGCGACGGCGGTCTC | 229 |
| TCCGAGCGACGGCGGTCTCG | 230 |
| TGGGTCTTCGCCCAGGTGGC | 231 |
| CTCGGACGCTCCTGCTCGGC | 232 |
| GCCCCGAGACCGCCGTCGCT | 233 |
| AACAGTGTAGACTTGATTGA | 234 |
| ACAGTGTAGACTTGATTGAC | 235 |
| GTAGACTTGATTGACGGGTT | 236 |
| AAGTCTACACTGTTCAAATA | 237 |
| TGGCAGCATATAATTTAACT | 238 |
| CTCCTACCCGGCAGGGAATG | 239 |
| CGGGTCTTCTCCTACCCGGC | 240 |
| TAGACATGGAGACCCCTGCC | 241 |

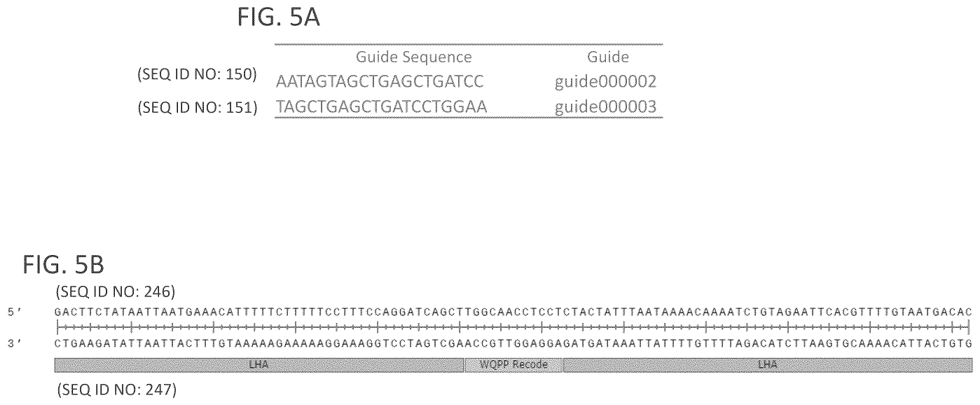

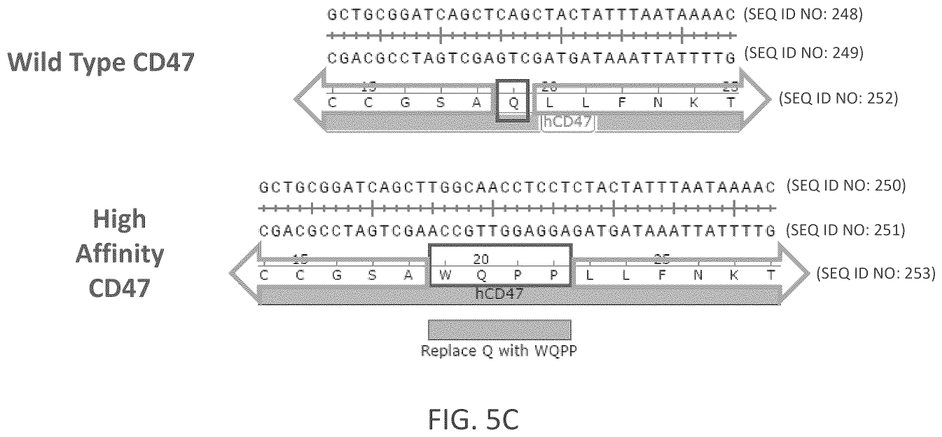

In addition to an antibody and T cell recognition of mismatched HLA, innate immune cells, including macrophages, dendritic cells and natural killer cells, may be able to recognize allogenic (donor) cells through a variety of receptors. Activated innate immune cells can destroy transplanted cells by phagocytosis, cytotoxicity and secretion of inflammatory cytokines. In some embodiments, activation signals can be mitigated by inhibitory receptors that are also present on innate immune cells. In some embodiments, CD47 gene modification increases inhibitory CD47/SIRPα signaling, thereby mitigating activation signals of innate immune cells. In some embodiments, the CD47 is the human CD47 gene identified by gene ID 961 and human CD47 GenBank cDNA accession number is NM_001777.4 and set forth in SEQ ID NO: 13. The human CD47 GenBank protein Accession No. is NP_001768.1 and is set forth in SEQ ID NO: 14. In some embodiments, the CD47 protein comprises the amino acid sequence of SEQ ID NO: 145. In some embodiments, the CD47 protein comprises the amino acid sequence of SEQ ID NO: 146. As with other genes of interest, there are variants listed on NCBI including human CD47 transcript variant X₅ (GenBank Accession No. XM_005247909) set forth in SEQ ID NO: 15 and the translated human CD47 protein isoform X₁ (GenBank Accession No. XP_005247966) set forth in SEQ ID NO: 16. In some embodiments, the CD47 protein comprises at least 3 amino acids added to the N-terminus of the mature CD47 protein wherein the 3 amino acids added has the formula X₃—X₂—X₁, where X₃ is W; X₂ is selected from Q, A and G; and X₁ is selected from R, P, L, T, F, I, and M. In some embodiments, the at least 3 amino acids added to the N-terminus of the mature CD47 protein are added by editing the CD47 gene such that the added amino acids are added between the CD47 leader sequence (e.g., the amino acid sequence of SEQ ID NO: 244) and the start of the mature CD47 amino acid sequence (e.g., an amino acid sequence that is at least 85%, 90%, 91%, 92%, 93%, 94%, 95%, 96%, 97%, 98%, 99%, or 100% identical to SEQ ID NO: 145 or 146) (see, e.g., FIG. 5C). In some embodiments, the at least three amino acids are added to the N-terminus of the mature CD47 protein by replacing the N-terminal Q of the mature CD47 protein (e.g., a mature CD47 protein having an amino acid sequence that is at least 85%, 90%, 91%, 92%, 93%, 94%, 95%, 96%, 97%, 98%, 99%, or 100% identical to SEQ ID NO: 145 or 146) with at least three amino acids. In some embodiments, the at least 3 amino acids added to the N-terminus of the mature CD47 protein are added by replacing the “Q” at the position corresponding to position 19 of an amino acid sequence that is at least 85%, 90%, 91%, 92%, 93%, 94%, 95%, 96%, 97%, 98%, 99%, or 100% identical to SEQ ID NO: 14 or 243 with at least three amino acids. See, e.g., FIG. 5C. In some embodiments, the at least three amino acids are selected from WQR, WAP, WQL, WQP, WQPP, WQT, WQF, WQI, WGP, and WQM. In some embodiments, the CD47 comprises substitutions at one or more of amino acids corresponding to amino acid positions Q1, L3, A53, and L54 of an amino acid sequence that is at least 85%, 90%, 91%, 92%, 93%, 94%, 95%, 96%, 97%, 98%, 99%, or 100% identical to SEQ ID NO: 145 or 146. In some embodiments, the CD47 protein comprises a Q1P or Q1L substitution. In some embodiments, the CD47 protein comprises a L3R, L3A, L3K, L3N, L3E, or L3V substitution. In some embodiments, the CD47 protein comprises a A53W, A53Y, A53D, A53Q, or A53V substitution. In some embodiments, the CD47 protein comprises a L54A, L54I, L54K, L54M, L54E, L54W, L54S, L54I, or L54V substitution. In particular embodiments, the CD47 protein comprises a Q1P substitution. In some embodiments, the disclosure provides for a cell encoding a CD47 protein comprising an amino acid sequence that is at least 85%, 90%, 91%, 92%, 93%, 94%, 95%, 96%, 97%, 98%, 99%, or 100% identical to SEQ ID NO: 245. In some embodiments, the CD47 protein comprises any of the modified CD47 amino acid sequences disclosed in U.S. Pat. No. 10,358,472, which is incorporated by reference herein in its entirety.

In some embodiments, the disclosure provides for a cell expressing a CD47 protein, wherein the CD47 protein comprises at least 3 amino acids added to the N-terminus of the mature CD47 protein (not including the leader sequence, e.g., not including the sequence of SEQ ID NO: 244) wherein the 3 amino acids added has the formula X₃—X₂—X₁, where X₃ is W; X₂ is selected from Q, A and G; and X₁ is selected from R, P, L, T, F, I, and M. In some embodiments, the CD47 protein comprises an amino acid other than Q at the amino acid corresponding to position 1 of an amino acid sequence that is at least 85%, 90%, 91%, 92%, 93%, 94%, 95%, 96%, 97%, 98%, 99%, or 100% identical to SEQ ID NO: 145 or 146. In some embodiments, the CD47 protein comprises a proline at the amino acid corresponding to position 1 of an amino acid sequence that is at least 85%, 90%, 91%, 92%, 93%, 94%, 95%, 96%, 97%, 98%, 99%, or 100% identical to SEQ ID NO: 145 or 146. In some embodiments, the three amino acids are selected from WQR, WAP, WQL, WQP, WQT, WQF, WQI, WGP, and WQM. In particular embodiments, the three amino acids are WQP or WGP. In some embodiments, the CD47 protein comprises any of the modified CD47 amino acid sequences disclosed in U.S. Pat. No. 10,358,472, which is incorporated by reference herein in its entirety. In some embodiments, the cell further comprises a disruption in the beta-2-microglobulin gene. In some embodiments, the cell further comprises a disruption in ABO. In some embodiments, the cell further comprises a disruption in renalase. In some embodiments, the cell further comprises a disruption in CXCL10. In some embodiments, the cell further comprises a disruption in F3.

In some embodiments, the cell does not encode an exogenous transgene encoding CD47. In some embodiments, any of the genetically engineered cells disclosed herein does not have increased expression of CD47 or expression of a mutant CD47. In some embodiments, the disclosure provides for a genetically engineered cell in which the endogenous CD47 gene has been mutated to express a mutant CD47 protein. In some embodiments, the mutated CD47 protein is expressed under the control of the cell's endogenous CD47 promoter. In some embodiments, the mutant CD47 is membrane-bound. In some embodiments, the mutant CD47 is secreted and is soluble. In some embodiments, the CD47 protein comprises an amino acid other than Q at the amino acid corresponding to position 1 of an amino acid sequence that is at least 85%, 90%, 91%, 92%, 93%, 94%, 95%, 96%, 97%, 98%, 99%, or 100% identical to SEQ ID NO: 145 or 146. In some embodiments, the CD47 protein comprises a proline at the amino acid corresponding to position 1 of an amino acid sequence that is at least 85%, 90%, 91%, 92%, 93%, 94%, 95%, 96%, 97%, 98%, 99%, or 100% identical to SEQ ID NO: 145 or 146. In some embodiments, the mutant CD47 protein comprises at least 3 amino acids added to the N-terminus of the mature CD47 protein (e.g., a CD47 protein comprising an amino acid sequence that is at least 85%, 90%, 91%, 92%, 93%, 94%, 95%, 96%, 97%, 98%, 99%, or 100% identical to SEQ ID NO: 145 or 146) wherein the 3 amino acids added has the formula X₃—X₂—X₁, where X₃ is W; X₂ is selected from Q, A and G; and X₁ is selected from R, P, L, T, F, I, and M. In some embodiments, the three amino acids are selected from WQR, WAP, WQL, WQP, WQT, WQF, WQI, WGP, and WQM. In particular embodiments, the three amino acids are WQP or WGP. In some embodiments, the mutant CD47 protein comprises an amino acid sequence that is at least 85%, 90%, 91%, 92%, 93%, 94%, 95%, 96%, 97%, 98%, 99%, or 100% identical to SEQ ID NO: 245, 162 or 163. In some embodiments, the CD47 protein comprises any of the modified CD47 amino acid sequences disclosed in U.S. Pat. No. 10,358,472, which is incorporated by reference herein in its entirety. In some embodiments, the cell further comprises a disruption in the beta-2-microglobulin gene. In some embodiments, the cell further comprises a disruption in ABO. In some embodiments, the cell further comprises a disruption in renalase. In some embodiments, the cell further comprises a disruption in CXCL10. In some embodiments, the cell further comprises a disruption in F3.

In some embodiments, the CD47 mutants are generated using a CRISPR/Cas system. In some embodiments, the CRISPR/Cas system utilizes Cas9 (e.g., SpCas9) and one or more guide RNAs, e.g., one or more single guide (sgRNAs). In some embodiments, the one or more sgRNAs target the CD47 gene and comprise the nucleotide sequence of any one of the sequences in Table 7. In some embodiments, the one or more sgRNAs target the CD47 gene and comprise the nucleotide sequence of any one of the sequences of SEQ ID Nos: 147-154. In some embodiments, any of the guides in Table 7 are used in combination with a Cas9 protein (e.g., SpCas9) to edit the CD47 gene by cutting the CD47, thereby facilitating the introduction of an exogenous template, such that following said edit the edited CD47 gene encodes any of the CD47 mutant proteins disclosed herein.

TABLE 7

| Sequence | SEQ ID NO |
|---|---|
| ATGCTTTGTTACTAATATGG | 147 |
| TCCATGCTTTGTTACTAATA | 148 |
| TCCATATTAGTAACAAAGCA | 149 |
| AATAGTAGCTGAGCTGATCC | 150 |
| TAGCTGAGCTGATCCTGGAA | 151 |
| CATTTTTCTTTTTCCTTTCC | 152 |
| AGTCTGTACTGTAAGATCTT | 153 |
| AAATGTTGCTGTTTATCTTA | 154 |

In some embodiments, a transgene encoding CD47 is inserted into a cell's genome such that the expression of the CD47 transgene is tied to the expression of an endogenous target gene in the cell. In some embodiments, the endogenous target gene is a housekeeping gene, such as ACTB, NANOG, or GAPDH. In some embodiments, the transgene is inserted such that the 3′UTR of the housekeeping gene (e.g., the 3′ UTR of the GAPDH gene) is intact. In some embodiments, the CD47 template is ssDNA, dsDNA, a nanocircle, or a plasmid.

In some embodiments, the CD47 transgene is inserted into a target gene, e.g., housekeeping gene (e.g., GAPDH gene). In some embodiments, the expression of the CD47 transgene is linked to that of the target gene (e.g., a housekeeping gene such as GAPDH) to form a bicistronic construct. In some embodiments, the expression of the CD47 transgene is linked to that of the target gene (e.g., a housekeeping gene such as GAPDH) by a ribosomal skipping element, such as a 2A or IRES sequence. In some embodiments, the bicistronic construct is flanked by a left homology arm and a right homology arm, wherein the homology arms are each homologous to an endogenous sequence of a target gene, e.g., a housekeeping gene such as GAPDH. In some embodiments, the left homology arm comprises a sequence that is at least 80%, 85%, 90%, 91%, 92%, 93%, 94%, 95%, 96%, 97%, 98%, 99% or 100% identical to the sequence of SEQ ID NO: 51, or complementary sequences and/or fragments thereof. In some embodiments, the right homology arm comprises a sequence that is at least 80%, 85%, 90%, 91%, 92%, 93%, 94%, 95%, 96%, 97%, 98%, 99% or 100% identical to the sequence of SEQ ID NO: 52, or complementary sequences and/or fragments thereof. In some embodiments, the left or right homology arms comprise a “codon wobble” sequence comprising “wobble bases” as compared to a reference wildtype gene sequence (e.g., a wildtype GAPDH gene sequence) in order to prevent a guide RNA that targets a portion of the reference wildtype gene sequence from targeting a portion of the codon wobble sequence. In some embodiments, the left homology arm further comprises at its 3′ end a sequence that comprises a codon wobble sequence comprising 1, 2, 3, 4, 5, 6, 7, 8, 9, 10, 11, 12, 13, 14, 15, 16, 17, 18, 19, 20 or 21 wobble bases as compared to a wildtype sequence. In some embodiments, the codon wobble sequence comprises a sequence that is at least 80%, 85%, 90%, 91%, 92%, 93%, 94%, 95%, 96%, 97%, 98%, 99% or 100% identical to the sequence of SEQ ID NO: 53, or complementary sequences and/or fragments thereof. In some embodiments, the codon wobble sequence comprises a sequence that is at least 80%, 85%, 90%, 91%, 92%, 93%, 94%, 95%, 96%, 97%, 98%, 99% or 100% identical to the sequence of SEQ ID NO: 53, or complementary sequences and/or fragments thereof, and comprises 1, 2, 3, 4, 5, 6, 7, 8, 9, 10, 11, 12, 13, 14, 15, 16, 17, 18, 19, 20 or 21 of the wobble bases of SEQ ID NO: 53 (represented by capital letters in SEQ ID NO: 53). In some embodiments the left homology arm further comprises at its 3′ end a sequence that comprises a “flex region” that may be 3′ to the codon wobble sequence. In some embodiments, the flex region sequence comprises a sequence that is at least 80%, 85%, 90%, 91%, 92%, 93%, 94%, 95%, 96%, 97%, 98%, 99% or 100% identical to the sequence of SEQ ID NO: 54, or complementary sequences and/or fragments thereof. In some embodiments, the left homology arm comprises, in 5′ to 3′ orientation, a sequence that is at least 80%, 85%, 90%, 91%, 92%, 93%, 94%, 95%, 96%, 97%, 98%, 99% or 100% identical to the sequence of SEQ ID NO: 51, followed by a sequence comprising a sequence that is at least 80%, 85%, 90%, 91%, 92%, 93%, 94%, 95%, 96%, 97%, 98%, 99% or 100% identical to the sequence of SEQ ID NO: 53, followed by a sequence comprising a sequence that is at least 80%, 85%, 90%, 91%, 92%, 93%, 94%, 95%, 96%, 97%, 98%, 99% or 100% identical to the sequence of SEQ ID NO: 54. In some embodiments, the 2A sequence encodes a polypeptide comprising any one of SEQ ID Nos: 55-58. In some embodiments, the 2A sequence further encodes an N-terminal GSG linker. In preferred embodiments, the 2A sequence facilitates expression of both the target gene (e.g., GAPDH) and the CD47 transgene, but wherein the ultimate protein products (e.g., CD47 and GAPDH) are separate.

SEQ ID NO: 51
ctgccaaggctgtgggcaaggtcatccctgagctgaacgggaagctcact
ggcatggccttccgtgtccccactgccaacgtgtcagtggtggacctgac
ctgccgtctagaaaaacctgccaaatatgatgacatcaagaaggtggtga
agcaggcgtcggagggccccctcaagggcatcctgggctacactgagcac
caggtggtctcctctgacttcaacagcgacacccactcctccacctttga
cgctggggctggcattgccctcaacgaccactttgtcaagctcatttcct
ggtatgtggctggggccagagactggctcttaaaaagtgcagggtctggc
gccctctggtggctggctcagaaaaagggccctgacaactcttttcatct
tctagg
SEQ ID NO: 52
gacccctggaccaccagccccagcaagagcacaagaggaagagagagacc
ctcactgctggggagtccctgccacactcagtcccccaccacactgaatc
tcccctcctcacagttgccatgtagaccccttgaagaggggaggggccta
gggagccgcaccttgtcatgtaccatcaataaagtaccctgtgctcaacc
agttacttgtcctgtcttattctagggtctggggcagaggggagggaagc
tgggcttgtgtcaaggtgagacattcttgctggggagggacctggtatgt
tctcctcagactgagggtagggcctccaaacagccttgcttgcttcgaga
accatttgettcccgctcagacgtcttgagtgctacaggaagctggcacc
(possible wobble bases are capitalized)
SEQ ID NO: 53
taCgaTaaTgaGttCggctaTagTaaTCGCgtAgtCgaTctAatggcAca
TatggcGtcAaaAgaA
SEQ ID NO: 54
ggaagcgga
SEQ ID NO: 55
ATNFSLLKQAGDVEENPGP
SEQ ID NO: 56
EGRGSLLTCGDVEENPGP
SEQ ID NO: 57
QCTNYALLKLAGDVESNPGP
SEQ ID NO: 58
VKQTLNFDLLKLAGDVESNPGP

In some embodiments, the CD47 transgene is inserted using a CRISPR/Cas system. In some embodiments, the CRISPR/Cas system utilizes Cas9 (e.g., SpCas9) and one or more guide RNAs, e.g., one or more sgRNAs. In some embodiments, the one or more sgRNAs target a GAPDH gene and comprise the nucleotide sequence of any one of the sequences in Table 8. In some embodiments, the one or more sgRNAs target a GAPDH gene and comprise the nucleotide sequence of any one of SEQ ID NOs: 90-116. In some embodiments, the one or more sgRNAs target a GAPDH gene and comprise the nucleotide sequence of SEQ ID NO: 111 or 112. In some embodiments, the exact sgRNA cut site and positing of template homology arms relative to the cut site may determine the resultant insertion structure. In some embodiments, the CD47 is inserted as a “scarless” insertion of CD47 at the target gene (e.g., GAPDH), i.e., that does not disrupt the target gene's regulatory sequences (e.g., 3′ UTR sequence). In some embodiments, the CD47 insertion pushes the wildtype exon 9 of GAPDH downstream of, but not in frame with, the stop codon of CD47. In this instance the homology arm may provide a recoded partial exon 9 of GAPDH to eliminate template cutting by the sgRNA and nuclease. The recoding results in the same GAPDH amino acid sequence but changes the nucleotide sequence to eliminate guide binding. In some embodiments, a poly adenylation (pA) sequence is included with the insertion.

TABLE 8

| Name | Sequence | SEQ ID NO: |
|---|---|---|
| C9_GAPDH_HDR_gRNA186 | ACAAGTAACTGGTTGAGCAC | 90 |
| C9_GAPDH_HDR_gRNA185 | CAAGTAACTGGTTGAGCACA | 91 |
| C9_GAPDH_HDR_gRNA060 | GCCATGTAGACCCCTTGAAG | 92 |
| C9_GAPDH_HDR_gRNA043 | GTCTGGCGCCCTCTGGTGGC | 93 |
| C9_GAPDH_HDR_gRNA147 | TTTTCTGAGCCAGCCACCAG | 94 |
| C9_GAPDH_HDR_gRNA146 | TTTCTGAGCCAGCCACCAGA | 95 |
| C9_GAPDH_HDR_gRNA042 | CAGGGTCTGGCGCCCTCTGG | 96 |
| C9_GAPDH_HDR_gRNA041 | GTGCAGGGTCTGGCGCCCTC | 97 |
| C9_GAPDH_HDR_gRNA040 | CTCTTAAAAAGTGCAGGGTC | 98 |
| C9_GAPDH_HDR_gRNA039 | ACTGGCTCTTAAAAAGTGCA | 99 |
| C9_GAPDH_HDR_gRNA038 | GACTGGCTCTTAAAAAGTGC | 100 |
| C9_GAPDH_HDR_gRNA145 | ACTTTTTAAGAGCCAGTCTC | 101 |
| C9_GAPDH_HDR_gRNA034 | CTCATTTCCTGGTATGTGGC | 102 |
| C9_GAPDH_HDR_gRNA033 | CAAGCTCATTTCCTGGTATG | 103 |
| C9_GAPDH_HDR_gRNA144 | TCTGGCCCCAGCCACATACC | 104 |
| C9_GAPDH_HDR_gRNA175 | TCTACATGGCAACTGTGAGG | 105 |
| C9_GAPDH_HDR_gRNA174 | CTACATGGCAACTGTGAGGA | 106 |
| C9_GAPDH_HDR_gRNA173 | TACATGGCAACTGTGAGGAG | 107 |
| C9_GAPDH_HDR_gRNA162 | TCTTCCTCTTGTGCTCTTGC | 108 |
| C9_GAPDH_HDR_gRNA161 | CTTCCTCTTGTGCTCTTGCT | 109 |
| C9_GAPDH_HDR_gRNA160 | TTCCTCTTGTGCTCTTGCTG | 110 |
| C9_GAPDH_HDR_gRNA047 | TCTAGGTATGACAACGAATT | 111 |
| C9_GAPDH_HDR_gRNA046 | GACAACTCTTTTCATCTTCT | 112 |
| C9_GAPDH_HDR_gRNA149 | AGAAGATGAAAAGAGTTGTC | 113 |
| C9_GAPDH_HDR_gRNA148 | GAAGATGAAAAGAGTTGTCA | 114 |
| C9_GAPDH_HDR_gRNA045 | TGGTGGCTGGCTCAGAAAAA | 115 |
| C9_GAPDH_HDR_gRNA044 | CTGGTGGCTGGCTCAGAAAA | 116 |

In some embodiments, any of the genetically engineered cells disclosed herein comprises a disruption of a gene encoding for any one or more of ABO, renalase, CXCL10, B2M, and/or F3, wherein the disruption results in reduced or eliminated expression of ABO, renalase, CXCL10, B2M, and/or F3. In some embodiments, the disruption comprises a deletion, an insertion, a translocation, an inversion, or a substitution of one or more nucleotides in a gene encoding for any one or more of ABO, renalase, CXCL10, B2M, and/or F3. In some embodiments, the disruption comprises an insertion of 1, 2, 3, 4, or 5 nucleotides in a gene encoding for any one or more of ABO, renalase, CXCL10, B2M, and/or F3 (e.g., as the result of using a CRISPR system and any one or more of any of the guide RNAs disclosed herein). In some embodiments, the disruption comprises a deletion of 1, 2, 3, 4, or 5 nucleotides in a gene encoding for any one or more of ABO, renalase, CXCL10, B2M, and/or F3 (e.g., as the result of using a CRISPR system and any one or more of any of the guide RNAs disclosed herein). In some embodiments, the disruption comprises a deletion, an insertion, a translocation, an inversion, or a substitution of at least 1, 2, 3, 4, 5, 6, 7, 8, 9, 10, 15, 20, 30, 40, 50, 75, 100, 150, 200, 250, 300, 400, 500, or 1000 nucleotides in a gene encoding for any one or more of ABO, renalase, CXCL10, B2M, and/or F3. In some embodiments, the disruption comprises a deletion, an insertion, a translocation, an inversion, or a substitution of 1-1000, 1-800, 1-600, 1-500, 1-300, 1-200, 1-100, 1-50, 1-10, 1-5, 1-3, 500-1000, 100-1000, 100-500, 100-300, 10-100, 10-80, 10-50, or 10-25 nucleotides in a gene encoding for any one or more of ABO, renalase, CXCL10, B2M, and/or F3. In some embodiments, the disruption is in one copy of the gene encoding for any one or more of ABO, renalase, CXCL10, B2M, and/or F3 in a cell. In some embodiments, the disruption is in both copies of the gene encoding for any one or more of ABO, renalase, CXCL10, B2M, and/or F3 in a cell. In some embodiments, the disruption of the gene encoding for any one or more of ABO, renalase, CXCL10, B2M, and/or F3 is a disruption of one or more introns, one or more exons, and/or one or more regulatory elements (e.g., a promoter or an enhancer) of the gene encoding for any one or more of ABO, renalase, CXCL10, B2M, and/or F3. In some embodiments, the disruption results in a decrease in expression of ABO, renalase, CXCL10, B2M, and/or F3. In some embodiments, the disruption results in a decrease of at least 5%, 10%, 15%, 20%, 25%, 30%, 35%, 40%, 45%, 50%, 55%, 60%, 65%, 70%, 75%, 80%, 85%, 90%, 95%, or 100% in expression of ABO, renalase, CXCL10, B2M, and/or F3 as compared to a cell of the same cell type lacking the disruption. In some embodiments, the disruption results in a decrease of 10-100%, 30-100%, 50-100%, 75-100%, 10-95%, 10-80%, 10-60%, 10-40%, 10-20%, 20-100%, 20-95%, 20-80%, 20-60%, 20-40%, 40-100%, 40-95%, 40-80%, 40-60%, 60-100%, 60-95%, 60-80%, 80-100%, 80-95%, or 90-98% in expression of ABO, renalase, CXCL10, B2M, and/or F3 as compared to a cell of the same cell type lacking the disruption. In some embodiments, the disclosure provides for a cell that does not have genetic disruption in the ABO gene. In some embodiments, the disclosure provides for a cell that does not have a genetic disruption in the CXCL10 gene. In some embodiments, the disclosure provides for a cell that does not have a genetic disruption in the renalase gene. In some embodiments, the disclosure provides for a cell that does not have a genetic disruption in the B2M gene. In some embodiments, the disclosure provides for a cell that does not have a genetic disruption in the tissue factor gene.

In some embodiments, the genetically engineered cell comprises an insertion of gene encoding CD47. In some embodiments, the insertion of the CD47 gene is into a safe harbor locus. In some embodiments, the insertion of the CD47 gene is in a housekeeping gene. In some embodiments, the insertion of the CD47 gene results in an increase of at least 10%, 25%, 30%, 50%, 60%, 70%, 80%, 90%, 100%, 150%, 200%, 250%, 300%, 400%, or 500% in expression of CD47 as compared to a cell lacking the insertion. In some embodiments, the insertion of the CD47 gene results in an increase of 10-500%, 50-500%, 100-500%, 200-500%, 300-500%, 10-300%, 50-300%, 100-300%, 100-200%, 10-200%, 50-200%, 100-200%, 10-100%, 10-75%, 10-50%, or 10-30% in expression of CD47 as compared to a cell lacking the insertion.

In some embodiments, the genetically engineered mammalian cell is engineered to have decreased or no expression of the renalase gene, and wherein the engineered mammalian cell has also been genetically engineered to have decreased or no expression of the ABO gene; decreased or no expression of the CXCL 10 gene, decreased or no expression of the beta-2 microglobulin (B2M), decreased or no expression for the tissue factor (F3) gene, and/or increased expression of CD47 when compared to the expression levels of the corresponding genes in the same cell type where the cell has not been genetically engineered. In some embodiments, the disclosure provides for a genetically engineered cell in which the endogenous CD47 gene has been mutated to express a mutant CD47 protein.

In some embodiments, the mammalian cell has been genetically engineered to have decreased or no expression of the renalase gene, and the cell has been further genetically engineered to have decreased or no expression of the ABO gene as compared to the expression level of the same cell type that has not been genetically engineered.

In some embodiments, the mammalian cell has been genetically engineered to have decreased or no expression of the renalase gene, and the cell has been further genetically engineered to have decreased or no expression of the ABO gene and/or the cell has been genetically engineered to have decreased or no expression of the CXCL10 gene as compared to the expression level of the same cell type that has not been genetically engineered.

In some embodiments, the mammalian cell has been genetically engineered to have decreased or no expression of the renalase gene, and the cell has been further genetically engineered to have decreased or no expression of the ABO gene; the cell has been genetically engineered to have decreased or no expression of the CXCL10 gene; and/or the cell has been genetically engineered to have decreased or no expression of the B2M gene as compared to the expression level of the same cell type that has not been genetically engineered.

In some embodiments, the mammalian cell has been genetically engineered to have decreased or no expression of the renalase gene, and the cell has been further genetically engineered to have decreased or no expression of the ABO gene; the cell has been genetically engineered to have decreased or no expression of the CXCL10 gene; the cell has been genetically engineered to have decreased or no expression of the B2M gene; and/or the cell has been genetically engineered to have decreased or no expression of the F3 gene as compared to the expression level of the same cell type that has not been genetically engineered.

In some embodiments, the mammalian cell has been genetically engineered to have decreased or no expression of the renalase gene, and the cell has been further genetically engineered to have decreased or no expression of the ABO gene; the cell has been genetically engineered to have decreased or no expression of the CXCL10 gene; the cell has been genetically engineered to have decreased or no expression of the B2M gene; the cell has been genetically engineered to have decreased or no expression of the F3 gene; and/or the cell has been genetically engineered to have increased expression of CD47 or expression of a mutant CD47 as compared to the expression level of the same cell type that has not been genetically engineered. In some embodiments the engineered cell comprises an insertion of an exogenous CD47 gene.

Some aspects of the present disclosure provide a mammalian cell that has been genetically engineered to have decreased or no expression of the CXCL10 gene, and wherein the cell also has been genetically engineered to have decreased or no expression of the ABO gene and/or the tissue factor (F3) gene.

In some embodiments, the mammalian cell has been genetically engineered to have decreased or no expression of the CXCL10 gene, and wherein the cell has further been genetically engineered to have decreased or no expression of the ABO gene as compared to the expression level of the same cell type that has not been genetically engineered.

In some embodiments, the mammalian cell has been genetically engineered to have decreased or no expression of the CXCL10 gene; the cell has further been genetically engineered to have decreased or no expression of the ABO gene; and/or the cell has been genetically engineered to have decreased or no expression of the tissue factor (TF3) gene as compared to the expression level of the same cell type that has not been genetically engineered.

In some embodiments, the mammalian cell has been has been genetically engineered to have decreased or no expression of the CXCL10 gene; the cell has further been genetically engineered to have decreased or no expression of the ABO gene; the cell has been genetically engineered to have decreased or no expression of the tissue factor (TF3) gene; and/or the cell has been genetically engineered to have decreased or no expression of the beta-2-microglobulin (B2M) gene as compared to the expression level of the same cell type that has not been genetically engineered.

In some embodiments, the mammalian cell has been has been genetically engineered to have decreased or no expression of the CXCL10 gene; the cell has further been genetically engineered to have decreased or no expression of the ABO gene; the cell has been genetically engineered to have decreased or no expression of the tissue factor (TF3) gene; the cell has been genetically engineered to have decreased or no expression of the beta-2-microglobulin (B2M) gene; and/or the cell has been genetically engineered to have decreased or no expression of the renalase gene as compared to the expression level of the same cell type that has not been genetically engineered.

In some embodiments, the mammalian cell has been has been genetically engineered to have decreased or no expression of the CXCL10 gene; the cell has further been genetically engineered to have decreased or no expression of the ABO gene; the cell has been genetically engineered to have decreased or no expression of the tissue factor (TF3) gene; the cell has been genetically engineered to have decreased or no expression of the beta-2-microglobulin (B2M) gene; the cell has been genetically engineered to have decreased or no expression of the renalase gene; and/or the cell has been genetically engineered to have increased expression of CD47 or expression of a mutant CD47 as compared to the expression level of the same cell type that has not been genetically engineered. In some embodiments the engineered cell comprises an insertion of an exogenous CD47 gene.

In some embodiments of the disclosure, the mammalian cell has been genetically engineered to have decreased or no expression of the B2M, CXCL 10, renalase, ABO, and F3 genes, and to have increased expression of CD47 or expression of a mutant CD47 as compared to the expression level of the same mammalian cell type that has not been genetically engineered.

Certain aspects of the disclosure comprise a mammalian cell which is ABO blood group type O, wherein the cell has been genetically engineered to have reduced or no expression of the renalase gene. In some embodiments, the cell has been genetically engineered to have reduced or no expression of the CXCL 10 gene.

In some embodiments, the mammalian cell is ABO blood group type O, wherein the cell has been genetically engineered to have reduced or no expression of the renalase gene; the cell has been genetically engineered to have reduced or no expression of the CXCL10 gene; the cell has been genetically engineered to have reduced or no expression of the B2M gene; the cell has been genetically engineered to have reduced or no expression of the F3 gene; and the cell has been genetically engineered to have increased expression of the CD47 gene as compared to the expression level of the same cell type that has not been genetically engineered.

Some aspects of the current disclosure are directed to a genetically modified mammalian cell, wherein the cell is a stem cell. In some embodiments, the modified mammalian cell is a pluripotent stem cell (PSC), an embryonic stem cell (ESC), an induced pluripotent stem cell (iPSC), or an embryonic germ stem cell (EGSC). In some embodiments, the modified mammalian cell is differentiated from a pluripotent stem cell. In some embodiments, the genetically engineered mammalian cell is a somatic cell. In some embodiments, the modified mammalian cell is a definitive endoderm cell. In some embodiments, the mammalian cell is a primitive gut tube cell. In some embodiments, the cell is a PDX1-positive pancreatic progenitor cell. In some embodiments, the cell is a NKX6.1-positive pancreatic progenitor cell. In some embodiments, the cell is an Ngn3-positive endocrine progenitor cell. In some embodiments, the cell is an insulin-positive endocrine cell. In some embodiments, the mammalian cell is a pancreatic SC-β cell. In some embodiments, the cell is NKX6.1-positive. In some embodiments, the cell is ISL1-negative. In some embodiments, the mammalian cell is NKX6.1-positive and ISL1-positive.

Certain aspects of the disclosure are directed to a mammalian cell that has been genetically modified as described herein, wherein the genetic manipulations are performed using using CRISPR/Cas, piggybac transposon, TALEN, zinc finger technology, homing endonucleases, or meganucleases. In some embodiments, at least one genetic modification is made in an intronic region of the gene. In some embodiments, at least one genetic modification is made in an exon of the gene. In some embodiments, at least one genetic modification is made in a regulatory region (e.g., promoter) of the gene.

Certain aspects of the current disclosure are directed to a mammalian cell, wherein the mammalian cell has been genetically engineered to have decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 3 and/or SEQ ID NO: 5 or a protein comprising an amino acid sequence that is at least 80%, 85%, 90%, 91%, 92%, 93%, 94%, 95%, 96%, 97%, 98%, 99% or 100% identical to SEQ ID NO: 4 and/or 6, and wherein the cell has also been genetically engineered to have decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 1 or a protein comprising an amino acid sequence that is at least 80%, 85%, 90%, 91%, 92%, 93%, 94%, 95%, 96%, 97%, 98%, 99% or 100% identical to SEQ ID NO: 2; decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 7 or a protein comprising an amino acid sequence that is at least 80%, 85%, 90%, 91%, 92%, 93%, 94%, 95%, 96%, 97%, 98%, 99% or 100% identical to SEQ ID NO: 8; decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 9 or a protein comprising an amino acid sequence that is at least 80%, 85%, 90%, 91%, 92%, 93%, 94%, 95%, 96%, 97%, 98%, 99% or 100% identical to SEQ ID NO: 10; decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 11 or a protein comprising an amino acid sequence that is at least 80%, 85%, 90%, 91%, 92%, 93%, 94%, 95%, 96%, 97%, 98%, 99% or 100% identical to SEQ ID NO: 12; and/or increased expression of a protein encoded by a nucleic acid that is at least 80%, 85%, 90%, 91%, 92%, 93%, 94%, 95%, 96%, 97%, 98%, 99% or 100% identical to SEQ ID NO: 13 and/or SEQ ID NO: 15 or a protein comprising an amino acid sequence that is at least 80%, 85%, 90%, 91%, 92%, 93%, 94%, 95%, 96%, 97%, 98%, 99% or 100% identical to SEQ ID NO: 14, SEQ ID NO: 16, SEQ ID NO: 145 and/or SEQ ID NO: 146, as compared to the expression level of the same cell type that has not been genetically engineered.

In some embodiments, a mammalian cell has been genetically engineered to have decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 7 or a protein comprising an amino acid sequence that is at least 80%, 85%, 90%, 91%, 92%, 93%, 94%, 95%, 96%, 97%, 98%, 99% or 100% identical to SEQ ID NO: 8, and the cell also has been genetically engineered to have decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 1 and/or SEQ ID NO: 11 or a protein comprising an amino acid sequence that is at least 80%, 85%, 90%, 91%, 92%, 93%, 94%, 95%, 96%, 97%, 98%, 99% or 100% identical to SEQ ID NO: 2 or 12, as compared to the expression level of the same cell type that has not been genetically engineered.

In some embodiments, the mammalian cell has been genetically engineered to have decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 7 or a protein comprising an amino acid sequence that is at least 80%, 85%, 90%, 91%, 92%, 93%, 94%, 95%, 96%, 97%, 98%, 99% or 100% identical to SEQ ID NO: 8, and the cell also has been genetically engineered to have decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 1 and/or SEQ ID NO: 11 or a protein comprising an amino acid sequence that is at least 80%, 85%, 90%, 91%, 92%, 93%, 94%, 95%, 96%, 97%, 98%, 99% or 100% identical to SEQ ID NO: 2 and/or 12 and is further genetically engineered to have decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 9 or a protein comprising an amino acid sequence that is at least 80%, 85%, 90%, 91%, 92%, 93%, 94%, 95%, 96%, 97%, 98%, 99% or 100% identical to SEQ ID NO: 10 as compared to the expression level of the same cell type that has not been genetically engineered.

In some embodiments, the mammalian cell has been genetically engineered to have decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 7 or a protein comprising an amino acid sequence that is at least 80%, 85%, 90%, 91%, 92%, 93%, 94%, 95%, 96%, 97%, 98%, 99% or 100% identical to SEQ ID NO: 8, decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 1 or a protein comprising an amino acid sequence that is at least 80%, 85%, 90%, 91%, 92%, 93%, 94%, 95%, 96%, 97%, 98%, 99% or 100% identical to SEQ ID NO: 2; decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 11 or a protein comprising an amino acid sequence that is at least 80%, 85%, 90%, 91%, 92%, 93%, 94%, 95%, 96%, 97%, 98%, 99% or 100% identical to SEQ ID NO: 12; decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 9 or a protein comprising an amino acid sequence that is at least 80%, 85%, 90%, 91%, 92%, 93%, 94%, 95%, 96%, 97%, 98%, 99% or 100% identical to SEQ ID NO: 10; and is further genetically engineered have decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 3 and/or SEQ ID NO: 5 or a protein comprising an amino acid sequence that is at least 80%, 85%, 90%, 91%, 92%, 93%, 94%, 95%, 96%, 97%, 98%, 99% or 100% identical to SEQ ID NO: 4 and/or SEQ ID NO: 6 as compared to the expression level of the same cell type that has not been genetically engineered.

In some embodiments, the mammalian cell has been genetically engineered to have decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 7 or a protein comprising an amino acid sequence that is at least 80%, 85%, 90%, 91%, 92%, 93%, 94%, 95%, 96%, 97%, 98%, 99% or 100% identical to SEQ ID NO: 8, decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 1 or a protein comprising an amino acid sequence that is at least 80%, 85%, 90%, 91%, 92%, 93%, 94%, 95%, 96%, 97%, 98%, 99% or 100% identical to SEQ ID NO: 2; decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 11 or a protein comprising an amino acid sequence that is at least 80%, 85%, 90%, 91%, 92%, 93%, 94%, 95%, 96%, 97%, 98%, 99% or 100% identical to SEQ ID NO: 12; decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 9 or a protein comprising an amino acid sequence that is at least 80%, 85%, 90%, 91%, 92%, 93%, 94%, 95%, 96%, 97%, 98%, 99% or 100% identical to SEQ ID NO: 10; decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 3 and/or SEQ ID NO: 5 or a protein comprising an amino acid sequence that is at least 80%, 85%, 90%, 91%, 92%, 93%, 94%, 95%, 96%, 97%, 98%, 99% or 100% identical to SEQ ID NO: 4 and/or SEQ ID NO: 6, wherein the cell has been genetically engineered to have increased expression of a protein encoded by a nucleic acid that is at least 80%, 85%, 90%, 91%, 92%, 93%, 94%, 95%, 96%, 97%, 98%, 99% or 100% identical to SEQ ID NO: 13 and/or SEQ ID NO: 15 or a protein comprising an amino acid sequence that is at least 80%, 85%, 90%, 91%, 92%, 93%, 94%, 95%, 96%, 97%, 98%, 99% or 100% identical to SEQ ID NO: 14, SEQ ID NO: 16, SEQ ID NO: 145 and/or SEQ ID NO: 146 as compared to the expression level of the same cell type that has not been genetically engineered.

In some embodiments, the mammalian cell has been genetically engineered to have decreased or no expression of proteins encoded by nucleic acid that are at least 80%, 85%, 90%, 91%, 92%, 93%, 94%, 95%, 96%, 97%, 98%, 99% or 100% identical to SEQ ID NO: 9, SEQ ID NO: 7, SEQ ID NO: 1, and SEQ ID NO: 11 or protein comprising amino acid sequences that are at least 80%, 85%, 90%, 91%, 92%, 93%, 94%, 95%, 96%, 97%, 98%, 99% or 100% identical to SEQ ID NO: 10, SEQ ID NO: 8, SEQ ID NO: 2 and SEQ ID NO: 12; have decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 3 and/or SEQ ID NO: 5 or a protein comprising an amino acid sequence that is at least 80%, 85%, 90%, 91%, 92%, 93%, 94%, 95%, 96%, 97%, 98%, 99% or 100% identical to SEQ ID NO: 4 and/or SEQ ID NO: 6; and have increased expression of a protein encoded by a nucleic acid that is at least 80%, 85%, 90%, 91%, 92%, 93%, 94%, 95%, 96%, 97%, 98%, 99% or 100% identical to SEQ ID NO: 13 and/or SEQ ID NO: 15 or a protein comprising an amino acid sequence that is at least 80%, 85%, 90%, 91%, 92%, 93%, 94%, 95%, 96%, 97%, 98%, 99% or 100% identical to SEQ ID NO: 14, SEQ ID NO: 16, SEQ ID NO: 145 and/or SEQ ID NO: 146, as compared to the expression level of the same cell type that has not been genetically engineered.

In some aspects of the current disclosure, a mammalian cell, wherein the cell is ABO blood group type O, has been genetically engineered to have reduced or no expression of a protein encoded by a nucleic acid that is at least 80%, 85%, 90%, 91%, 92%, 93%, 94%, 95%, 96%, 97%, 98%, 99% or 100% identical to SEQ ID NO: 3, SEQ ID NO: 5, and/or SEQ ID NO: 7 or a protein comprising an amino acid sequence that is at least 80%, 85%, 90%, 91%, 92%, 93%, 94%, 95%, 96%, 97%, 98%, 99% or 100% identical to SEQ ID NO: 4, SEQ ID NO: 6 and/or SEQ ID NO: 8, as compared to the expression level of the same cell type that has not been genetically engineered. In some embodiments, the mammalian ABO blood group type O cell has been genetically engineered to have decreased or no expression of proteins encoded by nucleic acids that are at least 80%, 85%, 90%, 91%, 92%, 93%, 94%, 95%, 96%, 97%, 98%, 99% or 100% identical to SEQ ID NO: 9, SEQ ID NO: 7, and SEQ ID NO: 11 or protein comprising amino acid sequences that are at least 80%, 85%, 90%, 91%, 92%, 93%, 94%, 95%, 96%, 97%, 98%, 99% or 100% identical to SEQ ID NO: 10, SEQ ID NO: 8 and SEQ ID NO: 12; have decreased or no expression of a protein encoded by a nucleic acid that is at least 80%, 85%, 90%, 91%, 92%, 93%, 94%, 95%, 96%, 97%, 98%, 99% or 100% identical to SEQ ID NO: 3 and/or SEQ ID NO: 5 or a protein comprising an amino acid sequence that is at least 80%, 85%, 90%, 91%, 92%, 93%, 94%, 95%, 96%, 97%, 98%, 99% or 100% identical to SEQ ID NO: 4 and/or SEQ ID NO: 6; and have increased expression of a protein encoded by a nucleic acid that is at least 80%, 85%, 90%, 91%, 92%, 93%, 94%, 95%, 96%, 97%, 98%, 99% or 100% identical to SEQ ID NO: 13 and/or SEQ ID NO: 15 or a protein comprising an amino acid sequence that is at least 80%, 85%, 90%, 91%, 92%, 93%, 94%, 95%, 96%, 97%, 98%, 99% or 100% identical to SEQ ID NO: 14, SEQ ID NO: 16, SEQ ID NO: 145 and/or SEQ ID NO: 146, as compared to the expression level of the same cell type that has not been genetically engineered.

Some aspects of the current disclosure describe a mammalian cell that has been genetically engineered to have decreased or no expression of the protein encoded by the renalase gene, and wherein the cell also has been genetically engineered to have decreased or no expression of the protein encoded by the ABO gene; decreased or no expression of the protein encoded by the CXCL 10 gene; decreased or no expression of the protein encoded by the beta-2 microglobulin (B2M) gene; decreased or no expression of the protein encoded by the tissue factor (F3) gene; and/or increased expression of the protein encoded by the CD47 gene, as compared to the protein expression level of the same cell type that has not been genetically engineered.

### Methods for Genetic Modification

In some embodiments of the disclosure, typical gene editing techniques are used to genetically modify the mammalian cells. In some embodiments, the gene is disrupted using CRISPR/Cas, piggybac transposon, TALEN, zinc finger technology, and/or homing endonucleases or meganucleases. Homing endonucleases generally cleave their DNA substrates as dimers, and do not have distinct binding and cleavage domains. ZFNs recognize target sites that consist of two zinc-finger binding sites that flank a 5- to 7-base pair (bp) spacer sequence recognized by the FokI cleavage domain. TALENs recognize target sites that consist of two TALE DNA-binding sites that flank a 12- to 20-bp spacer sequence recognized by the FokI cleavage domain. The Cas9 nuclease is targeted to DNA sequences complementary to the targeting sequence within the single guide RNA (gRNA) located immediately upstream of a compatible protospacer adjacent motif (PAM).

Piggybac transposon is a mobile genetic element that efficiently transposes between vectors and chromosomes via a “cut and paste” mechanism. During transposition, the PB transposase recognizes transposon-specific inverted terminal repeat sequences (ITRs) located on both ends of the transposon vector and efficiently moves the contents from the original sites and integrates them into TTAA chromosomal sites. The activity of the PiggyBac transposon system enables genes of interest between the two ITRs in the PB vector to be easily mobilized into target genomes.

Additionally, in embodiments where genes or their corresponding proteins are increased, exogenous genes may be inserted into the mammalian cells through methods known to those in the art. Such methods include transduction and transfection.

In some embodiments, a modified viral vector is used for clustered regularly interspaced short palindromic repeats-Cas endonuclease (CRISPR-Cas) gene editing in vitro and in vivo.

### Methods of Treatment

The term “treatment” or “treating” refers to preventing or delaying the onset, slowing down the progression, and/or or ameliorating the symptoms of the disorder. In some embodiments, any of the genetically engineered SC-islet cell compositions disclosed herein may be administered to a subject to treat diabetes, e.g., Type 1 Diabetes. In some embodiments, the composition is administered to the subject intravenously, e.g., via the hepatic portal vein. In some embodiments, the composition is administered to the subject in a device.

The term “subject” refers to any mammalian subject, including human.

### Disclosure of Sequences

The following is a listing and description of the sequences disclosed and used throughout the disclosure. These sequences are also included in the aforementioned electronic sequence listing (41822WO_SequenceListing.xml; Size: 333,386 bytes; and Date of Creation: Oct. 27, 2023) incorporated by reference in its entirety.

### EXEMPLIFICATION

### Example 1

hESCs were engineered by knocking-out the ABO gene using CRISPR/SpCas9 technology. ABO knockout stem cells were differentiated into ABO knockout SC-islet cells according to a protocol substantially the same as that described in Tables 7 and 9 of US2023218676, which is incorporated by reference herein in its entirety.

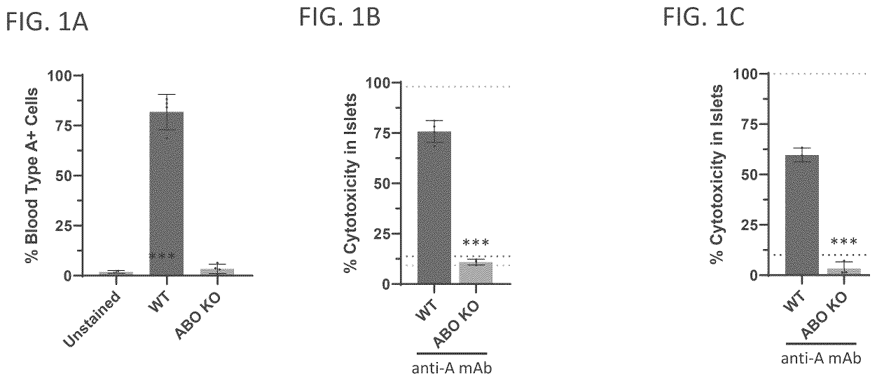

For the ADCC assay, wildtype or ABO knockout stage 6 SC-islets were pretreated with anti-A antigen antibody, washed, and then co-cultured with human NK cells at various E:T ratios for 4 hours. SC-islets for analysis by flow cytometry for viability or apoptosis with AnnexinV, where CD49a was used to mark SC-islets, and CD45 NK cells. For the CDC assay, wildtype or ABO knockout stage 6 SC-islets were pre-treated with anti-A antigen antibody, washed, and then treated with human serum for 3.5 hours. Cell viability was measured following serum treatment using a modified version of the Cell Titer Glo Luminescent reagent. Stem cell clones with ABO knockout showed no A-antigen expression by flow cytometry (FIG. 1A). ABO knockout SC-islets resisted anti-A-antigen antibody mediated NK cell based cytotoxicity in vitro (FIG. 1B). ABO knockout SC-islets also resisted complement mediated cell death when cultured in the presence of anti-A-antibody and human serum (FIG. 1C).

### Example 2

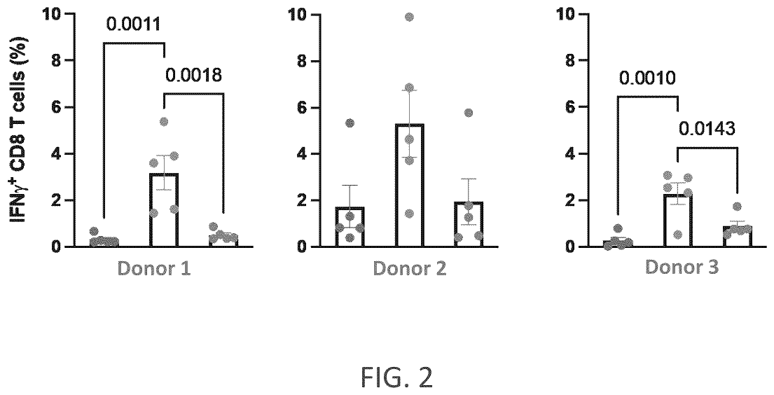

hESCs were engineered by knocking-out the B2M gene using CRISPR/SpCas9 technology. SC-islet cells were generated according to a six-stage protocol substantially the same as that described in Tables 7 and 9 of US2023218676, which is incorporated by reference herein in its entirety. Human monocyte-derived dendritic cells (MoDCs) were generated by incubating human monocytes isolated from PBMCs with GM-CSF and IL-4 for 7 days, followed by maturation and activation with heat-killed cytokine-treated human islets resuspended in a maturation cocktail containing R848, LPS, PolyIC, and IFNγ for 24 hrs. Autologous human T cells isolated from the same PBMCs were then subsequently added to the culture for a total of 7-day co-culture, with low dose IL-2, IL-7, and IL-15 supplemented at day 2 and day 4 to aid T cell expansion. On day 7 of co-culture, expanded T cells were re-stimulated with the same heat-killed cytokine-treated human islets in media containing anti-CD28 and anti-CD49d antibodies and then analyzed by flow cytometry for LAMP-1 (CD107a), a marker of degranulation, and intracellular production of cytokines IFNγ and TNFα. FIG. 2 shows the results with T cells from three donors all tested at the same time. A response was detectable with WT SC-islets in all three cases, and the B2M KO islets diminished the response to near background levels, as shown.

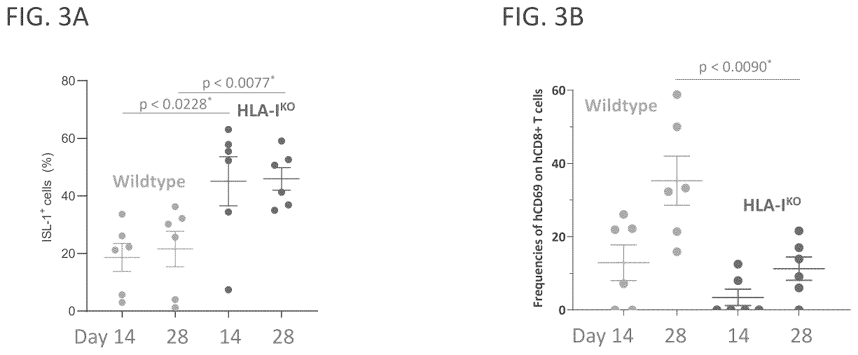

B2M knockout SC-islets were implanted into humanized hu-PBMC-NSG MHC I/II Double Knockout mice (Jackson Laboratories Cat No. 025216). Grafts that were removed at 2 and 4 weeks contained more SC-islets compared to wildtype (FIG. 3A). CD8+ T-cells recovered from the 2 and 4 week grafts also showed less activation marker CD69 (FIG. 3B).

### Example 3

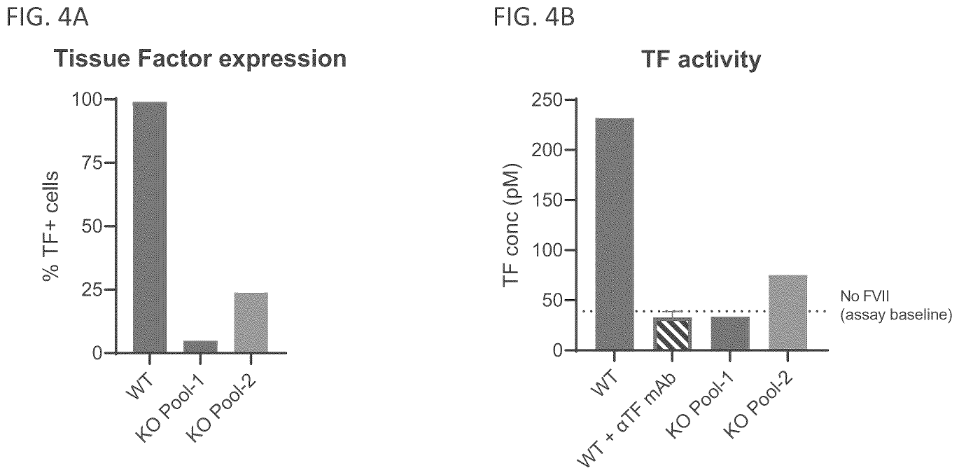

hESCs were engineered by knocking-out the CD142 gene using CRISPR/SpCas9 technology. Wildtype or CD142 knockout hESCs were stained with anti-CD142 PE conjugated antibody clone NY2 and then analyzed by flow cytometry. For the tissue factor activity assay, wildtype or CD142 knockout pool of hESC were tested for the ability to activate factor X, a sign of tissue factor presence on cells. The Abcam Tissue Factor Activity Assay Kit (cat #ab 108906) was used to compare activation levels between wildtype and CD142 knockout hESC. Briefly, the kit was prepared according to the manufacturer's recommendation. Dissociated hESC cells from either background were incubated for 30 minutes in the appropriate wells of the supplied 96-well assay plate. Absorbance was measured as the final step, and activity was calculated based on the generation of a standard curve. CD142 knockout hESCs displayed significantly reduced tissue factor pathway activation in vitro. CD142 knockout hESC pools showed very low CD142 expression following nucleofection (FIG. 4A). CD142 knockout pool hESCs showed lower tissue factor pathway activation compared to wildtype in vitro measured by the Factor Xa assay, a measure of CD142 pathway activation (FIG. 4B).

### Example 4

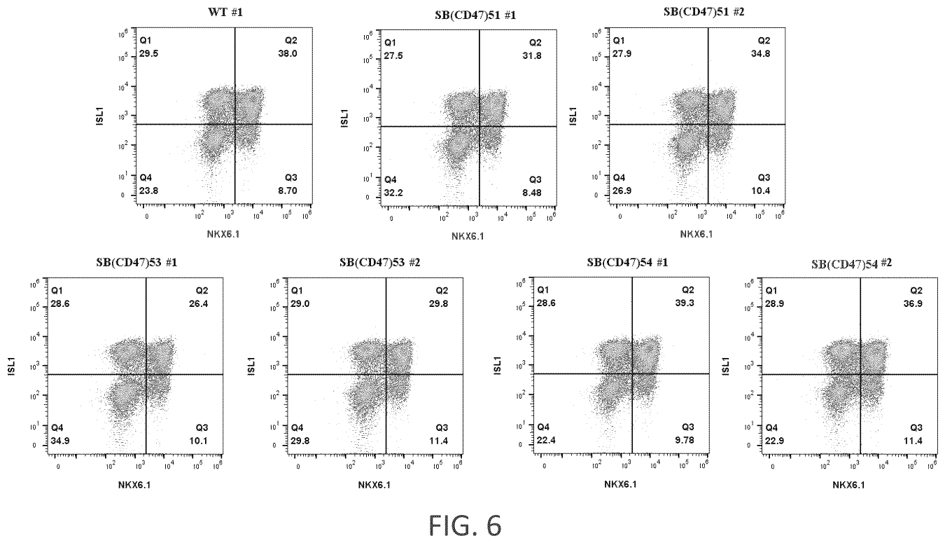

hESCs were edited to generate clones expressing membrane-bound high-affinity CD47 using the guide AATAGTAGCTGAGCTGATCC (SEQ ID NO: 150) and template indicated in FIGS. 5A-B, and according to the schematic in FIG. 5C. Clones were selected as homozygous for “KI” (meaning the insertion of the WQPP motif was detected by sequencing following editing), homozygous for “KO” (meaning a frameshifting INDEL that disrupted endogenous CD47), or heterozygous for KI/KO. Three clones were selected for SC-islet differentiation to test whether modification at CD47 precludes SC-islet differentiation. The edited hESC cells were then differentiated to SC-islet cells according to a six-stage protocol substantially the same as that described in Table 9 of US2023218676, which is incorporated by reference herein in its entirety. SC-islet cells were examined following Stage 5, and the results suggest that high affinity WQPP edits at CD47 do not disrupt normal differentiation (FIG. 6).

### Example 5

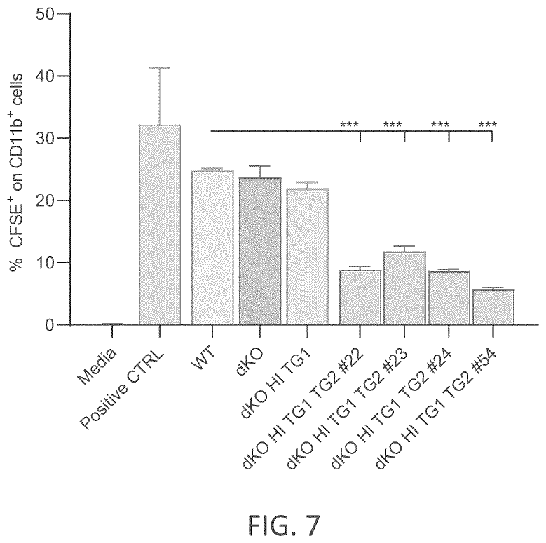

hESCs were engineered to disrupt B2M and CIITA (“dKO”), and in some instances the dKO cells were further engineered to insert a) PDL1 (TG1), or b) TG1 and CD47 (TG2). Monocytes were isolated from human donor PBMCs via positive selection using CD14 Microbeads (Miltenyi Biotec) and plated in adhesion for maturation into macrophages in presence of 50 ng/ml of M-CSF for 8-10 days. hESC edited lines were differentiated into endothelial cells using STEMdiff Endothelial Differentiation Kit (Stemcell Technologies) following manufacturer instruction. At day 7, endothelial cells were collected and plated for expansion until macrophages were matured. Expression of CD34 and CD144 expression was assessed by flow cytometry to confirm successful differentiation. For the assay, endothelial edited cell lines were labeled with CFSE and cocultured with monocyte derived macrophages (MDM) at a ratio of 1:1, the plate was spun down at 2,000 rpm for 1 min to achieve sufficient effector-to-target contact. MDM cultured in presence of FITC-dextran for 1 hour were considered a positive control for the assay. After 6 hours, cells were collected and stained for CD11b. Phagocytic engulfment was measured by flow cytometry to assess the percentage of CFSE positive CD11b positive cells. A significant reduction in the engulfment of endothelial edited cell lines expressing TG2 was observed (FIG. 7).

### Example 6

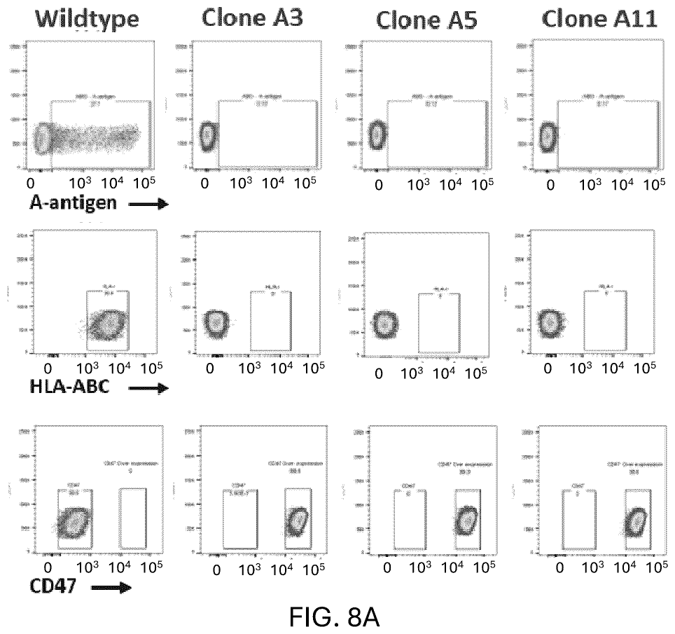

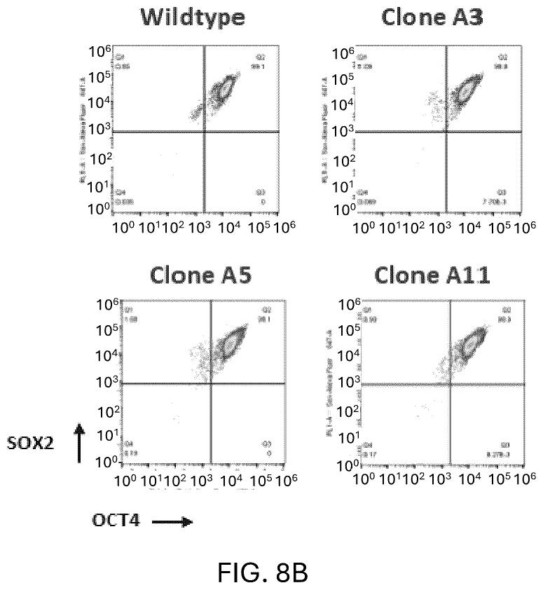

hESCs were edited using CRISPR-SpCas9 technology to disrupt ABO and B2M expression, and to overexpress hCD47 (using the sequence of SEQ ID NO: 242) from the GAPDH locus via the P2A element from GAPDH. The edited pool of triple-edited cells was single cell cloned, and three representative clones were tested by flow cytometry for loss of A-antigen via ABO knockout, loss of HLA class I via B2M knockout, and increased CD47 expression via GAPDH insertion (FIG. 8A). Representative clones were also examined for pluripotency markers to ensure maintenance of stemness following the editing and cloning process (FIG. 8B).

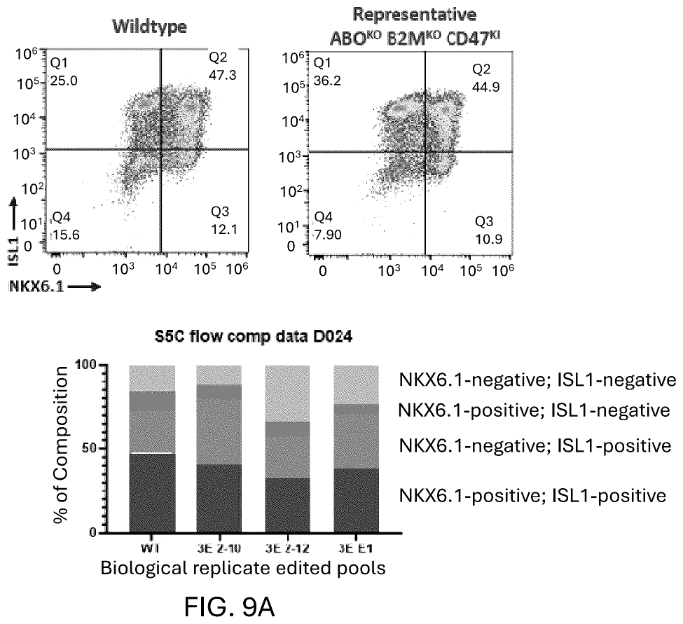

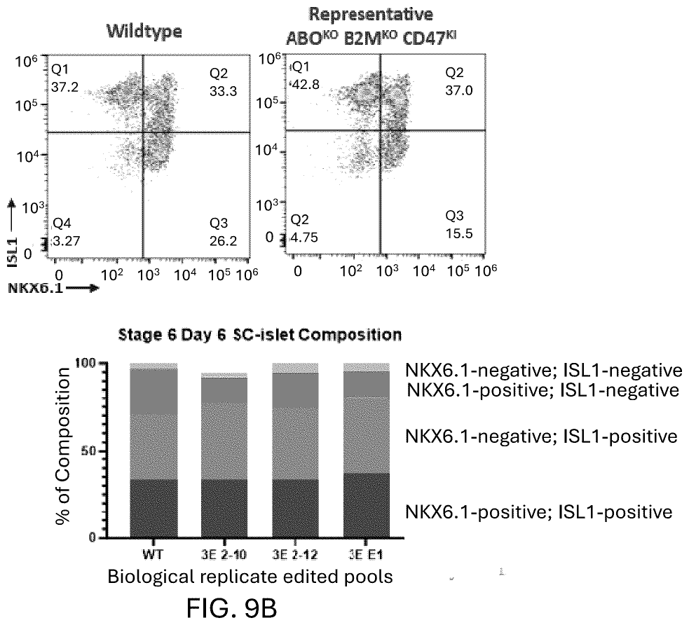

The triple-edited hESCs were bulk sorted to enrich the edited population to greater than 80%. The edited hESC cells in the bulk pool were then differentiated to SC-islet cells according to a six-stage protocol substantially the same as that described in Tables 7 and 9 of US2023218676, which is incorporated by reference herein in its entirety. SC-islet cells were examined following Stage 5 (FIG. 9A) and at day 6 of Stage 6 (FIG. 9B). The line successfully reached stage 5 complete and Stage 6 with a composition, yield and morphology (data not shown) similar to SC-islets differentiated from wildtype hESCs (FIGS. 9A and 9B).

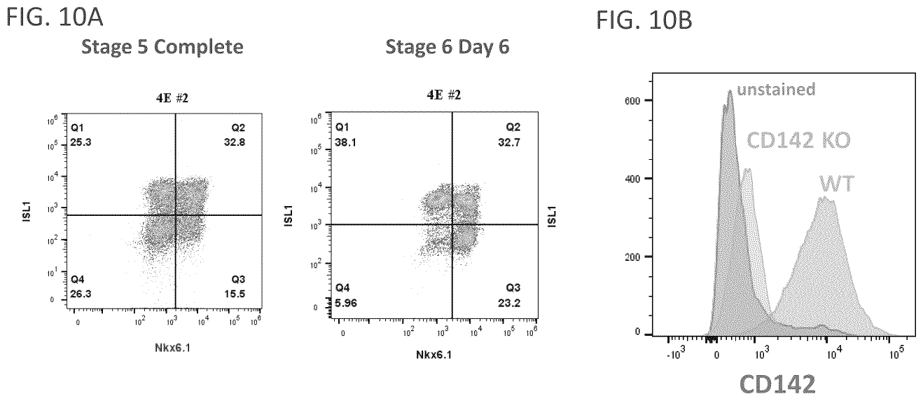

The triple-edited hESC cells were further edited to disrupt CD142 using CRISPR/SpCas9 technology. The pooled line exhibited >90% knockout at the stem cell state. The quadruple-edited hESC cells were then differentiated to SC-islet cells according to a six-stage protocol substantially the same as that described in Tables 7 and 9 of US2023218676, which is incorporated by reference herein in its entirety. SC-islet cells were examined following Stage 5 and at day 6 of Stage 6. Wildtype or quadruple edited stage 6 SC-islets were dissociated with accutase, washed, and then live stained for CD142 with CD142-PE (clone: NY2) and examined by flow cytometry. FIG. 10A-B demonstrates that quadruple-edited hESCs were differentiated successfully to Stage 5 complete SC-islets and into stage 6 SC-islets.

SEQ ID NO: 1
Homo sapiens ABO mRNA
Nucleic acid
AGACGCGGAGCCATGGCCGAGGTGTTGCGGACGCTGGCCGGAAAACCAAAATGCCA
CGCACTTCGACCTATGATCCTTTTCCTAATAATGCTTGTCTTGGTCTTGTTTGGTTAC
GGGGTCCTAAGCCCCAGAAGTCTAATGCCAGGAAGCCTGGAACGGGGGTTCTGCAT
GGCTGTTAGGGAACCTGACCATCTGCAGCGCGTCTCGTTGCCAAGGATGGTCTACCC
CCAGCCAAAGGTGCTGACACCGTGTAGGAAGGATGTCCTCGTGGTGACCCCTTGGCT
GGCTCCCATTGTCTGGGAGGGCACATTCAACATCGACATCCTCAACGAGCAGTTCAG
GCTCCAGAACACCACCATTGGGTTAACTGTGTTTGCCATCAAGAAATACGTGGCTTT
CCTGAAGCTGTTCCTGGAGACGGCGGAGAAGCACTTCATGGTGGGCCACCGTGTCC
ACTACTATGTCTTCACGACCAGCCGGCCGCGGTGCCCCGCGTGACGCTGGGGACCG
GTCGGCAGCTGTCAGTGCTGGAGGTGCGCGCCTACAAGCGCTGGCAGGACGTGTCC
ATGCGCCGCATGGAGATGATCAGTGACTTCTGCGAGCGGCGCTTCCTCAGCGAGGTG
GATTACCTGGTGTGCGTGGACGTGGACATGGAGTTCCGCGACCACGTGGGCGTGGA
GATCCTGACTCCGCTGTTCGGCACCCTGCACCCCGGCTTCTACGGAAGCAGCCGGGA
GGCCTTCACCTACGAGCGCCGGCCCCAGTCCCAGGCCTACATCCCCAAGGACGAGG
GCGATTTCTACTACCTGGGGGGGTTCTTCGGGGGGTCGGTGCAAGAGGTGCAGCGG
CTCACCAGGGCCTGCCACCAGGCCATGATGGTCGACCAGGCCAACGGCATCGAGGC
CGTGTGGCACGACGAGAGCCACCTGAACAAGTACCTGCTGCGCCACAAACCCACCA
AGGTGCTCTCCCCCGAGTACTTGTGGGACCAGCAGCTGCTGGGCTGGCCCGCCGTCC
TGAGGAAGCTGAGGTTCACTGCGGTGCCCAAGAACCACCAGGCGGTCCGGAACCCG
TGAGCGGCTGCCAGGGGCTCTGGGAGGGCTGCCGGCAGCCCCGTCCCCCTCCCGCC
CTTGGTTTTAGCAGAACGGGTAAACTCTGTTTCCTTTGTCCGTCCTGTTGTGAGTAAC
TGAAGCCTAGGCCCCGTCCCCACCTCAAATCACACACACCCCCTCCCCACCACAGAG
ACACCATTACATACACAGACACACACAGAAAGACACACACAGACACAAAATCACAC
ACACACCCTCCCCGCCACAGAGACACCATTACATACACAGACACACACAGAAAGAC
ACAGACACAAAATCACACACACACCCTCCCCGCCACAGAGACACACCATTACATAC
ACAGACACGCAATCGCAGATACGCCCTTCCGGCCACAGAAACACACCATTACACAC
ACATACACAGAAAGACACACACAGACACACAATCACACGCAGCCCCTCCCCGCCAC
AGAGACACACCATTACATACACAGACACACACAGAAAGACACACACAGACACAAA
ATCACACACACACCCTCCCCGCCACAGAGACACACCATTACATACACAGACACACA
CAGACACACAATCACACACAGCCCCTCCCCGCCACAGAGACACACCATTACATACA
CAGACACACACAGACACACAATCACAGATACCCCCTCCCGGCCATAGAGACACACC
GTTACACACACATACACAGAAAGACACACACAGCCCCTCCCTGCCACAGAGACACA
CCATTACATACACAGACACACACAGACATACACAGACACACACACAGACACACAAT
CACACACATCCCCTCCCCGCCACAGAGACACACCATTACACACACAGACACACACA
GAAAGACACACATACACACAATCACACACACCCTCTTCTTGCCACAGAGACGCACC
ATTACATACACAGACACAGACACACAGAGACACAGACACACAAACATAGACACAC
ACACACAGACACACACCAGACACGCAAAGACACACAGACACAGATACACAGATAC
AAAGACACAGACATATAGACACACAGACATGCACAGAGACACATGGAGACACATG
CAAAAATGCACAGAGAAAGACATACAGAAGTGTACACACAGACACATAGACCACA
CAGACACACAGACATGCATGCAAACACACAGACATGCAGACATGCACACAAACAC
AGACTCACGCACACAGACTTAGGCAGCCCAAATTCAGCGCCTGGGGCATAAGTTCC
TGGAGGGGTGGCCACCTTCAGCCCCCACGGTAAGGTCCTGAGGAACCTTCCCCTTAG
ACAAGGGATCATGGAGGAGGTCTCTTCCGGAGCCTGGAGGGAGGCCTCAAGTGGTC
CTTCCACCTCGGCATCCCAAAGTGCTAGGATTATAAGCATGAGCCACTGCACCTGGC
CCCAACATCATTTATTGAACAGACTGTCGTTTCCACATTGTGTTTCTTGGCATTTCTG
TCAAAAATCAGTTGACCGTAAATGCATGGATTCACTTCCGGGCTCTCTATTCTGTTTC
ATTGGTCTGTTTGTTTTTATGCCAATACCATGATGTTTTGATTATTATAGCTTTATAGT
ATATCTTGAAGTTAGGTAGTGTATTGTCTCCAGCTTTGTTCTTTTTGCTTAAGATTGC
TTTGGCTATTTAGGGGTCTTTGTGGCCATTACTGACAGTTAAAATGGCCCTGGCTCAT
GTCTCCTGGGCCTCTACAGTACTTTAGGTGGGGAAGTACTGTGGCCCACATGTCCCA
ACAGGCTGTGTGTGGCACCTGACAGTGCTTTGTGCCACTGTGGAGCAGGTGTTACTC
CCTCATACGAATTTTAGGATTATTTTATTTATTTCAGTGAAAAATGTCAATGAAATTT
TGATAGGGATTGCATTGAATCTGTAGATTGTTTGGGGTTGTATGGACATTTTAACAA
TATTAATTCTTCCAATTCATGAACATGGGATGTCTTTTTTTTGGCACCTGATCTCAGA
TATGGGACAGCTTTCTAATTATTTGTATCTTCAATTTCTTTCACAAATGTTTTATAGTT
TTCAGCATACAGATCTTTCACTTCCTTGGTTGGATTTATTTGCAAGTATTTTTTGTAG
CTATTGTAAATGACATTGTTTTCTTGATTTCTTTGTTAGGGTATAGAAATGCTACAGA
TTGTTGTATGTTAATTTTATATCCTGCAACTTTACTGATTTCATTTATTCTAACATCTT
ATTGGTGGAATCTTTAGGGTTTTCTATAAGATCATGTCATCTGTAAACAGGGACAAT
TTAAGTTCTTCCTTTCCAATTTGGATACCTTTTATTTCTTCCTCTTGCCTAATTATTTT
GGCTGAGGGCCAACTTGTTGGGTTTGTTGTTGTTGGTTTTTGTCTGTGTCTTTTGGTG
TTTCGTGTTGCTGGCTTTTCCAGCACACAGTCACAGATATATGAGGCAGAAAGAAAA
CCCAGGGAACTCCTGTCATGTCATTCCTCAGGTCCCAAGGTCCCTACATGGTCTGCC
TTCTTCTCTCCACCTTTTAGTGTTTGATATACATACGTTTATGTATGTAGTTATATATG
TATGTGTAATACCCGGGGAAATATACAGAGATTTTAACTGTCCTTAGTGTAGGGCGT
GGGGACAGGTGCATCTATTCTACCTTGCCTGGAAGTGGAAAAATCTTCAAGAAATCA
ATTTATCACCAACTGGCATGACAGAACTCCTGGTTTAGTGTCTAGCTGTCTCCACTA
AAATCAATCACCATGCCATCAGGCACTCTCTTCCCTCTGGTGTGTACTGGTACCTGA
AACAGCGTCCAGCACCCTGAGAGCACAGAACGCAGCTTCTTATGTGCCCCACCCAA
TAATCCCAAATCGAGCACAGACCTGGGAGCTGAAGAGACAAGCTGGAGGTCAGGCA
GTTCTATTACTTAAGCCTGGTTCTTCCTGACTCCAAAAGCCAGGCGCCACCCCATGC
TCCTTCCCAGCAGAAGCTGGTTTTCACCGGGTCAGGAGGACAGGATGGCTATTCCTG
ACCGTTGTACAGCAGTGCCACATCTCCTAGTTTCCAGAACATTTAATGTGTTGCTGTG
GAGGCCTGGCAGCTCTGAAGTAGCCCCTTGCGCCCAGGACAAACCGGGCCTGGAGG
GGTGGGGTGGGGTAAGCTGAAGCTTCCAGTATCCCACGTGGGCATTCTGTTGCTGAG
GGCAACCGCCCCTCCCTGCAAGGGAGGGGAAGGAGCAGGCAGGCACACCCTTCCTC
CCTCCACATTTCACTGGAGTTGACCTGTGTCCTACTGTGGTTGCACCTGGACCAGCC
CAGAGGGACTGGAGTGACTCTTCCCGCAGGTAGGGCACAGCCAGCCTGGGCACAGG
GCCTCACTGCAATCCCTGCGCCCCGCCTCCCTGGGGGATTTCCAGGGGTTGTCCTGC
TGCTTGGTGAGCTGGGGGTGTGGGGGACCACAGATGAGGCAGGCGCCTAAATTGCT
TGGGCGGAGTCCGGCTGCTCTCTCCCTCCAGGCCCTTTCCTGTGTCCTCGGGCCCCTC
GGGTCCCTGCACCAGGCCAGCAGCAATCGGGTGTCAGAGGTTCAAGTCCAGGCTGA
CTCCTGGGATGCACCGACTTTGCCCAGGAGGGCTGGGGAGAGGGCAGCCCCAAAGT
GTCATCTTTGATTATTCTTTGTCCCCAAAGCCAGCCTTGGGCATGTGCCCAGCACAG
GGAGAGCTGCTGCCTCTGTGGGCTTGAGTCATTTTTGCTTCCTTGCCCCTCTCTGCTC
TGTTTGGTTTAAAAAACAAAAACCAACACCCATGGGTGAGGCGAGGCGAGGCGAGG
CTTGCCTTGATCCACCCGCTTCCTCCCCTGCAGCCTCGGGCCCCATCCTCCCGCGTGG
CCGCACTAGGGGTGCTGGGGGCGGAGTGGGGACCAGACCTGAGTGGGTTCTGGGTT
CGGCTTCGCAGCAGGAGCAGCTGGGGCATCATCAGTTGGGCAGGTGACCCAGGATC
TGAGCAGGAATCTTCCAGGTGGAGGTGGGGCCGCCCCTCTGCTTGCCACAGCTGCCT
TCTGCTCTGGGGGCAGCACTGGCTGAGAATCCAGTGAAGTAAGGCCTTTGAAATGGT
TTTGATTGTAAAAGTAATGTTTATGTTTAGCCATTCCTTTTTTTATTTTTTATTTTTTG
AGACGGGTCTCCCTCTGTCACCCAGGCTGGAGTGCAGTCGTGAAATGTGGGCTCACT
GCAACCTCCACCTCCCAGGTTCAAGTGATTCTCCTGCCTCAGCCTCCTGAGTAGTTG
GGATTACAAGTGCCTGCCACCACACCTGACTAAGTTTTGTATTTTTTAGTAGAGATG
GAGTTTCACCATGTTGGCCAGGCTGGTCTCAAACTCATATCCTCAAGTGATCCACCC
TCCTCGTCCTCCCAAAGTGCTGGGATTTTGCCATGAGCCACCACGCTCAGCCATGTT
TAGCCATTTTTAAAAGGTGTGAACAGATAATTAAGTCTATTAGCAACATTAGAAAAT
TTAAGTTAAAATTTAAATGACTCTAGCAAGAATTAGCCTTTAGGTGCCCTGGGTCCC
CCTCCCCACCGCCCCCCACCCCGCCGCCTTCTGAGAAGCGTTTTGTAATTTTTCTTAT
TTATAAAATGGGTTTGATTCAAAACTGACTTTTCCTCTTCACAGAGTCTCACAGGTCC
TTCCCCAGGTCCAAGAGGCTCTTCTGTGGTCTAATGACAAGTAGCTGCCTAGCCGTG
GTGGCACCTCCTATCACATGTTAAGGGACCCCTCCCCAGGGCCACACCTGGCAGAA
GGTGGCTTATGATGTTCGCAGCTTGAAAGTAGTGTAAACCAAAGATAAAATTCTAAG
CCCACTCCCCCAGCCATCGGAATGGACCCCTCCTCTTGGCCAGGGCACTCCAAAGTT
AACCTGAAAAACCGGTTCAGGCTGTGAAGAGAAGGTGGAGTGGACATGCCTCATTT
ATGTCCTCCTCCCTTTTGGAATTCAGCAAAGCTGACCAGCATGAACATTAACACAGA
CCTTAAGTCTGATTAGTGGCATTTACAATCTATACTCTCTGAAGCGTGCTACCTGGA
GGCTTCCTTTGCATGATAAAACTTTGGTCTCCACAACCCCTTATCATAACCTAGACAC
TCCTTTCTAGTGATAATAACTCTTTCAACCAATTGCCAATAAAAAAATTTTGAATCTA
CCTATAACCTGGAACCTCCCTGCTCCACCTTCGAGTTGTCCTACCTTTCTGGACAGAA
GCAATGTGGATCTTGCATGTATTTGATTGATGTCTCATGTCTCCCTAAAATGTATACA
ATTAGGCTGTGCCCAGATCACCCTGGGCACATGTTCTCAGGCCCTCCTGAGGTCTCT
GTCTCGGGCCATTGGTCACTCAGATTCGGCTCAGAATAAATCTCTTCAAATATT
SEQ ID NO: 2
Homo sapiens ABO protein
Amino acids
MAEVLRTLAGKPKCHALRPMILFLIMLVLVLFGYGVLSPRSLMPGSLERGFCMAVREPD
HLQRVSLPRMVYPQPKVLTPCRKDVLVVTPWLAPIVWEGTFNIDILNEQFRLQNTTIGLT
VFAIKKYVAFLKLFLETAEKHFMVGHRVHYYVFTDQPAAVPRVTLGTGRQLSVLEVRA
YKRWQDVSMRRMEMISDFCERRFLSEVDYLVCVDVDMEFRDHVGVEILTPLFGTLHPG
FYGSSREAFTYERRPQSQAYIPKDEGDFYYLGGFFGGSVQEVQRLTRACHQAMMVDQA
NGIEAVWHDESHLNKYLLRHKPTKVLSPEYLWDQQLLGWPAVLRKLRFTAVPKNHQA
VRNP
SEQ ID NO: 3
Homo sapiens Renalase gene NM_001031709
Nucleic acids
AGGGCCCAGGTCGGCCCAGGGAGCACGGAACCAAAGAGCGCTAGCGCCGGTTCGG
CCGCCTTTCCAGAAAGCCCGGGCCGAACGGCCCCGCCGCAGAGACTCAGCGCGGAT
CGCTGCTCCCTCTCGCCATGGCGCAGGTGCTGATCGTGGGCGCCGGGATGACAGGA
AGCTTGTGCGCTGCGCTGCTGAGGAGGCAGACGTCCGGTCCCTTGTACCTTGCTGTG
TGGGACAAGGCTGAGGACTCAGGGGGAAGAATGACTACAGCCTGCAGTCCTCATAA
TCCTCAGTGCACAGCTGACTTGGGTGCTCAGTACATCACCTGCACTCCTCATTATGC
CAAAAAACACCAACGTTTTTATGATGAACTGTTAGCCTATGGCGTTTTGAGGCCTCT
AAGCTCGCCTATTGAAGGAATGGTGATGAAAGAAGGAGACTGTAACTTTGTGGCAC
CTCAAGGAATTTCTTCAATTATTAAGCATTACTTGAAAGAATCAGGTGCAGAAGTCT
ACTTCAGACATCGTGTGACACAGATCAACCTAAGAGATGACAAATGGGAAGTATCC
AAACAAACAGGCTCCCCTGAGCAGTTTGATCTTATTGTTCTCACAATGCCAGTTCCT
GAGATTCTGCAGCTTCAAGGTGACATCACCACCTTAATTAGTGAATGCCAAAGGCAG
CAACTGGAGGCTGTGAGCTACTCCTCTCGATATGCTCTGGGCCTCTTTTATGAAGCT
GGTACGAAGATTGATGTCCCTTGGGCTGGGCAGTACATCACCAGTAATCCCTGCATA
CGCTTCGTCTCCATTGATAATAAGAAGCGCAATATAGAGTCATCAGAAATTGGGCCT
TCCCTCGTGATTCACACCACTGTCCCATTTGGAGTTACATACTTGGAACACAGCATT
GAGGATGTGCAAGAGTTAGTCTTCCAGCAGCTGGAAAACATTTTGCCGGGTTTGCCT
CAGCCAATTGCTACCAAATGCCAAAAATGGAGACATTCACAGGTTACAAATGCTGC
TGCCAACTGTCCTGGCCAAATGACTCTGCATCACAAACCTTTCCTTGCATGTGGAGG
GGATGGATTTACTCAGTCCAACTTTGATGGCTGCATCACTTCTGCCCTATGTGTTCTG
GAAGCTTTAAAGAATTATATTTAGTGCCTATATCCTTATTCTCTACATGTGTATTGGG
TTTTTATTTTCACAATTTTCTGTTATTGATTATTTTGTTTTCTATTTTGCTAAGAAAAA
TTACTGGAAAATTGTTCTTCACTTATTATCATTTTTCATGTGGAGTATAAAATCAATT
TTGTAATTTTGATAGTTACAACCCATGCTAGAATGGAAATTCCTCACACCTTGCACCT
TCCCTACTTTTCTGAATTGCTATGACTACTCCTTGTTGGAGGAAAAGTGGTACTTAAA
AAATAACAAACGACTCTCTCAAAAAAATTACATTAAATCACAATAACAGTTTGTGTG
CCAAAAACTTGATTATCCTTATGAAAATTTCAATTCTGAATAAAGAATAATCACATT
ATCAAAGCCCCATCTTAAGTCTTCGGATGTGTCCTTGAATCAATATTTTTGCAAATTA
TACAAAACAAGATTTTTCCAAAATGTAGGTAACAGAGTGTAATTCTTATTTCTCATTT
ATCCCCCAAGTTATTAAGTGATCCTGAATTGTAGGTCATATATGTCATCATCTTAGTG
TGGAGGGCAACTTGACTGATAAAGAGACCTTCCTTCAGATTTTCAGAAAGTATAAGA
TTCCACATGATTTTCCCAGCCACACAGTACTTTTTAACTTTCAAACAAATTCCAGTCC
TAATATGAAAGATAAAAATTAAATAGAAACAGAGAGAAAGTATATCGATCCTTACC
TTTTGCTATATTTTATAGCTGTTGCTGTTACTTTATGGGTTCTCCAGTATGTGCTGTGG
CATTTAGACTGTGTCGAGTTTAATGAATTTAACACAACAAAAAATTTACTGAACCAG
AAAATAGATGCACTTAAAATAGTTCAATATTTGCCAAGTTGGTGGTTCAGCATATCA
CCCACATGCTTCAGTGACCTGACCCCACGACTTGCTAGCTGGAGAGAAATCAATCTC
CAGCCTTCCAAACCAGCTACCTGTTGCTAATTTGAAAAGCAAAATGATGAGTTCTAT
TTCAGCATTTTGAAAGGAGAAAAATCATTGCAGCCTCTCAAACTAACAAAAGTTCAA
CAAAAGACTTCTTACTGTAATAGTGTTTAAAGTTTCACACTTACATGTCCACTGTCAT
ACATACACATACACAGGCACAGGCAGAACTTGCTTCTATAGCTGCAAAGTGGGTTTT
ATGACCCTATAGCATATTATTATATGTTTCCTCTTAGCAATAAATTGGTGAAAAACTT
AAATGCCAA
SEQ ID NO: 4
Homo Sapiens Renalase Protein
Amino Acids
MAQVLIVGAGMTGSLCAALLRRQTSGPLYLAVWDKAEDSGGRMTTACSPHNPQCTAD
LGAQYITCTPHYAKKHQRFYDELLAYGVLRPLSSPIEGMVMKEGDCNFVAPQGISSIIKH
YLKESGAEVYFRHRVTQINLRDDKWEVSKQTGSPEQFDLIVLTMPVPEILQLQGDITTLIS
ECQRQQLEAVSYSSRYALGLFYEAGTKIDVPWAGQYITSNPCIRFVSIDNKKRNIESSEIG
PSLVIHTTVPFGVTYLEHSIEDVQELVFQQLENILPGLPQPIATKCQKWRHSQVTNAAAN
CPGQMTLHHKPFLACGGDGFTQSNFDGCITSALCVLEALKNYI
SEQ ID NO: 5
Homo sapiens Renalase Transcription variant
Nucleic acids
AGGGCCCAGGTCGGCCCAGGGAGCACGGAACCAAAGAGCGCTAGCGCCGGTTCGG
CCGCCTTTCCAGAAAGCCCGGGCCGAACGGCCCCGCCGCAGAGACTCAGCGCGGAT
CGCTGCTCCCTCTCGCCATGGCGCAGGTGCTGATCGTGGGCGCCGGGATGACAGGA
AGCTTGTGCGCTGCGCTGCTGAGGAGGCAGACGTCCGGTCCCTTGTACCTTGCTGTG
TGGGACAAGGCTGAGGACTCAGGGGGAAGAATGACTACAGCCTGCAGTCCTCATAA
TCCTCAGTGCACAGCTGACTTGGGTGCTCAGTACATCACCTGCACTCCTCATTATGC
CAAAAAACACCAACGTTTTTATGATGAACTGTTAGCCTATGGCGTTTTGAGGCCTCT
AAGCTCGCCTATTGAAGGAATGGTGATGAAAGAAGGAGACTGTAACTTTGTGGCAC
CTCAAGGAATTTCTTCAATTATTAAGCATTACTTGAAAGAATCAGGTGCAGAAGTCT
ACTTCAGACATCGTGTGACACAGATCAACCTAAGAGATGACAAATGGGAAGTATCC
AAACAAACAGGCTCCCCTGAGCAGTTTGATCTTATTGTTCTCACAATGCCAGTTCCT
GAGATTCTGCAGCTTCAAGGTGACATCACCACCTTAATTAGTGAATGCCAAAGGCAG
CAACTGGAGGCTGTGAGCTACTCCTCTCGATATGCTCTGGGCCTCTTTTATGAAGCT
GGTACGAAGATTGATGTCCCTTGGGCTGGGCAGTACATCACCAGTAATCCCTGCATA
CGCTTCGTCTCCATTGATAATAAGAAGCGCAATATAGAGTCATCAGAAATTGGGCCT
TCCCTCGTGATTCACACCACTGTCCCATTTGGAGTTACATACTTGGAACACAGCATT
GAGGATGTGCAAGAGTTAGTCTTCCAGCAGCTGGAAAACATTTTGCCGGGTTTGCCT
CAGCCAATTGCTACCAAATGCCAAAAATGGAGACATTCACAGGTACCAAGTGCTGG
TGTGATTCTAGGATGTGCGAAGAGCCCCTGGATGATGGCGATTGGATTTCCCATCTG
ACTTCCTGGAAATTGGAGCACACAGTCAGGTTTTATTTGATTTTTTTTTTTAAGGATA
CCACTTCACAGCCTTTAGGATAGCTATTATTTAGAAGCAAAACAGAAGATAAATGTT
GGCAAGGATGTGGAGATATTGGATTCCCTTGTGCAGTGCCGGTGGGAATGTAAAAT
GATGTAGCTACTATGGAAAATGATACGGCAATTTCTTTAGAAATGAAATATAGAATT
GCTGTATGATCTGCAGTTCCACATCTGGATATCTATCCAAAAGAAGTGAAAGTAGGG
ACTTGAACGAACATTTGTACACCAATGTTCACAGCGGCTTTATTCACAACAGCCAAA
AGGTGGAAGCAACCCAGTGTCCATGGATAGATGAATAGATAAATAAAATGTGGTAT
AAACATACAATGGGCTATTGTTTAGCCTTAAAAGGGAAGGAAATTCTGACATGCTGC
AATATGGATGAAGCTTAAAGTCATTATGCAAAGTGGAATAAGCCTATCACAAAAAA
TAATATTACATAATTCTACTTATATGAGGAATCTAGAGCAGTCAGTTTCACAGAGAC
AGAAAATAGAATGGTGGTTGCCAAGGGCTGGGAGAAGAGGGCAATGGAGAGTGAG
TGTTTAGTGGGTCAGAGTTTTAGTTTGGGAAGGTAAAAAGTTCTGGAGATGGATGAT
GGTTATGGGTGCTCAACAGTGTGAATGTACTTAATGCCACAGAACTGCACATTTAAA
TGTGGTTAAAATCATCACTTTTATGTTATGTATATTTACCACAATAAATAAAGAAGTT
GATATTTCTTATACTTACAAAGAGGAGAAGGGCATTTGCAAATCAACAAGAAGTGT
GAGGCCCCTCTCTCTAGCAGAAAAATAGACTAAATCTATTTCTTTATCTTTTAACATC
CTGTTTAAGGGAAATGCCAAAACAAATGGGAAAAAATACACACACACAAATATATA
TGAACATGTTTTGCCTCATGAGTAATCAAAATGTGTACATATGTATGTTTATGTATGT
GTGTTTATATTTAAAATCGTGTTCTGCCTTATGAGTAAACAAAAAGTATACAAATTA
AAAACTATAATGAAACGTAA
SEQ ID NO: 6
Homo sapiens Renalase Isoform X1 Protein
Amino acids
MAQVLIVGAGMTGSLCAALLRRQTSGPLYLAVWDKAEDSGGRMTTACSPHNPQCTAD
LGAQYITCTPHYAKKHQRFYDELLAYGVLRPLSSPIEGMVMKEGDCNFVAPQGISSIIKH
YLKESGAEVYFRHRVTQINLRDDKWEVSKQTGSPEQFDLIVLTMPVPEILQLQGDITTLIS
ECQRQQLEAVSYSSRYALGLFYEAGTKIDVPWAGQYITSNPCIRFVSIDNKKRNIESSEIG
PSLVIHTTVPFGVTYLEHSIEDVQELVFQQLENILPGLPQPIATKCQKWRHSQVPSAGVIL
GCAKSPWMMA IGFPI
SEQ ID NO: 7
Homo Sapiens CXCL10 gene
Nucleic Acids
GAGACATTCCTCAATTGCTTAGACATATTCTGAGCCTACAGCAGAGGAACCTCCAGT
CTCAGCACCATGAATCAAACTGCCATTCTGATTTGCTGCCTTATCTTTCTGACTCTAA
GTGGCATTCAAGGAGTACCTCTCTCTAGAACTGTACGCTGTACCTGCATCAGCATTA
GTAATCAACCTGTTAATCCAAGGTCTTTAGAAAAACTTGAAATTATTCCTGCAAGCC
AATTTTGTCCACGTGTTGAGATCATTGCTACAATGAAAAAGAAGGGTGAGAAGAGA
TGTCTGAATCCAGAATCGAAGGCCATCAAGAATTTACTGAAAGCAGTTAGCAAGGA
AAGGTCTAAAAGATCTCCTTAAAACCAGAGGGGAGCAAAATCGATGCAGTGCTTCC
AAGGATGGACCACACAGAGGCTGCCTCTCCCATCACTTCCCTACATGGAGTATATGT
CAAGCCATAATTGTTCTTAGTTTGCAGTTACACTAAAAGGTGACCAATGATGGTCAC
CAAATCAGCTGCTACTACTCCTGTAGGAAGGTTAATGTTCATCATCCTAAGCTATTC
AGTAATAACTCTACCCTGGCACTATAATGTAAGCTCTACTGAGGTGCTATGTTCTTA
GTGGATGTTCTGACCCTGCTTCAAATATTTCCCTCACCTTTCCCATCTTCCAAGGGTA
CTAAGGAATCTTTCTGCTTTGGGGTTTATCAGAATTCTCAGAATCTCAAATAACTAA
AAGGTATGCAATCAAATCTGCTTTTTAAAGAATGCTCTTTACTTCATGGACTTCCACT
GCCATCCTCCCAAGGGGCCCAAATTCTTTCAGTGGCTACCTACATACAATTCCAAAC
ACATACAGGAAGGTAGAAATATCTGAAAATGTATGTGTAAGTATTCTTATTTAATGA
AAGACTGTACAAAGTAGAAGTCTTAGATGTATATATTTCCTATATTGTTTTCAGTGTA
CATGGAATAACATGTAATTAAGTACTATGTATCAATGAGTAACAGGAAAATTTTAAA
AATACAGATAGATATATGCTCTGCATGTTACATAAGATAAATGTGCTGAATGGTTTT
CAAAATAAAAATGAGGTACTCTCCTGGAAATATTAAGAAAGACTATCTAAATGTTG
AAAGATCAAA AGGTTAATAA AGTAATTATA ACTAA
SEQ ID NO: 8
Homo sapiens CXCL10 Protein
Amino Acids
MNQTAILICCLIFLTLSGIQGVPLSRTVRCTCISISNQPVNPRSLEKLEIIPASQFCPRVEIIAT
MKKKGEKRCLNPESKAIKNLLKAVS KERSKRSP
SEQ ID NO: 9
Homo sapiens B2M mRNA
Nucleic Acids
ATTCCTGAAGCTGACAGCATTCGGGCCGAGATGTCTCGCTCCGTGGCCTTAGCTGTG
CTCGCGCTACTCTCTCTTTCTGGCCTGGAGGCTATCCAGCGTACTCCAAAGATTCAG
GTTTACTCACGTCATCCAGCAGAGAATGGAAAGTCAAATTTCCTGAATTGCTATGTG
TCTGGGTTTCATCCATCCGACATTGAAGTTGACTTACTGAAGAATGGAGAGAGAATT
GAAAAAGTGGAGCATTCAGACTTGTCTTTCAGCAAGGACTGGTCTTTCTATCTCTTG
TACTACACTGAATTCACCCCCACTGAAAAAGATGAGTATGCCTGCCGTGTGAACCAT
GTGACTTTGTCACAGCCCAAGATAGTTAAGTGGGATCGAGACATGTAAGCAGCATC
ATGGAGGTTTGAAGATGCCGCATTTGGATTGGATGAATTCCAAATTCTGCTTGCTTG
CTTTTTAATATTGATATGCTTATACACTTACACTTTATGCACAAAATGTAGGGTTATA
ATAATGTTAACATGGACATGATCTTCTTTATAATTCTACTTTGAGTGCTGTCTCCATG
TTTGATGTATCTGAGCAGGTTGCTCCACAGGTAGCTCTAGGAGGGCTGGCAACTTAG
AGGTGGGGAGCAGAGAATTCTCTTATCCAACATCAACATCTTGGTCAGATTTGAACT
CTTCAATCTCTTGCACTCAAAGCTTGTTAAGATAGTTAAGCGTGCATAAGTTAACTTC
CAATTTACATACTCTGCTTAGAATTTGGGGGAAAATTTAGAAATATAATTGACAGGA
TTATTGGAAATTTGTTATAATGAATGAAACATTTTGTCATATAAGATTCATATTTACT
TCTTATACATTTGATAAAGTAAGGCATGGTTGTGGTTAATCTGGTTTATTTTTGTTCC
ACAAGTTAAATAAATCATAAAACTTGA
SEQ ID NO: 10
Homo sapiens B2M Protein
Amino Acids
MSRSVALAVLALLSLSGLEAIQRTPKIQVYSRHPAENGKSNFLNCYVSGFHPSDIEVDLL
KNGERIEKVEHSDLSFSKDWSFYLLYYTEFTPTEKDEYACRVNHVTLSQPKIVKWDRD
M
SEQ ID NO: 11
Homo sapiens Tissue Factor 3 gene
Nucleic Acid
AAGACTGCGAGCTCCCCGCACCCCCTCGCACTCCCTCTGGCCGGCCCAGGGCGCCTT
CAGCCCAACCTCCCCAGCCCCACGGGCGCCACGGAACCCGCTCGATCTCGCCGCCA
ACTGGTAGACATGGAGACCCCTGCCTGGCCCCGGGTCCCGCGCCCCGAGACCGCCG
TCGCTCGGACGCTCCTGCTCGGCTGGGTCTTCGCCCAGGTGGCCGGCGCTTCAGGCA
CTACAAATACTGTGGCAGCATATAATTTAACTTGGAAATCAACTAATTTCAAGACAA
TTTTGGAGTGGGAACCCAAACCCGTCAATCAAGTCTACACTGTTCAAATAAGCACTA
AGTCAGGAGATTGGAAAAGCAAATGCTTTTACACAACAGACACAGAGTGTGACCTC
ACCGACGAGATTGTGAAGGATGTGAAGCAGACGTACTTGGCACGGGTCTTCTCCTAC
CCGGCAGGGAATGTGGAGAGCACCGGTTCTGCTGGGGAGCCTCTGTATGAGAACTC
CCCAGAGTTCACACCTTACCTGGAGACAAACCTCGGACAGCCAACAATTCAGAGTTT
TGAACAGGTGGGAACAAAAGTGAATGTGACCGTAGAAGATGAACGGACTTTAGTCA
GAAGGAACAACACTTTCCTAAGCCTCCGGGATGTTTTTGGCAAGGACTTAATTTATA
CACTTTATTATTGGAAATCTTCAAGTTCAGGAAAGAAAACAGCCAAAACAAACACT
AATGAGTTTTTGATTGATGTGGATAAAGGAGAAAACTACTGTTTCAGTGTTCAAGCA
GTGATTCCCTCCCGAACAGTTAACCGGAAGAGTACAGACAGCCCGGTAGAGTGTAT
GGGCCAGGAGAAAGGGGAATTCAGAGAAATATTCTACATCATTGGAGCTGTGGTAT
TTGTGGTCATCATCCTTGTCATCATCCTGGCTATATCTCTACACAAGTGTAGAAAGGC
AGGAGTGGGGCAGAGCTGGAAGGAGAACTCCCCACTGAATGTTTCATAAAGGAAGC
ACTGTTGGAGCTACTGCAAATGCTATATTGCACTGTGACCGAGAACTTTTAAGAGGA
TAGAATACATGGAAACGCAAATGAGTATTTCGGAGCATGAAGACCCTGGAGTTCAA
AAAACTCTTGATATGACCTGTTATTACCATTAGCATTCTGGTTTTGACATCAGCATTA
GTCACTTTGAAATGTAACGAATGGTACTACAACCAATTCCAAGTTTTAATTTTTAAC
ACCATGGCACCTTTTGCACATAACATGCTTTAGATTATATATTCCGCACTCAAGGAG
TAACCAGGTCGTCCAAGCAAAAACAAATGGGAAAATGTCTTAAAAAATCCTGGGTG
GACTTTTGAAAAGCTTTTTTTTTTTTTTTTTTTTTTTTGAGACGGAGTCTTGCTCTGTT
GCCCAGGCTGGAGTGCAGTAGCACGATCTCGGCTCACTGCACCCTCCGTCTCTCGGG
TTCAAGCAATTGTCTGCCTCAGCCTCCCGAGTAGCTGGGATTACAGGTGCGCACTAC
CACGCCAAGCTAATTTTTGTATTTTTTAGTAGAGATGGGGTTTCACCATCTTGGCCAG
GCTGGTCTTGAATTCCTGACCTCAGGTGATCCACCCACCTTGGCCTCCCAAAGTGCT
AGTATTATGGGCGTGAACCACCATGCCCAGCCGAAAAGCTTTTGAGGGGCTGACTTC
AATCCATGTAGGAAAGTAAAATGGAAGGAAATTGGGTGCATTTCTAGGACTTTTCTA
ACATATGTCTATAATATAGTGTTTAGGTTCTTTTTTTTTTCAGGAATACATTTGGAAA
TTCAAAACAATTGGCAAACTTTGTATTAATGTGTTAAGTGCAGGAGACATTGGTATT
CTGGGCACCTTCCTAATATGCTTTACAATCTGCACTTTAACTGACTTAAGTGGCATTA
AACATTTGAGAGCTAACTATATTTTTATAAGACTACTATACAAACTACAGAGTTTAT
GATTTAAGGTACTTAAAGCTTCTATGGTTGACATTGTATATATAATTTTTTAAAAAGG
TTTTCTATATGGGGATTTTCTATTTATGTAGGTAATATTGTTCTATTTGTATATATTGA
GATAATTTATTTAATATACTTTAAATAAAGGTGACTGGGAATTGTTACTGTTGTACTT
ATTCTATCTTCCATTTATTATTTATGTACAATTTGGTGTTTGTATTAGCTCTACTACAG
TAAATGACTGTAAAATTGTCAGTGGCTTACAACAACGTATCTTTTTCGCTTATAATAC
ATTTTGGTGACTGTA
SEQ ID NO: 12
Homo sapiens Tissue Factor 3 protein
Amino Acid
METPAWPRVPRPETAVARTLLLGWVFAQVAGASGTTNTVAAYNLTWKSTNFKTILEW
EPKPVNQVYTVQISTKSGDWKSKCFYTTDTECDLTDEIVKDVKQTYLARVFSYPAGNVE
STGSAGEPLYENSPEFTPYLETNLGQPTIQSFEQVGTKVNVTVEDERTLVRRNNTFLSLR
DVFGKDLIYTLYYWKSSSSGKKTAKTNTNEFLIDVDKGENYCFSVQAVIPSRTVNRKST
DSPVECMGQEKGEFREIFYIIGAVVFVVIILVIILAISLHKCRKAGVGQSWKENS PLNVS
SEQ ID NO: 13
Homo sapiens CD47 gene
Nucleic Acid
GCAGCCTGGGCAGTGGGTCCTGCCTGTGACGCGCGGCGGCGGTCGGTCCTGCCTGTA
ACGGCGGCGGCGGCTGCTGCTCCGGACACCTGCGGCGGCGGCGGCGACCCCGCGGC
GGGCGCGGAGATGTGGCCCCTGGTAGCGGCGCTGTTGCTGGGCTCGGCGTGCTGCG
GATCAGCTCAGCTACTATTTAATAAAACAAAATCTGTAGAATTCACGTTTTGTAATG
ACACTGTCGTCATTCCATGCTTTGTTACTAATATGGAGGCACAAAACACTACTGAAG
TATACGTAAAGTGGAAATTTAAAGGAAGAGATATTTACACCTTTGATGGAGCTCTAA
ACAAGTCCACTGTCCCCACTGACTTTAGTAGTGCAAAAATTGAAGTCTCACAATTAC
TAAAAGGAGATGCCTCTTTGAAGATGGATAAGAGTGATGCTGTCTCACACACAGGA
AACTACACTTGTGAAGTAACAGAATTAACCAGAGAAGGTGAAACGATCATCGAGCT
AAAATATCGTGTTGTTTCATGGTTTTCTCCAAATGAAAATATTCTTATTGTTATTTTC
CCAATTTTTGCTATACTCCTGTTCTGGGGACAGTTTGGTATTAAAACACTTAAATATA
GATCCGGTGGTATGGATGAGAAAACAATTGCTTTACTTGTTGCTGGACTAGTGATCA
CTGTCATTGTCATTGTTGGAGCCATTCTTTTCGTCCCAGGTGAATATTCATTAAAGAA
TGCTACTGGCCTTGGTTTAATTGTGACTTCTACAGGGATATTAATATTACTTCACTAC
TATGTGTTTAGTACAGCGATTGGATTAACCTCCTTCGTCATTGCCATATTGGTTATTC
AGGTGATAGCCTATATCCTCGCTGTGGTTGGACTGAGTCTCTGTATTGCGGCGTGTA
TACCAATGCATGGCCCTCTTCTGATTTCAGGTTTGAGTATCTTAGCTCTAGCACAATT
ACTTGGACTAGTTTATATGAAATTTGTGGCTTCCAATCAGAAGACTATACAACCTCC
TAGGAAAGCTGTAGAGGAACCCCTTAATGCATTCAAAGAATCAAAAGGAATGATGA
ATGATGAATAACTGAAGTGAAGTGATGGACTCCGATTTGGAGAGTAGTAAGACGTG
AAAGGAATACACTTGTGTTTAAGCACCATGGCCTTGATGATTCACTGTTGGGGAGAA
GAAACAAGAAAAGTAACTGGTTGTCACCTATGAGACCCTTACGTGATTGTTAGTTAA
GTTTTTATTCAAAGCAGCTGTAATTTAGTTAATAAAATAATTATGATCTATGTTGTTT
GCCCAATTGAGATCCAGTTTTTTGTTGTTATTTTTAATCAATTAGGGGCAATAGTAGA
ATGGACAATTTCCAAGAATGATGCCTTTCAGGTCCTAGGGCCTCTGGCCTCTAGGTA
ACCAGTTTAAATTGGTTCAGGGTGATAACTACTTAGCACTGCCCTGGTGATTACCCA
GAGATATCTATGAAAACCAGTGGCTTCCATCAAACCTTTGCCAACTCAGGTTCACAG
CAGCTTTGGGCAGTTATGGCAGTATGGCATTAGCTGAGAGGTGTCTGCCACTTCTGG
GTCAATGGAATAATAAATTAAGTACAGGCAGGAATTTGGTTGGGAGCATCTTGTATG
ATCTCCGTATGATGTGATATTGATGGAGATAGTGGTCCTCATTCTTGGGGGTTGCCA
TTCCCACATTCCCCCTTCAACAAACAGTGTAACAGGTCCTTCCCAGATTTAGGGTAC
TTTTATTGATGGATATGTTTTCCTTTTATTCACATAACCCCTTGAAACCCTGTCTTGTC
CTCCTGTTACTTGCTTCTGCTGTACAAGATGTAGCACCTTTTCTCCTCTTTGAACATG
GTCTAGTGACACGGTAGCACCAGTTGCAGGAAGGAGCCAGACTTGTTCTCAGAGCA
CTGTGTTCACACTTTTCAGCAAAAATAGCTATGGTTGTAACATATGTATTCCCTTCCT
CTGATTTGAAGGCAAAAATCTACAGTGTTTCTTCACTTCTTTTCTGATCTGGGGCATG
AAAAAAGCAAGATTGAAATTTGAACTATGAGTCTCCTGCATGGCAACAAAATGTGT
GTCACCATCAGGCCAACAGGCCAGCCCTTGAATGGGGATTTATTACTGTTGTATCTA
TGTTGCATGATAAACATTCATCACCTTCCTCCTGTAGTCCTGCCTCGTACTCCCCTTC
CCCTATGATTGAAAAGTAAACAAAACCCACATTTCCTATCCTGGTTAGAAGAAAATT
AATGTTCTGACAGTTGTGATCGCCTGGAGTACTTTTAGACTTTTAGCATTCGTTTTTT
ACCTGTTTGTGGATGTGTGTTTGTATGTGCATACGTATGAGATAGGCACATGCATCTT
CTGTATGGACAAAGGTGGGGTACCTACAGGAGAGCAAAGGTTAATTTTGTGCTTTTA
GTAAAAACATTTAAATACAAAGTTCTTTATTGGGTGGAATTATATTTGATGCAAATA
TTTGATCACTTAAAACTTTTAAAACTTCTAGGTAATTTGCCACGCTTTTTGACTGCTC
ACCAATACCCTGTAAAAATACGTAATTCTTCCTGTTTGTGTAATAAGATATTCATATT
TGTAGTTGCATTAATAATAGTTATTTCTTAGTCCATCAGATGTTCCCGTGTGCCTCTT
TTATGCCAAATTGATTGTCATATTTCATGTTGGGACCAAGTAGTTTGCCCATGGCAA
ACCTAAATTTATGACCTGCTGAGGCCTCTCAGAAAACTGAGCATACTAGCAAGACA
GCTCTTCTTGAAAAAAAAAATATGTATACACAAATATATACGTATATCTATATATAC
GTATGTATATACACACATGTATATTCTTCCTTGATTGTGTAGCTGTCCAAAATAATAA
CATATATAGAGGGAGCTGTATTCCTTTATACAAATCTGATGGCTCCTGCAGCACTTTT
TCCTTCTGAAAATATTTACATTTTGCTAACCTAGTTTGTTACTTTAAAAATCAGTTTT
GATGAAAGGAGGGAAAAGCAGATGGACTTGAAAAAGATCCAAGCTCCTATTAGAA
AAGGTATGAAAATCTTTATAGTAAAATTTTTTATAAACTAAAGTTGTACCTTTTAATA
TGTAGTAAACTCTCATTTATTTGGGGTTCGCTCTTGGATCTCATCCATCCATTGTGTT
CTCTTTAATGCTGCCTGCCTTTTGAGGCATTCACTGCCCTAGACAATGCCACCAGAG
ATAGTGGGGGAAATGCCAGATGAAACCAACTCTTGCTCTCACTAGTTGTCAGCTTCT
CTGGATAAGTGACCACAGAAGCAGGAGTCCTCCTGCTTGGGCATCATTGGGCCAGTT
CCTTCTCTTTAAATCAGATTTGTAATGGCTCCCAAATTCCATCACATCACATTTAAAT
TGCAGACAGTGTTTTGCACATCATGTATCTGTTTTGTCCCATAATATGCTTTTTACTC
CCTGATCCCAGTTTCTGCTGTTGACTCTTCCATTCAGTTTTATTTATTGTGTGTTCTCA
CAGTGACACCATTTGTCCTTTTCTGCAACAACCTTTCCAGCTACTTTTGCCAAATTCT
ATTTGTCTTCTCCTTCAAAACATTCTCCTTTGCAGTTCCTCTTCATCTGTGTAGCTGCT
CTTTTGTCTCTTAACTTACCATTCCTATAGTACTTTATGCATCTCTGCTTAGTTCTATT
AGTTTTTTGGCCTTGCTCTTCTCCTTGATTTTAAAATTCCTTCTATAGCTAGAGCTTTT
CTTTCTTTCATTCTCTCTTCCTGCAGTGTTTTGCATACATCAGAAGCTAGGTACATAA
GTTAAATGATTGAGAGTTGGCTGTATTTAGATTTATCACTTTTTAATAGGGTGAGCTT
GAGAGTTTTCTTTCTTTCTGTTTTTTTTTTTTGTTTTTTTTTTTTTTTTTTTTTTTTTTTTT
TTTGACTAATTTCACATGCTCTAAAAACCTTCAAAGGTGATTATTTTTCTCCTGGAAA
CTCCAGGTCCATTCTGTTTAAATCCCTAAGAATGTCAGAATTAAAATAACAGGGCTA
TCCCGTAATTGGAAATATTTCTTTTTTCAGGATGCTATAGTCAATTTAGTAAGTGACC
ACCAAATTGTTATTTGCACTAACAAAGCTCAAAACACGATAAGTTTACTCCTCCATC
TCAGTAATAAAAATTAAGCTGTAATCAACCTTCTAGGTTTCTCTTGTCTTAAAATGG
GTATTCAAAAATGGGGATCTGTGGTGTATGTATGGAAACACATACTCCTTAATTTAC
CTGTTGTTGGAAACTGGAGAAATGATTGTCGGGCAACCGTTTATTTTTTATTGTATTT
TATTTGGTTGAGGGATTTTTTTATAAACAGTTTTACTTGTGTCATATTTTAAAATTAC
TAACTGCCATCACCTGCTGGGGTCCTTTGTTAGGTCATTTTCAGTGACTAATAGGGAT
AATCCAGGTAACTTTGAAGAGATGAGCAGTGAGTGACCAGGCAGTTTTTCTGCCTTT
AGCTTTGACAGTTCTTAATTAAGATCATTGAAGACCAGCTTTCTCATAAATTTCTCTT
TTTGAAAAAAAGAAAGCATTTGTACTAAGCTCCTCTGTAAGACAACATCTTAAATCT
TAAAAGTGTTGTTATCATGACTGGTGAGAGAAGAAAACATTTTGTTTTTATTAAATG
GAGCATTATTTACAAAAAGCCATTGTTGAGAATTAGATCCCACATCGTATAAATATC
TATTAACCATTCTAAATAAAGAGAACTCCAGTGTTGCTATGTGCAAGATCCTCTCTT
GGAGCTTTTTTGCATAGCAATTAAAGGTGTGCTATTTGTCAGTAGCCATTTTTTTGCA
GTGATTTGAAGACCAAAGTTGTTTTACAGCTGTGTTACCGTTAAAGGTTTTTTTTTTT
ATATGTATTAAATCAATTTATCACTGTTTAAAGCTTTGAATATCTGCAATCTTTGCCA
AGGTACTTTTTTATTTAAAAAAAAACATAACTTTGTAAATATTACCCTGTAATATTAT
ATATACTTAATAAAACATTTTAAGCTATTTTGTTGGGCTATTTCTATTGCTGCTACAG
CAGACCACAAGCACATTTCTGAAAAATTTAATTTATTAATGTATTTTTAAGTTGCTTA
TATTCTAGGTAACAATGTAAAGAATGATTTAAAATATTAATTATGAATTTTTTGAGT
ATAATACCCAATAAGCTTTTAATTAGAGCAGAGTTTTAATTAAAAGTTTTAAATCAG
TCCAA
SEQ ID NO: 14
Homo sapiens CD47
Amino Acid
MWPLVAALLLGSACCGSAQLLFNKTKSVEFTFCNDTVVIPCFVTNMEAQNTTEVYVKW
KFKGRDIYTFDGALNKSTVPTDFSSAKIEVSQLLKGDASLKMDKSDAVSHTGNYTCEVT
ELTREGETIIELKYRVVSWFSPNENILIVIFPIFAILLFWGQFGIKTLKYRSGGMDEKTIALL
VAGLVITVIVIVGAILFVPGEYSLKNATGLGLIVTSTGILILLHYYVESTAIGLTSFVIAILVI
QVIAYILAVVGLSLCIAACIPMHGPLLISGLSILALAQLLGLVYMKFVASNQKTIQPPRKA
VEEPLNAFKESKGMMNDE
SEQ ID NO: 15
Homo sapiens CD47 gene Transcript Variant
Nucleic Acid
GCAGCCTGGGCAGTGGGTCCTGCCTGTGACGCGCGGCGGCGGTCGGTCCTGCCTGTA
ACGGCGGCGGCGGCTGCTGCTCCGGACACCTGCGGCGGCGGCGGCGACCCCGCGGC
GGGCGCGGAGATGTGGCCCCTGGTAGCGGCGCTGTTGCTGGGCTCGGCGTGCTGCG
GATCAGCTCAGCTACTATTTAATAAAACAAAATCTGTAGAATTCACGTTTTGTAATG
ACACTGTCGTCATTCCATGCTTTGTTACTAATATGGAGGCACAAAACACTACTGAAG
TATACGTAAAGTGGAAATTTAAAGGAAGAGATATTTACACCTTTGATGGAGCTCTAA
ACAAGTCCACTGTCCCCACTGACTTTAGTAGTGCAAAAATTGAAGTCTCACAATTAC
TAAAAGGAGATGCCTCTTTGAAGATGGATAAGAGTGATGCTGTCTCACACACAGGA
AACTACACTTGTGAAGTAACAGAATTAACCAGAGAAGGTGAAACGATCATCGAGCT
AAAATATCGTGTTGTTTCATGGTTTTCTCCAAATGAAAATATTCTTATTGTTATTTTC
CCAATTTTTGCTATACTCCTGTTCTGGGGACAGTTTGGTATTAAAACACTTAAATATA
GATCCGGTGGTATGGATGAGAAAACAATTGCTTTACTTGTTGCTGGACTAGTGATCA
CTGTCATTGTCATTGTTGGAGCCATTCTTTTCGTCCCAGGTGAATATTCATTAAAGAA
TGCTACTGGCCTTGGTTTAATTGTGACTTCTACAGGGATATTAATATTACTTCACTAC
TATGTGTTTAGTACAGCGATTGGATTAACCTCCTTCGTCATTGCCATATTGGTTATTC
AGGTGATAGCCTATATCCTCGCTGTGGTTGGACTGAGTCTCTGTATTGCGGCGTGTA
TACCAATGCATGGCCCTCTTCTGATTTCAGGTTTGAGTATCTTAGCTCTAGCACAATT
ACTTGGACTAGTTTATATGAAATTTGTGGAATAACTGAAGTGAAGTGATGGACTCCG
ATTTGGAGAGTAGTAAGACGTGAAAGGAATACACTTGTGTTTAAGCACCATGGCCTT
GATGATTCACTGTTGGGGAGAAGAAACAAGAAAAGTAACTGGTTGTCACCTATGAG
ACCCTTACGTGATTGTTAGTTAAGTTTTTATTCAAAGCAGCTGTAATTTAGTTAATAA
AATAATTATGATCTATGTTGTTTGCCCAATTGAGATCCAGTTTTTTGTTGTTATTTTTA
ATCAATTAGGGGCAATAGTAGAATGGACAATTTCCAAGAATGATGCCTTTCAGGTCC
TAGGGCCTCTGGCCTCTAGGTAACCAGTTTAAATTGGTTCAGGGTGATAACTACTTA
GCACTGCCCTGGTGATTACCCAGAGATATCTATGAAAACCAGTGGCTTCCATCAAAC
CTTTGCCAACTCAGGTTCACAGCAGCTTTGGGCAGTTATGGCAGTATGGCATTAGCT
GAGAGGTGTCTGCCACTTCTGGGTCAATGGAATAATAAATTAAGTACAGGCAGGAA
TTTGGTTGGGAGCATCTTGTATGATCTCCGTATGATGTGATATTGATGGAGATAGTG
GTCCTCATTCTTGGGGGTTGCCATTCCCACATTCCCCCTTCAACAAACAGTGTAACA
GGTCCTTCCCAGATTTAGGGTACTTTTATTGATGGATATGTTTTCCTTTTATTCACAT
AACCCCTTGAAACCCTGTCTTGTCCTCCTGTTACTTGCTTCTGCTGTACAAGATGTAG
CACCTTTTCTCCTCTTTGAACATGGTCTAGTGACACGGTAGCACCAGTTGCAGGAAG
GAGCCAGACTTGTTCTCAGAGCACTGTGTTCACACTTTTCAGCAAAAATAGCTATGG
TTGTAACATATGTATTCCCTTCCTCTGATTTGAAGGCAAAAATCTACAGTGTTTCTTC
ACTTCTTTTCTGATCTGGGGCATGAAAAAAGCAAGATTGAAATTTGAACTATGAGTC
TCCTGCATGGCAACAAAATGTGTGTCACCATCAGGCCAACAGGCCAGCCCTTGAAT
GGGGATTTATTACTGTTGTATCTATGTTGCATGATAAACATTCATCACCTTCCTCCTG
TAGTCCTGCCTCGTACTCCCCTTCCCCTATGATTGAAAAGTAAACAAAACCCACATT
TCCTATCCTGGTTAGAAGAAAATTAATGTTCTGACAGTTGTGATCGCCTGGAGTACT
TTTAGACTTTTAGCATTCGTTTTTTACCTGTTTGTGGATGTGTGTTTGTATGTGCATAC
GTATGAGATAGGCACATGCATCTTCTGTATGGACAAAGGTGGGGTACCTACAGGAG
AGCAAAGGTTAATTTTGTGCTTTTAGTAAAAACATTTAAATACAAAGTTCTTTATTG
GGTGGAATTATATTTGATGCAAATATTTGATCACTTAAAACTTTTAAAACTTCTAGGT
AATTTGCCACGCTTTTTGACTGCTCACCAATACCCTGTAAAAATACGTAATTCTTCCT
GTTTGTGTAATAAGATATTCATATTTGTAGTTGCATTAATAATAGTTATTTCTTAGTC
CATCAGATGTTCCCGTGTGCCTCTTTTATGCCAAATTGATTGTCATATTTCATGTTGG
GACCAAGTAGTTTGCCCATGGCAAACCTAAATTTATGACCTGCTGAGGCCTCTCAGA
AAACTGAGCATACTAGCAAGACAGCTCTTCTTGAAAAAAAAAATATGTATACACAA
ATATATACGTATATCTATATATACGTATGTATATACACACATGTATATTCTTCCTTGA
TTGTGTAGCTGTCCAAAATAATAACATATATAGAGGGAGCTGTATTCCTTTATACAA
ATCTGATGGCTCCTGCAGCACTTTTTCCTTCTGAAAATATTTACATTTTGCTAACCTA
GTTTGTTACTTTAAAAATCAGTTTTGATGAAAGGAGGGAAAAGCAGATGGACTTGA
AAAAGATCCAAGCTCCTATTAGAAAAGGTATGAAAATCTTTATAGTAAAATTTTTTA
TAAACTAAAGTTGTACCTTTTAATATGTAGTAAACTCTCATTTATTTGGGGTTCGCTC
TTGGATCTCATCCATCCATTGTGTTCTCTTTAATGCTGCCTGCCTTTTGAGGCATTCA
CTGCCCTAGACAATGCCACCAGAGATAGTGGGGGAAATGCCAGATGAAACCAACTC
TTGCTCTCACTAGTTGTCAGCTTCTCTGGATAAGTGACCACAGAAGCAGGAGTCCTC
CTGCTTGGGCATCATTGGGCCAGTTCCTTCTCTTTAAATCAGATTTGTAATGGCTCCC
AAATTCCATCACATCACATTTAAATTGCAGACAGTGTTTTGCACATCATGTATCTGTT
TTGTCCCATAATATGCTTTTTACTCCCTGATCCCAGTTTCTGCTGTTGACTCTTCCATT
CAGTTTTATTTATTGTGTGTTCTCACAGTGACCCATTTGTCCTTTTCTGCAACAACCTT
TCCAGCTACTTTTGCCAAATTCTATTTGTCTTCTCCTTCAAAACATTCTCCTTTGCAGT
TCCTCTTCATCTGTGTAGCTGCTCTTTTGTCTCTTAACTTACCATTCCTATAGTACTTT
ATGCATCTCTGCTTAGTTCTATTAGTTTTTTGGCCTTGCTCTTCTCCTTGATTTTAAAA
TTCCTTCTATAGCTAGAGCTTTTCTTTCTTTCATTCTCTCTTCCTGCAGTGTTTTGCAT
ACATCAGAAGCTAGGTACATAAGTTAAATGATTGAGAGTTGGCTGTATTTAGATTTA
TCACTTTTTAATAGGGTGAGCTTGAGAGTTTTCTTTCTTTCTGTTTTTTTTTTTTGTTTT
TTTTTTTTTTTTTTTTTTTTTTTTTTTTGACTAATTTCACATGCTCTAAAAACCTTCAAA
GGTGATTATTTTTCTCCTGGAAACTCCAGGTCCATTCTGTTTAAATCCCTAAGAATGT
CAGAATTAAAATAACAGGGCTATCCCGTAATTGGAAATATTTCTTTTTTCAGGATGC
TATAGTCAATTTAGTAAGTGACCACCAAATTGTTATTTGCACTAACAAAGCTCAAAA
CACGATAAGTTTACTCCTCCATCTCAGTAATAAAAATTAAGCTGTAATCAACCTTCT
AGGTTTCTCTTGTCTTAAAATGGGTATTCAAAAATGGGGATCTGTGGTGTATGTATG
GAAACACATACTCCTTAATTTACCTGTTGTTGGAAACTGGAGAAATGATTGTCGGGC
AACCGTTTATTTTTTATTGTATTTTATTTGGTTGAGGGATTTTTTTATAAACAGTTTTA
CTTGTGTCATATTTTAAAATTACTAACTGCCATCACCTGCTGGGGTCCTTTGTTAGGT
CATTTTCAGTGACTAATAGGGATAATCCAGGTAACTTTGAAGAGATGAGCAGTGAGT
GACCAGGCAGTTTTTCTGCCTTTAGCTTTGACAGTTCTTAATTAAGATCATTGAAGAC
CAGCTTTCTCATAAATTTCTCTTTTTGAAAAAAAGAAAGCATTTGTACTAAGCTCCTC
TGTAAGACAACATCTTAAATCTTAAAAGTGTTGTTATCATGACTGGTGAGAGAAGAA
AACATTTTGTTTTTATTAAATGGAGCATTATTTACAAAAAGCCATTGTTGAGAATTA
GATCCCACATCGTATAAATATCTATTAACCATTCTAAATAAAGAGAACTCCAGTGTT
GCTATGTGCAAGATCCTCTCTTGGAGCTTTTTTGCATAGCAATTAAAGGTGTGCTATT
TGTCAGTAGCCATTTTTTTGCAGTGATTTGAAGACCAAAGTTGTTTTACAGCTGTGTT
ACCGTTAAAGGTTTTTTTTTTTATATGTATTAAATCAATTTATCACTGTTTAAAGCTTT
GAATATCTGCAATCTTTGCCAAGGTACTTTTTTATTTAAAAAAAAACATAACTTTGTA
AATATTACCCTGTAATATTATATATACTTAATAAAACATTTTAAGCTATTTTGTTGGG
CTATTTCTATTGCTGCTACAGCAGACCACAAGCACATTTCTGAAAAATTTAATTTATT
AATGTATTTTTAAGTTGCTTATATTCTAGGTAACAATGTAAAGAATGATTTAAAATA
TTAATTATGAATTTTTTGAGTATAATACCCAATAAGCTTTTAATTAGAGCAGAGTTTT
AATTAAAAGTTTTAAATCAGTCCAA
SEQ ID NO: 16
Homo sapiens CD47 isoform x1 protein
Amino Acid
MWPLVAALLLGSACCGSAQLLFNKTKSVEFTFCNDTVVIPCFVTNMEAQNTTEVYVKW
KFKGRDIYTFDGALNKSTVPTDFSSAKIEVSQLLKGDASLKMDKSDAVSHTGNYTCEVT
ELTREGETIIELKYRVVSWFSPNENILIVIFPIFAILLFWGQFGIKTLKYRSGGMDEKTIALL
VAGLVITVIVIVGAILFVPGEYSLKNATGLGLIVTSTGILILLHYYVESTAIGLTSFVIAILVI
QVIAYILAVVGLSLCIAACIPMHGPLLISGLSILALAQLLGLVYMK FVE
SEQ ID NO: 17
Homo sapiens Inhibin beta A subunit (Activin A)
amino acid sequence:
GLECDGKVNICCKKQFFVSFKDIGWNDWIIAPSGYHANYCEGECPSHIAGTSGSSLSFHS
TVINHYRMRGHSPFANLKSCCVPTKLRPMSMLYYDDGQNIIKKDIQNMIVEECGCS
SEQ ID NO: 18
Homo sapiens Inhibin beta A chain (Activin A)
nucleic acid sequence:
GGCTTGGAGTGTGATGGCAAGGTCAACATCTGCTGTAAGAAACAGTTCTTTGTCAGT
TTCAAGGACATCGGCTGGAATGACTGGATCATTGCTCCCTCTGGCTATCATGCCAAC
TACTGCGAGGGTGAGTGCCCGAGCCATATAGCAGGCACGTCCGGGTCCTCACTGTCC
TTCCACTCAACAGTCATCAACCACTACCGCATGCGGGGCCATAGCCCCTTTGCCAAC
CTCAAATCGTGCTGTGTGCCCACCAAGCTGAGACCCATGTCCATGTTGTACTATGAT
GATGGTCAAAACATCATCAAAAAGGACATTCAGAACATGATCGTGGAGGAGTGTGG
GTGCTCATAG
SEQ ID NO: 19
Homo sapiens Erythroid differentiation protein (EDF) ovarian
amino acid sequence:
MPLLWLRGFLLASCWIIVRSSPTPGSEGHSAAPDCPSCALAALPKDVPNSQPEMVEAVK
KHILNMLHLKKRPDVTQPVPKAALLNAIRKLHVGKVGENGYVEIEDDIGRRAEMNELM
EQTSEIITFAESGTARKTLHFEISKEGSDLSVVERAEVWLFLKVPKANRTRTKVTIRLFQQ
QKHPQGSLDTGEEAEEVGLKGERSELLLSEKVVDARKSTWHVFPVSSSIQRLLDQGKSS
LDVRIACEQCQESGASLVLLGKKKKKEEEGEGKKKGGGEGGAGADEEKEQSHRPFLML
QARQSEDHPHRRRRRGLECDGKVNICCKKQFFVSFKDIGWNDWIIAPSGYHANYCEGE
CPSHIAGTSGSSLSFHSTVINHYRMRGHSPFANLKSCCVPTKLRPMSMLYYDDGQNIIKK
DIQNMIVEECGCS
SEQ ID NO: 20
Homo sapiens Inhibin B subunit
amino acid sequence:
ARQSEDHPHRRRRRGLECDGKVNICCKKQFFVSFKDIGWNDWIIAPSGYHANYCEGECP
SHIAGTSGSSLSFHSTVINHYRMRGHSPFANLKSCCVPTKLRPMSMLYYDDGQNIIKKDI
QNMIVEECGCS
SEQ ID NO: 21
Homo sapiens Inhibin B subunit in testis
amino acid sequence:
GLECDGKVNICCKKQFFVSFKDIGWNDWIIAPSGYHANYCEGECPSHIAGTSGSSLSFHS
TVINHYACGHSPFANLKSCCVPTKLRPMSMLYYDDGQNIIKKDIQNMIVEECGCS
SEQ ID NO: 22
Homo sapiens Inhibin B subunit erythroid differentiation protein (EDF),
amino acid sequence:
MPLLWLRGFLLASCWIIVRSSPTPGSEGHSAAPDCPSCALAALPKDVPNSQPEMVEAVK
KHILNMLHLKKRPDVTQPVPKAALLNAIRKLHVGKVGENGYVEIEDDIGRRAEMNELM
EQTSEIITFAESGTARKTLHFEISKEGSDLSVVERAEVWLFLKVPKANRTRTKVTIRLFQQ
QKHPQGSLDTGEEAEEVGLKGERSELLLSEKVVDARKSTWHVFPVSSSIQRLLDQGKSS
LDVRIACEQCQESGASLVLLGKKKKKEEEGEGKKKGGGEGGAGADEEKEQSHRPFLML
QARQSEDHPHRRRRRGLECDGKVNICCKKQFFVSFKDIGWNDWIIAPSGYHANYCEGE
CPSHIAGTSGSSLSFHSTVINHYRMRGHSPFANLKSCCVPTKLRPMSMLYYDDGQNIIKK
DIQNMIVEECGCS
SEQ ID NO: 23
Mus musculus (Mouse) Inhibin beta A chain (Activin beta-A chain)
amino acid sequence:
MPLLWLRGFLLASCWIIVRSSPTPGSEGHGSAPDCPSCALATLPKDGPNSQPEMVEAVK
KHILNMLHLKKRPDVTQPVPKAALLNAIRKLHVGKVGENGYVEIEDDIGRRAEMNELM
EQTSEIITFAESGTARKTLHFEISKEGSDLSVVERAEVWLFLKVPKANRTRTKVTIRLFQQ
QKHPQGSLDTGDEAEEMGLKGERSELLLSEKVVDARKSTWHIFPVSSSIQRLLDQGKSS
LDVRIACEQCQESGASLVLLGKKKKKEVDGDGKKKDGSDGGLEEEKEQSHRPFLMLQA
RQSEDHPHRRRRRGLECDGKVNICCKKQFFVSFKDIGWNDWIIAPSGYHANYCEGECPS
HIAGTSGSSLSFHSTVINHYRMRGHSPFANLKSCCVPTKLRPMSMLYYDDGQNIIKKDIQ
NMIVEECGCS
SEQ ID NO: 24
Rattus norvegicus (Rat) Inhibin beta A chain (Activin beta-A chain)
amino acid sequence:
MPLLWLRGFLLASCWIIVRSSPTPGSEGHGAAPDCPSCALATLPKDGPNSQPEMVEAVK
KHILNMLHLKKRPDVTQPVPKAALLNAIRKLHVGKVGENGYVEIEDDIGRRAEMNELM
EQTSEIITFAESGTARKTLHFEISKEGSDLSVVERAEVWLFLKVPKANRTRTKVTIRLFQQ
QKHPQGSLDMGDEAEEMGLKGERSELLLSEKVVDARKSTWHIFPVSSSIQRLLDQGKSS
LDVRIACEQCQESGASLVLLGKKKKKEVDGDGKKKDGSDGGLEEEKEQSHRPFLMLQA
RQSEDHPHRRRRRGLECDGKVNICCKKQFFVSFKDIGWNDWIIAPSGYHANYCEGECPS
HIAGTSGSSLSFHSTVINHYRMRGHSPFANLKSCCVPTKLRPMSMLYYDDGQNIIKKDIQ
NMIVEECGCS
SEQ ID NO: 25
Gallus gallus (Chicken) Inhibin beta A chain (Activin beta-A chain)
amino acid sequence:
MPLLWKRGFLLVICWIIVRSSPTPGSEGHSSVADCPSCALTTLSKDVPSSQPEMVEAVKK
HILNMLHLRDRPNITQPVPKAALLNATKKLHVGKVGDDGYVEIEDDVGRRAEMNEVVE
QTSEIITFAESGTPKKTLHFEISKEGSELSVVEHAEVWLFLKVSKANRSRTKVTIRLFQQQ
RQPKGNSEAAEDMEDMGLKGERSETLISEKAVDARKSTWHIFPISSSVQRLLDQGQSSL
DVRIACDLCQETGASLVLLGKKKKKEDDGEGKEKDGGELTGEEEKEQSHRPFLMMLAR
HSEDRQHRRRERGLECDGKVNICCKKQFFVSFKDIGWSDWIIAPTGYHANYCEEECPSHI
AGTSGSSLSFHSTVINHYRMRGHSPFANLKSCCVPTKLRPMSMLYYDDGQNIIKKDIQN
MIVEECGCS
SEQ ID NO: 26
Bos taurus (Bovine) Inhibin beta A chain (Activin beta-A chain)
amino acid sequence:
MPLLWLRGFLLASCWIIVRSSPTPGSEGHSAAPDCPSCALATLPKDVPNSQPEMVEAVK
KHILNMLHLKKRPDVTQPVPKAALLNAIRKLHVGKVGENGYVEIEDDIGRRAEMNELM
EQTSEIITFAESGTARKTLHFEISKEGSDLSVVERAEIWLFLKVPKANRTRSKVTIRLFQQQ
KHLQGSLDAGEEAEEVGLKGEKSEMLISEKVVDARKSTWHIFPVSSCIQRLLDQGKSSL
DIRIACEQCQETGASLVLLGKKKKKEEEGEGKKRDGEGGAGGDEEKEQSHRPFLMLQA
RQSEDHPHRRRRRGLECDGKVNICCKKQFFVSFKDIGWNDWIIAPSGYHANYCEGECPS
HIAGTSGSSLSFHSTVINHYRMRGHSPFANLKSCCVPTKLRPMSMLYYDDGQNIIKKDIQ
NMIVEECGCS
SEQ ID NO: 27
Equus caballus (Horse) Inhibin beta A chain (Activin beta-A chain)
amino acid sequence:
MPLLWLRGFLLASCWIIVKSSPTPGSEGHSAAPNCPSCALATLPKDVPNAQPEMVEAVK
KHILNMLHLKKRPDVTQPVPKAALLNAIRKLHVGKVGENGYVEIEDDIGRRAEMNELM
EQTSEIITFAESGTARKTLHFEISKEGSDLSVVERAEVWLFLKVPKANRTRSKVTIRLLQQ
QKHPQGSSDTREEAEEADLMEERSEQLISEKVVDARKSTWHIFPVSSSIQRLLDQGKSSL
DIRIACDQCHETGASLVLLGKKKKKEEEGEGKKKDGGEAGAGVDEEKEQSHRPFLMLQ
ARQSEDHPHRRRRRGLECDGKVNICCKKQFFVSFKDIGWNDWIIAPSGYHANYCEGECP
SHIAGTSGSSLSFHSTVINQYRLRGHNPFANLKSCCVPTKLRPMSMLYYDDGQNIIKKDI
QNMIVEECGCS
SEQ ID NO: 28
Sus scrofa (Pig) Inhibin beta A chain (Activin beta-A chain)
amino acid sequence:
MPLLWLRGFLLASCWIIVRSSPTPGSGGHSAAPDCPSCALATLPKDVPNSQPEMVEAVK
KHILNMLHLKKRPDVTQPVPKAALLNAIRKLHVGKVGENGYVELEDDIGRRAEMNELM
EQTSEIITFAEAGTARKTLRFEISKEGSDLSVVERAEIWLFLKVPKANRTRTKVSIRLFQQ
QRRPQGSADAGEEAEDVGFPEEKSEVLISEKVVDARKSTWHIFPVSSSIQRLLDQGKSAL
DIRTACEQCHETGASLVLLGKKKKKEEEAEGRKRDGEGAGVDEEKEQSHRPFLMLQAR
QSEEHPHRRRRRGLECDGKVNICCKKQFFVSFKDIGWNDWIIAPSGYHANYCEGECPSHI
AGTSGSSLSFHSTVINHYRMRGHSPFANLKSCCVPTKLRPMSMLYYDDGQNIIKKDIQN
MIVEECGCS
SEQ ID NO: 29
Ovis aries (Sheep) Inhibin beta A chain (Activin beta-A chain)
amino acid sequence:
MPLLWLRGFLLASCWIIVRSSPTPGSEGHSAAPDCPSCALATLPKDVPNSQPEMVEAVK
KHILNMLHLKKRPDVTQPVPKAALLNAIRKLHVGKVGENGYVEIEDDIGRRAEMNELM
EQTSEIITFAESGTARKTLHFEISQEGSDLSVVERAEIWLFLKVPKANRTRSKVTIRLFQQQ
KHLQGSLDAGEEAEEVGLKGEKSEMLISEKVVDARKSTWHIFPVSSCIQRLLDQGKSSL
DIRIACEQCQETGASLVLLGKKKRKEEEGEGKKRDGEGGAGGDEEKEQSHRPFLMLQA
RQSEDHPHRRRRRGLECDGKVNICCKKQFYVSFKDIGWNDWIIAPSGYHANYCEGECPS
HIAGTSGSSLSFHSTVINHYRMRGHSPFANLKSCCVPTKLRPMSMLYYDDGQNIIKKDIQ
NMIVEECGCS
SEQ ID NO: 30
Felis catus (cat) Inhibin beta A chain (Activin beta-A chain)
amino acid sequence:
MPLLWLRGFLLASCWIIVRSSPTPGSEGPGAAPDCPSCALATLPKDVPNSQPEMVEAVK
KHILNMLHLKKRPEVTQPVPKAALLNAIRKLHVGKVGENGYVEIEDDIGRRAEMNELM
EQTSEIITFAESGTARKTLHFEISKEGSDLSVVERAEVWLFLKVPKANRTRTKVTIQLLQK
QPQGGVDAGEEAEEMGLMEERNEVLISEKVVDARKSTWHIFPVSSSIQRLLDQGKSSLD
VRIACEQCHETGASLVLLGKKKKKEEEGEGKKKDGGDGGAGADEDKEQSHRPFLMLQ
ARQSEDHPHRRRRRGLECDGKVNICCKKQFFVSFKDIGWNDWIIAPSGYHANYCEGECP
SHIAGTSGSSLSFHSTVINHYRMRGHSPFANLKSCCVPTKLRPMSMLYYDDGQNIIKKDI
QNMIVEECGCS
SEQ ID NO: 31
Danio rerio (zebrafish) Inhibin beta A chain (Activin beta-A chain)
amino acid sequence:
MSPLPLLSGILLLLIRSCSLSAMVTKGSLPMSEQQAGATVCPSCALARFRKGVSESEDEG
AQQDVVEAVKRHILNMLHLQERPNITHPVPRAALLNAIRKVHVGRVAKDGSVLIEDEAS
NRAETEQAEQTEIITFAETGEAPGIVNFLISKEGGEMSVVDQANVWIFLRLPKGNRTRAN
VNIRLLLQQGAGEKILAEKSVDTRRSGWHTFPASESVQSLLQRGGSTLSLRVSCPLCADA
RATPVLVSPGGSEREQSHRPFLMAVVRQMDELSLRRRRKRGLECDGKARVCCKRQFYV
NFKDIGWNDWIIAPSGYHANYCEGDCASNVASITGNSLSFHSTVISHYRIRGYSPFTNIKS
CCVPTRLRAMSMLYYNEEQKIVKKDIQNMIVEECGCS
SEQ ID NO: 32
Carassius auratus (goldfish) Inhibin beta A chain (Activin beta-A chain)
amino acid sequence:
MSSLTLVNRGTAALRLFVRGLLTHSSREWLSGDGEPDDPVTPCPSCALAQRQKDSEEQT
DMVEAVKRHILNMLHLNTRPNVTHPVPRAALLNAIRRLHVGRVGEDGTVEMEEDGGG
LGEHREQSEEQPFEIITFAEPGDAPDIMKFDISMEGNTLSVVEQANVWLLLKVAKGSRGK
GKVSVQLLQHGKADPGSADGPQEAVVSEKTVDTRRSGWHTLPVSRTVQTLLDGDSSM
LSLRVSCPMCAEAGAVPILVPTESNKGKEREQSHRPFLMVVLKPAEEHPHRRSKRGLEC
DGKIRVCCKRQFYVNFKDIGWSDWIIAPSGYHANYCEGDCPSHVASITGSALSFHSTVIN
HYRMRGYSPFNNIKSCCVPTRLRAMSMLYYNEEQKIIKKDIQNMIVEECGCS
SEQ ID NO: 33
Recombinant Inhibin B subunit
nucleic acid sequence
GCCCGGCAGTCTGAAGACCACCCTCATCGCCGGCGTCGGCGGGGCTTGGAGTGTGA
TGGCAAGGTCAACATCTGCTGTAAGAAACAGTTCTTTGTCAGTTTCAAGGACATCGG
CTGGAATGACTGGATCATTGCTCCCTCTGGCTATCATGCCAACTACTGCGAGGGTGA
GTGCCCGAGCCATATAGCAGGCACGTCCGGGTCCTCACTGTCCTTCCACTCAACAGT
CATCAACCACTACCGCATGCGGGGCCATAGCCCCTTTGCCAACCTCAAATCGTGCTG
TGTGCCCACCAAGCTGAGACCCATGTCCATGTTGTACTATGATGATGGTCAAAACAT
CATCAAAAAGGACATTCAGAACATGATCGTGGAGGAGTGTGGGTGCTCATAGAGTT
GCCCAGCCCAGGGGGAAAGGGAGCAAGA
SEQ ID NO: 34
Homo sapiens mature subunit beta(A) inhibin in testis
nucleic acid sequence
GGCCTGGAGTGCGACGGCAAGGTCAACATCTGCTGTAAGAAACAGTTCTTTGTCAGT
TTCAAGGACATCGGCTGGAATGACTGGATCATTGCTCCCTCTGGCTATCATGCCAAC
TACTGCGAGGGTGAGTGCCCGAGCCATATAGCAGGCACGTCCGGGTCCTCACTGTCC
TTCCACTCAACAGTCATCAACCACTACGCATGCGGCCATAGCCCCTTTGCCAACCTC
AAATCGTGCTGTGTGCCCACCAAGCTGAGACCCATGTCCATGTTGTACTATGATGAT
GGTCAAAACATCATCAAAAAGGACATTCAGAACATGATCGTGGAGGAGTGCGGGTG
CTCCTAA
SEQ ID NO: 35
Homo Sapiens Renalase Clone
Nucleic Acid
GCGGATCGCTGCTCCCTCTCGCCATGGCGCAGGTGCTGATCGTGGGCGCCGGGATGA
CAGGAAGCTTGTGCGCTGCGCTGCTGAGGAGGCAGACGTCCGGTCCCTTGTACCTTG
CTGTGTGGGACAAGGCTGACGACTCAGGGGGAAGAATGACTACAGCCTGCAGTCCT
CATAATCCTCAGTGCACAGCTGACTTGGGTGCTCAGTACATCACCTGCACTCCTCAT
TATGCCAAAAAACACCAACGTTTTTATGATGAACTGTTAGCCTATGGCGTTTTGAGG
CCTCTAAGCTCGCCTATTGAAGGAATGGTGATGAAAGAAGGAGACTGTAACTTTGTG
GCACCTCAAGGAATTTCTTCAATTATTAAGCATTACTTGAAAGAATCAGGTGCAGAA
GTCTACTTCAGACATCGTGTGACACAGATCAACCTAAGAGATGACAAATGGGAAGT
ATCCAAACAAACAGGCTCCCCTGAGCAGTTTGATCTTATTGTTCTCACAATGCCAGT
TCCTGAGATTCTGCAGCTTCAAGGTGACATCACCACCTTAATTAGTGAATGCCAAAG
GCAGCAACTGGAGGCTGTGAGCTACTCCTCTCGATATGCTCTGGGCCTCTTTTATGA
AGCTGGTACGAAGATTGATGTCCCTTGGGCTGGGCAGTACATCACCAGTAATCCCTG
CATACGCTTCGTCTCCATTGATAATAAGAAGCGCAATATAGAGTCATCAGAAATTGG
GCCTTCCCTCGTGATTCACACCACTGTCCCATTTGGAGTTACATACTTGGAACACAG
CATTGAGGATGTGCAAGAGTTAGTCTTCCAGCAGCTGGAAAACATTTTGCCGGGTTT
GCCTCAGCCAATTGCTACCAAATGCCAAAAATGGAGACATTCACAGGTTACAAATG
CTGCTGCCAACTGTCCTGGCCAAATGACTCTGCATCACAAACCTTTCCTTGCATGTG
GAGGGGATGGATTTACTCAGTCCAACTTTGATGGCTGCATCACTTCTGCCCTATGTG
TTCTGGAAGCTTTAAAGAATTATATTTAGTGCCTATATCCTTATTCTCTATATGTGTA
TTGGGTTTTTATTTTCACAATTTTCTGTTATTGATTATTTTGTTTTCTATTTTGCTAAG
AAAAATTACTGGAAAATTGTTCTTCACTTATTATCATTTTTCATGTGGAGTATAAAAT
CAATTTTGTAATTTTGATAGTTACAACCCATGCTAGAATGGAAATTCCTCACACCTTG
CACCTTCCCTACTTTTCTGAATTGCTATGACTACTCCTTGTTGGAGGAAAAGTGGTAC
TTAAAAAATAACAAACGACTCTCTCAAAAAAATTACATTAAATCACAATAACAGTTT
GTATGCCAAAAACTTGATTATCCTTATGAAAATTTCAATTCTGAATAAAGAATAATC
ACATTATCAAAGCCCCATCAAAAAAAAAAAAAAAAAAAAAAAAAAAAAA
SEQ ID NO: 36
Homo Sapiens Renalase Clone
Nucleic acids
AAAAGCCCGGGCCGAACGGCCCCGCCGCAGAGACTCAGCGCGGATCGCTGCTCCCT
CTCGCCATGGCGCAGGTGCTGATCGTGGGCGCCGGGATGACAGGAAGCTTGTGCGC
TGCGCTGCTGAGGAGGCAGACGTCCGGTCCCTTGTACCTTGCTGTGTGGGACAAGGC
TGAGGACTCAGGGGGAAGAATGACTACAGCCTGCAGTCCTCATAATCCTCAGTGCA
CAGCTGACTTGGGTGCTCAGTACATCACCTGCACTCCTCATTATGCCAAAAAACACC
AACGTTTTTATGATGAACTGTTAGCCTATGGCGTTTTGAGGCCTCTAAGCTCGCCTAT
TGAAGGAATGGTGATGAAAGAAGGAGACTGTAACTTTGTGGCACCTCAAGGAATTT
CTTCAATTATTAAGCATTACTTGAAAGAATCAGGTGCAGAAGTCTACTTCAGACATC
GTGTGACACAGATCAACCTAAGAGATGACAAATGGGAAGTATCCAAACAAACAGGC
TCCCCTGAGCAGTTTGATCTTATTGTTCTCACAATGCCAGTTCCTGAGATTCTGCAGC
TTCAAGGTGACATCACCACCTTAATTAGTGAATGCCAAAGGCAGCAACTGGAGGCT
GTGAGCTACTCCTCTCGATATGCTCTGGGCCTCTTTTATGAAGCTGGTACGAAGATT
GATGTCCCTTGGGCTGGGCAGTACATCACCAGTAATCCCTGCATACGCTTCGTCTCC
ATTGATAATAAGAAGCGCAATATAGAGTCATCAGAAATTGGGCCTTCCCTCGTGATT
CACACCACTGTCCCATTTGGAGTTACATACTTGGAACACAGCATTGAGGATGTGCAA
GAGTTAGTCTTCCAGCAGCTGGAAAACATTTTGCCGGGTTTGCCTCAGCCAATTGCT
ACCAAATGCCAAAAATGGAGACATTCACAGGTACCAAGTGCTGGTGTGATTCTAGG
ATGTGCGAAGAGCCCCTGGATGATGGCGATTGGATTTCCCATCTGACTTCCTGGAAA
TTGGAGCACACAGTCAGGTTTTATTTGATTTTTTTTTTTAAGGATACCACTTCACAGC
CTTTAGGATAGCTATTATTTAGAAGCAAAACAGAAGATAAATGTTGGCAAGGATGT
GGAGATATTGGATTCCCTTGTGCAGTGCCGGTGGGAATGTAAAATGATGTAGCTACT
ATGGAAAATGATACGGCAATTTCTTTAGAAATGAAATATAGAATTGCCGTATGATCT
GCAGTTCCACATCTGGATATCTATCCAAAAGAAGTGAAAGTAGGGACTTGAACGAA
CATTTGTACACCAATGTTCACAGCGGCTTTATTCACAACAGCCAAAAGGTGGAAGCA
ACCCAGTGTCCATGGATAGATGAATAGATAAATAAAATGTGGTATAAACATACAAT
GGGCTATTGTTTAGCCTTAAAAGGGAAGGAAATTCTGACATGCTGCAATATGGATGA
AGCTTAAAGTCATTATGCAAAGTGGAATAAGCCTATCACAAAAAATAATATTACAT
AATTCTACTTATATGAGGAATCTAGAGCAGTCAGTTTCACAGAGACAGAAAATAGA
ATGGTGGTTGCCAAGGGCTGGGAGAAGAGGGCAATGGAGAGTGAGTGTTTAGTGGG
TCAGAGTTTTAGTTTGGGAAGGTAAAAAGTTCTGGAGATGGATGATGGTTATGGGTG
CTCAACAGTGTGAATGTACTTAATGCCACAGAACTGCACATTTAAATGTGGTTAAAA
TCATCACTTTTATGTTATGTATATTTACCACAATAAATAAAGAAGTTGATATTTCTTA
TACTTACAAAGAGGAGAAGGGCATTTGCAAATCAACAAGAAGTGTGAGGCCCCTCT
CTCTAGCAGAAAAATAGACTAAATCTATTTCTTTATCTTTTAACATCCTGTTTAAGGG
AAATGCCAAAACAAATGGGAAAAAATACACACACACAAATATATATGAACATGTTT
TGCCTCATGAGTAATCAAAATGTGTACATATGTATGTTTATGTATGTGTGTTTATATT
TAAAATCGTGTTCTGCCTTATGAGTAAACAAAAAGTATACAAATTAAAAACTATAAT
GAAACGT
SEQ ID NO: 37
Human KGF amino acid sequence (GenBank Accession AAB21431;)
MHKWILTWILPTLLYRSCFHIICLVGTISLACNDMTPEQMATNVNCSSPERHTRSYDYME
GGDIRVRRLFCRTQWYLRIDKRGKVKGTQEMKNNYNIMEIRTVAVGIVAIKGVESEFYL
AMNKEGKLYAKKECNEDCNFKELILENHYNTYASAKWTHNGGEMFVALNQKGIPVRG
KKTKKEQKTAHFLPMAIT
SEQ ID NO: 38
Human betacellulin amino acid sequence (GenBank: AAB25452.1)
MDRAARCSGASSLPLLLALALGLVILHCVVADGNSTRSPETNGLLCGDPEENCAATTTQ
SKRKGHFSRCPKQYKHYCIKGRCRFVVAEQTPSCVCDEGYIGARCERVDLFYLRGDRG
QILVICLIAVMVVFIILVIGVCTCCHPLRKRRKRKKKEEEMETLGKDITPINEDIEETN
SEQ ID NO: 39
Homo sapiens epidermal growth factor (WT) (GenBank: AAS83395.1)
NSDSECPLSHDGYCLHDGVCMYIEALDKYACNCVVGYIGERCQYRDLKWWELR
SEQ ID NO: 40
Homo sapiens epidermal growth factor (mutant)
NSDSECPLSHDGYCLHGGVCMYIKAVDRYACNCVVGYIGERCQYRDLTWWGPR
SEQ ID NO: 41
Homo sapiens epidermal growth factor (mutant)
NSDSECPLSHDGYCLHDGVCMYIKALDKYACNCVVGYTGERCQYRDLRWWGRR
SEQ ID NO: 42
Homo sapiens epidermal growth factor (mutant)
NSNSECPLSHDGYCLHDGVCRYIEALDRYACNCVVGYIGERCQYGDLRWWGRR
SEQ ID NO: 43
Homo sapiens epidermal growth factor (mutant)
NSDSGCPLSHSGYCLHDGVCMYIKALDRYACNCVVGYAGERCQYRDLRWWARR
SEQ ID NO: 44
Homo sapiens epidermal growth factor (mutant)
TRGSECPLSHDGYCLHDGVCMYIGALDRYACNCVVGYTGERCQYRDLRWWARR
SEQ ID NO: 45
Homo sapiens epidermal growth factor (mutant)
NSDFGCPLSYDGYCLHDGVCMYIKALDKYACNCVVGYAGERCQYRDLRWWGRR
SEQ ID NO: 46
Homo sapiens epidermal growth factor (mutant)
SRGSKCPPSHDGYCLHDGVCMYIEALDRYACNCVVGYAGERCQYRDLRWWARR
SEQ ID NO: 47
Homo sapiens epidermal growth factor (mutant)
SSGSECPSSHDGYCLHDGACMYIEALDRYACNCAVGYAGERCQYRDLRWWGRR
SEQ ID NO: 48
Homo sapiens epidermal growth factor (mutant)
SSNSECPPSHDGYCLHDGVCMYIEALDRYACNCVVGYAGERCQYRDLRWWARR
SEQ ID NO: 49
Homo sapiens epidermal growth factor (mutant)
NSYSECPPSYDGYCLHDGVCRYIEALDSYACNCVVGYAGERCQYRDLRWWGRR
SEQ ID NO: 50
Homo sapiens epidermal growth factor (mutant)
SSGSECPLSHDGYCLNDGVCMYIEALDKYACNCVVGYVGERCQYRDLRWWARR
SEQ ID NO: 145
Mature CD47 Protein
QLLFNKTKSVEFTFCNDTVVIPCFVTNMEAQNTTEVYVKWKFKGRDIYTFDGALNKSTV
PTDFSSAKIEVSQLLKGDASLKMDKSDAVSHTGNYTCEVTELTREGETIIELKYRVVSWF
SPNENILIVIFPIFAILLFWGQFGIKTLKYRSGGMDEKTIALLVAGLVITVIVIVGAILFVPG
EYSLKNATGLGLIVTSTGILILLHYYVFSTAIGLTSFVIAILVIQVIAYILAVVGLSLCIAACI
PMHGPLLISGLSILALAQLLGLVYMKFVE
SEQ ID NO: 146
CD47 Extracellular Domain
QLLFNKTKSVEFTFCNDTVVIPCFVTNMEAQNTTEVYVKWKFKGRDIYTFDGALNKSTV
PTDFSSAKIEVSQLLKGDASLKMDKSDAVSHTGNYTCEVTELTREGETIIELKYRVVSWF
SPNENI
SEQ ID NO: 162
Mutant CD47 Protein
WQPPLLFNKTKSVEFTFCNDTVVIPCFVTNMEAQNTTEVYVKWKFKGRDIYTFDGALN
KSTVPTDFSSAKIEVSQLLKGDASLKMDKSDAVSHTGNYTCEVTELTREGETIIELKYRV
VSWFSPNENILIVIFPIFAILLFWGQFGIKTLKYRSGGMDEKTIALLVAGLVITVIVIVGAIL
FVPGEYSLKNATGLGLIVTSTGILILLHYYVFSTAIGLTSFVIAILVIQVIAYILAVVGLSLCI
AACIPMHGPLLISGLSILALAQLLGLVYMKFVE
SEQ ID NO: 163
Mutant CD47 Extracellular Domain
WQPPLLFNKTKSVEFTFCNDTVVIPCFVTNMEAQNTTEVYVKWKFKGRDIYTFDGALN
KSTVPTDESSAKIEVSQLLKGDASLKMDKSDAVSHTGNYTCEVTELTREGETIIELKYRV
VSWFSPNENI
SEQ ID NO: 242
CD47 Sequence
tctccagaacatcatccctgcctctactggcgctgccaaggctgtgggcaaggtcatccctgagctgaacgggaagctcactggcatggcc
ttccgtgtccccactgccaacgtgtcagtggtggacctgacctgccgtctagaaaaacctgccaaatatgatgacatcaagaaggtggtgaa
gcaggcgtcggagggccccctcaagggcatcctgggctacactgagcaccaggtggtctcctctgacttcaacagcgacacccactcctc
cacctttgacgctggggctggcattgccctcaacgaccactttgtcaagctcatttcctggtatgtggctggggccagagactggctcttaa
aaagtgcagggtctggcgccctctggtggctggctcagaaaaagggccctgacaactcttttcatcttctaggtatgacaacgaatttggct
acagcaacagggtggtggacctcatggcccacatggcctccaaggagGGATCCGGAGCCACGAACTTCTCTCTGTT
AAAGCAAGCAGGAGACGTGGAAGAAAACCCCGGTCCTatgtggcccctggtagcggcgctgttgctg
ggctcggcgtgctgcggatcagctcagctactatttaataaaacaaaatctgtagagttcacgttttgtaatgacactgtcgtcattccatg
ctttgttactaatatggaggcacaaaacactactgaagtatacgtaaagtggaaatttaaaggaagagatatttacacctttgatggagctc
taaacaagtccactgtccccactgactttagtagtgcaaaaattgaagtctcacaattactaaaaggagatgcctctttgaagatggataag
agtgatgctgtctcacacacaggaaactacacttgtgaagtaacagaattaaccagagaaggtgaaacgatcatcgagctaaaatatcgtgt
tgtttcatggttttctccaaatgaaaatattcttattgttattttcccaatttttgctatactcctgttctggggacagtttggtattaaaa
cacttaaatatagatccggtggtatggatgagaaaacaattgctttacttgttgctggactagtgatcactgtcattgtcattgttggagcc
attcttttcgtcccaggtgaatattcattaaagaatgctactggccttggtttaattgtgacttctacagggatattaatattacttcacta
ctatgtgtttagtacagcgattggattaacctccttcgtcattgccatattggttattcaggtgatagcctatatcctcgctgtggttggac
tgagtctctgtattgcggcgtgtataccaatgcatggccctcttctgatttcaggtttgagtatcttagctctagcacaattacttggacta
gtttatatgaaatttgtggcttccaatcagaagactatacaacctcctaggaaagctgtagaggaaccccttaatgcattcaaagaatcaaa
aggaatgatgaatgatgaataaGGATCCCTGTGCCTTCTAGTTGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGCATTGTCTGA
GTAGGTGTCATTCTATTCTGGGGGGTGGGGTGGGGCAGGACAGCAAGGGGGAGGAT
TGGGAAGACAATAGCAGGCATGCTGGGGATGCGGTGGGCTCTATGGactcagtcccccacca
cactgaatctcccctcctcacagttgccatgtagaccccttgaagaggggaggggcctagggagccgcaccttgtcatgtaccatcaataaa
gtaccctgtgctcaaccagttacttgtcctgtcttattctagggtctggggcagaggggagggaagctgggcttgtgtcaaggtgagacatt
cttgctggggagggacctggtatgttctcctcagactgagggtagggcctccaaacagccttgcttgcttcgagaaccatttgcttcccgct
cagacgtcttgagtgctacaggaagctggcaccactacttcagagaacaaggccttttcctctcctcgctccagtcctaggctatctgctgt
tggccaaacatggaagaagctattctgtgggcagccccagggaggctgacaggtggaggaagtcagggctcgcactg
SEQ ID NO: 243
CD47 Amino Acid Sequence with Leader Sequence
MWPLVAALLLGSACCGSAQLLFNKTKSVEFTFCNDTVVIPCFVTNMEAQNTTEVYVKW
KFKGRDIYTFDGALNKSTVPTDFSSAKIEVSQLLKGDASLKMDKSDAVSHTGNYTCEVT
ELTREGETIIELKYRVVSWFSPNENILIVIFPIFAILLFWGQFGIKTLKYRSGGMDEKTIALL
VAGLVITVIVIVGAILFVPGEYSLKNATGLGLIVTSTGILILLHYYVFSTAIGLTSFVIAILVI
QVIAYILAVVGLSLCIAACIPMHGPLLISGLSILALAQLLGLVYMKFV
SEQ ID NO: 244
CD47 Leader Sequence
MWPLVAALLLGSACCGSA
SEQ ID NO: 245
Mutant CD47 Amino Acid Sequence with Leader Sequence
MWPLVAALLLGSACCGSAWQPPLLFNKTKSVEFTFCNDTVVIPCFVTNMEAQNTTEVY
VKWKFKGRDIYTFDGALNKSTVPTDESSAKIEVSQLLKGDASLKMDKSDAVSHTGNYT
CEVTELTREGETIIELKYRVVSWFSPNENILIVIFPIFAILLFWGQFGIKTLKYRSGGMDEK
TIALLVAGLVITVIVIVGAILFVPGEYSLKNATGLGLIVTSTGILILLHYYVFSTAIGLTSFVI
AILVIQVIAYILAVVGLSLCIAACIPMHGPLLISGLSILALAQLLGLVYMKFV

1. A mammalian cell that has been genetically engineered to have decreased or no expression of the renalase gene, and wherein the cell also has been genetically engineered to have:
a) decreased or no expression of the ABO gene;
b) decreased or no expression of the CXCL10 gene;
c) decreased or no expression of the beta-2 microglobulin (B2M) gene;
d) decreased or no expression of the tissue factor (F3) gene; and/or
e) increased expression of CD47 or expression of a mutant CD47,
as compared to the expression level of the same cell type that has not been genetically engineered.

2. The cell of claim 1, wherein the cell has been genetically engineered to have decreased or no expression of the ABO gene as compared to the expression level of the same cell type that has not been genetically engineered.

3. The cell of claim 1, wherein the cell has been genetically engineered to have decreased or no expression of the CXCL10 gene as compared to the expression level of the same cell type that has not been genetically engineered.

4. The cell of claim 1, wherein the cell has been genetically engineered to have decreased or no expression of the B2M gene as compared to the expression level of the same cell type that has not been genetically engineered.

5. The cell of claim 1, wherein the cell has been genetically engineered to have decreased or no expression of the F3 gene as compared to the expression level of the same cell type that has not been genetically engineered.

6. The cell of claim 1, wherein the cell has been genetically engineered to have increased expression of CD47 as compared to the expression level of the same cell type that has not been genetically engineered.

7. The cell of claim 6, wherein the cell comprises an insertion of an exogenous CD47 gene.

8. The cell of claim 1, wherein the cell has been genetically engineered to express a mutant CD47.

9. A mammalian cell that has been genetically engineered to have decreased or no expression of the CXCL10 gene, and wherein the cell also has been genetically engineered to have decreased or no expression of:
a) the ABO gene; and/or
b) the tissue factor (F3) gene.

10.-29. (canceled)

30. A mammalian cell, wherein the cell is ABO blood group type O, wherein the cell has been genetically engineered to:
a) have reduced or no expression of the renalase gene and/or the CXCL10 gene; and/or
b) express a mutant CD47 protein.

31.-34. (canceled)

35. A mammalian cell, wherein the cell expresses a membrane-bound CD47 protein, wherein the CD47 protein comprises an amino acid sequence that is at least 85%, 90%, 91%, 92%, 93%, 94%, 95%, 96%, 97%, 98%, 99%, or 100% identical to the amino acid sequence of SEQ ID NO: 145 or 146, but wherein the Q at position 1 is replaced with at least 3 amino acids.

36.-54. (canceled)

55. The cell of claim 1, wherein the cell is a stem cell.

56. The cell of claim 1, wherein the cell is a pluripotent stem cell (PSC), an embryonic stem cell (ESC), an induced pluripotent stem cell (iPSC), or an embryonic germ stem cell (EGSC).

57. The cell of claim 1, wherein the cell is differentiated from a pluripotent stem cell.

58.-67. (canceled)

68. The cell of claim 1, wherein the cell is NKX6.1-positive and ISL1-positive.

69. (canceled)

70. The cell of claim 1, wherein the cell is ISL1-positive.

71.-75. (canceled)

76. The cell of claim 1, wherein the mammalian cell has been genetically engineered to have decreased or no expression of proteins encoded by nucleic acids that are at least 80%, 85%, 90%, 91%, 92%, 93%, 94%, 95%, 96%, 97%, 98%, 99% or 100% identical to SEQ ID NO: 3 and/or SEQ ID NO: 5, and wherein the cell has also been genetically engineered to have:
a) decreased or no expression of proteins encoded by nucleic acid that are at least 80%, 85%, 90%, 91%, 92%, 93%, 94%, 95%, 96%, 97%, 98%, 99% or 100% identical to SEQ ID NO: 1;
b) decreased or no expression of proteins encoded by nucleic acid that are at least 80%, 85%, 90%, 91%, 92%, 93%, 94%, 95%, 96%, 97%, 98%, 99% or 100% identical to SEQ ID NO: 7;
c) decreased or no expression of proteins encoded by nucleic acid that are at least 80%, 85%, 90%, 91%, 92%, 93%, 94%, 95%, 96%, 97%, 98%, 99% or 100% identical to SEQ ID NO: 9;
d) decreased or no expression of proteins encoded by nucleic acid that are at least 80%, 85%, 90%, 91%, 92%, 93%, 94%, 95%, 96%, 97%, 98%, 99% or 100% identical to SEQ ID NO: 11; and/or
e) increased expression of proteins encoded by nucleic acid that are at least 80%, 85%, 90%, 91%, 92%, 93%, 94%, 95%, 96%, 97%, 98%, 99% or 100% identical to SEQ ID NO: 13 and/or SEQ ID NO: 15,
as compared to the expression level of the same cell type that has not been genetically engineered.

77.-95. (canceled)

96. A composition comprising one or more of the cells of claim 1.

97. The composition of claim 96, wherein the composition comprises a plurality of non-native cells; wherein:
a) at least 30% of the cells in the composition are NKX6.1-positive, ISL1-positive cells;
b) at least 25% of the cells in the composition are NKX6.1-negative, ISL1-positive cells;
c) there are more NKX6.1-positive, ISL1-positive cells than NKX6.1-negative, ISL1-positive cells in the composition;
d)
i) less than 12% of the cells in the composition are NKX6.1-negative, ISL1-negative cells; and/or
ii) between 9-25% of the cells in the composition are NKX6.1-positive, ISL1-negative cells; and
e) less than 40% of the cells in the composition are VMAT1-positive cells.

98. A method of administering the composition of claim 96 to a subject.

99. (canceled)


SEQUENCE LISTING (253 sequences)

SEQ ID NO: 1 - DNA, 6478 bp, Homo sapiens
  source 1..6478: mol_type = other DNA
agacgcggagccatggccgaggtgttgcggacgctggccggaaaaccaaaatgccacgcacttcgacctatgatccttttcctaataatgcttgtcttggtcttgtttggttacggggtcctaagccccagaagtctaatgccaggaagcctggaacgggggttctgcatggctgttagggaacctgaccatctgcagcgcgtctcgttgccaaggatggtctacccccagccaaaggtgctgacaccgtgtaggaaggatgtcctcgtggtgaccccttggctggctcccattgtctgggagggcacattcaacatcgacatcctcaacgagcagttcaggctccagaacaccaccattgggttaactgtgtttgccatcaagaaatacgtggctttcctgaagctgttcctggagacggcggagaagcacttcatggtgggccaccgtgtccactactatgtcttcaccgaccagccggccgcggtgccccgcgtgacgctggggaccggtcggcagctgtcagtgctggaggtgcgcgcctacaagcgctggcaggacgtgtccatgcgccgcatggagatgatcagtgacttctgcgagcggcgcttcctcagcgaggtggattacctggtgtgcgtggacgtggacatggagttccgcgaccacgtgggcgtggagatcctgactccgctgttcggcaccctgcaccccggcttctacggaagcagccgggaggccttcacctacgagcgccggccccagtcccaggcctacatccccaaggacgagggcgatttctactacctgggggggttcttcggggggtcggtgcaagaggtgcagcggctcaccagggcctgccaccaggccatgatggtcgaccaggccaacggcatcgaggccgtgtggcacgacgagagccacctgaacaagtacctgctgcgccacaaacccaccaaggtgctctcccccgagtacttgtgggaccagcagctgctgggctggcccgccgtcctgaggaagctgaggttcactgcggtgcccaagaaccaccaggcggtccggaacccgtgagcggctgccaggggctctgggagggctgccggcagccccgtccccctcccgcccttggttttagcagaacgggtaaactctgtttcctttgtccgtcctgttgtgagtaactgaagcctaggccccgtccccacctcaaatcacacacaccccctccccaccacagagacaccattacatacacagacacacacagaaagacacacacagacacaaaatcacacacacaccctccccgccacagagacaccattacatacacagacacacacagaaagacacagacacaaaatcacacacacaccctccccgccacagagacacaccattacatacacagacacgcaatcgcagatacgcccttccggccacagaaacacaccattacacacacatacacagaaagacacacacagacacacaatcacacgcagcccctccccgccacagagacacaccattacatacacagacacacacagaaagacacacacagacacaaaatcacacacacaccctccccgccacagagacacaccattacatacacagacacacacagacacacaatcacacacagcccctccccgccacagagacacaccattacatacacagacacacacagacacacaatcacagataccccctcccggccatagagacacaccgttacacacacatacacagaaagacacacacagcccctccctgccacagagacacaccattacatacacagacacacacagacatacacagacacacacacagacacacaatcacacacatcccctccccgccacagagacacaccattacacacacagacacacacagaaagacacacatacacacaatcacacacaccctcttcttgccacagagacgcaccattacatacacagacacagacacacagagacacagacacacaaacatagacacacacacacagacacacaccagacacgcaaagacacacagacacagatacacagatacaaagacacagacatatagacacacagacatgcacagagacacatggagacacatgcaaaaatgcacagagaaagacatacagaagtgtacacacagacacatagaccacacagacacacagacatgcatgcaaacacacagacatgcagacatgcacacaaacacagactcacgcacacagacttaggcagcccaaattcagcgcctggggcataagttcctggaggggtggccaccttcagcccccacggtaaggtcctgaggaaccttccccttagacaagggatcatggaggaggtctcttccggagcctggagggaggcctcaagtggtccttccacctcggcatcccaaagtgctaggattataagcatgagccactgcacctggccccaacatcatttattgaacagactgtcgtttccacattgtgtttcttggcatttctgtcaaaaatcagttgaccgtaaatgcatggattcacttccgggctctctattctgtttcattggtctgtttgtttttatgccaataccatgatgttttgattattatagctttatagtatatcttgaagttaggtagtgtattgtctccagctttgttctttttgcttaagattgctttggctatttaggggtctttgtggccattactgacagttaaaatggccctggctcatgtctcctgggcctctacagtactttaggtggggaagtactgtggcccacatgtcccaacaggctgtgtgtggcacctgacagtgctttgtgccactgtggagcaggtgttactccctcatacgaattttaggattattttatttatttcagtgaaaaatgtcaatgaaattttgatagggattgcattgaatctgtagattgtttggggttgtatggacattttaacaatattaattcttccaattcatgaacatgggatgtcttttttttggcacctgatctcagatatgggacagctttctaattatttgtatcttcaatttctttcacaaatgttttatagttttcagcatacagatctttcacttccttggttggatttatttgcaagtattttttgtagctattgtaaatgacattgttttcttgatttctttgttagggtatagaaatgctacagattgttgtatgttaattttatatcctgcaactttactgatttcatttattctaacatcttattggtggaatctttagggttttctataagatcatgtcatctgtaaacagggacaatttaagttcttcctttccaatttggataccttttatttcttcctcttgcctaattattttggctgagggccaacttgttgggtttgttgttgttggtttttgtctgtgtcttttggtgtttcgtgttgctggcttttccagcacacagtcacagatatatgaggcagaaagaaaacccagggaactcctgtcatgtcattcctcaggtcccaaggtccctacatggtctgccttcttctctccaccttttagtgtttgatatacatacgtttatgtatgtagttatatatgtatgtgtaatacccggggaaatatacagagattttaactgtccttagtgtagggcgtggggacaggtgcatctattctaccttgcctggaagtggaaaaatcttcaagaaatcaatttatcaccaactggcatgacagaactcctggtttagtgtctagctgtctccactaaaatcaatcaccatgccatcaggcactctcttccctctggtgtgtactggtacctgaaacagcgtccagcaccctgagagcacagaacgcagcttcttatgtgccccacccaataatcccaaatcgagcacagacctgggagctgaagagacaagctggaggtcaggcagttctattacttaagcctggttcttcctgactccaaaagccaggcgccaccccatgctccttcccagcagaagctggttttcaccgggtcaggaggacaggatggctattcctgaccgttgtacagcagtgccacatctcctagtttccagaacatttaatgtgttgctgtggaggcctggcagctctgaagtagccccttgcgcccaggacaaaccgggcctggaggggtggggtggggtaagctgaagcttccagtatcccacgtgggcattctgttgctgagggcaaccgcccctccctgcaagggaggggaaggagcaggcaggcacacccttcctccctccacatttcactggagttgacctgtgtcctactgtggttgcacctggaccagcccagagggactggagtgactcttcccgcaggtagggcacagccagcctgggcacagggcctcactgcaatccctgcgccccgcctccctgggggatttccaggggttgtcctgctgcttggtgagctgggggtgtgggggaccacagatgaggcaggcgcctaaattgcttgggcggagtccggctgctctctccctccaggccctttcctgtgtcctcgggcccctcgggtccctgcaccaggccagcagcaatcgggtgtcagaggttcaagtccaggctgactcctgggatgcaccgactttgcccaggagggctggggagagggcagccccaaagtgtcatctttgattattctttgtccccaaagccagccttgggcatgtgcccagcacagggagagctgctgcctctgtgggcttgagtcatttttgcttccttgcccctctctgctctgtttggtttaaaaaacaaaaaccaacacccatgggtgaggcgaggcgaggcgaggcttgccttgatccacccgcttcctcccctgcagcctcgggccccatcctcccgcgtggccgcactaggggtgctgggggcggagtggggaccagacctgagtgggttctgggttcggcttcgcagcaggagcagctggggcatcatcagttgggcaggtgacccaggatctgagcaggaatcttccaggtggaggtggggccgcccctctgcttgccacagctgccttctgctctgggggcagcactggctgagaatccagtgaagtaaggcctttgaaatggttttgattgtaaaagtaatgtttatgtttagccattcctttttttattttttattttttgagacgggtctccctctgtcacccaggctggagtgcagtcgtgaaatgtgggctcactgcaacctccacctcccaggttcaagtgattctcctgcctcagcctcctgagtagttgggattacaagtgcctgccaccacacctgactaagttttgtattttttagtagagatggagtttcaccatgttggccaggctggtctcaaactcatatcctcaagtgatccaccctcctcgtcctcccaaagtgctgggattttgccatgagccaccacgctcagccatgtttagccatttttaaaaggtgtgaacagataattaagtctattagcaacattagaaaatttaagttaaaatttaaatgactctagcaagaattagcctttaggtgccctgggtccccctccccaccgccccccaccccgccgccttctgagaagcgttttgtaatttttcttatttataaaatgggtttgattcaaaactgacttttcctcttcacagagtctcacaggtccttccccaggtccaagaggctcttctgtggtctaatgacaagtagctgcctagccgtggtggcacctcctatcacatgttaagggacccctccccagggccacacctggcagaaggtggcttatgatgttcgcagcttgaaagtagtgtaaaccaaagataaaattctaagcccactcccccagccatcggaatggacccctcctcttggccagggcactccaaagttaacctgaaaaaccggttcaggctgtgaagagaaggtggagtggacatgcctcatttatgtcctcctcccttttggaattcagcaaagctgaccagcatgaacattaacacagaccttaagtctgattagtggcatttacaatctatactctctgaagcgtgctacctggaggcttcctttgcatgataaaactttggtctccacaaccccttatcataacctagacactcctttctagtgataataactctttcaaccaattgccaataaaaaaattttgaatctacctataacctggaacctccctgctccaccttcgagttgtcctacctttctggacagaagcaatgtggatcttgcatgtatttgattgatgtctcatgtctccctaaaatgtatacaattaggctgtgcccagatcaccctgggcacatgttctcaggccctcctgaggtctctgtctcgggccattggtcactcagattcggctcagaataaatctcttcaaatatt

SEQ ID NO: 2 - protein, 354 aa, Homo sapiens
MAEVLRTLAGKPKCHALRPMILFLIMLVLVLFGYGVLSPRSLMPGSLERGFCMAVREPDHLQRVSLPRMVYPQPKVLTPCRKDVLVVTPWLAPIVWEGTFNIDILNEQFRLQNTTIGLTVFAIKKYVAFLKLFLETAEKHFMVGHRVHYYVFTDQPAAVPRVTLGTGRQLSVLEVRAYKRWQDVSMRRMEMISDFCERRFLSEVDYLVCVDVDMEFRDHVGVEILTPLFGTLHPGFYGSSREAFTYERRPQSQAYIPKDEGDFYYLGGFFGGSVQEVQRLTRACHQAMMVDQANGIEAVWHDESHLNKYLLRHKPTKVLSPEYLWDQQLLGWPAVLRKLRFTAVPKNHQAVRNP

SEQ ID NO: 3 - DNA, 2409 bp, Homo sapiens
  source 1..2409: mol_type = other DNA
agggcccaggtcggcccagggagcacggaaccaaagagcgctagcgccggttcggccgcctttccagaaagcccgggccgaacggccccgccgcagagactcagcgcggatcgctgctccctctcgccatggcgcaggtgctgatcgtgggcgccgggatgacaggaagcttgtgcgctgcgctgctgaggaggcagacgtccggtcccttgtaccttgctgtgtgggacaaggctgaggactcagggggaagaatgactacagcctgcagtcctcataatcctcagtgcacagctgacttgggtgctcagtacatcacctgcactcctcattatgccaaaaaacaccaacgtttttatgatgaactgttagcctatggcgttttgaggcctctaagctcgcctattgaaggaatggtgatgaaagaaggagactgtaactttgtggcacctcaaggaatttcttcaattattaagcattacttgaaagaatcaggtgcagaagtctacttcagacatcgtgtgacacagatcaacctaagagatgacaaatgggaagtatccaaacaaacaggctcccctgagcagtttgatcttattgttctcacaatgccagttcctgagattctgcagcttcaaggtgacatcaccaccttaattagtgaatgccaaaggcagcaactggaggctgtgagctactcctctcgatatgctctgggcctcttttatgaagctggtacgaagattgatgtcccttgggctgggcagtacatcaccagtaatccctgcatacgcttcgtctccattgataataagaagcgcaatatagagtcatcagaaattgggccttccctcgtgattcacaccactgtcccatttggagttacatacttggaacacagcattgaggatgtgcaagagttagtcttccagcagctggaaaacattttgccgggtttgcctcagccaattgctaccaaatgccaaaaatggagacattcacaggttacaaatgctgctgccaactgtcctggccaaatgactctgcatcacaaacctttccttgcatgtggaggggatggatttactcagtccaactttgatggctgcatcacttctgccctatgtgttctggaagctttaaagaattatatttagtgcctatatccttattctctacatgtgtattgggtttttattttcacaattttctgttattgattattttgttttctattttgctaagaaaaattactggaaaattgttcttcacttattatcatttttcatgtggagtataaaatcaattttgtaattttgatagttacaacccatgctagaatggaaattcctcacaccttgcaccttccctacttttctgaattgctatgactactccttgttggaggaaaagtggtacttaaaaaataacaaacgactctctcaaaaaaattacattaaatcacaataacagtttgtgtgccaaaaacttgattatccttatgaaaatttcaattctgaataaagaataatcacattatcaaagccccatcttaagtcttcggatgtgtccttgaatcaatatttttgcaaattatacaaaacaagatttttccaaaatgtaggtaacagagtgtaattcttatttctcatttatcccccaagttattaagtgatcctgaattgtaggtcatatatgtcatcatcttagtgtggagggcaacttgactgataaagagaccttccttcagattttcagaaagtataagattccacatgattttcccagccacacagtactttttaactttcaaacaaattccagtcctaatatgaaagataaaaattaaatagaaacagagagaaagtatatcgatccttaccttttgctatattttatagctgttgctgttactttatgggttctccagtatgtgctgtggcatttagactgtgtcgagtttaatgaatttaacacaacaaaaaatttactgaaccagaaaatagatgcacttaaaatagttcaatatttgccaagttggtggttcagcatatcacccacatgcttcagtgacctgaccccacgacttgctagctggagagaaatcaatctccagccttccaaaccagctacctgttgctaatttgaaaagcaaaatgatgagttctatttcagcattttgaaaggagaaaaatcattgcagcctctcaaactaacaaaagttcaacaaaagacttcttactgtaatagtgtttaaagtttcacacttacatgtccactgtcatacatacacatacacaggcacaggcagaacttgcttctatagctgcaaagtgggttttatgaccctatagcatattattatatgtttcctcttagcaataaattggtgaaaaacttaaatgccaa

SEQ ID NO: 4 - protein, 342 aa, Homo sapiens
MAQVLIVGAGMTGSLCAALLRRQTSGPLYLAVWDKAEDSGGRMTTACSPHNPQCTADLGAQYITCTPHYAKKHQRFYDELLAYGVLRPLSSPIEGMVMKEGDCNFVAPQGISSIIKHYLKESGAEVYFRHRVTQINLRDDKWEVSKQTGSPEQFDLIVLTMPVPEILQLQGDITTLISECQRQQLEAVSYSSRYALGLFYEAGTKIDVPWAGQYITSNPCIRFVSIDNKKRNIESSEIGPSLVIHTTVPFGVTYLEHSIEDVQELVFQQLENILPGLPQPIATKCQKWRHSQVTNAAANCPGQMTLHHKPFLACGGDGFTQSNFDGCITSALCVLEALKNYI

SEQ ID NO: 5 - DNA, 2175 bp, Homo sapiens
  source 1..2175: mol_type = other DNA
agggcccaggtcggcccagggagcacggaaccaaagagcgctagcgccggttcggccgcctttccagaaagcccgggccgaacggccccgccgcagagactcagcgcggatcgctgctccctctcgccatggcgcaggtgctgatcgtgggcgccgggatgacaggaagcttgtgcgctgcgctgctgaggaggcagacgtccggtcccttgtaccttgctgtgtgggacaaggctgaggactcagggggaagaatgactacagcctgcagtcctcataatcctcagtgcacagctgacttgggtgctcagtacatcacctgcactcctcattatgccaaaaaacaccaacgtttttatgatgaactgttagcctatggcgttttgaggcctctaagctcgcctattgaaggaatggtgatgaaagaaggagactgtaactttgtggcacctcaaggaatttcttcaattattaagcattacttgaaagaatcaggtgcagaagtctacttcagacatcgtgtgacacagatcaacctaagagatgacaaatgggaagtatccaaacaaacaggctcccctgagcagtttgatcttattgttctcacaatgccagttcctgagattctgcagcttcaaggtgacatcaccaccttaattagtgaatgccaaaggcagcaactggaggctgtgagctactcctctcgatatgctctgggcctcttttatgaagctggtacgaagattgatgtcccttgggctgggcagtacatcaccagtaatccctgcatacgcttcgtctccattgataataagaagcgcaatatagagtcatcagaaattgggccttccctcgtgattcacaccactgtcccatttggagttacatacttggaacacagcattgaggatgtgcaagagttagtcttccagcagctggaaaacattttgccgggtttgcctcagccaattgctaccaaatgccaaaaatggagacattcacaggtaccaagtgctggtgtgattctaggatgtgcgaagagcccctggatgatggcgattggatttcccatctgacttcctggaaattggagcacacagtcaggttttatttgatttttttttttaaggataccacttcacagcctttaggatagctattatttagaagcaaaacagaagataaatgttggcaaggatgtggagatattggattcccttgtgcagtgccggtgggaatgtaaaatgatgtagctactatggaaaatgatacggcaatttctttagaaatgaaatatagaattgctgtatgatctgcagttccacatctggatatctatccaaaagaagtgaaagtagggacttgaacgaacatttgtacaccaatgttcacagcggctttattcacaacagccaaaaggtggaagcaacccagtgtccatggatagatgaatagataaataaaatgtggtataaacatacaatgggctattgtttagccttaaaagggaaggaaattctgacatgctgcaatatggatgaagcttaaagtcattatgcaaagtggaataagcctatcacaaaaaataatattacataattctacttatatgaggaatctagagcagtcagtttcacagagacagaaaatagaatggtggttgccaagggctgggagaagagggcaatggagagtgagtgtttagtgggtcagagttttagtttgggaaggtaaaaagttctggagatggatgatggttatgggtgctcaacagtgtgaatgtacttaatgccacagaactgcacatttaaatgtggttaaaatcatcacttttatgttatgtatatttaccacaataaataaagaagttgatatttcttatacttacaaagaggagaagggcatttgcaaatcaacaagaagtgtgaggcccctctctctagcagaaaaatagactaaatctatttctttatcttttaacatcctgtttaagggaaatgccaaaacaaatgggaaaaaatacacacacacaaatatatatgaacatgttttgcctcatgagtaatcaaaatgtgtacatatgtatgtttatgtatgtgtgtttatatttaaaatcgtgttctgccttatgagtaaacaaaaagtatacaaattaaaaactataatgaaacgtaa

SEQ ID NO: 6 - protein, 315 aa, Homo sapiens
MAQVLIVGAGMTGSLCAALLRRQTSGPLYLAVWDKAEDSGGRMTTACSPHNPQCTADLGAQYITCTPHYAKKHQRFYDELLAYGVLRPLSSPIEGMVMKEGDCNFVAPQGISSIIKHYLKESGAEVYFRHRVTQINLRDDKWEVSKQTGSPEQFDLIVLTMPVPEILQLQGDITTLISECQRQQLEAVSYSSRYALGLFYEAGTKIDVPWAGQYITSNPCIRFVSIDNKKRNIESSEIGPSLVIHTTVPFGVTYLEHSIEDVQELVFQQLENILPGLPQPIATKCQKWRHSQVPSAGVILGCAKSPWMMAIGFPI

SEQ ID NO: 7 - DNA, 1175 bp, Homo sapiens
  source 1..1175: mol_type = other DNA
gagacattcctcaattgcttagacatattctgagcctacagcagaggaacctccagtctcagcaccatgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaaaaccagaggggagcaaaatcgatgcagtgcttccaaggatggaccacacagaggctgcctctcccatcacttccctacatggagtatatgtcaagccataattgttcttagtttgcagttacactaaaaggtgaccaatgatggtcaccaaatcagctgctactactcctgtaggaaggttaatgttcatcatcctaagctattcagtaataactctaccctggcactataatgtaagctctactgaggtgctatgttcttagtggatgttctgaccctgcttcaaatatttccctcacctttcccatcttccaagggtactaaggaatctttctgctttggggtttatcagaattctcagaatctcaaataactaaaaggtatgcaatcaaatctgctttttaaagaatgctctttacttcatggacttccactgccatcctcccaaggggcccaaattctttcagtggctacctacatacaattccaaacacatacaggaaggtagaaatatctgaaaatgtatgtgtaagtattcttatttaatgaaagactgtacaaagtagaagtcttagatgtatatatttcctatattgttttcagtgtacatggaataacatgtaattaagtactatgtatcaatgagtaacaggaaaattttaaaaatacagatagatatatgctctgcatgttacataagataaatgtgctgaatggttttcaaaataaaaatgaggtactctcctggaaatattaagaaagactatctaaatgttgaaagatcaaaaggttaataaagtaattataactaa

SEQ ID NO: 8 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP

SEQ ID NO: 9 - DNA, 943 bp, Homo sapiens
  source 1..943: mol_type = other DNA
attcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttga

SEQ ID NO: 10 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 11 - DNA, 2298 bp, Homo sapiens
  source 1..2298: mol_type = other DNA
aagactgcgagctccccgcaccccctcgcactccctctggccggcccagggcgccttcagcccaacctccccagccccacgggcgccacggaacccgctcgatctcgccgccaactggtagacatggagacccctgcctggccccgggtcccgcgccccgagaccgccgtcgctcggacgctcctgctcggctgggtcttcgcccaggtggccggcgcttcaggcactacaaatactgtggcagcatataatttaacttggaaatcaactaatttcaagacaattttggagtgggaacccaaacccgtcaatcaagtctacactgttcaaataagcactaagtcaggagattggaaaagcaaatgcttttacacaacagacacagagtgtgacctcaccgacgagattgtgaaggatgtgaagcagacgtacttggcacgggtcttctcctacccggcagggaatgtggagagcaccggttctgctggggagcctctgtatgagaactccccagagttcacaccttacctggagacaaacctcggacagccaacaattcagagttttgaacaggtgggaacaaaagtgaatgtgaccgtagaagatgaacggactttagtcagaaggaacaacactttcctaagcctccgggatgtttttggcaaggacttaatttatacactttattattggaaatcttcaagttcaggaaagaaaacagccaaaacaaacactaatgagtttttgattgatgtggataaaggagaaaactactgtttcagtgttcaagcagtgattccctcccgaacagttaaccggaagagtacagacagcccggtagagtgtatgggccaggagaaaggggaattcagagaaatattctacatcattggagctgtggtatttgtggtcatcatccttgtcatcatcctggctatatctctacacaagtgtagaaaggcaggagtggggcagagctggaaggagaactccccactgaatgtttcataaaggaagcactgttggagctactgcaaatgctatattgcactgtgaccgagaacttttaagaggatagaatacatggaaacgcaaatgagtatttcggagcatgaagaccctggagttcaaaaaactcttgatatgacctgttattaccattagcattctggttttgacatcagcattagtcactttgaaatgtaacgaatggtactacaaccaattccaagttttaatttttaacaccatggcaccttttgcacataacatgctttagattatatattccgcactcaaggagtaaccaggtcgtccaagcaaaaacaaatgggaaaatgtcttaaaaaatcctgggtggacttttgaaaagcttttttttttttttttttttttttgagacggagtcttgctctgttgcccaggctggagtgcagtagcacgatctcggctcactgcaccctccgtctctcgggttcaagcaattgtctgcctcagcctcccgagtagctgggattacaggtgcgcactaccacgccaagctaatttttgtattttttagtagagatggggtttcaccatcttggccaggctggtcttgaattcctgacctcaggtgatccacccaccttggcctcccaaagtgctagtattatgggcgtgaaccaccatgcccagccgaaaagcttttgaggggctgacttcaatccatgtaggaaagtaaaatggaaggaaattgggtgcatttctaggacttttctaacatatgtctataatatagtgtttaggttcttttttttttcaggaatacatttggaaattcaaaacaattggcaaactttgtattaatgtgttaagtgcaggagacattggtattctgggcaccttcctaatatgctttacaatctgcactttaactgacttaagtggcattaaacatttgagagctaactatatttttataagactactatacaaactacagagtttatgatttaaggtacttaaagcttctatggttgacattgtatatataattttttaaaaaggttttctatatggggattttctatttatgtaggtaatattgttctatttgtatatattgagataatttatttaatatactttaaataaaggtgactgggaattgttactgttgtacttattctatcttccatttattatttatgtacaatttggtgtttgtattagctctactacagtaaatgactgtaaaattgtcagtggcttacaacaacgtatctttttcgcttataatacattttggtgactgta

SEQ ID NO: 12 - protein, 295 aa, Homo sapiens
METPAWPRVPRPETAVARTLLLGWVFAQVAGASGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFREIFYIIGAVVFVVIILVIILAISLHKCRKAGVGQSWKENSPLNVS

SEQ ID NO: 13 - DNA, 5292 bp, Homo sapiens
  source 1..5292: mol_type = other DNA
gcagcctgggcagtgggtcctgcctgtgacgcgcggcggcggtcggtcctgcctgtaacggcggcggcggctgctgctccggacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtccaa

SEQ ID NO: 14 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 15 - DNA, 5202 bp, Homo sapiens
  source 1..5202: mol_type = other DNA
gcagcctgggcagtgggtcctgcctgtgacgcgcggcggcggtcggtcctgcctgtaacggcggcggcggctgctgctccggacacctgcggcggcggcggcgaccccgcggcgggcgcggagatgtggcccctggtagcggcgctgttgctgggctcggcgtgctgcggatcagctcagctactatttaataaaacaaaatctgtagaattcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggaataactgaagtgaagtgatggactccgatttggagagtagtaagacgtgaaaggaatacacttgtgtttaagcaccatggccttgatgattcactgttggggagaagaaacaagaaaagtaactggttgtcacctatgagacccttacgtgattgttagttaagtttttattcaaagcagctgtaatttagttaataaaataattatgatctatgttgtttgcccaattgagatccagttttttgttgttatttttaatcaattaggggcaatagtagaatggacaatttccaagaatgatgcctttcaggtcctagggcctctggcctctaggtaaccagtttaaattggttcagggtgataactacttagcactgccctggtgattacccagagatatctatgaaaaccagtggcttccatcaaacctttgccaactcaggttcacagcagctttgggcagttatggcagtatggcattagctgagaggtgtctgccacttctgggtcaatggaataataaattaagtacaggcaggaatttggttgggagcatcttgtatgatctccgtatgatgtgatattgatggagatagtggtcctcattcttgggggttgccattcccacattcccccttcaacaaacagtgtaacaggtccttcccagatttagggtacttttattgatggatatgttttccttttattcacataaccccttgaaaccctgtcttgtcctcctgttacttgcttctgctgtacaagatgtagcaccttttctcctctttgaacatggtctagtgacacggtagcaccagttgcaggaaggagccagacttgttctcagagcactgtgttcacacttttcagcaaaaatagctatggttgtaacatatgtattcccttcctctgatttgaaggcaaaaatctacagtgtttcttcacttcttttctgatctggggcatgaaaaaagcaagattgaaatttgaactatgagtctcctgcatggcaacaaaatgtgtgtcaccatcaggccaacaggccagcccttgaatggggatttattactgttgtatctatgttgcatgataaacattcatcaccttcctcctgtagtcctgcctcgtactccccttcccctatgattgaaaagtaaacaaaacccacatttcctatcctggttagaagaaaattaatgttctgacagttgtgatcgcctggagtacttttagacttttagcattcgttttttacctgtttgtggatgtgtgtttgtatgtgcatacgtatgagataggcacatgcatcttctgtatggacaaaggtggggtacctacaggagagcaaaggttaattttgtgcttttagtaaaaacatttaaatacaaagttctttattgggtggaattatatttgatgcaaatatttgatcacttaaaacttttaaaacttctaggtaatttgccacgctttttgactgctcaccaataccctgtaaaaatacgtaattcttcctgtttgtgtaataagatattcatatttgtagttgcattaataatagttatttcttagtccatcagatgttcccgtgtgcctcttttatgccaaattgattgtcatatttcatgttgggaccaagtagtttgcccatggcaaacctaaatttatgacctgctgaggcctctcagaaaactgagcatactagcaagacagctcttcttgaaaaaaaaaatatgtatacacaaatatatacgtatatctatatatacgtatgtatatacacacatgtatattcttccttgattgtgtagctgtccaaaataataacatatatagagggagctgtattcctttatacaaatctgatggctcctgcagcactttttccttctgaaaatatttacattttgctaacctagtttgttactttaaaaatcagttttgatgaaaggagggaaaagcagatggacttgaaaaagatccaagctcctattagaaaaggtatgaaaatctttatagtaaaattttttataaactaaagttgtaccttttaatatgtagtaaactctcatttatttggggttcgctcttggatctcatccatccattgtgttctctttaatgctgcctgccttttgaggcattcactgccctagacaatgccaccagagatagtgggggaaatgccagatgaaaccaactcttgctctcactagttgtcagcttctctggataagtgaccacagaagcaggagtcctcctgcttgggcatcattgggccagttccttctctttaaatcagatttgtaatggctcccaaattccatcacatcacatttaaattgcagacagtgttttgcacatcatgtatctgttttgtcccataatatgctttttactccctgatcccagtttctgctgttgactcttccattcagttttatttattgtgtgttctcacagtgacaccatttgtccttttctgcaacaacctttccagctacttttgccaaattctatttgtcttctccttcaaaacattctcctttgcagttcctcttcatctgtgtagctgctcttttgtctcttaacttaccattcctatagtactttatgcatctctgcttagttctattagttttttggccttgctcttctccttgattttaaaattccttctatagctagagcttttctttctttcattctctcttcctgcagtgttttgcatacatcagaagctaggtacataagttaaatgattgagagttggctgtatttagatttatcactttttaatagggtgagcttgagagttttctttctttctgttttttttttttgttttttttttttttttttttttttttttttttgactaatttcacatgctctaaaaaccttcaaaggtgattatttttctcctggaaactccaggtccattctgtttaaatccctaagaatgtcagaattaaaataacagggctatcccgtaattggaaatatttcttttttcaggatgctatagtcaatttagtaagtgaccaccaaattgttatttgcactaacaaagctcaaaacacgataagtttactcctccatctcagtaataaaaattaagctgtaatcaaccttctaggtttctcttgtcttaaaatgggtattcaaaaatggggatctgtggtgtatgtatggaaacacatactccttaatttacctgttgttggaaactggagaaatgattgtcgggcaaccgtttattttttattgtattttatttggttgagggatttttttataaacagttttacttgtgtcatattttaaaattactaactgccatcacctgctggggtcctttgttaggtcattttcagtgactaatagggataatccaggtaactttgaagagatgagcagtgagtgaccaggcagtttttctgcctttagctttgacagttcttaattaagatcattgaagaccagctttctcataaatttctctttttgaaaaaaagaaagcatttgtactaagctcctctgtaagacaacatcttaaatcttaaaagtgttgttatcatgactggtgagagaagaaaacattttgtttttattaaatggagcattatttacaaaaagccattgttgagaattagatcccacatcgtataaatatctattaaccattctaaataaagagaactccagtgttgctatgtgcaagatcctctcttggagcttttttgcatagcaattaaaggtgtgctatttgtcagtagccatttttttgcagtgatttgaagaccaaagttgttttacagctgtgttaccgttaaaggtttttttttttatatgtattaaatcaatttatcactgtttaaagctttgaatatctgcaatctttgccaaggtacttttttatttaaaaaaaaacataactttgtaaatattaccctgtaatattatatatacttaataaaacattttaagctattttgttgggctatttctattgctgctacagcagaccacaagcacatttctgaaaaatttaatttattaatgtatttttaagttgcttatattctaggtaacaatgtaaagaatgatttaaaatattaattatgaattttttgagtataatacccaataagcttttaattagagcagagttttaattaaaagttttaaatcagtccaa

SEQ ID NO: 16 - protein, 293 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVE

SEQ ID NO: 17 - protein, 116 aa, Homo sapiens [sequence omitted]

SEQ ID NO: 18 - DNA, 351 bp, Homo sapiens
  source 1..351: mol_type = other DNA
ggcttggagtgtgatggcaaggtcaacatctgctgtaagaaacagttctttgtcagtttcaaggacatcggctggaatgactggatcattgctccctctggctatcatgccaactactgcgagggtgagtgcccgagccatatagcaggcacgtccgggtcctcactgtccttccactcaacagtcatcaaccactaccgcatgcggggccatagcccctttgccaacctcaaatcgtgctgtgtgcccaccaagctgagacccatgtccatgttgtactatgatgatggtcaaaacatcatcaaaaaggacattcagaacatgatcgtggaggagtgtgggtgctcatag

SEQ ID NO: 19 - protein, 426 aa, Homo sapiens
MPLLWLRGFLLASCWIIVRSSPTPGSEGHSAAPDCPSCALAALPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGEEAEEVGLKGERSELLLSEKVVDARKSTWHVFPVSSSIQRLLDQGKSSLDVRIACEQCQESGASLVLLGKKKKKEEEGEGKKKGGGEGGAGADEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 20 - protein, 130 aa, Homo sapiens
ARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 21 - protein, 115 aa, Homo sapiens
GLECDGKVNICCKKQFFVSFKDIGWNDWIIAPSGYHANYCEGECPSHIAGTSGSSLSFHSTVINHYACGHSPFANLKSCCVPTKLRPMSMLYYDDGQNIIKKDIQNMIVEECGCS

SEQ ID NO: 22 - protein, 426 aa, Homo sapiens; identical to SEQ ID NO: 19

SEQ ID NO: 23 - protein, 424 aa, Homo sapiens
MPLLWLRGFLLASCWIIVRSSPTPGSEGHGSAPDCPSCALATLPKDGPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDTGDEAEEMGLKGERSELLLSEKVVDARKSTWHIFPVSSSIQRLLDQGKSSLDVRIACEQCQESGASLVLLGKKKKKEVDGDGKKKDGSDGGLEEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 24 - protein, 424 aa, Rattus norvegicus
MPLLWLRGFLLASCWIIVRSSPTPGSEGHGAAPDCPSCALATLPKDGPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIRLFQQQKHPQGSLDMGDEAEEMGLKGERSELLLSEKVVDARKSTWHIFPVSSSIQRLLDQGKSSLDVRIACEQCQESGASLVLLGKKKKKEVDGDGKKKDGSDGGLEEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 25 - protein, 424 aa, Gallus gallus
MPLLWKRGFLLVICWIIVRSSPTPGSEGHSSVADCPSCALTTLSKDVPSSQPEMVEAVKKHILNMLHLRDRPNITQPVPKAALLNATKKLHVGKVGDDGYVEIEDDVGRRAEMNEVVEQTSEIITFAESGTPKKTLHFEISKEGSELSVVEHAEVWLFLKVSKANRSRTKVTIRLFQQQRQPKGNSEAAEDMEDMGLKGERSETLISEKAVDARKSTWHIFPISSSVQRLLDQGQSSLDVRIACDLCQETGASLVLLGKKKKKEDDGEGKEKDGGELTGEEEKEQSHRPFLMMLARHSEDRQHRRRERGLECDGKVNICCKKQFFVSFKDIGWSDWIIAPTGYHANYCEEECPSHIAGTSGSSLSFHSTVINHYRMRGHSPFANLKSCCVPTKLRPMSMLYYDDGQNIIKKDIQNMIVEECGCS

SEQ ID NO: 26 - protein, 425 aa, Bos taurus
MPLLWLRGFLLASCWIIVRSSPTPGSEGHSAAPDCPSCALATLPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEIWLFLKVPKANRTRSKVTIRLFQQQKHLQGSLDAGEEAEEVGLKGEKSEMLISEKVVDARKSTWHIFPVSSCIQRLLDQGKSSLDIRIACEQCQETGASLVLLGKKKKKEEEGEGKKRDGEGGAGGDEEKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 27 - protein, 426 aa, Equus caballus
MPLLWLRGFLLASCWIIVKSSPTPGSEGHSAAPNCPSCALATLPKDVPNAQPEMVEAVKKHILNMLHLKKRPDVTQPVPKAALLNAIRKLHVGKVGENGYVEIEDDIGRRAEMNELMEQTSEIITFAESGTARKTLHFEISKEGSDLSVVERAEVWLFLKVPKANRTRSKVTIRLLQQQKHPQGSSDTREEAEEADLMEERSEQLISEKVVDARKSTWHIFPVSSSIQRLLDQGKSSLDIRIACDQCHETGASLVLLGKKKKKEEEGEGKKKDGGEAGAGVDEEKEQSHRPFLMLQARQSEDHPHRRRRRGLECDGKVNICCKKQFFVSFKDIGWNDWIIAPSGYHANYCEGECPSHIAGTSGSSLSFHSTVINQYRLRGHNPFANLKSCCVPTKLRPMSMLYYDDGQNIIKKDIQNMIVEECGCS

SEQ ID NO: 28 - protein, 424 aa, Sus scrofa
MPLLWLRGFLLASCWIIVRSSPTPGSGGHSAAPDCPSCALATLPKDVPNSQPEMVEAVKKHILNMLHLKKRPDVTQPVPKAALLNAIRKLHVGKVGENGYVELEDDIGRRAEMNELMEQTSEIITFAEAGTARKTLRFEISKEGSDLSVVERAEIWLFLKVPKANRTRTKVSIRLFQQQRRPQGSADAGEEAEDVGFPEEKSEVLISEKVVDARKSTWHIFPVSSSIQRLLDQGKSALDIRTACEQCHETGASLVLLGKKKKKEEEAEGRKRDGEGAGVDEEKEQSHRPFLMLQARQSEEHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 29 - protein, 425 aa, Ovis aries
MPLLWLRGFLLASCWIIVRSSPTPGSEGHSAAPDCPSCALATLPKDVPNSQPEMVEAVKKHILNMLHLKKRPDVTQPVPKAALLNAIRKLHVGKVGENGYVEIEDDIGRRAEMNELMEQTSEIITFAESGTARKTLHFEISQEGSDLSVVERAEIWLFLKVPKANRTRSKVTIRLFQQQKHLQGSLDAGEEAEEVGLKGEKSEMLISEKVVDARKSTWHIFPVSSCIQRLLDQGKSSLDIRIACEQCQETGASLVLLGKKKRKEEEGEGKKRDGEGGAGGDEEKEQSHRPFLMLQARQSEDHPHRRRRRGLECDGKVNICCKKQFYVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 30 - protein, 424 aa, Felis catus
MPLLWLRGFLLASCWIIVRSSPTPGSEGPGAAPDCPSCALATLPKDVPNSQPEMVEAVKKHILNMLHLKKRPEVTQPVPKAALLNAIRKLHVGKVGENGYVEIEDDIGRRAEMNELMEQTSEIITFAESGTARKTLHFEISKEGSDLSVVERAEVWLFLKVPKANRTRTKVTIQLLQKQPQGGVDAGEEAEEMGLMEERNEVLISEKVVDARKSTWHIFPVSSSIQRLLDQGKSSLDVRIACEQCHETGASLVLLGKKKKKEEEGEGKKKDGGDGGAGADEDKEQSHRPFLMLQARQSEDHPHRRRRRGLECDGKVNICCKKQFFVSFKDIGWNDWIIAPSGYHANYCEGECPSHIAGTSGSSLSFHSTVINHYRMRGHSPFANLKSCCVPTKLRPMSMLYYDDGQNIIKKDIQNMIVEECGCS

SEQ ID NO: 31 - protein, 395 aa, Danio rerio
MSPLPLLSGILLLLIRSCSLSAMVTKGSLPMSEQQAGATVCPSCALARFRKGVSESEDEGAQQDVVEAVKRHILNMLHLQERPNITHPVPRAALLNAIRKVHVGRVAKDGSVLIEDEASNRAETEQAEQTEIITFAETGEAPGIVNFLISKEGGEMSVVDQANVWIFLRLPKGNRTRANVNIRLLLQQGAGEKILAEKSVDTRRSGWHTFPASESVQSLLQRGGSTLSLRVSCPLCADARATPVLVSPGGSEREQSHRPFLMAVVRQMDELSLRRRRKRGLECDGKARVCCKRQFYVNFKDIGWNDWIIAPSGYHANYCEGDCASNVASITGNSLSFHSTVISHYRIRGYSPFTNIKSCCVPTRLRAMSMLYYNEEQKIVKKDIQNMIVEECGCS

SEQ ID NO: 32 - protein, 404 aa, Carassius auratus
MSSLTLVNRGTAALRLFVRGLLTHSSREWLSGDGEPDDPVTPCPSCALAQRQKDSEEQTDMVEAVKRHILNMLHLNTRPNVTHPVPRAALLNAIRRLHVGRVGEDGTVEMEEDGGGLGEHREQSEEQPFEIITFAEPGDAPDIMKFDISMEGNTLSVVEQANVWLLLKVAKGSRGKGKVSVQLLQHGKADPGSADGPQEAVVSEKTVDTRRSGWHTLPVSRTVQTLLDGDSSMLSLRVSCPMCAEAGAVPILVPTESNKGKEREQSHRPFLMVVLKPAEEHPHRRSKRGLECDGKIRVCCKRQFYVNFKDIGWSDWIIAPSGYHANYCEGDCPSHVASITGSALSFHSTVINHYRMRGYSPFNNIKSCCVPTRLRAMSMLYYNEEQKIIKKDIQNMIVEECGCS

SEQ ID NO: 33 - protein, 425 aa, synthetic construct
  REGION 1..425: note = Recombinant Inhibin B subunit
GCCCGGCAGTCTGAAGACCACCCTCATCGCCGGCGTCGGCGGGGCTTGGAGTGTGATGGCAAGGTCAACATCTGCTGTAAGAAACAGTTCTTTGTCAGTTTCAAGGACATCGGCTGGAATGACTGGATCATTGCTCCCTCTGGCTATCATGCCAACTACTGCGAGGGTGAGTGCCCGAGCCATATAGCAGGCACGTCCGGGTCCTCACTGTCCTTCCACTCAACAGTCATCAACCACTACCGCATGCGGGGCCATAGCCCCTTTGCCAACCTCAAATCGTGCTGTGTGCCCACCAAGCTGAGACCCATGTCCATGTTGTACTATGATGATGGTCAAAACATCATCAAAAAGGACATTCAGAACATGATCGTGGAGGAGTGTGGGTGCTCATAGAGTTGCCCAGCCCAGGGGGAAAGGGAGCAAGA

SEQ ID NO: 34 - protein, 348 aa, Homo sapiens
GGCCTGGAGTGCGACGGCAAGGTCAACATCTGCTGTAAGAAACAGTTCTTTGTCAGTTTCAAGGACATCGGCTGGAATGACTGGATCATTGCTCCCTCTGGCTATCATGCCAACTACTGCGAGGGTGAGTGCCCGAGCCATATAGCAGGCACGTCCGGGTCCTCACTGTCCTTCCACTCAACAGTCATCAACCACTACGCATGCGGCCATAGCCCCTTTGCCAACCTCAAATCGTGCTGTGTGCCCACCAAGCTGAGACCCATGTCCATGTTGTACTATGATGATGGTCAAAACATCATCAAAAAGGACATTCAGAACATGATCGTGGAGGAGTGCGGGTGCTCCTAA

SEQ ID NO: 35 - DNA, 1477 bp, Homo sapiens
  source 1..1477: mol_type = other DNA
gcggatcgctgctccctctcgccatggcgcaggtgctgatcgtgggcgccgggatgacaggaagcttgtgcgctgcgctgctgaggaggcagacgtccggtcccttgtaccttgctgtgtgggacaaggctgacgactcagggggaagaatgactacagcctgcagtcctcataatcctcagtgcacagctgacttgggtgctcagtacatcacctgcactcctcattatgccaaaaaacaccaacgtttttatgatgaactgttagcctatggcgttttgaggcctctaagctcgcctattgaaggaatggtgatgaaagaaggagactgtaactttgtggcacctcaaggaatttcttcaattattaagcattacttgaaagaatcaggtgcagaagtctacttcagacatcgtgtgacacagatcaacctaagagatgacaaatgggaagtatccaaacaaacaggctcccctgagcagtttgatcttattgttctcacaatgccagttcctgagattctgcagcttcaaggtgacatcaccaccttaattagtgaatgccaaaggcagcaactggaggctgtgagctactcctctcgatatgctctgggcctcttttatgaagctggtacgaagattgatgtcccttgggctgggcagtacatcaccagtaatccctgcatacgcttcgtctccattgataataagaagcgcaatatagagtcatcagaaattgggccttccctcgtgattcacaccactgtcccatttggagttacatacttggaacacagcattgaggatgtgcaagagttagtcttccagcagctggaaaacattttgccgggtttgcctcagccaattgctaccaaatgccaaaaatggagacattcacaggttacaaatgctgctgccaactgtcctggccaaatgactctgcatcacaaacctttccttgcatgtggaggggatggatttactcagtccaactttgatggctgcatcacttctgccctatgtgttctggaagctttaaagaattatatttagtgcctatatccttattctctatatgtgtattgggtttttattttcacaattttctgttattgattattttgttttctattttgctaagaaaaattactggaaaattgttcttcacttattatcatttttcatgtggagtataaaatcaattttgtaattttgatagttacaacccatgctagaatggaaattcctcacaccttgcaccttccctacttttctgaattgctatgactactccttgttggaggaaaagtggtacttaaaaaataacaaacgactctctcaaaaaaattacattaaatcacaataacagtttgtatgccaaaaacttgattatccttatgaaaatttcaattctgaataaagaataatcacattatcaaagccccatcaaaaaaaaaaaaaaaaaaaaaaaaaaaaaa

SEQ ID NO: 36 - DNA, 2107 bp, Homo sapiens
  source 1..2107: mol_type = other DNA
aaaagcccgggccgaacggccccgccgcagagactcagcgcggatcgctgctccctctcgccatggcgcaggtgctgatcgtgggcgccgggatgacaggaagcttgtgcgctgcgctgctgaggaggcagacgtccggtcccttgtaccttgctgtgtgggacaaggctgaggactcagggggaagaatgactacagcctgcagtcctcataatcctcagtgcacagctgacttgggtgctcagtacatcacctgcactcctcattatgccaaaaaacaccaacgtttttatgatgaactgttagcctatggcgttttgaggcctctaagctcgcctattgaaggaatggtgatgaaagaaggagactgtaactttgtggcacctcaaggaatttcttcaattattaagcattacttgaaagaatcaggtgcagaagtctacttcagacatcgtgtgacacagatcaacctaagagatgacaaatgggaagtatccaaacaaacaggctcccctgagcagtttgatcttattgttctcacaatgccagttcctgagattctgcagcttcaaggtgacatcaccaccttaattagtgaatgccaaaggcagcaactggaggctgtgagctactcctctcgatatgctctgggcctcttttatgaagctggtacgaagattgatgtcccttgggctgggcagtacatcaccagtaatccctgcatacgcttcgtctccattgataataagaagcgcaatatagagtcatcagaaattgggccttccctcgtgattcacaccactgtcccatttggagttacatacttggaacacagcattgaggatgtgcaagagttagtcttccagcagctggaaaacattttgccgggtttgcctcagccaattgctaccaaatgccaaaaatggagacattcacaggtaccaagtgctggtgtgattctaggatgtgcgaagagcccctggatgatggcgattggatttcccatctgacttcctggaaattggagcacacagtcaggttttatttgatttttttttttaaggataccacttcacagcctttaggatagctattatttagaagcaaaacagaagataaatgttggcaaggatgtggagatattggattcccttgtgcagtgccggtgggaatgtaaaatgatgtagctactatggaaaatgatacggcaatttctttagaaatgaaatatagaattgccgtatgatctgcagttccacatctggatatctatccaaaagaagtgaaagtagggacttgaacgaacatttgtacaccaatgttcacagcggctttattcacaacagccaaaaggtggaagcaacccagtgtccatggatagatgaatagataaataaaatgtggtataaacatacaatgggctattgtttagccttaaaagggaaggaaattctgacatgctgcaatatggatgaagcttaaagtcattatgcaaagtggaataagcctatcacaaaaaataatattacataattctacttatatgaggaatctagagcagtcagtttcacagagacagaaaatagaatggtggttgccaagggctgggagaagagggcaatggagagtgagtgtttagtgggtcagagttttagtttgggaaggtaaaaagttctggagatggatgatggttatgggtgctcaacagtgtgaatgtacttaatgccacagaactgcacatttaaatgtggttaaaatcatcacttttatgttatgtatatttaccacaataaataaagaagttgatatttcttatacttacaaagaggagaagggcatttgcaaatcaacaagaagtgtgaggcccctctctctagcagaaaaatagactaaatctatttctttatcttttaacatcctgtttaagggaaatgccaaaacaaatgggaaaaaatacacacacacaaatatatatgaacatgttttgcctcatgagtaatcaaaatgtgtacatatgtatgtttatgtatgtgtgtttatatttaaaatcgtgttctgccttatgagtaaacaaaaagtatacaaattaaaaactataatgaaacgt

SEQ ID NO: 37 - protein, 194 aa, synthetic construct
  REGION 1..194: note = Human KGF (GenBank Accession AAB21431)
MHKWILTWILPTLLYRSCFHIICLVGTISLACNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 38 - protein, 176 aa, synthetic construct
  REGION 1..176: note = Human betacellulin (GenBank: AAB25452.1)
MDRAARCSGASSLPLLLALALGLVILHCVVADGNSTRSPETNGLLCGDPEENCAATTTQSKRKGHFSRCPKQYKHYCIKGRCRFVVAEQTPSCVCDEGYIGARCERVDLFYLRGDRGQILVICLIAVMVVFIILVIGVCTCCHPLRKRRKRKKKEEEMETLGKDITPINEDIEETN

SEQ ID NO: 39 - protein, 53 aa, Homo sapiens
NSDSECPLSHDGYCLHDGVCMYIEALDKYACNCVVGYIGERCQYRDLKWWELR

SEQ ID NO: 40 - protein, 53 aa, Homo sapiens
NSDSECPLSHDGYCLHGGVCMYIKAVDRYACNCVVGYIGERCQYRDLTWWGPR

SEQ ID NO: 41 - protein, 53 aa, Homo sapiens
NSDSECPLSHDGYCLHDGVCMYIKALDKYACNCVVGYTGERCQYRDLRWWGRR

SEQ ID NO: 42 - protein, 53 aa, Homo sapiens
NSNSECPLSHDGYCLHDGVCRYIEALDRYACNCVVGYIGERCQYGDLRWWGRR

SEQ ID NO: 43 - protein, 53 aa, Homo sapiens
NSDSGCPLSHSGYCLHDGVCMYIKALDRYACNCVVGYAGERCQYRDLRWWARR

SEQ ID NO: 44 - protein, 53 aa, Homo sapiens
TRGSECPLSHDGYCLHDGVCMYIGALDRYACNCVVGYTGERCQYRDLRWWARR

SEQ ID NO: 45 - protein, 53 aa, Homo sapiens
NSDFGCPLSYDGYCLHDGVCMYIKALDKYACNCVVGYAGERCQYRDLRWWGRR

SEQ ID NO: 46 - protein, 53 aa, Homo sapiens
SRGSKCPPSHDGYCLHDGVCMYIEALDRYACNCVVGYAGERCQYRDLRWWARR

SEQ ID NO: 47 - protein, 53 aa, Homo sapiens
SSGSECPSSHDGYCLHDGACMYIEALDRYACNCAVGYAGERCQYRDLRWWGRR

SEQ ID NO: 48 - protein, 53 aa, Homo sapiens
SSNSECPPSHDGYCLHDGVCMYIEALDRYACNCVVGYAGERCQYRDLRWWARR

SEQ ID NO: 49 - protein, 53 aa, Homo sapiens
NSYSECPPSYDGYCLHDGVCRYIEALDSYACNCVVGYAGERCQYRDLRWWGRR

SEQ ID NO: 50 - protein, 53 aa, Homo sapiens
SSGSECPLSHDGYCLNDGVCMYIEALDKYACNCVVGYVGERCQYRDLRWWARR

SEQ ID NO: 51 - DNA, 406 bp, synthetic construct
  misc_feature 1..406: note = Engineered sequence
  source 1..406: mol_type = other DNA
ctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgtggctggggccagagactggctcttaaaaagtgcagggtctggcgccctctggtggctggctcagaaaaagggccctgacaactcttttcatcttctagg

SEQ ID NO: 52 - DNA, 400 bp, synthetic construct
  misc_feature 1..400: note = Engineered sequence
  source 1..400: mol_type = other DNA
gacccctggaccaccagccccagcaagagcacaagaggaagagagagaccctcactgctggggagtccctgccacactcagtcccccaccacactgaatctcccctcctcacagttgccatgtagaccccttgaagaggggaggggcctagggagccgcaccttgtcatgtaccatcaataaagtaccctgtgctcaaccagttacttgtcctgtcttattctagggtctggggcagaggggagggaagctgggcttgtgtcaaggtgagacattcttgctggggagggacctggtatgttctcctcagactgagggtagggcctccaaacagccttgcttgcttcgagaaccatttgcttcccgctcagacgtcttgagtgctacaggaagctggcacc

SEQ ID NO: 53 - DNA, 66 bp, synthetic construct
  misc_feature 1..66: note = Engineered sequence
  source 1..66: mol_type = other DNA
tacgataatgagttcggctatagtaatcgcgtagtcgatctaatggcacatatggcgtcaaaagaa

SEQ ID NO: 54 - skipped

SEQ ID NO: 55 - protein, 19 aa, synthetic construct
  REGION 1..19: note = Engineered sequence
ATNFSLLKQAGDVEENPGP

SEQ ID NO: 56 - protein, 18 aa, synthetic construct
  REGION 1..18: note = Engineered sequence
EGRGSLLTCGDVEENPGP

SEQ ID NO: 57 - protein, 20 aa, synthetic construct
  REGION 1..20: note = Engineered sequence
QCTNYALLKLAGDVESNPGP

SEQ ID NO: 58 - protein, 22 aa, synthetic construct
  REGION 1..22: note = Engineered sequence
VKQTLNFDLLKLAGDVESNPGP

SEQ ID NO: 59 - DNA, 20 bp, synthetic construct
  misc_feature 1..20: note = Engineered sequence
  source 1..20: mol_type = other DNA
ggatcataggtcgaagtgcg

SEQ ID NO: 60 - DNA, 20 bp, synthetic construct
  misc_feature 1..20: note = Engineered sequence
  source 1..20: mol_type = other DNA
agccaaaggtgctgacaccg

SEQ ID NO: 61 - DNA, 20 bp, synthetic construct
  misc_feature 1..20: note = Engineered sequence
  source 1..20: mol_type = other DNA
ggccagcgtccgcaacacct

SEQ ID NO: 62 - DNA, 20 bp, synthetic construct
  misc_feature 1..20: note = Engineered sequence
  source 1..20: mol_type = other DNA
gaggtgttgcggacgctggc

SEQ ID NO: 63 - DNA, 20 bp, synthetic construct
  misc_feature 1..20: note = Engineered sequence
  source 1..20: mol_type = other DNA
acttcctttctgcagttacg

SEQ ID NO: 64 - DNA, 20 bp, synthetic construct
  misc_feature 1..20: note = Engineered sequence
  source 1..20: mol_type = other DNA
gagacgcgctgcagatggtc

SEQ ID NO: 65 - DNA, 20 bp, synthetic construct
  misc_feature 1..20: note = Engineered sequence
  source 1..20: mol_type = other DNA
gcttcctggcattagacttc

SEQ ID NO: 66 - DNA, 20 bp, synthetic construct
  misc_feature 1..20: note = Engineered sequence
  source 1..20: mol_type = other DNA
cggtgtcagcacctttggct

SEQ ID NO: 67 - DNA, 20 bp, synthetic construct
  misc_feature 1..20: note = Engineered sequence
  source 1..20: mol_type = other DNA
gtgtcagcacctttggctgg

SEQ ID NO: 68 - DNA, 20 bp, synthetic construct
  misc_feature 1..20: note = Engineered sequence
  source 1..20: mol_type = other DNA
ggagcctgaactgctcgttg

SEQ ID NO: 69 - DNA, 20 bp, synthetic construct
  misc_feature 1..20: note = Engineered sequence
  source 1..20: mol_type = other DNA
aacccccgttccaggcttcc

SEQ ID NO: 70 - DNA, 20 bp, synthetic construct
  misc_feature 1..20: note = Engineered sequence
  source 1..20: mol_type = other DNA
cagacgcggagccatggccg

SEQ ID NO: 71 - DNA, 20 bp, synthetic construct
  misc_feature 1..20: note = Engineered sequence
  source 1..20: mol_type = other DNA
cagtaggaaggatgtcctcg

SEQ ID NO: 72 - DNA, 20 bp, synthetic construct
  misc_feature 1..20: note = Engineered sequence
  source 1..20: mol_type = other DNA
acatcctcaacgagcagttc

SEQ ID NO: 73 - DNA, 20 bp, synthetic construct
  misc_feature 1..20: note = Engineered sequence
  source 1..20: mol_type = other DNA
ggtgtcagcacctttggctg

SEQ ID NO: 74 - DNA, 20 bp, synthetic construct
  misc_feature 1..20: note = Engineered sequence
  source 1..20: mol_type = other DNA
cgtccgcaacacctcggcca

SEQ ID NO: 75 - DNA, 20 bp, synthetic construct
  misc_feature 1..20: note = Engineered sequence
  source 1..20: mol_type = other DNA
aatgtgccctcccagacaat

SEQ ID NO: 76 - DNA, 20 bp, synthetic construct
  misc_feature 1..20: note = Engineered sequence
  source 1..20: mol_type = other DNA
tgcagcgcgtctcgttgcca

SEQ ID NO: 77 - DNA, 20 bp, synthetic construct
  misc_feature 1..20: note = Engineered sequence
  source 1..20: mol_type = other DNA
agccccagaagtctaatgcc

SEQ ID NO: 78 - DNA, 20 bp, synthetic construct
  misc_feature 1..20: note = Engineered sequence
  source 1..20: mol_type = other DNA
tcctcgtggtaccccttggc

SEQ ID NO: 79 - DNA, 20 bp, synthetic construct
  misc_feature 1..20: note = Engineered sequence
  source 1..20: mol_type = other DNA
acggtgtcagcacctttggc

SEQ ID NO: 80 - DNA, 20 bp, synthetic construct
  misc_feature 1..20: note = Engineered sequence
  source 1..20: mol_type = other DNA
gcattagacttctggggctt

SEQ ID NO: 81 - DNA, 20 bp, synthetic construct
  misc_feature 1..20: note = Engineered sequence
  source 1..20: mol_type = other DNA
ggccgaggtgttgcggacgc

SEQ ID NO: 82 - DNA, 20 bp, synthetic construct
  misc_feature 1..20: note = Engineered sequence
  source 1..20: mol_type = other DNA
caccacggtgtcagcacctt

SEQ ID NO: 83 - DNA, 20 bp, synthetic construct
  misc_feature 1..20: note = Engineered sequence
  source 1..20: mol_type = other DNA
ttaacccaatggtggtgttc

SEQ ID NO: 84 - DNA, 20 bp, synthetic construct
  misc_feature 1..20: note = Engineered sequence
  source 1..20: mol_type = other DNA
aagtctaatgccaggaagcc

SEQ ID NO: 85 - DNA, 20 bp, synthetic construct
  misc_feature 1..20: note = Engineered sequence
  source 1..20: mol_type = other DNA
gcaacgagacgcgctgcaga

SEQ ID NO: 86 - DNA, 20 bp, synthetic construct
  misc_feature 1..20: note = Engineered sequence
  source 1..20: mol_type = other DNA
ggtcgaagtgcgtggcattt

SEQ ID NO: 87 - DNA, 20 bp, synthetic construct
  misc_feature 1..20: note = Engineered sequence
  source 1..20: mol_type = other DNA
gatggtctacccccagccaa

SEQ ID NO: 88 - DNA, 20 bp, synthetic construct
  misc_feature 1..20: note = Engineered sequence
  source 1..20: mol_type = other DNA
gccagccaaggggtaccacg

SEQ ID NO: 89 - DNA, 20 bp, synthetic construct
  misc_feature 1..20: note = Engineered sequence
  source 1..20: mol_type = other DNA
gatgtcctcgtggtacccct

SEQ ID NO: 90 - DNA, 20 bp, synthetic construct
  misc_feature 1..20: note = Engineered sequence
  source 1..20: mol_type = other DNA
acaagtaactggttgagcac

SEQ ID NO: 91 - DNA, 20 bp, synthetic construct
  misc_feature 1..20: note = Engineered sequence
  source 1..20: mol_type = other DNA
caagtaactggttgagcaca

SEQ ID NO: 92 - DNA, 20 bp, synthetic construct
  misc_feature 1..20: note = Engineered sequence
  source 1..20: mol_type = other DNA
gccatgtagaccccttgaag

SEQ ID NO: 93 - DNA, 20 bp, synthetic construct
  misc_feature 1..20: note = Engineered sequence
  source 1..20: mol_type = other DNA
gtctggcgccctctggtggc

SEQ ID NO: 94 - DNA, 20 bp, synthetic construct
  misc_feature 1..20: note = Engineered sequence
  source 1..20: mol_type = other DNA
gtctggcgccctctggtggc

SEQ ID NO: 95 - DNA, 20 bp, synthetic construct
  misc_feature 1..20: note = Engineered sequence
  source 1..20: mol_type = other DNA
tttctgagccagccaccaga

SEQ ID NO: 96 - DNA, 20 bp, synthetic construct
  misc_feature 1..20: note = Engineered sequence
  source 1..20: mol_type = other DNA
cagggtctggcgccctctgg

SEQ ID NO: 97 - DNA, 20 bp, synthetic construct
  misc_feature 1..20: note = Engineered sequence
  source 1..20: mol_type = other DNA
gtgcagggtctggcgccctc

SEQ ID NO: 98 - DNA, 20 bp, synthetic construct
  misc_feature 1..20: note = Engineered sequence
  source 1..20: mol_type = other DNA
ctcttaaaaagtgcagggtc

SEQ ID NO: 99 - DNA, 20 bp, synthetic construct
  misc_feature 1..20: note = Engineered sequence
  source 1..20: mol_type = other DNA
actggctcttaaaaagtgca

SEQ ID NO: 100 - DNA, 20 bp, synthetic construct
  misc_feature 1..20: note = Engineered sequence
  source 1..20: mol_type = other DNA
gactggctcttaaaaagtgc

SEQ ID NO: 101 - DNA, 20 bp, synthetic construct
  misc_feature 1..20: note = Engineered sequence
  source 1..20: mol_type = other DNA
actttttaagagccagtctc

SEQ ID NO: 102 - DNA, 20 bp, synthetic construct
  misc_feature 1..20: note = Engineered sequence
  source 1..20: mol_type = other DNA
ctcatttcctggtatgtggc

SEQ ID NO: 103 - DNA, 20 bp, synthetic construct
  misc_feature 1..20: note = Engineered sequence
  source 1..20: mol_type = other DNA
caagctcatttcctggtatg

SEQ ID NO: 104 - DNA, 20 bp, synthetic construct
  misc_feature 1..20: note = Engineered sequence
  source 1..20: mol_type = other DNA
tctggccccagccacatacc

SEQ ID NO: 105 - DNA, 20 bp, synthetic construct
  misc_feature 1..20: note = Engineered sequence
  source 1..20: mol_type = other DNA
tctacatggcaactgtgagg

SEQ ID NO: 106 - DNA, 20 bp, synthetic construct
  misc_feature 1..20: note = Engineered sequence
  source 1..20: mol_type = other DNA
ctacatggcaactgtgagga

SEQ ID NO: 107 - DNA, 20 bp, synthetic construct
  misc_feature 1..20: note = Engineered sequence
  source 1..20: mol_type = other DNA
tacatggcaactgtgaggag

SEQ ID NO: 108 - DNA, 20 bp, synthetic construct
  misc_feature 1..20: note = Engineered sequence
  source 1..20: mol_type = other DNA
tcttcctcttgtgctcttgc

SEQ ID NO: 109 - DNA, 20 bp, synthetic construct
  misc_feature 1..20: note = Engineered sequence
  source 1..20: mol_type = other DNA
cttcctcttgtgctcttgct

SEQ ID NO: 110 - DNA, 20 bp, synthetic construct
  misc_feature 1..20: note = Engineered sequence
  source 1..20: mol_type = other DNA
ttcctcttgtgctcttgctg

SEQ ID NO: 111 - DNA, 20 bp, synthetic construct
  misc_feature 1..20: note = Engineered sequence
  source 1..20: mol_type = other DNA
tctggccccagccacatacc

SEQ ID NO: 112 - DNA, 20 bp, synthetic construct
  misc_feature 1..20: note = Engineered sequence
  source 1..20: mol_type = other DNA
gacaactcttttcatcttct

SEQ ID NO: 113 - DNA, 20 bp, synthetic construct
  misc_feature 1..20: note = Engineered sequence
  source 1..20: mol_type = other DNA
agaagatgaaaagagttgtc

SEQ ID NO: 114 - DNA, 20 bp, synthetic construct
  misc_feature 1..20: note = Engineered sequence
  source 1..20: mol_type = other DNA
gaagatgaaaagagttgtca

SEQ ID NO: 115 - DNA, 20 bp, synthetic construct
  misc_feature 1..20: note = Engineered sequence
  source 1..20: mol_type = other DNA
tggtggctggctcagaaaaa

SEQ ID NO: 116 - DNA, 20 bp, synthetic construct
  misc_feature 1..20: note = Engineered sequence
  source 1..20: mol_type = other DNA
ctggtggctggctcagaaaa

SEQ ID NO: 117 - DNA, 20 bp, synthetic construct
  misc_feature 1..20: note = sgRNA
  source 1..20: mol_type = other DNA
agtcacatggttcacacggc

SEQ ID NO: 118 - DNA, 20 bp, synthetic construct
  misc_feature 1..20: note = sgRNA
  source 1..20: mol_type = other DNA
cgtgagtaaacctgaatctt

SEQ ID NO: 119 - DNA, 20 bp, synthetic construct
  misc_feature 1..20: note = sgRNA
  source 1..20: mol_type = other DNA
cagtaagtcaacttcaatgt

SEQ ID NO: 120 - DNA, 20 bp, synthetic construct
  misc_feature 1..20: note = sgRNA
  source 1..20: mol_type = other DNA
tcacgtcatccagcagagaa

SEQ ID NO: 121 - DNA, 20 bp, synthetic construct
  misc_feature 1..20: note = sgRNA
  source 1..20: mol_type = other DNA
cacagcccaagatagttaag

SEQ ID NO: 122 - DNA, 20 bp, synthetic construct
  misc_feature 1..20: note = sgRNA
  source 1..20: mol_type = other DNA
cagcccaagatagttaagtg

SEQ ID NO: 123 - DNA, 20 bp, synthetic construct
  misc_feature 1..20: note = sgRNA
  source 1..20: mol_type = other DNA
acagcccaagatagttaagt

SEQ ID NO: 124 - DNA, 20 bp, synthetic construct
  misc_feature 1..20: note = sgRNA
  source 1..20: mol_type = other DNA
cttaccccacttaactatct

SEQ ID NO: 125 - DNA, 20 bp, synthetic construct
  misc_feature 1..20: note = sgRNA
  source 1..20: mol_type = other DNA
tcctgaattgctatgtgtct

SEQ ID NO: 126 - DNA, 20 bp, synthetic construct
  misc_feature 1..20: note = sgRNA
  source 1..20: mol_type = other DNA
catactcatctttttcagtg

SEQ ID NO: 127 - DNA, 20 bp, synthetic construct
  misc_feature 1..20: note = sgRNA
  source 1..20: mol_type = other DNA
acaaagtcacatggttcaca

SEQ ID NO: 128 - DNA, 20 bp, synthetic construct
  misc_feature 1..20: note = sgRNA
  source 1..20: mol_type = other DNA
gtcgctcggacgctcctgct

SEQ ID NO: 129 - DNA, 20 bp, synthetic construct
  misc_feature 1..20: note = sgRNA
  source 1..20: mol_type = other DNA
ccttcacaatctcgtcggtg

SEQ ID NO: 130 - DNA, 20 bp, synthetic construct
  misc_feature 1..20: note = sgRNA
  source 1..20: mol_type = other DNA
cctcaccgacgagattgtga

SEQ ID NO: 131 - DNA, 20 bp, synthetic construct
  misc_feature 1..20: note = sgRNA
  source 1..20: mol_type = other DNA
ggcacgggtcttctcctacc

SEQ ID NO: 132 - DNA, 20 bp, synthetic construct
  misc_feature 1..20: note = sgRNA
  source 1..20: mol_type = other DNA
ggatgtgaagcagacgtact

SEQ ID NO: 133 - DNA, 20 bp, synthetic construct
  misc_feature 1..20: note = sgRNA
  source 1..20: mol_type = other DNA
tcggacgctcctgctcggct

SEQ ID NO: 134 - DNA, 20 bp, synthetic construct
  misc_feature 1..20: note = sgRNA
  source 1..20: mol_type = other DNA
gcccaggtggccggcgcttc

SEQ ID NO: 135 - DNA, 20 bp, synthetic construct
  misc_feature 1..20: note = sgRNA
  source 1..20: mol_type = other DNA
cacatccttcacaatctcgt

SEQ ID NO: 136 - DNA, 20 bp, synthetic construct
  misc_feature 1..20: note = sgRNA
  source 1..20: mol_type = other DNA
gaagcagacgtacttggcac

SEQ ID NO: 137 - DNA, 20 bp, synthetic construct
  misc_feature 1..20: note = sgRNA
  source 1..20: mol_type = other DNA
gggcgaagacccagccgagc

SEQ ID NO: 138 - DNA, 20 bp, synthetic construct
  misc_feature 1..20: note = sgRNA
  source 1..20: mol_type = other DNA
acctgaagcgccggccacct

SEQ ID NO: 139 - DNA, 20 bp, synthetic construct
  misc_feature 1..20: note = sgRNA
  source 1..20: mol_type = other DNA
gtgccactcacctgaagcgc

SEQ ID NO: 140 - DNA, 20 bp, synthetic construct
  misc_feature 1..20: note = sgRNA
  source 1..20: mol_type = other DNA
gctcggctgggtcttcgccc

SEQ ID NO: 141 - DNA, 20 bp, synthetic construct
  misc_feature 1..20: note = sgRNA
  source 1..20: mol_type = other DNA
taaaggcactacaaatactg

SEQ ID NO: 142 - DNA, 20 bp, synthetic construct
  misc_feature 1..20: note = sgRNA
  source 1..20: mol_type = other DNA
tagacttgattgacgggttt

SEQ ID NO: 143 - DNA, 80 bp, synthetic construct
  misc_feature 1..80: note = scaffold sequence
  source 1..80: mol_type = other DNA
gttttagagctagaaatagcaagttaaaataaggctagtccgttatcaacttgaaaaagtggcaccgagtcggtgctttt

SEQ ID NO: 144 - protein, 1368 aa, synthetic construct [sequence omitted]

SEQ ID NO: 145 - protein, 275 aa, synthetic construct
  REGION 1..275: note = Mature CD47 Protein
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVE

SEQ ID NO: 146 - protein, 125 aa, synthetic construct
  REGION 1..125: note = CD47 Extracellular Domain
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENI

SEQ ID NO: 147 - DNA, 20 bp, synthetic construct
  misc_feature 1..20: note = sgRNA
  source 1..20: mol_type = other DNA
atgctttgttactaatatgg

SEQ ID NO: 148 - DNA, 20 bp, synthetic construct
  misc_feature 1..20: note = sgRNA
  source 1..20: mol_type = other DNA
tccatgctttgttactaata

SEQ ID NO: 149 - DNA, 20 bp, synthetic construct
  misc_feature 1..20: note = sgRNA
  source 1..20: mol_type = other DNA
tccatattagtaacaaagca

SEQ ID NO: 150 - DNA, 20 bp, synthetic construct
  misc_feature 1..20: note = sgRNA
  source 1..20: mol_type = other DNA
aatagtagctgagctgatcc

SEQ ID NO: 151 - DNA, 20 bp, synthetic construct
  misc_feature 1..20: note = sgRNA
  source 1..20: mol_type = other DNA
tagctgagctgatcctggaa

SEQ ID NO: 152 - DNA, 20 bp, synthetic construct
  misc_feature 1..20: note = sgRNA
  source 1..20: mol_type = other DNA
catttttctttttcctttcc

SEQ ID NO: 153 - DNA, 20 bp, synthetic construct
  misc_feature 1..20: note = sgRNA
  source 1..20: mol_type = other DNA
agtctgtactgtaagatctt

SEQ ID NO: 154 - DNA, 20 bp, synthetic construct
  misc_feature 1..20: note = sgRNA
  source 1..20: mol_type = other DNA
aaatgttgctgtttatctta

SEQ ID NO: 155 - DNA, 20 bp, synthetic construct
  misc_feature 1..20: note = sgRNA
  source 1..20: mol_type = other DNA
gtaatcaacctgttaatcca

SEQ ID NO: 156 - DNA, 20 bp, synthetic construct
  misc_feature 1..20: note = sgRNA
  source 1..20: mol_type = other DNA
cagcgtacagttctagagag

SEQ ID NO: 157 - DNA, 20 bp, synthetic construct
  misc_feature 1..20: note = sgRNA
  source 1..20: mol_type = other DNA
ttgattactaatgctgatgc

SEQ ID NO: 158 - DNA, 20 bp, synthetic construct
  misc_feature 1..20: note = sgRNA
  source 1..20: mol_type = other DNA
ctgactctaagtggcattca

SEQ ID NO: 159 - DNA, 20 bp, synthetic construct
  misc_feature 1..20: note = sgRNA
  source 1..20: mol_type = other DNA
cagaatggcagtttgattca

SEQ ID NO: 160 - DNA, 20 bp, synthetic construct
  misc_feature 1..20: note = sgRNA
  source 1..20: mol_type = other DNA
tctaaagaccttggattaac

SEQ ID NO: 161 - DNA, 20 bp, synthetic construct
  misc_feature 1..20: note = sgRNA
  source 1..20: mol_type = other DNA
gataaggcagcaaatcagaa

SEQ ID NO: 162 - protein, 278 aa, synthetic construct
  REGION 1..278: note = Mutant CD47 protein
WQPPLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVE

SEQ ID NO: 163 - protein, 128 aa, synthetic construct
  REGION 1..128: note = Mutant CD47 Extracellular Domain
WQPPLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENI

SEQ ID NO: 164 - DNA, 20 bp, synthetic construct
  misc_feature 1..20: note = sgRNA
  source 1..20: mol_type = other DNA
tgtgcgctgcgctgctgagg

SEQ ID NO: 165 - DNA, 20 bp, synthetic construct
  misc_feature 1..20: note = sgRNA
  source 1..20: mol_type = other DNA
gcttgtgcgctgcgctgctg

SEQ ID NO: 166 - DNA, 20 bp, synthetic construct
  misc_feature 1..20: note = sgRNA
  source 1..20: mol_type = other DNA
atcgtgggcgccgggatgac

SEQ ID NO: 167 - DNA, 20 bp, synthetic construct
  misc_feature 1..20: note = sgRNA
  source 1..20: mol_type = other DNA
aggtgctgatcgtgggcgcc

SEQ ID NO: 168 - DNA, 20 bp, synthetic construct
  misc_feature 1..20: note = sgRNA
  source 1..20: mol_type = other DNA
atggcgcaggtgctgatcgt

SEQ ID NO: 169 - DNA, 20 bp, synthetic construct
  misc_feature 1..20: note = sgRNA
  source 1..20: mol_type = other DNA
catggcgcaggtgctgatcg

SEQ ID NO: 170 - DNA, 20 bp, synthetic construct
  misc_feature 1..20: note = GCUCCCUCUCGCCAUGGCGC
  source 1..20: mol_type = other DNA
gctccctctcgccatggcgc

SEQ ID NO: 171 - DNA, 20 bp, synthetic construct
  misc_feature 1..20: note = sgRNA
  source 1..20: mol_type = other DNA
cacgatcagcacctgcgcca

SEQ ID NO: 172 - DNA, 20 bp, synthetic construct
  misc_feature 1..20: note = sgRNA
  source 1..20: mol_type = other DNA
gataataagaagcgcaatat

SEQ ID NO: 173 - DNA, 20 bp, synthetic construct
  misc_feature 1..20: note = sgRNA
  source 1..20: mol_type = other DNA
agattgatgtcccttgggct

SEQ ID NO: 174 - DNA, 20 bp, synthetic construct
  misc_feature 1..20: note = sgRNA
  source 1..20: mol_type = other DNA
aagattgatgtcccttgggc

SEQ ID NO: 175 - DNA, 20 bp, synthetic construct
  misc_feature 1..20: note = sgRNA
  source 1..20: mol_type = other DNA
gtgatgtactgcccagccca

SEQ ID NO: 176 - DNA, 20 bp, synthetic construct
  misc_feature 1..20: note = sgRNA
  source 1..20: mol_type = other DNA
tcgtaccagcttcataaaag

SEQ ID NO: 177 - DNA, 20 bp, synthetic construct
  misc_feature 1..20: note = sgRNA
  source 1..20: mol_type = other DNA
tcgtaccagcttcataaaag

SEQ ID NO: 178 - DNA, 20 bp, synthetic construct
  misc_feature 1..20: note = sgRNA
  source 1..20: mol_type = other DNA
gaggcccagagcatatcgag

SEQ ID NO: 179 - DNA, 20 bp, synthetic construct
  misc_feature 1..20: note = sgRNA
  source 1..20: mol_type = other DNA
cagcctccagttgctgcctt

SEQ ID NO: 180 - DNA, 20 bp, synthetic construct
  misc_feature 1..20: note = sgRNA
  source 1..20: mol_type = other DNA
gataataagaagcgcaatat

SEQ ID NO: 181 - DNA, 20 bp, synthetic construct
  misc_feature 1..20: note = sgRNA
  source 1..20: mol_type = other DNA
agattgatgtcccttgggct

SEQ ID NO: 182 - DNA, 20 bp, synthetic construct
  misc_feature 1..20: note = sgRNA
  source 1..20: mol_type = other DNA
aagattgatgtcccttgggc

SEQ ID NO: 183 - DNA, 20 bp, synthetic construct
  misc_feature 1..20: note = sgRNA
  source 1..20: mol_type = other DNA
tacgaagattgatgtccctt

SEQ ID NO: 184 - DNA, 20 bp, synthetic construct
  misc_feature 1..20: note = sgRNA
  source 1..20: mol_type = other DNA
gtacgaagattgatgtccct

SEQ ID NO: 185 - DNA, 20 bp, synthetic construct
  misc_feature 1..20: note = sgRNA
  source 1..20: mol_type = other DNA
ctgggcctcttttatgaagc

SEQ ID NO: 186 - DNA, 20 bp, synthetic construct
  misc_feature 1..20: note = sgRNA
  source 1..20: mol_type = other DNA
tactcctctcgatatgctct

SEQ ID NO: 187 - DNA, 20 bp, synthetic construct
  misc_feature 1..20: note = sgRNA
  source 1..20: mol_type = other DNA
ctactcctctcgatatgctc

SEQ ID NO: 188 - DNA, 20 bp, synthetic construct
  misc_feature 1..20: note = sgRNA
  source 1..20: mol_type = other DNA
atgccaaaggcagcaactgg

SEQ ID NO: 189 - DNA, 20 bp, synthetic construct
  misc_feature 1..20: note = sgRNA
  source 1..20: mol_type = other DNA
tgaatgccaaaggcagcaac

SEQ ID NO: 190 - DNA, 20 bp, synthetic construct
  misc_feature 1..20: note = sgRNA
  source 1..20: mol_type = other DNA
cagtaattagtgaatgccaa

SEQ ID NO: 191 - DNA, 20 bp, synthetic construct
  misc_feature 1..20: note = sgRNA
  source 1..20: mol_type = other DNA
attgcgcttcttattatcaa

SEQ ID NO: 192 - DNA, 20 bp, synthetic construct
  misc_feature 1..20: note = sgRNA
  source 1..20: mol_type = other DNA
aatggagacgaagcgtatgc

SEQ ID NO: 193 - DNA, 20 bp, synthetic construct
  misc_feature 1..20: note = sgRNA
  source 1..20: mol_type = other DNA
atggagacgaagcgtatgca

SEQ ID NO: 194 - DNA, 20 bp, synthetic construct
  misc_feature 1..20: note = sgRNA
  source 1..20: mol_type = other DNA
tgaatgccaaaggcagcaac

SEQ ID NO: 195 - DNA, 20 bp, synthetic construct
  misc_feature 1..20: note = sgRNA
  source 1..20: mol_type = other DNA
tgatgtactgcccagcccaa

SEQ ID NO: 196 - DNA, 20 bp, synthetic construct
  misc_feature 1..20: note = sgRNA
  source 1..20: mol_type = other DNA
cttatctttctgactctaag

SEQ ID NO: 197 - DNA, 20 bp, synthetic construct
  misc_feature 1..20: note = sgRNA
  source 1..20: mol_type = other DNA
actcacatgatctcaacagc

SEQ ID NO: 198 - DNA, 20 bp, synthetic construct
  misc_feature 1..20: note = sgRNA
  source 1..20: mol_type = other DNA
tctcaacacgtggacaaaat

SEQ ID NO: 199 - DNA, 20 bp, synthetic construct
  misc_feature 1..20: note = sgRNA
  source 1..20: mol_type = other DNA
ttcttgatggccttcgattc

SEQ ID NO: 200 - DNA, 20 bp, synthetic construct
  misc_feature 1..20: note = sgRNA
  source 1..20: mol_type = other DNA
tgctttcagtaaattcttga

SEQ ID NO: 201 - DNA, 20 bp, synthetic construct
  misc_feature 1..20: note = sgRNA
  source 1..20: mol_type = other DNA
agcagttagcaaggaaaggt

SEQ ID NO: 202 - DNA, 20 bp, synthetic construct
  misc_feature 1..20: note = sgRNA
  source 1..20: mol_type = other DNA
tgaaagcagttagcaaggaa

SEQ ID NO: 203 - DNA, 20 bp, synthetic construct
  misc_feature 1..20: note = sgRNA
  source 1..20: mol_type = other DNA
atgtctgaatccagaatcga

SEQ ID NO: 204 - DNA, 20 bp, synthetic construct
  misc_feature 1..20: note = sgRNA
  source 1..20: mol_type = other DNA
cgtggacaaaattggcttgc

SEQ ID NO: 205 - DNA, 20 bp, synthetic construct
  misc_feature 1..20: note = sgRNA
  source 1..20: mol_type = other DNA
actctctctttctggcctgg

SEQ ID NO: 206 - DNA, 20 bp, synthetic construct
  misc_feature 1..20: note = sgRNA
  source 1..20: mol_type = other DNA
gcccgaatgctgtcagcttc

SEQ ID NO: 207 - DNA, 20 bp, synthetic construct
  misc_feature 1..20: note = sgRNA
  source 1..20: mol_type = other DNA
ggccacggagcgagacatct

SEQ ID NO: 208 - DNA, 20 bp, synthetic construct
  misc_feature 1..20: note = sgRNA
  source 1..20: mol_type = other DNA
cgcgagcacagctaaggcca

SEQ ID NO: 209 - DNA, 20 bp, synthetic construct
  misc_feature 1..20: note = sgRNA
  source 1..20: mol_type = other DNA
gagtagcgcgagcacagcta

SEQ ID NO: 210 - DNA, 20 bp, synthetic construct
  misc_feature 1..20: note = sgRNA
  source 1..20: mol_type = other DNA
actcacgctggatagcctcc

SEQ ID NO: 211 - DNA, 20 bp, synthetic construct
  misc_feature 1..20: note = sgRNA
  source 1..20: mol_type = other DNA
ggccgagatgtctcgctccg

SEQ ID NO: 212 - DNA, 20 bp, synthetic construct
  misc_feature 1..20: note = sgRNA
  source 1..20: mol_type = other DNA
ctcgcgctactctctctttc

SEQ ID NO: 213 - DNA, 20 bp, synthetic construct
  misc_feature 1..20: note = sgRNA
  source 1..20: mol_type = other DNA
gctactctctctttctggcc

SEQ ID NO: 214 - DNA, 20 bp, synthetic construct
  misc_feature 1..20: note = sgRNA
  source 1..20: mol_type = other DNA
tttgactttccattctctgc

SEQ ID NO: 215 - DNA, 20 bp, synthetic construct
  misc_feature 1..20: note = sgRNA
  source 1..20: mol_type = other DNA
acccagacacatagcaattc

SEQ ID NO: 216 - DNA, 20 bp, synthetic construct
  misc_feature 1..20: note = sgRNA
  source 1..20: mol_type = other DNA
aagtcaacttcaatgtcgga

SEQ ID NO: 217 - DNA, 20 bp, synthetic construct
  misc_feature 1..20: note = sgRNA
  source 1..20: mol_type = other DNA
atactcatctttttcagtgg

SEQ ID NO: 218 - DNA, 20 bp, synthetic construct
  misc_feature 1..20: note = sgRNA
  source 1..20: mol_type = other DNA
gcatactcatctttttcagt

SEQ ID NO: 219 - DNA, 20 bp, synthetic construct
  misc_feature 1..20: note = sgRNA
  source 1..20: mol_type = other DNA
ggcatactcatctttttcag

SEQ ID NO: 220 - DNA, 20 bp, synthetic construct
  misc_feature 1..20: note = sgRNA
  source 1..20: mol_type = other DNA
tgggctgtgacaaagtcaca

SEQ ID NO: 221 - DNA, 20 bp, synthetic construct
  misc_feature 1..20: note = sgRNA
  source 1..20: mol_type = other DNA
ttaccccacttaactatctt

SEQ ID NO: 222 - DNA, 20 bp, synthetic construct
  misc_feature 1..20: note = sgRNA
  source 1..20: mol_type = other DNA
ttcctgaattgctatgtgtc

SEQ ID NO: 223 - DNA, 20 bp, synthetic construct
  misc_feature 1..20: note = sgRNA
  source 1..20: mol_type = other DNA
gaagttgacttactgaagaa

SEQ ID NO: 224 - DNA, 20 bp, synthetic construct
  misc_feature 1..20: note = sgRNA
  source 1..20: mol_type = other DNA
ttcagacttgtctttcagca

SEQ ID NO: 225 - DNA, 20 bp, synthetic construct
  misc_feature 1..20: note = sgRNA
  source 1..20: mol_type = other DNA
acttgtctttcagcaaggac

SEQ ID NO: 226 - DNA, 20 bp, synthetic construct
  misc_feature 1..20: note = sgRNA
  source 1..20: mol_type = other DNA
gagcaggagcgtccgagcga

SEQ ID NO: 227 - DNA, 20 bp, synthetic construct
  misc_feature 1..20: note = sgRNA
  source 1..20: mol_type = other DNA
caggagcgtccgagcgacgg

SEQ ID NO: 228 - DNA, 20 bp, synthetic construct
  misc_feature 1..20: note = sgRNA
  source 1..20: mol_type = other DNA
cgtccgagcgacggcggtct

SEQ ID NO: 229 - DNA, 20 bp, synthetic construct
  misc_feature 1..20: note = sgRNA
  source 1..20: mol_type = other DNA
gtccgagcgacggcggtctc

SEQ ID NO: 230 - DNA, 20 bp, synthetic construct
  misc_feature 1..20: note = sgRNA
  source 1..20: mol_type = other DNA
tccgagcgacggcggtctcg

SEQ ID NO: 231 - DNA, 20 bp, synthetic construct
  misc_feature 1..20: note = sgRNA
  source 1..20: mol_type = other DNA
tgggtcttcgcccaggtggc

SEQ ID NO: 232 - DNA, 20 bp, synthetic construct
  misc_feature 1..20: note = sgRNA
  source 1..20: mol_type = other DNA
ctcggacgctcctgctcggc

SEQ ID NO: 233 - DNA, 20 bp, synthetic construct
  misc_feature 1..20: note = sgRNA
  source 1..20: mol_type = other DNA
gccccgagaccgccgtcgct

SEQ ID NO: 234 - DNA, 20 bp, synthetic construct
  misc_feature 1..20: note = sgRNA
  source 1..20: mol_type = other DNA
aacagtgtagacttgattga

SEQ ID NO: 235 - DNA, 20 bp, synthetic construct
  misc_feature 1..20: note = sgRNA
  source 1..20: mol_type = other DNA
acagtgtagacttgattgac

SEQ ID NO: 236 - DNA, 20 bp, synthetic construct
  misc_feature 1..20: note = sgRNA
  source 1..20: mol_type = other DNA
gtagacttgattgacgggtt

SEQ ID NO: 237 - DNA, 20 bp, synthetic construct
  misc_feature 1..20: note = sgRNA
  source 1..20: mol_type = other DNA
aagtctacactgttcaaata

SEQ ID NO: 238 - DNA, 20 bp, synthetic construct
  misc_feature 1..20: note = sgRNA
  source 1..20: mol_type = other DNA
tggcagcatataatttaact

SEQ ID NO: 239 - DNA, 20 bp, synthetic construct
  misc_feature 1..20: note = sgRNA
  source 1..20: mol_type = other DNA
ctcctacccggcagggaatg

SEQ ID NO: 240 - DNA, 20 bp, synthetic construct
  misc_feature 1..20: note = sgRNA
  source 1..20: mol_type = other DNA
cgggtcttctcctacccggc

SEQ ID NO: 241 - DNA, 20 bp, synthetic construct
  misc_feature 1..20: note = sgRNA
  source 1..20: mol_type = other DNA
tagacatggagacccctgcc

SEQ ID NO: 242 - DNA, 2234 bp, Homo sapiens
  source 1..2234: mol_type = other DNA
tctccagaacatcatccctgcctctactggcgctgccaaggctgtgggcaaggtcatccctgagctgaacgggaagctcactggcatggccttccgtgtccccactgccaacgtgtcagtggtggacctgacctgccgtctagaaaaacctgccaaatatgatgacatcaagaaggtggtgaagcaggcgtcggagggccccctcaagggcatcctgggctacactgagcaccaggtggtctcctctgacttcaacagcgacacccactcctccacctttgacgctggggctggcattgccctcaacgaccactttgtcaagctcatttcctggtatgtggctggggccagagactggctcttaaaaagtgcagggtctggcgccctctggtggctggctcagaaaaagggccctgacaactcttttcatcttctaggtatgacaacgaatttggctacagcaacagggtggtggacctcatggcccacatggcctccaaggagggatccggagccacgaacttctctctgttaaagcaagcaggagacgtggaagaaaaccccggtcctatgtggcccctggtagcggcgctgttgctgggctcggcgtgctgcggatcagctcagctactatttaataaaacaaaatctgtagagttcacgttttgtaatgacactgtcgtcattccatgctttgttactaatatggaggcacaaaacactactgaagtatacgtaaagtggaaatttaaaggaagagatatttacacctttgatggagctctaaacaagtccactgtccccactgactttagtagtgcaaaaattgaagtctcacaattactaaaaggagatgcctctttgaagatggataagagtgatgctgtctcacacacaggaaactacacttgtgaagtaacagaattaaccagagaaggtgaaacgatcatcgagctaaaatatcgtgttgtttcatggttttctccaaatgaaaatattcttattgttattttcccaatttttgctatactcctgttctggggacagtttggtattaaaacacttaaatatagatccggtggtatggatgagaaaacaattgctttacttgttgctggactagtgatcactgtcattgtcattgttggagccattcttttcgtcccaggtgaatattcattaaagaatgctactggccttggtttaattgtgacttctacagggatattaatattacttcactactatgtgtttagtacagcgattggattaacctccttcgtcattgccatattggttattcaggtgatagcctatatcctcgctgtggttggactgagtctctgtattgcggcgtgtataccaatgcatggccctcttctgatttcaggtttgagtatcttagctctagcacaattacttggactagtttatatgaaatttgtggcttccaatcagaagactatacaacctcctaggaaagctgtagaggaaccccttaatgcattcaaagaatcaaaaggaatgatgaatgatgaataaggatccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggactcagtcccccaccacactgaatctcccctcctcacagttgccatgtagaccccttgaagaggggaggggcctagggagccgcaccttgtcatgtaccatcaataaagtaccctgtgctcaaccagttacttgtcctgtcttattctagggtctggggcagaggggagggaagctgggcttgtgtcaaggtgagacattcttgctggggagggacctggtatgttctcctcagactgagggtagggcctccaaacagccttgcttgcttcgagaaccatttgcttcccgctcagacgtcttgagtgctacaggaagctggcaccactacttcagagaacaaggccttttcctctcctcgctccagtcctaggctatctgctgttggccaaacatggaagaagctattctgtgggcagccccagggaggctgacaggtggaggaagtcagggctcgcactg

SEQ ID NO: 243 - protein, 292 aa, synthetic construct
  REGION 1..292: note = CD47 Amino Acid Sequence with Leader Sequence
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFV

SEQ ID NO: 244 - protein, 18 aa, synthetic construct
  REGION 1..18: note = CD47 Leader Sequence
MWPLVAALLLGSACCGSA

SEQ ID NO: 245 - protein, 295 aa, synthetic construct
  REGION 1..295: note = Mutant CD47 Amino Acid Sequence with Leader Sequence
MWPLVAALLLGSACCGSAWQPPLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFV

SEQ ID NO: 246 - DNA, 112 bp, synthetic construct
  source 1..112: mol_type = other DNA
gacttctataattaatgaaacatttttctttttcctttccaggatcagcttggcaacctcctctactatttaataaaacaaaatctgtagaattcacgttttgtaatgacac

SEQ ID NO: 247 - DNA, 112 bp, synthetic construct
  source 1..112: mol_type = other DNA
ctgaagatattaattactttgtaaaaagaaaaaggaaaggtcctagtcgaaccgttggaggagatgataaattattttgttttagacatcttaagtgcaaaacattactgtg

SEQ ID NO: 248 - DNA, 34 bp, synthetic construct
  source 1..34: mol_type = other DNA
gctgcggatcagctcagctactatttaataaaac

SEQ ID NO: 249 - DNA, 34 bp, synthetic construct
  source 1..34: mol_type = other DNA
cgacgcctagtcgagtcgatgataaattattttg

SEQ ID NO: 250 - DNA, 43 bp, synthetic construct
  source 1..43: mol_type = other DNA
gctgcggatcagcttggcaacctcctctactatttaataaaac

SEQ ID NO: 251 - DNA, 43 bp, synthetic construct
  source 1..43: mol_type = other DNA
cgacgcctagtcgaaccgttggaggagatgataaattattttg

SEQ ID NO: 252 - protein, 12 aa, synthetic construct
CCGSAQLLFNKT

SEQ ID NO: 253 - protein, 15 aa, synthetic construct
CCGSAWQPPLLFNKT